(19) United States
(12) Patent Application Publication
(10) Pub. No.: US 2009/0054392 A1
(43) Pub. Date: Feb. 26, 2009

(54) NAPHTHYLPYRIMIDINE, NAPHTHYLPYRAZINE AND NAPHTHYLPYRIDAZINE ANALOGS AND THEIR USE AS AGONISTS OF THE WNT-BETA-CATENIN CELLULAR MESSAGING SYSTEM

(75) Inventors: Jeffrey Claude Pelletier, Lafayette Hill, PA (US); Luciana de Araujo Felix, Broomall, PA (US); Daniel Michael Green, Ambler, PA (US); Diane Barbara Hauze, Wayne, PA (US); Joseph Theodore Lundquist, IV, Limerick, PA (US); Charles William Mann, Plymouth Meeting, PA (US); John Francis Mehlmann, King of Prussia, PA (US); John Francis Rogers, JR., Bryn Mawr, PA (US); Matthew Douglas Vera, Collegeville, PA (US); Albert John Molinari, Pottstown, PA (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/194,235

(22) Filed: Aug. 19, 2008

Related U.S. Application Data
(60) Provisional application No. 60/965,420, filed on Aug. 20, 2007.

(51) Int. Cl. A61K 31/397; C07D 239/24; A61K 31/505; C07D 243/08; C07D 413/14; A61K 31/496; A61P 19/08; A61P 25/28; A61P 35/00; A61K 31/5377; A61K 31/551; C07D 403/02; A61K 31/55

(52) U.S. Cl. 514/210.2; 544/330; 514/275; 540/575; 514/218; 540/601; 514/217.06; 544/121; 514/235.8; 514/252.14

(57) ABSTRACT

The present invention relates to naphthylpyrimidine analogs, methods of making naphthylpyrimidine analogs, compositions comprising a naphthylpyrimidine analog, and methods for treating canonical Wnt-β-catenin cellular messaging system-related disorders comprising administering to a subject in need thereof an effective amount of a naphthylpyrimidine, naphthylpyrazine and naphthylpyridazine analog.

### CROSS-REFERENCE TO RELATED APPLICATIONS

This application claims priority benefit of U.S. provisional patent application No. 60/965,420, filed Aug. 20, 2007, the entire disclosure of which is hereby incorporated by reference.

### FIELD OF THE INVENTION

The invention relates to naphthylpyrimidine analogs, compositions comprising a naphthylpyrimidine analog, and methods for treating or preventing disease involving the canonical Wnt-β-catenin cellular messaging system comprising the administration of an effective amount of a naphthylpyrimidine analog.

### BACKGROUND OF THE INVENTION

The Wnt-β-catenin cellular messaging system is essential in many biological processes. It regulates the fate of as-yet undeveloped cells in embryo form. The signals in the Wnt-β-catenin cellular messaging system also direct the development of stem cells in adult organisms (e.g. skin cell, bone cell, liver cell, etc.). At the cellular level, the canonical Wnt-β-catenin cellular messaging system regulates morphology, proliferation, motility and cell fate. The Wnt-β-catenin messaging system has a central role in tumorigenesis and inappropriate activation of this system is observed in several human cancers.

Wnt-β-catenin was first described in humans as a protein which interacts with the cytoplasmic domain of E-cadherin and with Wnt-β-catenin, anchoring the cadherin complex to the actin cytoskeleton. Then, an additional role for mammalian Wnt-β-catenin was discovered; namely, as the key mediator of Wnt-β-catenin messaging.

Chronic activation of the Wnt-β-catenin cellular messaging system has been implicated in the development of a variety of human malignancies, including colorectal carcinomas, hepatocellular carcinomas (HCCs), melanomas, and uterine and ovarian carcinomas.

The Wnt-β-catenin cellular messaging system also plays a role in degenerative diseases such as Alzheimer's disease (AD) and bone disorders.

AD is the most common age-related neurodegenerative disorder. A massive accumulation of beta-amyloid (Abeta) peptide aggregates is likely the pivotal event in AD. Abeta-induced toxicity is accompanied by a varied combination of events including oxidative stress. The Wnt-β-catenin pathway has multiple actions in the cascade of events triggered by Abeta, and drugs with Wnt-β-catenin activity can be therapeutics for AD treatment.

Various bone disorders are also associated with defects in the Wnt-P-catenin messaging system. Signaling through the Wnt-β-catenin pathway increases bone mass through a number of mechanisms, including renewal of stem cells, stimulation of preosteoblast replication, induction of osteoblastogenesis, and inhibition of osteoblast and osteocyte apoptosis.

As discussed above, agonists of the Wnt-β-catenin messaging system are expected to be medicaments useful against cell proliferation disorders, bone disorders, and Alzheimer's disease. Thus, it would be advantageous to have novel agonists of the Wnt-β-catenin messaging system as potential treatment regimens for Wnt-β-catenin messaging system-related diseases. The instant invention is directed to these and other important ends.

### SUMMARY OF THE INVENTION

In one aspect, the invention provides compounds of Formula (A):

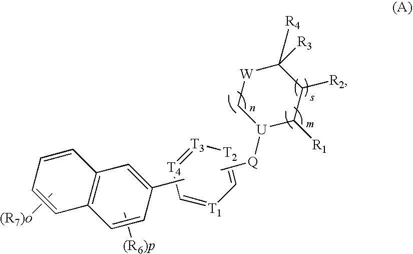

or a pharmaceutically acceptable salt thereof, wherein

T₁, T₂, T₃ and T₄ are independently CH or N, wherein two of T₁, T₂, T₃ and T₄ are N and the remaining two of T₁, T₂, T₃ and T₄ are CH;

Q is a bond, O, N(CH₂)ᵣR₈ or CR₈R₉;

U is N or CR₁₀;

W is CHR₅, O, or NR₅;

each R₁ is independently H or C₁-C₆ alkyl;

R₂ is C₁-C₁₀ alkyl optionally substituted with one or two substitutents independently selected form a group consisting of NR₁₁R₁₂, COR₁₁, CO₂R₁₁, CONR₁₁R₁₂, OR₁₁, SOₓR₁₁ and SO₂NR₁₁R₁₂; or R₁ and R₂ when taken together with the ring to which they are attached form a C₈-C₁₂ bicyclic cycloakyl or an 8- to 12-membered bicyclic heterocycle;

R₃ is H, halogen, OR₁₁ or C₁-C₁₀ alkyl optionally substituted with one or two substitutents independently selected form a group consisting of NO₂, NR₁₁R₁₂, COR₁₁, CO₂R₁₁, CONR₁₁R₁₂, OR₁₁, SOₓR₁₁ and SO₂NR₁₁R₁₂; or R₂ and R₃ when taken together with the ring to which they are attached form a C₈-C₁₂ bicyclic cycloakyl or an 8- to 12-membered bicyclic heterocycle;

R₄ is H, halogen, OR₁₁, NR₁₁R₁₂, C₁₋₆ alkyl optionally substituted with at least one and up to two substitutents independently selected form a group consisting of NR₁₀R₁₁, COR₁₀, CO₂R₁₀, CONR₁₀R₁₁, OR₁₀, SOₓR₁₀ and SO₂NR₁₀R₁₁; or R₃ and R₄ when taken together with the carbon to which they are attached to form a C₃-C₈ monocyclic cycloalkyl, or a 3- to 7-membered monocyclic heterocycle;

R₅ is independently H, 5-12-membered heteroaryl, OH, CN, OR₁₀, NR₁₁R₁₂, COR₁₁, CO₂R₁₁, CONR₁₁R₁₂, CSNR₁₁R₁₂, SOₓR₁₁, SO₂NR₁₁R₁₂, NHSO₂R₁₁, NHSO₂NR₁₁R₁₂, NHCONR₁₁R₁₂, NHC(═NR₁₁)NR₁₁R₁₂, N₃ or C₁-C₆ alkyl optionally substituted with halogen, R₁₁, OR₁₀, or NR₁₁R₁₂; or and R₄ when taken together with the carbon to which they are attached to form a C₃-C₈ monocyclic cycloalkyl or a 3- to 7-membered monocyclic heterocycle;

or R₅ and R₂ when taken together with the ring to which they are attached to form an C₈-C₁₂ bicyclic cycloalkyl or an 8- to 12-membered bicyclic heterocycle; or R₅ and R₁ when taken together with the ring to which they are attached form an C₈-C₁₂ bicyclic cycloalkyl or an 8- to 12-membered bicyclic heterocycle;

R₆ and R₇ are independently H, halogen, CN, NO₂, R₁₁, OR₁₁, SOₓR₁₁, NR₁₁R₁₂;

R₈, R₉ and R₁₀ are independently H, C₁-C₆ alkyl optionally substituted with aryl or with CO₂R₁₃, or R₈ and R₉ taken together are ═O;

R₁₁ is H; C₂-C₆ alkenyl; C₁-C₆ alkyl optionally substituted with OR₁₃, N R₁₃R₁₄, halogen or with 3,7-membered monocyclic heterocycle; cycloalkyl or monocyclic or bicyclic heterocycle; aryl optionally substituted with halogen, NR₁₃R₁₄, CN or C₁-C₆ alkyl; arylalkyl, COR₁₃, CO₂R₁₃, CONR₁₃R₁₄, SO₂R₁₃, SO₂NR₁₃R₁₄ or C(═NR₁₃)NR₁₃R₁₄;

R₁₂ is H; C₁-C₆ alkyl; aryl optionally substituted with C₁-C₆ alkyl; arylalkyl, COR₁₃, CO₂R₁₃, CONR₁₃R₁₄, SO₂R₁₃, SO₂NR₁₃R₁₄ or C(═NR₁₃)NR₁₃R₁₄; or R₁₁, and R₁₂ when taken together with N to which they are attached form a 3- to 7-membered monocyclic heterocycle; or 8-12-membered bicyclic heterocycle, wherein the monocyclic heterocycle, or the bicyclic heterocycle is optionally substituted with one or two alkyl, ═O, NR₁₃R₁₄, OR₁₃ or CH₂OR₁₃;

R₁₃ is H, C₁-C₆ alkyl optionally substituted with halogen, CO—C₁-C₆ alkyl optionally substituted with halogen, CO-aryl, SO₂C₁-C₆ alkyl, SO₂-aryl, SO₂-di(C₁-C₆)alkylamino, di(C₁-C₆)alkylamino, COO—C₁-C₆ alkyl, COO-aryl optionally substituted with alkyl, NHCOO-arylalkyl, aryl optionally substituted with alkyl;

R₁₄ is H or C₁-C₆ alkyl; or R₁₃ and R₁₄ when taken together with the N to which they are attached to form a 3- to 7-membered monocyclic heterocycle;

m, n, o, p, and x are independently 0,1 or 2;

s is 0 or 1; and

r is 0, 1, 2 or 3.

In one aspect, the invention provides compounds of the Formula I:

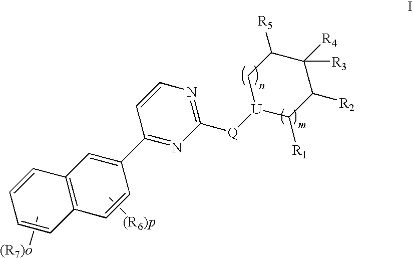

or pharmaceutically acceptable salts thereof,
wherein

Q is a bond or CR₈R₉;

U is N or CR₁₀;

R₁ is H or C₁-C₆ alkyl;

R₂ is C₁-C₁₀ alkyl substituted with 0, 1 or 2 of NR₁₁R₁₂, COR₁₁, CO₂R₁₁, CONR₁₁R₁₂, OR₁₁, S(O)ₓR₁₁, or SO₂NR₁₁R₁₂; or R₁ and R₂ when taken together with the ring to which they are attached form an 8- to 12-membered bicyclic heterocycle;

R₃ is H, halogen, C₁-C₁₀ alkyl substituted with 0, 1 or 2 of NR₁₁R₁₂, COR₁₁, CO₂R₁₁, CONR₁₁R₁₂, OR₁₁, S(O)ₓR₁₁, or SO₂NR₁₁R₁₂; or R₂ and R₃ when taken together with the ring to which they are attached form an 8- to 12-membered bicyclic heterocycle or homocycle;

R₄ is H, halogen, OR₁₁, NR₁₁R₁₂, C₁-C₆ alkyl substituted with at least one and up to two of NR₁₀R₁₁, COR₁₀, CO₂R₁₀, CONR₁₀R₁₁, OR₁₀, S(O)ₓR₁₀, or SO₂NR₁₀R₁₁; or R₃ and R₄ when taken together with the carbon to which they are attached form a C₃-C₈ monocyclic cycloalkyl or a 3- to 7-membered monocyclic heterocycle;

R₅ is H, OR₁₀, NR₁₀R₁₁ or C₁-C₆ alkyl optionally substituted with OR₁₀, or NR₁₀R₁₁; or R₅ and R₄ when taken together form a C₃-C₈ monocyclic cycloalkyl or a 3- to 7-membered monocyclic heterocycle; or R₅ and R₂, as well as R₅ and R₁, when taken together along with the ring to which they are attached form an C₈-C₁₂ bicyclic cycloalkyl or an 8- to 12-membered bicyclic heterocycle;

R₆ and R₇ are independently H, halogen, CN, NO₂, R₁₁, OR₁₁, S(O)ₓR₁₁, or NR₁₁R₁₂;

R₈ and R₉ are ═O or independently H or C₁-C₆ alkyl;

R₁₀ is H or C₁-C₆ alkyl;

R₁₁ is H, C₁-C₆ alkyl, aryl, or alkylaryl;

R₁₂ is H, C₁-C₆ alkyl, aryl, alkylaryl, COR₁₃, CO₂R₁₃, CONR₁₃R₁₄, SO₂R₁₃; or R₁₁ and R₁₂ when taken together with the N to which they are attached form a C₃-C₈ monocyclic cycloalkyl, a 3- to 7-membered monocyclic heterocycle, an C₈-C₁₂ bicyclic cycloalkyl, or an 8- to 12-membered bicyclic heterocycle, all optionally substituted with R₁₁ and OR₁₁;

R₁₃ is H or C₁-C₆ alkyl;

R₁₄ is H or C₁-C₆ alkyl; or R₁₃ and R₁₄ when taken together with the N to which they are attached form a C₃-C₈ monocyclic cycloalkyl or a 3- to 7-membered monocyclic heterocycle;

m, n, o and p are independently 0, 1 or 2; and

x is 0, 1, or 2.

In another aspect, the invention provides compounds of the Formula II

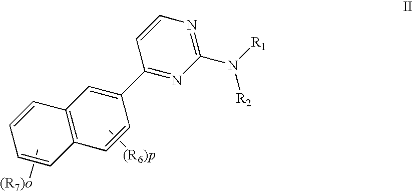

or pharmaceutically acceptable salts thereof,
wherein

R₁ and R₂ are independently —H, C₁-C₆ alkyl, C(O)C₁-C₆ alkyl, C(O)NC₁-C₆ alkyl, a C₃-C₈ monocyclic cycloalkyl, or a 3- to 7-membered monocyclic heterocycle;

R₆ and R₇ are independently H, halogen, CN, NO₂, R₁₁, OR₁₁, S(O)ₓR₁₁, or NR₁₁R₁₂;

R₁₁ is H, C₁-C₆ alkyl, aryl or alkylaryl;

R₁₂ is H, C₁-C₆ alkyl, aryl, alkylaryl, COR₁₃, CO₂R₁₃, CONR₁₃ R₁₄, or SO₂R₁₃; or R₁₁ and R₁₂ when taken together with the N to which they are attached form a 3- to 7-membered monocyclic heterocycle or an 8- to 12-membered bicyclic heterocycle;

R₁₃ is H or C₁-C₆ alkyl;

R₁₄ is H or C₁-C₆ alkyl; or R₁₃ and R₁₄ when taken together with the N to which they are attached form a 3- to 7-membered monocyclic heterocycle;

o and p are independently 0, 1 or 2; and

x is 0, 1, or 2.

In other aspects, the invention provides pharmaceutical compositions comprising compounds or pharmaceutically acceptable salts of compounds of Formula A, Formula I, and Formula II, and a pharmaceutically acceptable carrier.

In one aspect, the compounds or pharmaceutically acceptable salts of the compounds of Formula I and Formula II are useful as canonical Wnt-β-catenin cellular messaging system agonists.

In some embodiments, the invention provides methods for treating a canonical Wnt-β-catenin cellular messaging system related disorder, comprising administering to a mammal in need thereof a compound or a pharmaceutically acceptable salt of a compound of Formula A, Formula I, and Formula II in an amount effective to treat a canonical Wnt-β-catenin cellular messaging system related disorder.

### DETAILED DESCRIPTION OF THE INVENTION

The following definitions are used in connection with the naphthylpyrimidine analogs of the present invention:

“Alkyl” refers to a hydrocarbon chain that may be a straight chain or branched chain, containing the indicated number of carbon atoms. For example, C₁-C₆ indicates that the group may have from 1 to 6 (inclusive) carbon atoms in it.

“Aryl” refers to cyclic aromatic carbon ring systems made from 6 to 18 carbons. Examples of an aryl group include, but are not limited to, phenyl, naphthyl, anthracenyl, tetracenyl, and phenanthrenyl. An aryl group can be unsubstituted or substituted with one or more of the following groups: OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Heteroaryl” refers to mono and bicyclic aromatic groups of 5 to 14 atoms containing at least one heteroatom. Heteroatom as used in the term heteroaryl refers to oxygen, sulfur and nitrogen. Examples of monocyclic heteroaryls include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, and pyrimidinyl. Examples of bicyclic heteroaryls include but are not limited to, benzimidazolyl, indolyl, isoquinolinyl, indazolyl, quinolinyl, quinazolinyl, purinyl, benzisoxazolyl, benzoxazolyl, benzthiazolyl, benzodiazolyl, benzotriazolyl, isoindolyl and indazolyl. A heteroaryl group can be unsubstituted or substituted with one or more of the following groups: OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Arylalkyl” refers to an aryl group with at least one alkyl substitution. Examples of arylalkyl include, but are not limited to, toluenyl, phenylethyl, xylenyl, phenylbutyl, phenylpentyl, and ethylnaphthyl. An arylalkyl group can be unsubstituted or substituted with one or more of the following groups: OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“Heteroarylalkyl” refers to a heteroaryl group with at least one alkyl substitution. A heteroarylalkyl group can be unsubstituted or substituted with one or more of the following: H, OH, ═O, halogen, CN, C₁-C₆ alkyl, C₃-C₆ alkenyl, C₃-C₆ alkynyl, C₁-C₆ alkoxy, C₁-C₃ fluorinated alkyl, NO₂, NH₂, NHC₁-C₆ alkyl, N(C₁-C₆ alkyl)₂, NHC(O)C₁-C₆ alkyl, NHC(O)NHC₁-C₆ alkyl, SO₂NH₂, SO₂NH₂, SO₂NHC₁-C₆ alkyl, SO₂N(C₁-C₆ alkyl)₂, NHSO₂C₁-C₆ alkyl, CO₂C₁-C₆ alkyl, CONHC₁-C₆ alkyl, CON(C₁-C₆ alkyl)₂, or C₁-C₆ alkyl optionally substituted with C₁-C₆ alkyl, C₃-C₆ alkenyl, C₃-C₆ alkynyl, C₁-C₆ alkoxy, CO₂C₁-C₆ alkyl, CN, OH, cycloalkyl, CONH₂, aryl, heteroaryl, COaryl, or trifluoroacetyl.

“C₁-C₆ alkyl” as used herein refers to a straight or branched chain, saturated hydrocarbon having from 1 to 6 carbon atoms. Representative C₁-C₆ alkyl groups include, but are not limited to methyl, ethyl, propyl, isopropyl, butyl, sec-butyl, tert-butyl, pentyl, isopentyl, neopentyl, hexyl, isohexyl, and neohexyl. In one embodiment, the C₁-C₆ alkyl group is substituted with one or more of the following groups: -halo, —O—(C₁-C₆ alkyl), —OH, —CN, —COOR′, —OC(O)R′, aryl, alkylaryl, —N(R′)₂, —NHC(O)R′, —C(O)NHR′, —NHC(O)OR′, NH(SO₂R′), or NH(SO₂N(R′)₂) groups wherein each R′ is independently —H or unsubstituted —C₁-C₆ alkyl. Unless indicated, the C₁-C₆ alkyl group is unsubstituted.

“C₁-C₁₀ alkyl” as used herein refers to a straight or branched chain, saturated hydrocarbon having from 1 to 10 carbon atoms. Representative C₁-C₁₀ alkyl groups include, but are not limited to methyl, ethyl, propyl, isopropyl, butyl, sec-butyl, tert-butyl, pentyl, isopentyl, neopentyl, hexyl, isohexyl, neohexyl, heptyl, isoheptyl, neoheptyl, octyl, isooctyl, neooctyl, nonyl, isononyl, neononyl, decyl, isodecyl and neodecyl. In one embodiment, the C₁-C₁₀ alkyl group is substituted with one or more of the following groups: -halo, —O—(C₁-C₆ alkyl), —OH, —CN, —COO′, —OC(O)R′, aryl, alkylaryl, —N(R′)₂, —NHC(O)R′, —C(O)NHR′, —NHC(O)OR′, NH(SO₂R′), or NH(SO₂N(R′)₂, or NH(SO₂N(R′)₂) groups wherein each R′ is independently —H or unsubstituted —C₁-C₆ alkyl.

“C₂-C₆ alkenyl” refers to a straight or branched chain unsaturated hydrocarbon containing 2-6 carbon atoms and at least one double bond. Examples of a C₂-C₆ alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, and isohexene.

“C₂-C₆ alkynyl” refers to a straight or branched chain unsaturated hydrocarbon containing 2-6 carbon atoms and at least one triple bond. Examples of a C₂-C₆ alkynyl group include, but are not limited to, acetylene, propyne, 1-butyne, 2-butyne, isobutyne, sec-butyne, 1-pentyne, 2-pentyne, isopentyne, 1-hexyne, 2-hexyne, and 3-hexyne.

“C₃-C₆ alkynyl” refers to a straight or branched chain unsaturated hydrocarbon containing 3-6 carbon atoms and at least one triple bond. Examples of a C₃-C₆ alkynyl group include, but are not limited to, propyne, 1-butyne, 2-butyne, isobutyne, sec-butyne, 1-pentyne, 2-pentyne, isopentyne, 1-hexyne, 2-hexyne, and 3-hexyne.

“C₁-C₆ alkoxy” refers to a straight or branched chain saturated or unsaturated hydrocarbon containing 1-6 carbon atoms and at least one oxygen atom. Examples of a C₁-C₆ alkoxy include, but are not limited to, methoxy, ethoxy, isopropoxy, butoxy, n-pentoxy, isopentoxy, neopentoxy, and hexoxy

The term “C₃-C₈ monocyclic cycloalkyl” as used herein is a 3-, 4-, 5-, 6-, 7- or 8-membered saturated non-aromatic monocyclic cycloalkyl ring. Representative C₃-C₈ monocyclic cycloalkyl groups include, but are not limited to, cyclopropyl, cyclobutyl, cyclopentyl, cyclohexyl, cycloheptyl and cyclooctyl. In one embodiment, the C₃-C₈ monocyclic cycloalkyl group is substituted with one or more of the following groups: -halo, —O—(C₁-C₆ alkyl), —OH, —CN, —COOR′, —OC(O)R′, aryl, alkylaryl, —N(R′)₂, —NHC(O)R′—C(O)NHR′, —NHC(O)OR′, NH(SO₂R′), or NH(SO₂N(R′)₂) groups wherein each R′ is independently —H, aryl, or unsubstituted —C₁-C₆-alkyl.

The term “C₈-C₁₂ bicyclic cycloalkyl” as used herein is a 8-, 9-, 10-, 11- or 1 2-membered saturated, non-aromatic bicyclic cycloalkyl ring system. Representative C₈-C₁₂ bicyclic cycloalkyl groups include, but are not limited to, decahydronaphthalene, octahydroindene, decahydrobenzocycloheptene, and dodecahydroheptalene. In one embodiment, the C₈-C₁₂ bicyclic cycloalkyl group is substituted with one or more of the following groups: -halo, —O—(C₁-C₆ alkyl), —OH, —CN, —COOR′, —OC(O)R′, aryl, alkylaryl, —N(R′)₂, —NHC(O)R′, —C(O)NHR′, —NHC(O)OR′, NH(SO₂R′), or NH(SO₂N(R′)₂) groups wherein each R′ is independently —H, aryl, or unsubstituted —C₁-C₆ alkyl.

The term “3- to 7-membered monocyclic heterocycle” refers to: (i) a 3- or 4-membered non-aromatic monocyclic cycloalkyl in which 1 of the ring carbon atoms has been replaced with an N, O or S atom; or (ii) a 5-, 6-, or 7-membered aromatic or non-aromatic monocyclic cycloalkyl in which 1-4 of the ring carbon atoms have been independently replaced with a N, O or S atom. The non-aromatic 3- to 7-membered monocyclic heterocycles can be attached via a ring nitrogen, sulfur, or carbon atom. The aromatic 3- to 7-membered monocyclic heterocycles are attached via a ring carbon atom. Representative examples of a 3- to 7-membered monocyclic heterocycle group include, but are not limited to furanyl, furazanyl, imidazolidinyl, imidazolinyl, imidazolyl, isothiazolyl, isoxazolyl, morpholinyl, oxadiazolyl, oxazolidinyl, oxazolyl, oxazolidinyl, pyrimidinyl, phenanthridinyl, phenanthrolinyl, piperazinyl, piperidinyl, pyranyl, pyrazinyl, pyrazolidinyl, pyrazolinyl, pyrazolyl, pyridazinyl, pyridooxazole, pyridoimidazole, pyridothiazole, pyridinyl, pyrimidinyl, pyrrolidinyl, pyrrolinyl, quinuclidinyl, tetrahydrofuranyl, thiadiazinyl, thiadiazolyl, thienyl, thienothiazolyl, thienooxazolyl, thienoimidazolyl, thiomorpholinyl, thiophenyl, triazinyl, triazolyl, In one embodiment, the 3- to 7-membered monocyclic heterocycle group is substituted with one or more of the following groups: -halo, —O—(C₁-C₆ alkyl), —OH, —CN, —COOR′, —OC(O)R′, aryl, alkylaryl, —N(R′)₂, —NHC(O)R′, —C(O)NHR′, —NHC(O)OR′, NH(SO₂R′), or NH(SO₂N(R′)₂) groups wherein each R′ is independently —H or unsubstituted —C₁-C₆ alkyl. In another embodiment, one or more of the ring nitrogens is substituted with R′, C(O)R′, C(O)H, C(NH)N(R′), C(O)OR′, C(O)N(R′), SO₂R′, heteroaryl, C(O)CF₃.

The term “8- to 12-membered bicyclic heterocycle” refers to a bicyclic 8- to 12-membered aromatic or non-aromatic bicyclic cycloalkyl in which one or both of the of the rings of the bicyclic ring system have 1-4 of its ring carbon atoms independently replaced with a N, O or S atom. Included in this class are 3- to 7-membered monocyclic heterocycles that are fused to a benzene ring. A non-aromatic ring of an 8- to 12-membered monocyclic heterocycle is attached via a ring nitrogen, sulfur, or carbon atom. An aromatic 8- to 12-membered monocyclic heterocycles are attached via a ring carbon atom. Examples of 8- to 12-membered bicyclic heterocycles include, but are not limited to, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazolinyl, cinnolinyl, decahydroquinolinyl, 1H-indazolyl, indolenyl, indolinyl, indolizinyl, indolyl, isobenzofuranyl, isoindazolyl, isoindolyl, isoindolinyl, isoquinolinyl, naphthyridinyl, octahydroisoquinolinyl, phthalazinyl, pteridinyl, purinyl, quinoxalinyl, tetrahydroisoquinolinyl, tetrahydroquinolinyl, and xanthenyl. In one embodiment, each ring of the -8- to 12-membered bicyclic heterocycle group can substituted with one or more of the following groups: -halo, —O—(C₁-C₆ alkyl), —OH, —CN, —COOR′, —OC(O)R′, aryl, alkylaryl, —N(R′)₂, —NHC(O)R′, —C(O)NHR—, —NHC(O)OR′, NH(SO₂R′), or NH(SO₂N(R′)₂) groups wherein each R′ is independently —H or unsubstituted —C₁-C₆ alkyl. In another embodiment, one or more of the ring nitrogens is substituted with R′, C(O)R′, C(O)H, C(NH)N(R′), C(O)OR′, C(O)N(R′), SO₂R′, heteroaryl, C(O)CF₃.

A “subject” is a mammal; e.g., a human, mouse, rat, guinea pig, dog, cat, horse, cow, pig, or non-human primate, such as a chimpanzee, baboon or monkey such as rhesus or cynomolgus monkey.

The invention also provides pharmaceutical compositions comprising an effective amount of a naphthylpyrimidine analog and a pharmaceutically acceptable carrier. The invention provides a naphthylpyrimidine analog when provided as a pharmaceutically acceptable prodrug, hydrated salt, such as a pharmaceutically acceptable salt, or mixtures thereof.

Representative “pharmaceutically acceptable salts” include, e.g., water-soluble and water-insoluble salts, such as the acetate, amsonate (4,4-diaminostilbene-2,2-disulfonate), benzenesulfonate, benzoate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

An “effective amount” when used in connection an naphthylpyrimidine analog is an amount effective for treating or preventing a disease associated with the canonical Wnt-β-catenin cellular messaging system.

The following abbreviations are used herein and have the indicated definitions: ACN is acetonitrile, HOAc is acetic acid, n-BuLi is normal butyl lithium, DDQ is 2,3-dicyano-5,6-dichloro-parabenzoquinone, DIEA is diisopropylethylamine, DMF is N,N-dimethylformamide, DMSO is dimethylsulfoxide, EtOAc is ethyl acetate, EtOH is ethanol, FBS is fetal bovine serum, HPLC is high pressure liquid chromatography, I—Pr₂NEt is diisopropylethylamine, MeCN is acetonitrile, MeOH is methanol, MS is mass spectrometry, NEt₃ is triethylamine, NMP is N-methyl-2-pyrrolidone, NMR is nuclear magnetic resonance, PBS is phosphate-buffered saline (pH 7.4), RPMI is Roswell Park Memorial Institute, T-BuOK is potassium tert-Butoxide, THF is tetrahydrofuran, TFA is trifluoroacetic acid, and TLC is thin-layer chromatography, VLUX is a device for measuring luminescence.

### The Naphthylpyrimidine, Naphthylpyrazine And Naphthylpyridazine Analogs

In one aspect, the invention provides compounds of Formula (A):

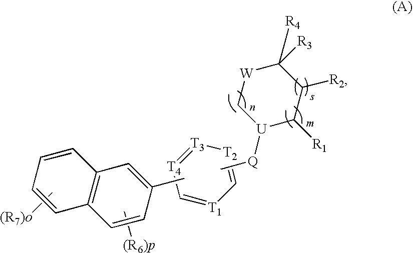

or pharmaceutically acceptable salts thereof, wherein
T₁, T₂, T₃, T₄, Q, U, W, R₁, R₂, R₃, R₄, R₅, R₆, R₇, R₈, R₉, R₁₀, R₁₁, R₁₂, R₁₃, R₁₄, s, m, n o, p and r are as defined above for compounds of Formula (A).

In one embodiment, Q is a bond. In one embodiment, Q is O. In one embodiment, Q is N(CH₂)ᵣR₈ In one embodiment, Q is CR₈R₉.

In one embodiment, U is N. In one embodiment, U is CR₁₀.

In one embodiment, W is CHR₅. In one embodiment, W is O. In one embodiment, W is NR₅.

In one embodiment, R₁ is H. In one embodiment, R₁ is C₁-C₆ alkyl;

In one embodiment, R₂ is C₁-C₁₀ alkyl optionally substituted with one or two substitutents independently selected form a group consisting of NR₁₁R₁₂, COR₁₁, CO₂R₁₁, CONR₁₁R₁₂, OR₁₁, SOₓR₁₁ and SO₂NR₁₁R₁₂.

In one embodiment, R₁ and R₂ when taken together with the ring to which they are attached form a C₈-C₁₂ bicyclic cycloakyl or an 8- to 12-membered bicyclic heterocycle.

In one embodiment, R₃ is OR₁₁. In one embodiment, R₃ is C₁-C₁₀ alkyl optionally substituted with one or two substitutents independently selected form a group consisting of NO₂, NR₁₁R₁₂, COR₁₁, CO₂R₁₁, CONR₁₁R₁₂, OR₁₁, SOₓR₁₁ and SO₂NR₁₁R₁₂.

In one embodiment, R₂ and R₃ when taken together with the ring to which they are attached form a C₈-C₁₂ bicyclic cycloakyl or an 8- to 12-membered bicyclic heterocycle.

In one embodiment, R₄ is H. In one embodiment. R₄ is OR₁₁. In one embodiment, R₄ is NR₁₁R₁₂. In one embodiment, R₄ is C₁₋₆ alkyl optionally substituted with at least one and up to two substitutents independently selected form a group consisting of NR₁₀R₁₁, COR₁₀, CO₂R₁₀, CONR₁₀R₁₁, OR_{, 10}SOₓR₁₀ and SO₂NR₁₀R₁₁.

In one embodiment, R₃ and R₄ when taken together with the carbon to which they are attached to form a C₃-C₈ monocyclic cycloalkyl, or a 3- to 7-membered monocyclic heterocycle.

In one embodiment, R₅ is independently H. In one embodiment, R₅ is 5-12-membered heteroaryl. In one embodiment, R₅ is OH. In one embodiment, R₅ is CN. In one embodiment, R₅ is OR₁₀. In one embodiment, R₅ is NR₁₁R₁₂. In one embodiment, R₅ is COR₁₁. In one embodiment, R₅ is CO₂R₁₁. In one embodiment, R₅ is CONR₁₁R₁₂. In one embodiment, R₅ is CSNR₁₁R₁₂. In one embodiment, R₅ is SOₓR₁₁. In one embodiment, R₅ is SO₂NR₁₁R₁₂. In one embodiment, R₅ is NHSO₂R₁₁. In one embodiment, R₅ is NHSO₂NR₁₁R₁₂. In one embodiment, R₅ is NHCONR₁₁R₁₂. In one embodiment, R₅ is NHC(═NR₁₁)NR₁₁R₁₂. In one embodiment, R₅ is N₃. In one embodiment, R₅ is C₁-C₆ alkyl optionally substituted with halogen, R₁₁, OR₁₀, or NR₁₁R₁₂.

In one embodiment, R₅ and R₄ when taken together with the carbon to which they are attached to form a C₃-C₈ monocyclic cycloalkyl or a 3- to 7-membered monocyclic heterocycle.

In one embodiment, R₅ and R₂ when taken together with the ring to which they are attached to form an C₈-C₁₂ bicyclic cycloalkyl or an 8- to 12-membered bicyclic heterocycle; or R₅ and R₁ when taken together with the ring to which they are attached form an C₈-C₁₂ bicyclic cycloalkyl or an 8- to 12-membered bicyclic heterocycle.

In one embodiment, R₆ and R₇ are independently H. In one embodiment, R₆ and R₇ are independently halogen. In one embodiment, R₆ and R₇ are independently CN. In one embodiment, R₆ and R₇ are independently NO₂. In one embodiment, R₆ and R₇ are independently R₁₁. In one embodiment, R₆ and R₇ are independently OR₁₁. In one embodiment, R₆ and R₇ are independently SOₓR₁₁. In one embodiment, R₆ and R₇ are independently NR₁₁R₁₂.

In one embodiment, R₈, R₉ and R₁₀ are independently H. In one embodiment, R₈, R₉ and R₁₀ are independently C₁-C₆ alkyl optionally substituted with aryl or with CO₂R₁₃;

In one embodiment, R₁₁ is H. In one embodiment, R₁₁ is C₂-C₆ alkenyl. In one embodiment, R₁₁ is C₁-C₆ alkyl optionally substituted with OR₁₃, N R₁₃R₁₄, halogen or with 3-7-membered monocyclic heterocycle; cycloalkyl or monocyclic or bicyclic heterocycle; aryl optionally substituted with halogen, NR₁₃R₁₄, CN or C₁-C₆ alkyl; arylalkyl, COR₁₃, CO₂R₁₃, CONR₁₃R₁₄, SO₂R₁₃, SO₂NR₁₃R₁₄ or C(═NR13)NR₁₃R₁₄.

In one embodiment, R₁₂ is H. In one embodiment, R₁₂ is C₁-C₆ alkyl. In one embodiment, R₁₂ is aryl optionally substituted with C₁-C₆ alkyl; arylalkyl, COR₁₃, CO₂R₁₃, CONR₁₃R₁₄, SO₂NR₁₃R₁₄ or C(═NR13)NR₁₃R₁₄; or R₁₁ and R₁₂ when taken together with N to which they are attached form a 3- to 7-membered monocyclic heterocycle; or 8-12-membered bicyclic heterocycle, wherein the monocyclic heterocycle, or the bicyclic heterocycle is optionally substituted with one or two alkyl, ═O, NR₁₃R₁₄, OR₁₃ or CH₂OR₁₃.

In one embodiment, R₁₃ is H. In one embodiment, R₁₃ is C₁-C₆ alkyl optionally substituted with halogen, CO—C₁-C₆ alkyl optionally substituted with halogen, CO-aryl, SO₂C₁-C₆ alkyl, SO₂-aryl, SO₂-di(C₁-C₆)alkylamino, di(C₁-C₆)alkylamino, COO—C₁-C₆ alkyl, COO-aryl optionally substituted with alkyl, NHCOO-arylalkyl, aryl optionally substituted with alkyl.

In one embodiment, R₁₄ is H. In one embodiment, R₁₄ is C₁-C₆ alkyl.

In one embodiment, R₁₃ and R₁₄ when taken together with the N to which they are attached to form a 3- to 7-membered monocyclic heterocycle.

In one embodiment, m, n, o, p, and x are independently 0. In one embodiment, m, n, o, p, and x are independently 1. In one embodiment, m, n, o, p, and x are independently 2.

In one embodiment, s is 0. In one embodiment, s is 1.

In one embodiment, r is 0. In one embodiment, r is 1. In one embodiment, r is 2. In one embodiment, r is 3.

In one embodiment, the 3- to 7-membered heterocycle formed by R₃ and R₄ is a 5, 6, or 7-membered heteroaryl.

In one embodiment, the 3- to 7-membered heterocycle formed by R₅ and R₄ is a 5, 6, or 7-membered heteroaryl.

In one embodiment, the 3- to 7-membered heterocycle formed by R₁₁ and R₁₂ is a 5, 6, or 7-membered heteroaryl.

In one embodiment, the 3- to 7-membered heterocycle formed by R₁₃ and R₁₄ is a 5, 6, or 7-membered heteroaryl.

In one embodiment, R₅ is a 5-10 membered heteroaryl. In another embodiment, R₅ is a 5-7 membered heteroaryl.

In one embodiment, R₁₁, is a C₁-C₆ alkyl substituted with a 5-7 membered heteroaryl.

In one embodiment, the ring of Formula (A)

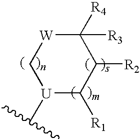

is selected from the group consisting of

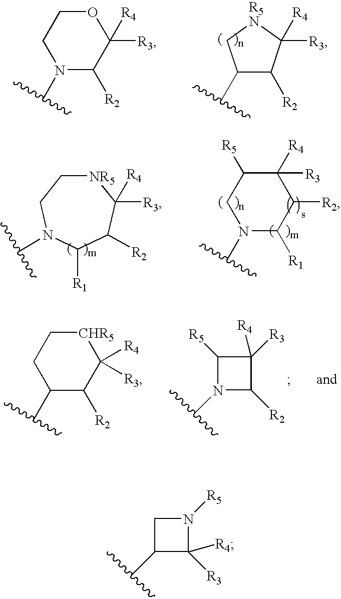

wherein R₁, R₂, R₃, R₄, R₅, m, n and s are as defined above in Formula (A).

In one embodiment,

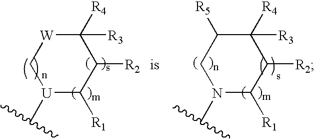

wherein R₁, R₂, R₃, R₄, R₅, m, and n are as defined above in Formula (A).

In one embodiment, n=1 and m=1.

In one embodiment, n=1 and m=0.

In one embodiment, n =2 and m=0.

In one embodiment, R₂ is CH₂OR₁₁

In one embodiment, R₃ is OR₁₁ or CH₂OR₁₁.

In one embodiment, R₄ is OR₁₁. In one embodiment, R₅ is CN; NR₁₁R₁₂; C(S)NR₁₁R₁₂; or alkyl optionally substituted with NR₁₁R₁₂, monocyclic heterocycle, or bicyclic heterocycle.

In one embodiment,

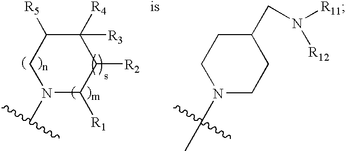

wherein R₁, R₂, R₃, R₅, R₁₁, R₁₂, s, m, and n are as defined in Formula (A)

In one embodiment,

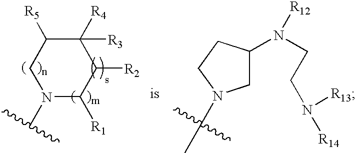

wherein R₁, R₂, R₃, R₄, R₅, R₁₂, R₁₃, R₁₄, s, m, and n are as defined in Formula (A).

In one embodiment,

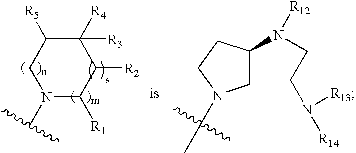

wherein R₁, R₂, R₃, R₅, R₁₂, R₁₃, R₁₄, s, m, and n are as defined Formula (A).

In one embodiment,

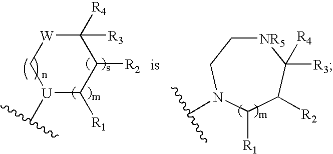

wherein R₁, R₂, R₃, R₄, R₅, s and n are as defined in Formula (A) and wherein m is 0 or 1.

In one embodiment, Formula (A) is Formula (AII):

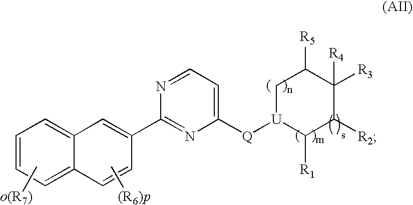

wherein Q, U, R₁, R₂, R₃, R₄, R₅, R₆, R₇, m, n, o, p and s are as defined in Formula (A).

In one embodiment, Formula (A) is Formula (AIII):

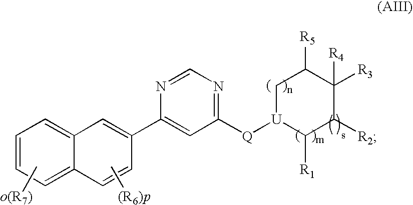

wherein Q, U, R₁, R₂, R₃, R₄, R₅, R₆, R₇, m, n, o, p and s are as defined in Formula (A).

In one embodiment, Formula (A) is Formula (AIV):

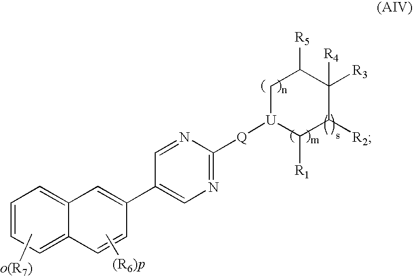

wherein Q, U, R₁, R₂, R₃, R₄, R₅, R₆, R₇, m, n, o, p and s are as defined in Formula (A).

In one embodiment, Formula (A) is Formula (AV):

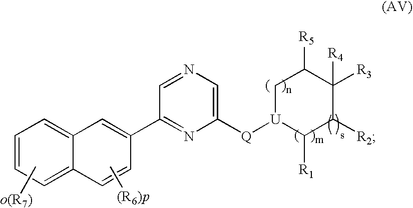

wherein Q, U, R₁, R₂, R₃, R₄, R₅, R₆, R₇, m, n, o, p and s are as defined in Formula (A).

In one embodiment, Formula (A) is Formula (AVI):

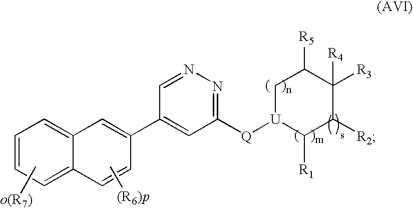

wherein Q, U, R₁, R₂, R₃, R₄, R₅, R₆, R₇, m, n, o, p and s are as defined in Formula (A).

Illustrative compounds of Formula A, are exemplified by the following compounds:

Compound

1-{1-[2-(2-naphthyl)pyrimidin-4-yl]piperidin-4-yl}methanamine
1-{1-[6-(2-naphthyl)pyrimidin-4-yl]piperidin-4-yl}methanamine
1-{1-[5-(2-naphthyl)pyrimidin-2-yl]piperidin-4-yl}methanamine
1-{1-[6-(2-naphthyl)pyrazin-2-yl]piperidin-4-yl}methanamine
1-{1-[5-(2-naphthyl)pyridazin-3-yl]piperidin-4-yl}methanamine
1-{1-[4-(2-naphthyl)pyrimidin-2-yl]-1,2,3,4-tetrahydroquinolin-4-yl}methanamine
1-{(4S)-1-[4-(2-naphthyl)pyrimidin-2-yl]azepan-4-yl}methanamine
1-{(4R)-1-[4-(2-naphthyl)pyrimidin-2-yl]azepan-4-yl}methanamine
1-[4-(2-naphthyl)pyrimidin-2-yl]azepan-4-amine
N-{1-[4-(2-naphthyl)pyrimidin-2-yl]azepan-4-yl}acetamide
2,2,2-trifluoro-N-{1-[4-(2-naphthyl)pyrimidin-2-yl]azepan-4-yl}acetamide
N,N-dimethyl-N′-{1-[4-(2-naphthyl)pyrimidin-2-yl]azepan-4-yl}urea
N-{1-[4-(2-naphthyl)pyrimidin-2-yl]azepan-4-yl}benzamide
N-{1-[4-(2-naphthyl)pyrimidin-2-yl]azepan-4-yl}urea
N-ethyl-N′-{1-[4-(2-naphthyl)pyrimidin-2-yl]azepan-4-yl}urea
N-{1-[4-(2-naphthyl)pyrimidin-2-yl]azepan-4-yl}methanesulfonamide
4-methyl-N-{1-[4-(2-naphthyl)pyrimidin-2-yl]azepan-4-yl}benzenesulfonamide
N,N-dimethyl-N′-{1-[4-(2-naphthyl)pyrimidin-2-yl]azepan-4-yl}sulfamide
{(1S,4R)-2-[4-(2-naphthyl)pyrimidin-2-yl]-2-azabicyclo[2.2.1]heptane-5,5-diyl}dimethanamine
1-{(1R,4R,5R)-2-[4-(2-naphthyl)pyrimidin-2-yl]-2-azabicyclo[2.2.1]hept-5-yl}methanamine
1-{(1R,4R,5S)-2-[4-(2-naphthyl)pyrimidin-2-yl]-2-azabicyclo[2.2.1]hept-5-yl}methanamine
1-{(1S,4S,5S)-2-[4-(2-naphthyl)pyrimidin-2-yl]-2-azabicyclo[2.2.1]hept-5-yl}methanamine
1-{(1S,4S,5R)-2-[4-(2-naphthyl)pyrimidin-2-yl]-2-azabicyclo[2.2.1]hept-5-yl}methanamine
1-{8-[4-(2-naphthyl)pyrimidin-2-yl]-8-azabicyclo[3.2.1]oct-3-yl}methanamine
1-{8-[4-(2-naphthyl)pyrimidin-2-yl]-8-azabicyclo[3.2.1]oct-3-yl}methanamine
2-[4-(2-naphthyl)pyrimidin-2-yl]-2,8-diazaspiro[4.5]decane
N,N,N′-trimethyl-N′-{(3R)-1-[4-(2-naphthyl)pyrimidin-2-yl]pyrrolidin-3-yl}ethane-1,2-diamine
(3aR*,6aS*)-2-[4-(2-naphthyl)pyrimidin-2-yl]octahydropyrrolo[3,4-c]pyrrole
5-(4-naphthalen-2-ylpyrimidin-2-yl)octahydropyrrolo[3,4-b]pyrrole
1-({1-[4-(2-naphthyl)pyrimidin-2-yl]pyrrolidin-3-yl}methyl)piperidin-4-ol
(3S)-1-({1-[4-(2-naphthyl)pyrimidin-2-yl]pyrrolidin-3-yl}methyl)piperidin-3-ol
(3R)-1-({1-[4-(2-naphthyl)pyrimidin-2-yl]pyrrolidin-3-yl}methyl)piperidin-3-ol
(3S)-1-({1-[4-(2-naphthyl)pyrimidin-2-yl]pyrrolidin-3-yl}methyl)pyrrolidin-3-ol
(3R)-1-({1-[4-(2-naphthyl)pyrimidin-2-yl]pyrrolidin-3-yl}methyl)pyrrolidin-3-ol
(3S,3′R)-1′-[4-(2-naphthyl)pyrimidin-2-yl]-1,3′-bipyrrolidin-3-ol
2-{(3R)-1-[4-(2-naphthyl)pyrimidin-2-yl]pyrrolidin-3-yl}octahydro-2H-pyrido[1,2-a]pyrazine
(3S,3′R)-1′-[4-(2-naphthyl)pyrimidin-2-yl]-1,3′-bipyrrolidin-3-amine
3-(methyl{(3R)-1-[4-(2-naphthyl)pyrimidin-2-yl]pyrrolidin-3-yl}amino)propan-1-ol
N,N-dimethyl-N′-{(3R)-1-[4-(2-naphthyl)pyrimidin-2-yl]pyrrolidin-3-yl}ethane-1,2-diamine
N,N′-dimethyl-N-{(3R)-1-[4-(2-naphthyl)pyrimidin-2-yl]pyrrolidin-3-yl}ethane-1,2-diamine
(3R,3′R)-1′-[4-(2-naphthyl)pyrimidin-2-yl]-1,3′-bipyrrolidin-3-ol
(3R,3′R)-1′-[4-(2-naphthyl)pyrimidin-2-yl]-1,3′-bipyrrolidin-3-amine
(1S,4S)-2-methyl-5-{(3R)-1-[4-(2-naphthyl)pyrimidin-2-yl]pyrrolidin-3-yl}-2,5-
diazabicyclo[2.2.1]heptane
5-{(3R)-1-[4-(2-naphthyl)pyrimidin-2-yl]pyrrolidin-3-yl}octahydropyrrolo[3,4-b]pyrrole
N,N,N′-trimethyl-N′-{(3R)-1-[4-(2-naphthyl)pyrimidin-2-yl]pyrrolidin-3-yl}propane-1,3-diamine
(3R,3′S)-1′-[4-(2-naphthyl)pyrimidin-2-yl]-1,3′-bipyrrolidin-3-ol
(3S,3′S)-1′-[4-(2-naphthyl)pyrimidin-2-yl]-1,3′-bipyrrolidin-3-ol
N,N-dimethyl-N′-{(3S)-1-[4-(2-naphthyl)pyrimidin-2-yl]pyrrolidin-3-yl}ethane-1,2-diamine
N,N,N′-trimethyl-N′-{(3S)-1-[4-(2-naphthyl)pyrimidin-2-yl]pyrrolidin-3-yl}ethane-1,2-diamine
3-(methyl{(3S)-1-[4-(2-naphthyl)pyrimidin-2-yl]pyrrolidin-3-yl}amino)propan-1-ol
5-{(3S)-1-[4-(2-naphthyl)pyrimidin-2-yl]pyrrolidin-3-yl}octahydropyrrolo[3,4-b]pyrrole
N,N,N′-trimethyl-N′-{(3S)-1-[4-(2-naphthyl)pyrimidin-2-yl]pyrrolidin-3-yl}propane-1,3-diamine
2-(ethyl{(3S)-1-[4-(2-naphthyl)pyrimidin-2-yl]pyrrolidin-3-yl}amino)ethanol
2-[(3S)-3-(1H-imidazol-1-yl)pyrrolidin-1-yl]-4-(2-naphthyl)pyrimidine
(3S)—N-cyclohexyl-N-methyl-1-[4-(2-naphthyl)pyrimidin-2-yl]pyrrolidin-3-amine
(3S)—N-(tert-butyl)-1-[4-(2-naphthyl)pyrimidin-2-yl]pyrrolidin-3-amine
4-(2-naphthyl)-2-[(3S)-3-piperazin-1-ylpyrrolidin-1-yl]pyrimidine
4-{(3S)-1-[4-(2-naphthyl)pyrimidin-2-yl]pyrrolidin-3-yl}morpholine
4-{(3S)-1-[4-(2-naphthyl)pyrimidin-2-yl]pyrrolidin-3-yl}piperazin-2-one
1-{(3S)-1-[4-(2-naphthyl)pyrimidin-2-yl]pyrrolidin-3-yl}azepane
(3′S)-1′-[4-(2-naphthyl)pyrimidin-2-yl]-1,3′-bipyrrolidine
4-(2-naphthyl)-2-[(3S)-3-piperidin-1-ylpyrrolidin-1-yl]pyrimidine
2-[(3S)-3-(4-methylpiperazin-1-yl)pyrrolidin-1-yl]-4-(2-naphthyl)pyrimidine
(1-{(3S)-1-[4-(2-naphthyl)pyrimidin-2-yl]pyrrolidin-3-yl}piperidin-3-yl)methanol
(1-{(3S)-1-[4-(2-naphthyl)pyrimidin-2-yl]pyrrolidin-3-yl}piperidin-4-yl)methanol
1-{(3S)-1-[4-(2-naphthyl)pyrimidin-2-yl]pyrrolidin-3-yl}piperidin-3-ol
(3S)—N-(2-morpholin-4-ylethyl)-1-[4-(2-naphthyl)pyrimidin-2-yl]pyrrolidin-3-amine
1-{(3S)-1-[4-(2-naphthyl)pyrimidin-2-yl]pyrrolidin-3-yl}piperidin-4-ol
(3R,3′S)-N,N-dimethyl-1′-[4-(2-naphthyl)pyrimidin-2-yl]-1,3′-bipyrrolidin-3-amine
(3S,3′S)-N,N-dimethyl-1′-[4-(2-naphthyl)pyrimidin-2-yl]-1,3′-bipyrrolidin-3-amine
(3R)-1-{(3S)-1-[4-(2-naphthyl)pyrimidin-2-yl]pyrrolidin-3-yl}piperidin-3-ol
(3S)-1-{(3S)-1-[4-(2-naphthyl)pyrimidin-2-yl]pyrrolidin-3-yl}piperidin-3-ol
2-(ethyl{(3R)-1-[4-(2-naphthyl)pyrimidin-2-yl]pyrrolidin-3-yl}amino)ethanol
2-[(3R)-3-(1H-imidazol-1-yl)pyrrolidin-1-yl]-4-(2-naphthyl)pyrimidine
(3R)—N-cyclohexyl-N-methyl-1-[4-(2-naphthyl)pyrimidin-2-yl]pyrrolidin-3-amine
(3R)—N-(tert-butyl)-1-[4-(2-naphthyl)pyrimidin-2-yl]pyrrolidin-3-amine
4-(2-naphthyl)-2-[(3R)-3-piperazin-1-ylpyrrolidin-1-yl]pyrimidine
4-{(3R)-1-[4-(2-naphthyl)pyrimidin-2-yl]pyrrolidin-3-yl}morpholine
4-{(3R)-1-[4-(2-naphthyl)pyrimidin-2-yl]pyrrolidin-3-yl}piperazin-2-one
1-{(3R)-1-[4-(2-naphthyl)pyrimidin-2-yl]pyrrolidin-3-yl}azepane
(3′R)-1′-[4-(2-naphthyl)pyrimidin-2-yl]-1,3′-bipyrrolidine
4-(2-naphthyl)-2-[(3R)-3-piperidin-1-ylpyrrolidin-1-yl]pyrimidine
2-[(3R)-3-(4-methylpiperazin-1-yl)pyrrolidin-1-yl]-4-(2-naphthyl)pyrimidine
(1-{(3R)-1-[4-(2-naphthyl)pyrimidin-2-yl]pyrrolidin-3-yl}piperidin-3-yl)methanol
(1-{(3R)-1-[4-(2-naphthyl)pyrimidin-2-yl]pyrrolidin-3-yl}piperidin-4-yl)methanol
1-{(3R)-1-[4-(2-naphthyl)pyrimidin-2-yl]pyrrolidin-3-yl}piperidin-3-ol
(3R)—N-(2-morpholin-4-ylethyl)-1-[4-(2-naphthyl)pyrimidin-2-yl]pyrrolidin-3-amine
1-{(3R)-1-[4-(2-naphthyl)pyrimidin-2-yl]pyrrolidin-3-yl}piperidin-4-ol
(3R,3′R)-N,N-dimethyl-1′-[4-(2-naphthyl)pyrimidin-2-yl]-1,3′-bipyrrolidin-3-amine
(3S,3′R)-N,N-dimethyl-1′-[4-(2-naphthyl)pyrimidin-2-yl]-1,3′-bipyrrolidin-3-amine
(3R)-1-{(3R)-1-[4-(2-naphthyl)pyrimidin-2-yl]pyrrolidin-3-yl}piperidin-3-ol
(3S)-1-{(3R)-1-[4-(2-naphthyl)pyrimidin-2-yl]pyrrolidin-3-yl}piperidin-3-ol
{1-[4-(2-naphthyl)pyrimidin-2-yl]pyrrolidin-3-yl}methanol
1-({1-[4-(2-naphthyl)pyrimidin-2-yl]pyrrolidin-3-yl}methyl)azepane
4-(2-naphthyl)-2-[3-(piperidin-1-ylmethyl)pyrrolidin-1-yl]pyrimidine
4-(2-naphthyl)-2-[3-(pyrrolidin-1-ylmethyl)pyrrolidin-1-yl]pyrimidine
2-[3-(azetidin-1-ylmethyl)pyrrolidin-1-yl]-4-(2-naphthyl)pyrimidine
4-({1-[4-(2-naphthyl)pyrimidin-2-yl]pyrrolidin-3-yl}methyl)morpholine
4-({1-[4-(2-naphthyl)pyrimidin-2-yl]pyrrolidin-3-yl}methyl)thiomorpholine
N-ethyl-N-({1-[4-(2-naphthyl)pyrimidin-2-yl]pyrrolidin-3-yl}methyl)ethanamine
2-methyl-N-({1-[4-(2-naphthyl)pyrimidin-2-yl]pyrrolidin-3-yl}methyl)propan-2-amine
2-[ethyl({1-[4-(2-naphthyl)pyrimidin-2-yl]pyrrolidin-3-yl}methyl)amino]ethanol
2-{3-[(4-methylpiperazin-1-yl)methyl]pyrrolidin-1-yl}-4-(2-naphthyl)pyrimidine
4-({1-[4-(2-naphthyl)pyrimidin-2-yl]pyrrolidin-3-yl}methyl)piperazin-2-one
2-[3-(1H-imidazol-1-ylmethyl)pyrrolidin-1-yl]-4-(2-naphthyl)pyrimidine
2-[3-(chloromethyl)pyrrolidin-1-yl]-4-(2-naphthyl)pyrimidine
N,N-dimethyl-1-{(3S)-1-[4-(2-naphthyl)pyrimidin-2-yl]pyrrolidin-3-yl}methanamine
4-(2-naphthyl)-2-[(3S)-3-(piperidin-1-ylmethyl)pyrrolidin-1-yl]pyrimidine
4-(2-naphthyl)-2-[(3S)-3-(pyrrolidin-1-ylmethyl)pyrrolidin-1-yl]pyrimidine
N-methyl-1-{(3S)-1-[4-(2-naphthyl)pyrimidin-2-yl]pyrrolidin-3-yl}methanamine
N-methyl-N-({(3S)-1-[4-(2-naphthyl)pyrimidin-2-yl]pyrrolidin-3-yl}methyl)cyclohexanamine
1-({(3R)-1-[4-(2-naphthyl)pyrimidin-2-yl]pyrrolidin-3-yl}methyl)piperidin-2-one
tert-butyl 4-({(3S)-1-[4-(2-naphthyl)pyrimidin-2-yl]pyrrolidin-3-yl}methyl)piperazine-1-carboxylate
2-methyl-N-({(3S)-1-[4-(2-naphthyl)pyrimidin-2-yl]pyrrolidin-3-yl}methyl)propan-2-amine
2-[ethyl({(3S)-1-[4-(2-naphthyl)pyrimidin-2-yl]pyrrolidin-3-yl}methyl)amino]ethanol
2-{(3S)-3-[(4-methylpiperazin-1-yl)methyl]pyrrolidin-1-yl}-4-(2-naphthyl)pyrimidine
(3S)—N,N-dimethyl-1-({(3S)-1-[4-(2-naphthyl)pyrimidin-2-yl]pyrrolidin-3-yl}methyl)pyrrolidin-3-amine
2-[(3R)-3-(1H-imidazol-1-ylmethyl)pyrrolidin-1-yl]-4-(2-naphthyl)pyrimidine
(3R)—N,N-dimethyl-1-({(3S)-1-[4-(2-naphthyl)pyrimidin-2-yl]pyrrolidin-3-yl}methyl)pyrrolidin-3-amine
N,N-dimethyl-1-{(3R)-1-[4-(2-naphthyl)pyrimidin-2-yl]pyrrolidin-3-yl}methanamine
4-(2-naphthyl)-2-[(3R)-3-(piperidin-1-ylmethyl)pyrrolidin-1-yl]pyrimidine
4-(2-naphthyl)-2-[(3R)-3-(pyrrolidin-1-ylmethyl)pyrrolidin-1-yl]pyrimidine
N-methyl-1-{(3R)-1-[4-(2-naphthyl)pyrimidin-2-yl]pyrrolidin-3-yl}methanamine
N-methyl-N-({(3R)-1-[4-(2-naphthyl)pyrimidin-2-yl]pyrrolidin-3-yl}methyl)cyclohexanamine
1-({(3S)-1-[4-(2-naphthyl)pyrimidin-2-yl]pyrrolidin-3-yl}methyl)piperidin-2-one
tert-butyl 4-({(3R)-1-[4-(2-naphthyl)pyrimidin-2-yl]pyrrolidin-3-yl}methyl)piperazine-1-carboxylate
2-methyl-N-({(3R)-1-[4-(2-naphthyl)pyrimidin-2-yl]pyrrolidin-3-yl}methyl)propan-2-amine
2-[ethyl({(3R)-1-[4-(2-naphthyl)pyrimidin-2-yl]pyrrolidin-3-yl}methyl)amino]ethanol
2-{(3R)-3-[(4-methylpiperazin-1-yl)methyl]pyrrolidin-1-yl}-4-(2-naphthyl)pyrimidine
(3S)—N,N-dimethyl-1-({(3R)-1-[4-(2-naphthyl)pyrimidin-2-yl]pyrrolidin-3-yl}methyl)pyrrolidin-3-amine
2-[(3S)-3-(1H-imidazol-1-ylmethyl)pyrrolidin-1-yl]-4-(2-naphthyl)pyrimidine
(3R)—N,N-dimethyl-1-({(3R)-1-[4-(2-naphthyl)pyrimidin-2-yl]pyrrolidin-3-yl}methyl)pyrrolidin-3-amine
N,N-dimethyl-1-[4-(2-naphthyl)pyrimidin-2-yl]-L-prolinamide
4-(2-naphthyl)-2-[(2S)-2-(piperidin-1-ylcarbonyl)pyrrolidin-1-yl]pyrimidine
4-(2-naphthyl)-2-[(2S)-2-(pyrrolidin-1-ylcarbonyl)pyrrolidin-1-yl]pyrimidine
N-tert-butyl-1-[4-(2-naphthyl)pyrimidin-2-yl]-L-prolinamide
N-ethyl-N-(2-hydroxyethyl)-1-[4-(2-naphthyl)pyrimidin-2-yl]-L-prolinamide
2-{(2S)-2-[(4-methylpiperazin-1-yl)carbonyl]pyrrolidin-1-yl}-4-(2-naphthyl)pyrimidine
(3S)—N,N-dimethyl-1-{1-[4-(2-naphthyl)pyrimidin-2-yl]-L-prolyl}pyrrolidin-3-amine
(3R)—N,N-dimethyl-1-{1-[4-(2-naphthyl)pyrimidin-2-yl]-L-prolyl}pyrrolidin-3-amine
N,N-dimethyl-1-[4-(2-naphthyl)pyrimidin-2-yl]-D-prolinamide
4-(2-naphthyl)-2-[(2R)-2-(piperidin-1-ylcarbonyl)pyrrolidin-1-yl]pyrimidine
4-(2-naphthyl)-2-[(2R)-2-(pyrrolidin-1-ylcarbonyl)pyrrolidin-1-yl]pyrimidine
N-methyl-1-[4-(2-naphthyl)pyrimidin-2-yl]-D-prolinamide
N-tert-butyl-1-[4-(2-naphthyl)pyrimidin-2-yl]-D-prolinamide
2-{(2R)-2-[(4-methylpiperazin-1-yl)carbonyl]pyrrolidin-1-yl}-4-(2-naphthyl)pyrimidine
(3S)—N,N-dimethyl-1-{1-[4-(2-naphthyl)pyrimidin-2-yl]-D-prolyl}pyrrolidin-3-amine
(3R)—N,N-dimethyl-1-{1-[4-(2-naphthyl)pyrimidin-2-yl]-D-prolyl}pyrrolidin-3-amine
(3′S)-1′-[4-(2-naphthyl)pyrimidin-2-yl]-1,3′-bipyrrolidin-2-one
(3′R)-1′-[4-(2-naphthyl)pyrimidin-2-yl]-1,3′-bipyrrolidin-2-one
tert-butyl {(3S)-1-[4-(2-naphthyl)pyrimidin-2-yl]pyrrolidin-3-yl}carbamate
2-[(3R)-3-methoxypyrrolidin-1-yl]-4-(2-naphthyl)pyrimidine
2-[(3S)-3-methoxypyrrolidin-1-yl]-4-(2-naphthyl)pyrimidine
2,2,2-trifluoro-N-(2-{(2S)-1-[4-(2-naphthyl)pyrimidin-2-yl]pyrrolidin-2-yl}ethyl)acetamide
2,2,2-trifluoro-N-(2-{(2R)-1-[4-(2-naphthyl)pyrimidin-2-yl]pyrrolidin-2-yl}ethyl)acetamide
(3R)—N-methyl-1-[4-(2-naphthyl)pyrimidin-2-yl]pyrrolidin-3-amine
(3S)—N-methyl-1-[4-(2-naphthyl)pyrimidin-2-yl]pyrrolidin-3-amine
(3R)—N,N-dimethyl-1-[4-(2-naphthyl)pyrimidin-2-yl]pyrrolidin-3-amine
(3S)-1-[4-(2-naphthyl)pyrimidin-2-yl]-N-(2,2,2-trifluoroethyl)pyrrolidin-3-amine
N-{(3S)-1-[4-(2-naphthyl)pyrimidin-2-yl]pyrrolidin-3-yl}-N-(2,2,2-trifluoroethyl)acetamide
2-{(2R)-1-[4-(2-naphthyl)pyrimidin-2-yl]pyrrolidin-2-yl}ethanamine
2-{(2S)-1-[4-(2-naphthyl)pyrimidin-2-yl]pyrrolidin-2-yl}ethanamine
2-{1-[4-(2-naphthyl)pyrimidin-2-yl]pyrrolidin-3-yl}ethanamine
N-methyl-N-{(3R)-1-[4-(2-naphthyl)pyrimidin-2-yl]pyrrolidin-3-yl}acetamide
N-ethyl-N,N′-dimethyl-N′-{(3R)-1-[4-(2-naphthyl)pyrimidin-2-yl]pyrrolidin-3-yl}ethane-1,2-diamine
N,N′-dimethyl-N-{(3R)-1-[4-(2-naphthyl)pyrimidin-2-yl]pyrrolidin-3-yl}-N′-propylethane-1,2-diamine
N-isopropyl-N,N′-dimethyl-N′-{(3R)-1-[4-(2-naphthyl)pyrimidin-2-yl]pyrrolidin-3-yl}ethane-1,2-diamine
N-benzyl-N,N′-dimethyl-N′-{(3R)-1-[4-(2-naphthyl)pyrimidin-2-yl]pyrrolidin-3-yl}ethane-1,2-diamine
N-methyl-N-[2-(methyl{(3R)-1-[4-(2-naphthyl)pyrimidin-2-yl]pyrrolidin-3-yl}amino)ethyl]acetamide
2,2,2-trifluoro-N-methyl-N-[2-(methyl{(3R)-1-[4-(2-naphthyl)pyrimidin-2-yl]pyrrolidin-3-
yl}amino)ethyl]acetamide
N-methyl-N-[2-(methyl{(3R)-1-[4-(2-naphthyl)pyrimidin-2-yl]pyrrolidin-3-yl}amino)ethyl]propanamide
N,2-dimethyl-N-[2-(methyl{(3R)-1-[4-(2-naphthyl)pyrimidin-2-yl]pyrrolidin-3-
yl}amino)ethyl]propanamide
N-methyl-N-[2-(methyl{(3R)-1-[4-(2-naphthyl)pyrimidin-2-yl]pyrrolidin-3-
yl}amino)ethyl]methanesulfonamide
1,1-diethyl-3-methyl-3-[2-(methyl{(3R)-1-[4-(2-naphthyl)pyrimidin-2-yl]pyrrolidin-3-
yl}amino)ethyl]urea
methyl methyl[2-(methyl{(3R)-1-[4-(2-naphthyl)pyrimidin-2-yl]pyrrolidin-3-yl}amino)ethyl]carbamate
N-methyl-N-[2-(methyl{(3R)-1-[4-(2-naphthyl)pyrimidin-2-yl]pyrrolidin-3-
yl}amino)ethyl]ethanesulfonamide
N,N,N′-trimethyl-N′-[2-(methyl{(3R)-1-[4-(2-naphthyl)pyrimidin-2-yl]pyrrolidin-3-
yl}amino)ethyl]sulfamide
N-methyl-N-[2-(methyl{(3R)-1-[4-(2-naphthyl)pyrimidin-2-yl]pyrrolidin-3-yl}amino)ethyl]benzamide
N-methyl-N-[2-(methyl{(3R)-1-[4-(2-naphthyl)pyrimidin-2-yl]pyrrolidin-3-
yl}amino)ethyl]benzenesulfonamide
tert-butyl ({1-[4-(2-naphthyl)pyrimidin-2-yl]azetidin-3-yl}methyl)carbamate
1-{1-[4-(2-naphthyl)pyrimidin-2-yl]azetidin-3-yl}methanamine
methyl ({1-[4-(2-naphthyl)pyrimidin-2-yl]azetidin-3-yl}methyl)carbamate
2,2,2-trifluoro-N-({1-[4-(2-naphthyl)pyrimidin-2-yl]azetidin-3-yl}methyl)acetamide
N,N-dimethyl-N′-({1-[4-(2-naphthyl)pyrimidin-2-yl]azetidin-3-yl}methyl)urea
N-({1-[4-(2-naphthyl)pyrimidin-2-yl]azetidin-3-yl}methyl)urea
N-ethyl-N′-({1-[4-(2-naphthyl)pyrimidin-2-yl]azetidin-3-yl}methyl)urea
N-({1-[4-(2-naphthyl)pyrimidin-2-yl]azetidin-3-yl}methyl)methanesulfonamide
4-methyl-N-({1-[4-(2-naphthyl)pyrimidin-2-yl]azetidin-3-yl}methyl)benzenesulfonamide
tert-butyl {2-[1-(4-naphthalen-2-ylpyrimidin-2-yl)azetidin-3-yl]ethyl}carbamate
2-[1-(4-naphthalen-2-ylpyrimidin-2-yl)azetidin-3-yl]ethanamine
tert-butyl [(trans-4-{[4-(2-naphthyl)pyrimidin-2-yl]oxy}cyclohexyl)methyl]carbamate
1-(trans-4-{[4-(2-naphthyl)pyrimidin-2-yl]oxy}cyclohexyl)methanamine
tert-butyl ({cis-4-[(4-naphthalen-2-ylpyrimidin-2-yl)oxy]cyclohexyl}methyl)carbamate
1-{cis-4-[(4-naphthalen-2-ylpyrimidin-2-yl)oxy]cyclohexyl}methanamine
N-[trans-4-(aminomethyl)cyclohexyl]-N-methyl-4-(2-naphthyl)pyrimidin-2-amine
benzyl [(cis-4-{methyl[4-(2-naphthyl)pyrimidin-2-yl]amino}cyclohexyl)methyl]carbamate
N-[cis-4-(aminomethyl)cyclohexyl]-N-methyl-4-(2-naphthyl)pyrimidin-2-amine
1-{4-[4-(2-naphthyl)pyrimidin-2-yl]phenyl}methanamine
1-[trans-4-(4-naphthalen-2-ylpyrimidin-2-yl)cyclohexyl]methanamine
1-{cis-4-[4-(2-naphthyl)pyrimidin-2-yl]cyclohexyl}methanamine
1-{1-[4-(6-methoxy-2-naphthyl)pyrimidin-2-yl]piperidin-4-yl}methanamine
1-{1-[4-(6-propoxy-2-naphthyl)pyrimidin-2-yl]piperidin-4-yl}methanamine
1-{1-[4-(6-isobutoxy-2-naphthyl)pyrimidin-2-yl]piperidin-4-yl}methanamine
6-{2-[4-(aminomethyl)piperidin-1-yl]pyrimidin-4-yl}-2-naphthyl acetate
6-{2-[4-(aminomethyl)piperidin-1-yl]pyrimidin-4-yl}-2-naphthol
2,2,2-trifluoro-N-({1-[4-(6-hydroxy-2-naphthyl)pyrimidin-2-yl]piperidin-4-yl}methyl)acetamide
2,2,2-trifluoro-N-({1-[4-(6-methoxy-2-naphthyl)pyrimidin-2-yl]piperidin-4-yl}methyl)acetamide
1-(1-{4-[6-(2-thienyl)-2-naphthyl]pyrimidin-2-yl}piperidin-4-yl)methanamine
1-(1-{4-[6-(2-methoxyphenyl)-2-naphthyl]pyrimidin-2-yl}piperidin-4-yl)methanamine
1-(1-{4-[6-(4-methoxyphenyl)-2-naphthyl]pyrimidin-2-yl}piperidin-4-yl)methanamine
1-{1-[4-(6-phenyl-2-naphthyl)pyrimidin-2-yl]piperidin-4-yl}methanamine
2,2,2-trifluoro-N-({1-[4-(6-formyl-2-naphthyl)pyrimidin-2-yl]piperidin-4-yl}methyl)acetamide
1-{1-[4-(6-vinyl-2-naphthyl)pyrimidin-2-yl]piperidin-4-yl}methanamine
1-{1-[4-(6-methyl-2-naphthyl)pyrimidin-2-yl]piperidin-4-yl}methanamine
1-(1-{4-[6-(piperidin-1-ylmethyl)-2-naphthyl]pyrimidin-2-yl}piperidin-4-yl)methanamine
1-(1-{4-[6-(morpholin-4-ylmethyl)-2-naphthyl]pyrimidin-2-yl}piperidin-4-yl)methanamine
2-{4-[(4-methylpiperazin-1-yl)methyl]piperidin-1-yl}-4-(2-naphthyl)pyrimidine
N-methyl-N-({1-[4-(2-naphthyl)pyrimidin-2-yl]piperidin-4-yl}methyl)cyclohexanamine
1-({1-[4-(2-naphthyl)pyrimidin-2-yl]piperidin-4-yl}methyl)azepane
2-[ethyl({1-[4-(2-naphthyl)pyrimidin-2-yl]piperidin-4-yl}methyl)amino]ethanol
2-methyl-N-({1-[4-(2-naphthyl)pyrimidin-2-yl]piperidin-4-yl}methyl)propan-2-amine
1-({1-[4-(2-naphthyl)pyrimidin-2-yl]piperidin-4-yl}methyl)pyrrolidin-2-one
1-({1-[4-(2-naphthyl)pyrimidin-2-yl]piperidin-4-yl}methyl)piperidin-2-one
4-(2-naphthyl)-2-[4-(piperazin-1-ylmethyl)piperidin-1-yl]pyrimidine
4-({1-[4-(2-naphthyl)pyrimidin-2-yl]piperidin-4-yl}methyl)morpholine
4-({1-[4-(2-naphthyl)pyrimidin-2-yl]piperidin-4-yl}methyl)piperazin-2-one
1-(1-{[4-(2-naphthyl)pyrimidin-2-yl]carbonyl}piperidin-4-yl)methanamine
(3aR,7aS)-5-[4-(2-naphthyl)pyrimidin-2-yl]octahydro-1H-pyrrolo[3,4-c]pyridine

In one aspect, the invention provides compounds of the Formula I:

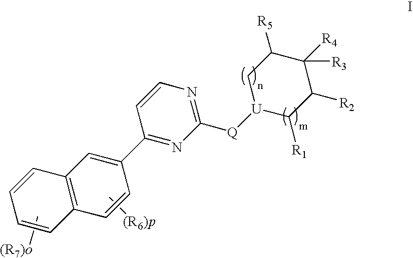

or pharmaceutically acceptable salts thereof,
wherein

R₁, R₂, R₃, R₄, R₅, R₆, R₇, R₈, R₉, R₁₀, R₁₁, R₁₂, R₁₃, R₁₄, Q, U, m, n, o, and p are as defined above for the compounds of Formula I.

In one embodiment, Q is a bond. In one embodiment, Q is CR₈R₉.

In one embodiment, U is N.

In one embodiment, N is CR₁₀.

In one embodiment, R₁ is H. In one embodiment, R₁ is C₁-C₆ alkyl.

In one embodiment, R₂ is C₁-C₁₀ alkyl substituted with 0, 1 or 2 of NR₁₁R₁₂, COR₁₁, CO₂R₁₁, CONR₁₁R₁₂, OR₁₁, S(O)ₓR₁₁, or SO₂NR₁₁R₁₂.

In one embodiment, R₁ and R₂ are taken together with the ring to which they are attached to form an 8- to 12-membered bicyclic heterocycle.

In one embodiment, R₃ is H or halogen.

In one embodiment, R₃ is C₁-C₁₀ alkyl substituted with 0, 1 or 2 of NR₁₁R₁₂, COR₁₁, CO₂R₁₁, CONR₁₁R₁₂, OR₁₁, S(O)ₓR₁₁, or SO₂NR₁₁R₁₂.

In one embodiment, R₂ and R₃ are taken together with the ring to which they are attached to form an 8- to 12-membered bicyclic heterocycle.

In one embodiment, R₄ is H or halogen. In one embodiment, R₄ is OR₁₁ or NR₁₁R₁₂. In one embodiment, R₄ is C₁-C₆ alkyl substituted with at least one and up to two of NR₁₀R₁₁, COR₁₀, CO₂R₁₀, CONR₁₀R₁₁, OR₁₀, S(O)ₓR₁₀, or SO₂NR₁₀R₁₁.

In one embodiment, R₃ and R₄ are taken together with the carbon to which they are attached to form a C₃-C₈ monocyclic cycloalkyl. In one embodiment, R₃ and R₄ are taken together with the carbon to which they are attached to form a 3- to 7-membered monocyclic heterocycle.

In one embodiment, R₅is H, OR₁₀ or NR₁₀R₁₁ In one embodiment, R₅is C₁-C₆ alkyl optionally substituted with OR₁₀, or NR₁₀R₁₁.

In one embodiment, R₅ and R₄ are taken together to form a C₃-C₈ monocyclic cycloalkyl.

In one embodiment, R₅ and R₄ are taken together to form a 3- to 7-membered monocyclic heterocycle.

In one embodiment, R₅ and R₂, are taken together along with the ring to which they are attached to form an C₈-C₁₂ bicyclic cycloalkyl. In one embodiment, R₅ and R₂, are taken together along with the ring to which they are attached to form an 8- to 12-membered bicyclic heterocycle.

In one embodiment, R₅ and R₁, are taken together along with the ring to which they are attached to form an C₈-C₁₂ bicyclic cycloalkyl. In one embodiment, R₅ and R₁, are taken together along with the ring to which they are attached to form an 8- to 12-membered bicyclic heterocycle.

In one embodiment, R₆ is H, halogen, CN, NO₂, R₁₁, OR₁₁, S(O)ₓR₁₁, or NR₁₁R₁₂.

In one embodiment, R₇ is H, halogen, CN, NO₂, R₁₁, OR₁₁, S(O)ₓR₁₁, or NR₁₁R₁₂.

In one embodiment, R₈ and R₉ form an ═O.

In one embodiment, R₈ is H or C₁-C₆ alkyl.

In one embodiment, R₉ is H or C₁-C₆ alkyl.

In one embodiment, R₁₀ is H. In one embodiment, R₁₀ is C₁₋C₆ alkyl.

In one embodiment, R₁₁ is H. In one embodiment, R₁₁ is C₁₁₋C₆ alkyl. In one embodiment, R₁₁ is aryl. In one embodiment, R₁₁ alkylaryl.

In one embodiment, R₁₂ is H, C₁₋C₆ alkyl, aryl, alkylaryl, COR₁₃, CO₂R₁₃, CONR₁₃R₁₄, SO₂R₁₃.

In one embodiment, R₁₁ and R₁₂ are taken together with the N to which they are attached to form a C₃-C₈ monocyclic cycloalkyl optionally substituted with R₁₁ and OR₁₁.

In one embodiment, R₁₁ and R₁₂ are taken together with the N to which they are attached to form a 3- to 7-membered monocyclic heterocycle optionally substituted with R₁₁ and OR₁₁.

In one embodiment, R₁₁ and R₁₂ are taken together with the N to which they are attached to form an C₈-C₁₂ bicyclic cycloalkyl optionally substituted with R₁₁ and OR₁₁.

In one embodiment, R₁₁ and R₁₂ are taken together with the N to which they are attached to form an 8- to 12-membered bicyclic heterocycle optionally substituted with R₁₁ and OR₁₁.

In one embodiment, R₁₃ is H or C₁₋C₆ alkyl.

In one embodiment, R₁₄ is H or C₁₋C₆ alkyl.

In one embodiment, R₁₃ and R₁₄ are taken together with the N to which they are attached to form a C₃-C₈ monocyclic cycloalkyl. In one embodiment, R₁₃ and R₁₄ are taken together with the N to which they are attached to form a 3- to 7-membered monocyclic heterocycle.

In one embodiment, m is 0. In one embodiment, m is 1. In one embodiment, m is 2.

In one embodiment, n is 0. In one embodiment, n is 1. In one embodiment, n is 2.

In one embodiment, o is 0. In one embodiment, o is 1. In one embodiment, o is 2.

In one embodiment, p is 0. In one embodiment, p is 1. In one embodiment, p is 2.

In one embodiment, x is 0. In one embodiment, x is 1.

In one embodiment, m is 2.

Illustrative compounds of Formula I are exemplified by the following compounds:

Compound

tert-butyl {(3S)-1-[4-(2-naphthyl)pyrimidin-2-yl]pyrrolidin-3-yl}carbamate
(3S)-1-[4-(2-naphthyl)pyrimidin-2-yl]pyrrolidin-3-amine
4-[4-(2-naphthyl)pyrimidin-2-yl]-1,4-diazepane-1-carbaldehyde
1-[4-(2-naphthyl)pyrimidin-2-yl]-1,4-diazepane
1-[4-(2-naphthyl)pyrimidin-2-yl]azepan-4-amine
tert-butyl {1-[4-(2-naphthyl)pyrimidin-2-yl]azepan-4-yl}carbamate
tert-butyl ({1-[4-(2-naphthyl)pyrimidin-2-yl]azetidin-3-yl}methyl)carbamate
1-{1-[4-(2-naphthyl)pyrimidin-2-yl]azetidin-3-yl}methanamine
tert-butyl ({1-[4-(2-naphthyl)pyrimidin-2-yl]pyrrolidin-3-yl}methyl)carbamate
1-{1-[4-(2-naphthyl)pyrimidin-2-yl]pyrrolidin-3-yl}methanamine
4-(2-{4-[4-(2-naphthyl)pyrimidin-2-yl]piperazin-1-yl}ethyl)morpholine
4-({4-[4-(2-naphthyl)pyrimidin-2-yl]piperazin-1-yl}acetyl)morpholine
N,N-dimethyl-2-{4-[4-(2-naphthyl)pyrimidin-2-yl]piperazin-1-yl}ethanamine
2-(4-methylpiperazin-1-yl)-4-(2-naphthyl)pyrimidine
4-(2-naphthyl)-2-(4-pyridin-2-ylpiperazin-1-yl)pyrimidine
4-(2-naphthyl)-2-(4-pyrimidin-2-ylpiperazin-1-yl)pyrimidine
1-{1-[4-(2-naphthyl)pyrimidin-2-yl]piperidin-4-yl}-1,3-dihydro-2H-benzimidazol-2-one
2-[4-(2-naphthyl)pyrimidin-2-yl]-1,2,3,4-tetrahydroisoquinoline
(2-{4-[4-(2-naphthyl)pyrimidin-2-yl]piperazin-1-yl}ethyl)amine
N,N-dimethyl-3-{4-[4-(2-naphthyl)pyrimidin-2-yl]piperazin-1-yl}propan-1-amine
4-(2-naphthyl)-2-(4-pyridin-4-ylpiperazin-1-yl)pyrimidine
4-(3-{4-[4-(2-naphthyl)pyrimidin-2-yl]piperazin-1-yl}propyl)morpholine
2-[4-(2-furoyl)piperazin-1-yl]-4-(2-naphthyl)pyrimidine
tert-butyl {(3R)-1-[4-(2-naphthyl)pyrimidin-2-yl]pyrrolidin-3-yl}carbamate
(3R)-1-[4-(2-naphthyl)pyrimidin-2-yl]pyrrolidin-3-amine
N,N-dimethyl-1-[4-(2-naphthyl)pyrimidin-2-yl]pyrrolidin-3-amine
N,N-diethyl-1-[4-(2-naphthyl)pyrimidin-2-yl]pyrrolidin-3-amine
(3R)—N-methyl-1-[4-(2-naphthyl)pyrimidin-2-yl]pyrrolidin-3-amine
(3S)—N-methyl-1-[4-(2-naphthyl)pyrimidin-2-yl]pyrrolidin-3-amine
(3S)—N,N-dimethyl-1-[4-(2-naphthyl)pyrimidin-2-yl]pyrrolidin-3-amine
(3R)—N,N-dimethyl-1-[4-(2-naphthyl)pyrimidin-2-yl]pyrrolidin-3-amine
N-methyl-N-{(3S)-1-[4-(2-naphthyl)pyrimidin-2-yl]pyrrolidin-3-yl}acetamide
{1-[4-(2-naphthyl)pyrimidin-2-yl]piperidin-4-yl}methanol
2-{1-[4-(2-naphthyl)pyrimidin-2-yl]piperidin-4-yl}ethanol
ethyl 1-[4-(2-naphthyl)pyrimidin-2-yl]piperidine-4-carboxylate
ethyl {1-[4-(2-naphthyl)pyrimidin-2-yl]piperidin-4-yl}acetate
{(2S)-1-[4-(2-naphthyl)pyrimidin-2-yl]pyrrolidin-2-yl}methanol
1-[4-(2-naphthyl)pyrimidin-2-yl]pyrrolidin-3-ol
4-[4-(2-naphthyl)pyrimidin-2-yl]morpholine
{1-[4-(2-naphthyl)pyrimidin-2-yl]piperidin-2-yl}methanol
{1-[4-(2-naphthyl)pyrimidin-2-yl]piperidin-3-yl}methanol
1-[4-(2-naphthyl)pyrimidin-2-yl]piperidin-4-ol
1-[4-(2-naphthyl)pyrimidin-2-yl]piperidin-3-ol
{(2R)-1-[4-(2-naphthyl)pyrimidin-2-yl]pyrrolidin-2-yl}methanol
(1-(4-(naphthalen-2-yl)pyrimidin-2-yl)piperidin-4-yl)methanol
4-(2-naphthyl)-2-pyrrolidin-1-ylpyrimidine
4-(2-naphthyl)-2-piperidin-1-ylpyrimidine
2-(4-methylpiperidin-1-yl)-4-(2-naphthyl)pyrimidine
1-[4-(2-naphthyl)pyrimidin-2-yl]azepane
tert-butyl {1-[4-(2-naphthyl)pyrimidin-2-yl]piperidin-4-yl}carbamate
tert-butyl 4-[4-(2-naphthyl)pyrimidin-2-yl]piperazine-1-carboxylate
1-[4-(2-naphthyl)pyrimidin-2-yl]piperidin-4-amine
4-(2-naphthyl)-2-piperazin-1-ylpyrimidine
tert-butyl ({1-[4-(2-naphthyl)pyrimidin-2-yl]piperidin-3-yl}methyl)carbamate
({1-[4-(2-naphthyl)pyrimidin-2-yl]piperidin-3-yl}methyl)amine
(1R,5S,6s)-3-[4-(2-naphthyl)pyrimidin-2-yl]-3-azabicyclo[3.1.0]hexan-6-amine
({1-[4-(2-naphthyl)pyrimidin-2-yl]piperidin-4-yl}methyl)amine
1-[4-(2-naphthyl)pyrimidin-2-yl]piperidine-4-carboxamide
8-[4-(2-naphthyl)pyrimidin-2-yl]-1,4-dioxa-8-azaspiro[4.5]decane
methyl 1-[4-(2-naphthyl)pyrimidin-2-yl]piperidine-4-carboxylate
1-[4-(2-naphthyl)pyrimidin-2-yl]piperidine-3-carboxamide
{(3S)-1-[4-(2-naphthyl)pyrimidin-2-yl]piperidin-3-yl}methanol
{(3R)-1-[4-(2-naphthyl)pyrimidin-2-yl]piperidin-3-yl}methanol
N-{(3S)-1-[4-(2-naphthyl)pyrimidin-2-yl]pyrrolidin-3-yl}acetamide
N-({1-[4-(2-naphthyl)pyrimidin-2-yl]piperidin-4-yl}methyl)guanidine
N-ethyl-4-[4-(2-naphthyl)pyrimidin-2-yl]-1,4-diazepane-1-carboxamide
methyl 4-[4-(2-naphthyl)pyrimidin-2-yl]-1,4-diazepane-1-carboxylate
ethyl 4-[4-(2-naphthyl)pyrimidin-2-yl]-1,4-diazepane-1-carboxylate
1-acetyl-4-[4-(2-naphthyl)pyrimidin-2-yl]-1,4-diazepane
1-[4-(2-naphthyl)pyrimidin-2-yl]-4-propionyl-1,4-diazepane
1-[4-(2-naphthyl)pyrimidin-2-yl]-4-(trifluoroacetyl)-1,4-diazepane
N,N-diethyl-4-[4-(2-naphthyl)pyrimidin-2-yl]-1,4-diazepane-1-carboxamide
1-(methylsulfonyl)-4-[4-(2-naphthyl)pyrimidin-2-yl]-1,4-diazepane
N,N-dimethyl-4-[4-(2-naphthyl)pyrimidin-2-yl]-1,4-diazepane-1-sulfonamide
N,N-dimethyl-4-[4-(2-naphthyl)pyrimidin-2-yl]-1,4-diazepane-1-carboxamide
4-[4-(2-naphthyl)pyrimidin-2-yl]-1,4-diazepane-1-carboxamide
1-benzoyl-4-[4-(2-naphthyl)pyrimidin-2-yl]-1,4-diazepane
1-[(4-methylphenyl)sulfonyl]-4-[4-(2-naphthyl)pyrimidin-2-yl]-1,4-diazepane
N-{1-[4-(2-naphthyl)pyrimidin-2-yl]azepan-4-yl}acetamide
2,2,2-trifluoro-N-{1-[4-(2-naphthyl)pyrimidin-2-yl]azepan-4-yl}acetamide
N,N-dimethyl-N′-{1-[4-(2-naphthyl)pyrimidin-2-yl]azepan-4-yl}urea
N-{1-[4-(2-naphthyl)pyrimidin-2-yl]azepan-4-yl}benzamide
N-{1-[4-(2-naphthyl)pyrimidin-2-yl]azepan-4-yl}urea
N-ethyl-N′-{1-[4-(2-naphthyl)pyrimidin-2-yl]azepan-4-yl}urea
N-{1-[4-(2-naphthyl)pyrimidin-2-yl]azepan-4-yl}methanesulfonamide
4-methyl-N-{1-[4-(2-naphthyl)pyrimidin-2-yl]azepan-4-yl}benzenesulfonamide
N,N-dimethyl-N′-{1-[4-(2-naphthyl)pyrimidin-2-yl]azepan-4-yl}sulfamide
N-({1-[4-(2-naphthyl)pyrimidin-2-yl]azetidin-3-yl}methyl)acetamide
2,2,2-trifluoro-N-({1-[4-(2-naphthyl)pyrimidin-2-yl]azetidin-3-yl}methyl)acetamide
N,N-dimethyl-N′-({1-[4-(2-naphthyl)pyrimidin-2-yl]azetidin-3-yl}methyl)urea
methyl ({1-[4-(2-naphthyl)pyrimidin-2-yl]azetidin-3-yl}methyl)carbamate
N-({1-[4-(2-naphthyl)pyrimidin-2-yl]azetidin-3-yl}methyl)urea
N-ethyl-N′-({1-[4-(2-naphthyl)pyrimidin-2-yl]azetidin-3-yl}methyl)urea
N-({1-[4-(2-naphthyl)pyrimidin-2-yl]azetidin-3-yl}methyl)methanesulfonamide
4-methyl-N-({1-[4-(2-naphthyl)pyrimidin-2-yl]azetidin-3-yl}methyl)benzenesulfonamide
{1-[4-(2-naphthyl)pyrimidin-2-yl]azepan-4-yl}formamide
N,N-dimethyl-N′-(2-{4-[4-(2-naphthyl)pyrimidin-2-yl]piperazin-1-yl}ethyl)urea
N-(2-{4-[4-(2-naphthyl)pyrimidin-2-yl]piperazin-1-yl}ethyl)methanesulfonamide
N-(2-{4-[4-(2-naphthyl)pyrimidin-2-yl]piperazin-1-yl}ethyl)dicarbonimidic diamide
N-ethyl-N′-(2-{4-[4-(2-naphthyl)pyrimidin-2-yl]piperazin-1-yl}ethyl)urea
N-isopropyl-N′-(2-{4-[4-(2-naphthyl)pyrimidin-2-yl]piperazin-1-yl}ethyl)urea
diethyl (2-{4-[4-(2-naphthyl)pyrimidin-2-yl]piperazin-1-yl}ethyl)imidodicarbonate
N-{(3R)-1-[4-(2-naphthyl)pyrimidin-2-yl]pyrrolidin-3-yl}acetamide
2,2,2-trifluoro-N-{(3R)-1-[4-(2-naphthyl)pyrimidin-2-yl]pyrrolidin-3-yl}acetamide
methyl {(3R)-1-[4-(2-naphthyl)pyrimidin-2-yl]pyrrolidin-3-yl}carbamate
N,N-dimethyl-N′-{(3R)-1-[4-(2-naphthyl)pyrimidin-2-yl]pyrrolidin-3-yl}urea
N-{(3R)-1-[4-(2-naphthyl)pyrimidin-2-yl]pyrrolidin-3-yl}pyrrolidine-1-carboxamide
N-{(3R)-1-[4-(2-naphthyl)pyrimidin-2-yl]pyrrolidin-3-yl}urea
N,N-dimethyl-N′-{(3R)-1-[4-(2-naphthyl)pyrimidin-2-yl]pyrrolidin-3-yl}sulfamide
N-{(3R)-1-[4-(2-naphthyl)pyrimidin-2-yl]pyrrolidin-3-yl}methanesulfonamide
4-methyl-N-{(3R)-1-[4-(2-naphthyl)pyrimidin-2-yl]pyrrolidin-3-yl}benzenesulfonamide
N-{(3R)-1-[4-(2-naphthyl)pyrimidin-2-yl]pyrrolidin-3-yl}morpholine-4-carboxamide
ethyl {(3R)-1-[4-(2-naphthyl)pyrimidin-2-yl]pyrrolidin-3-yl}carbamate
N-isopropyl-N′-{(3R)-1-[4-(2-naphthyl)pyrimidin-2-yl]pyrrolidin-3-yl}urea
N,N-diethyl-N′-{(3R)-1-[4-(2-naphthyl)pyrimidin-2-yl]pyrrolidin-3-yl}urea
N-{(3R)-1-[4-(2-naphthyl)pyrimidin-2-yl]pyrrolidin-3-yl}guanidine
4-chloro-N-{(3R)-1-[4-(2-naphthyl)pyrimidin-2-yl]pyrrolidin-3-yl}benzamide
4-cyano-N-{(3R)-1-[4-(2-naphthyl)pyrimidin-2-yl]pyrrolidin-3-yl}benzamide
N-{(3S)-1-[4-(2-naphthyl)pyrimidin-2-yl]pyrrolidin-3-yl}acetamide
2,2,2-trifluoro-N-{(3S)-1-[4-(2-naphthyl)pyrimidin-2-yl]pyrrolidin-3-yl}acetamide
N-{(3S)-1-[4-(2-naphthyl)pyrimidin-2-yl]pyrrolidin-3-yl}benzamide
methyl {(3S)-1-[4-(2-naphthyl)pyrimidin-2-yl]pyrrolidin-3-yl}carbamate
N,N-dimethyl-N′-{(3S)-1-[4-(2-naphthyl)pyrimidin-2-yl]pyrrolidin-3-yl}urea
N-{(3S)-1-[4-(2-naphthyl)pyrimidin-2-yl]pyrrolidin-3-yl}pyrrolidine-1-carboxamide
N-ethyl-N′-{(3S)-1-[4-(2-naphthyl)pyrimidin-2-yl]pyrrolidin-3-yl}urea
N-{(3S)-1-[4-(2-naphthyl)pyrimidin-2-yl]pyrrolidin-3-yl}urea
N,N-dimethyl-N′-{(3S)-1-[4-(2-naphthyl)pyrimidin-2-yl]pyrrolidin-3-yl}sulfamide
N-{(3S)-1-[4-(2-naphthyl)pyrimidin-2-yl]pyrrolidin-3-yl}methanesulfonamide
4-methyl-N-{(3S)-1-[4-(2-naphthyl)pyrimidin-2-yl]pyrrolidin-3-yl}benzenesulfonamide
N-{(3S)-1-[4-(2-naphthyl)pyrimidin-2-yl]pyrrolidin-3-yl}morpholine-4-carboxamide
ethyl {(3S)-1-[4-(2-naphthyl)pyrimidin-2-yl]pyrrolidin-3-yl}carbamate
N-isopropyl-N′-{(3S)-1-[4-(2-naphthyl)pyrimidin-2-yl]pyrrolidin-3-yl}urea
N,N-diethyl-N′-{(3S)-1-[4-(2-naphthyl)pyrimidin-2-yl]pyrrolidin-3-yl}urea
4-chloro-N-{(3S)-1-[4-(2-naphthyl)pyrimidin-2-yl]pyrrolidin-3-yl}benzamide
4-cyano-N-{(3S)-1-[4-(2-naphthyl)pyrimidin-2-yl]pyrrolidin-3-yl}benzamide
N-({1-[4-(2-naphthyl)pyrimidin-2-yl]pyrrolidin-3-yl}methyl)acetamide
N-({1-[4-(2-naphthyl)pyrimidin-2-yl]pyrrolidin-3-yl}methyl)benzamide
methyl ({1-[4-(2-naphthyl)pyrimidin-2-yl]pyrrolidin-3-yl}methyl)carbamate
N,N-dimethyl-N′-({1-[4-(2-naphthyl)pyrimidin-2-yl]pyrrolidin-3-yl}methyl)urea
N-ethyl-N′-({1-[4-(2-naphthyl)pyrimidin-2-yl]pyrrolidin-3-yl}methyl)urea
N-({1-[4-(2-naphthyl)pyrimidin-2-yl]pyrrolidin-3-yl}methyl)urea
N,N-dimethyl-N′-({1-[4-(2-naphthyl)pyrimidin-2-yl]pyrrolidin-3-yl}methyl)sulfamide
N-({1-[4-(2-naphthyl)pyrimidin-2-yl]pyrrolidin-3-yl}methyl)methanesulfonamide
N-({1-[4-(2-naphthyl)pyrimidin-2-yl]pyrrolidin-3-yl}methyl)formamide
N-(2-{1-[4-(2-naphthyl)pyrimidin-2-yl]piperidin-4-yl}ethyl)acetamide
2,2,2-trifluoro-N-(2-{1-[4-(2-naphthyl)pyrimidin-2-yl]piperidin-4-yl}ethyl)acetamide
methyl (2-{1-[4-(2-naphthyl)pyrimidin-2-yl]piperidin-4-yl}ethyl)carbamate
N,N-dimethyl-N′-(2-{1-[4-(2-naphthyl)pyrimidin-2-yl]piperidin-4-yl}ethyl)urea
N,N-diethyl-N′-(2-{1-[4-(2-naphthyl)pyrimidin-2-yl]piperidin-4-yl}ethyl)urea
N-ethyl-N′-(2-{1-[4-(2-naphthyl)pyrimidin-2-yl]piperidin-4-yl}ethyl)urea
N-isopropyl-N′-(2-{1-[4-(2-naphthyl)pyrimidin-2-yl]piperidin-4-yl}ethyl)urea
N-(2-{1-[4-(2-naphthyl)pyrimidin-2-yl]piperidin-4-yl}ethyl)urea
N-cyclohexyl-N′-(2-{1-[4-(2-naphthyl)pyrimidin-2-yl]piperidin-4-yl}ethyl)urea
N-(2-{1-[4-(2-naphthyl)pyrimidin-2-yl]piperidin-4-yl}ethyl)pyrrolidine-1-carboxamide
N-(2-{1-[4-(2-naphthyl)pyrimidin-2-yl]piperidin-4-yl}ethyl)morpholine-4-carboxamide
N,N-dimethyl-N′-(2-{1-[4-(2-naphthyl)pyrimidin-2-yl]piperidin-4-yl}ethyl)sulfamide
N-(2-{1-[4-(2-naphthyl)pyrimidin-2-yl]piperidin-4-yl}ethyl)methanesulfonamide
benzyl (2-{1-[4-(2-naphthyl)pyrimidin-2-yl]piperidin-4-yl}ethyl)carbamate
N-(2-{1-[4-(2-naphthyl)pyrimidin-2-yl]piperidin-4-yl}ethyl)-N′-phenylurea
N-(2-{1-[4-(2-naphthyl)pyrimidin-2-yl]piperidin-4-yl}ethyl)benzamide
4-{methyl[4-(2-naphthyl)pyrimidin-2-yl]amino}piperidine-1-carbaldehyde
4-{methyl[4-(2-naphthyl)pyrimidin-2-yl]amino}piperidine-1-carboximidamide
N-({1-[4-(2-naphthyl)pyrimidin-2-yl]piperidin-4-yl}methyl)acetamide
N-({1-[4-(2-naphthyl)pyrimidin-2-yl]piperidin-4-yl}methyl)benzamide
methyl ({1-[4-(2-naphthyl)pyrimidin-2-yl]piperidin-4-yl}methyl)carbamate
N,N-dimethyl-N′-({1-[4-(2-naphthyl)pyrimidin-2-yl]piperidin-4-yl}methyl)urea
N-({1-[4-(2-naphthyl)pyrimidin-2-yl]piperidin-4-yl}methyl)pyrrolidine-1-carboxamide
N-ethyl-N′-({1-[4-(2-naphthyl)pyrimidin-2-yl]piperidin-4-yl}methyl)urea
N-({1-[4-(2-naphthyl)pyrimidin-2-yl]piperidin-4-yl}methyl)urea
N,N-dimethyl-N′-({1-[4-(2-naphthyl)pyrimidin-2-yl]piperidin-4-yl}methyl)sulfamide
N-({1-[4-(2-naphthyl)pyrimidin-2-yl]piperidin-4-yl}methyl)methanesulfonamide
4-methyl-N-({1-[4-(2-naphthyl)pyrimidin-2-yl]piperidin-4-yl}methyl)benzenesulfonamide
N-({1-[4-(2-naphthyl)pyrimidin-2-yl]piperidin-4-yl}methyl)pyrimidin-2-amine
di-tert-butyl {(Z)-[({1-[4-(2-naphthyl)pyrimidin-2-yl]piperidin-4-
yl}methyl)amino]methylylidene}biscarbamate
di-tert-butyl ((E)-{4-[4-(2-naphthyl)pyrimidin-2-yl]piperazin-1-yl}methylylidene)biscarbamate
4-[4-(2-naphthyl)pyrimidin-2-yl]piperazine-1-carboximidamide
{1-[4-(2-naphthyl)pyrimidin-2-yl]piperidin-4-yl}methyl 4-methylbenzenesulfonate
2-{1-[4-(2-naphthyl)pyrimidin-2-yl]piperidin-4-yl}ethyl methanesulfonate
2-[4-(2-azidoethyl)piperidin-1-yl]-4-(2-naphthyl)pyrimidine
N,N-dimethyl-1-{1-[4-(2-naphthyl)pyrimidin-2-yl]piperidin-4-yl}methanamine
N-methyl-1-{1-[4-(2-naphthyl)pyrimidin-2-yl]piperidin-4-yl}methanamine
2-[4-(1H-imidazol-1-ylmethyl)piperidin-1-yl]-4-(2-naphthyl)pyrimidine
4-(2-naphthyl)-2-[4-(pyrrolidin-1-ylmethyl)piperidin-1-yl]pyrimidine
N-ethyl-N-({1-[4-(2-naphthyl)pyrimidin-2-yl]piperidin-4-yl}methyl)ethanamine
4-(2-naphthyl)-2-[4-(piperidin-1-ylmethyl)piperidin-1-yl]pyrimidine
N-methyl-1-{1-[4-(2-naphthyl)pyrimidin-2-yl]piperidin-4-yl}methanamine
N-({1-[4-(2-naphthyl)pyrimidin-2-yl]piperidin-4-yl}methyl)ethanamine
2-methyl-N-({1-[4-(2-naphthyl)pyrimidin-2-yl]piperidin-4-yl}methyl)propan-1-amine
2-{1-[4-(2-naphthyl)pyrimidin-2-yl]piperidin-4-yl}ethanamine
1-[4-(2-naphthyl)pyrimidin-2-yl]piperidin-4-one
1-[4-(2-naphthyl)pyrimidin-2-yl]piperidine-4-carbaldehyde
1-[4-(2-naphthyl)pyrimidin-2-yl]piperidine-4-carboxylic acid
4-methyl-1-[4-(2-naphthyl)pyrimidin-2-yl]piperidin-4-ol
1-[4-(2-naphthyl)pyrimidin-2-yl]-4-(nitromethyl)piperidin-4-ol
4-(aminomethyl)-1-[4-(2-naphthyl)pyrimidin-2-yl]piperidin-4-ol
1-[4-(2-naphthyl)pyrimidin-2-yl]-4-(1-nitroethyl)piperidin-4-ol
4-(2-naphthyl)-2-[4-(1-nitroethyl)piperidin-1-yl]pyrimidine
4-(2-naphthyl)-2-[4-(nitromethyl)piperidin-1-yl]pyrimidine
tert-butyl ((1S)-1-{1-[4-(2-naphthyl)pyrimidin-2-yl]piperidin-4-yl}ethyl)carbamate
tert-butyl ((1R)-1-{1-[4-(2-naphthyl)pyrimidin-2-yl]piperidin-4-yl}ethyl)carbamate
tert-butyl (1-{1-[4-(2-naphthyl)pyrimidin-2-yl]piperidin-4-yl}ethyl)carbamate
1-{1-[4-(2-naphthyl)pyrimidin-2-yl]piperidin-4-yl}ethanamine
(1R)-1-{1-[4-(2-naphthyl)pyrimidin-2-yl]piperidin-4-yl}ethanamine
(1S)-1-{1-[4-(2-naphthyl)pyrimidin-2-yl]piperidin-4-yl}ethanamine
4-{[4-(2-naphthyl)pyrimidin-2-yl]methyl}-1,4-diazepane-1-carbaldehyde
1-(1-{[4-(2-naphthyl)pyrimidin-2-yl]methyl}piperidin-4-yl)methanamine
1-[4-(2-naphthyl)pyrimidin-2-yl]piperidine-4-carbonitrile
1-[4-(2-naphthyl)pyrimidin-2-yl]piperidine-4-carbothioamide
2-azetidin-1-yl-4-(2-naphthyl)pyrimidine
2-[4-(azidomethyl)piperidin-1-yl]-4-(2-naphthyl)pyrimidine
1-[4-(2-naphthyl)pyrimidin-2-yl]piperidine-4-carboximidamide
methyl [(tert-butoxycarbonyl)amino]{1-[4-(2-naphthyl)pyrimidin-2-yl]piperidin-4-ylidene}acetate
methyl [(tert-butoxycarbonyl)amino]{1-[4-(2-naphthyl)pyrimidin-2-yl]piperidin-4-yl}acetate
methyl (2R)-[(tert-butoxycarbonyl)amino]{1-[4-(2-naphthyl)pyrimidin-2-yl]piperidin-4-yl}acetate
methyl (2S)-[(tert-butoxycarbonyl)amino]{1-[4-(2-naphthyl)pyrimidin-2-yl]piperidin-4-yl}acetate
methyl amino{1-[4-(2-naphthyl)pyrimidin-2-yl]piperidin-4-yl}acetate
methyl (2R)-amino{1-[4-(2-naphthyl)pyrimidin-2-yl]piperidin-4-yl}acetate
methyl (2S)-amino{1-[4-(2-naphthyl)pyrimidin-2-yl]piperidin-4-yl}acetate
(2R)-2-amino-2-{1-[4-(2-naphthyl)pyrimidin-2-yl]piperidin-4-yl}ethanol
tert-butyl ((1R)-2-hydroxy-1-{1-[4-(2-naphthyl)pyrimidin-2-yl]piperidin-4-yl}ethyl)carbamate
tert-butyl ((1S)-2-hydroxy-1-{1-[4-(2-naphthyl)pyrimidin-2-yl]piperidin-4-yl}ethyl)carbamate
tert-butyl ((1R)-2-hydroxy-2-methyl-1-{1-[4-(2-naphthyl)pyrimidin-2-yl]piperidin-4-yl}propyl)carbamate
(1R)-1-amino-2-methyl-1-{1-[4-(2-naphthyl)pyrimidin-2-yl]piperidin-4-yl}propan-2-ol
(4R)-5,5-dimethyl-4-{1-[4-(2-naphthyl)pyrimidin-2-yl]piperidin-4-yl}-1,3-oxazolidin-2-one
(4R)-4-{1-[4-(2-naphthyl)pyrimidin-2-yl]piperidin-4-yl}-1,3-oxazolidin-2-one

The invention also relates to compounds of Formula II:

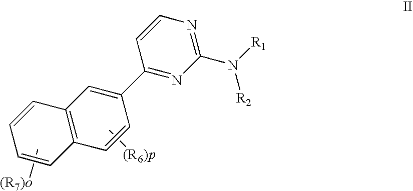

or pharmaceutically acceptable salts thereof,
wherein

R₁, R₂, R₆, R₇, R₁₁, R₁₂, R₁₃, R₁₄, o, p, and x are as defined above for the compounds of Formula II.

In one embodiment, R₁ is H. In one embodiment, R₁ is C₁-C₆ alkyl. In one embodiment, R₁ is C(O)C₁-C₆ alkyl. In one embodiment, R₁ is C(O)NC₁-C₆ alkyl. In one embodiment, R₁ is C₃-C₈ monocyclic cycloalkyl. In one embodiment, R₁ is a 3- to 7-membered monocyclic heterocycle.

In one embodiment, R₂ is H. In one embodiment, R₂ is C₁-C₆ alkyl. In one embodiment, R₂ is C(O)C₁-C₆ alkyl. In one embodiment, R₂ is C(O)NC₁-C₆ alkyl. In one embodiment, R₂ is C₃-C₈ monocyclic cycloalkyl. In one embodiment, R₂ is a 3- to 7-membered monocyclic heterocycle.

In one embodiment, R₆ is H. In one embodiment, R₆ is halogen. In one embodiment, R₆ is CN.

In one embodiment, R₆ is NO₂. In one embodiment, R₆ is OR₁₁. In one embodiment, R₆ is aryl.

In one embodiment, R₆ is alkylaryl. In one embodiment, R₆ is S(O)ₓR₁₁. In one embodiment, R₆ is NR₁₁R₁₂.

In one embodiment, R₁₁ is H. In one embodiment, R₁₁ is C₁₋C₆ alkyl. In one embodiment, R₁₁ is aryl. In one embodiment, R₁₁ is alkylaryl.

In one embodiment, R₁₂ is H. In one embodiment, R₁₂ is C₁₋C₆ alkyl. In one embodiment, R₁₂ is aryl. In one embodiment, R₁₂ is alkylaryl. In one embodiment, R₁₂ is COR₁₃. In one embodiment, R₁₂ is CO₂R₁₃. In one embodiment, R₁₂ is CONR₁₃R₁₄. In one embodiment, R₁₂ is SO₂R₁₃.

In one embodiment, R₁₁ and R₁₂ are taken together with the N to which they are attached to form a 3- to 7-membered monocyclic heterocycle. In one embodiment, R₁₁ and R₁₂ are taken together with the N to which they are attached to form an 8- to 12-membered bicyclic heterocycle.

In one embodiment, R₁₃ is H. In one embodiment, R₁₃ is C₁₋C₆ alkyl.

In one embodiment, R₁₄ is H. In one embodiment, R₁₄ is C₁₋C₆ alkyl.

In one embodiment, R₁₃ and R₁₄ are taken together with the N to which they are attached to form a 3- to 7-membered monocyclic heterocycle.

In one embodiment, o is 0. In one embodiment, o is 1.

In one embodiment, o is 2. In one embodiment, p is 0.

In one embodiment, p is 1. In one embodiment, p is 2.

In one embodiment, x is 0. In one embodiment, x is 1. In one embodiment, x is 2.

Illustrative compounds of Formula II are exemplified by the following compounds:

Compound

N-[4-(2-naphthyl)pyrimidin-2-yl]azepan-4-amine
tert-butyl 3-({[4-(2-naphthyl)pyrimidin-2-yl]amino}methyl)azetidine-1-carboxylate
N-(azetidin-3-ylmethyl)-4-(2-naphthyl)pyrimidin-2-amine
tert-butyl (3S)-3-({[4-(2-naphthyl)pyrimidin-2-yl]amino}methyl)pyrrolidine-1-carboxylate
4-(2-naphthyl)-N-[(3R)-pyrrolidin-3-ylmethyl]pyrimidin-2-amine
N-(2-morpholin-4-ylethyl)-4-(2-naphthyl)pyrimidin-2-amine
N-(3-morpholin-4-ylpropyl)-4-(2-naphthyl)pyrimidin-2-amine
4-(2-naphthyl)-N-(pyridin-3-ylmethyl)pyrimidin-2-amine
4-(2-naphthyl)-N-(pyridin-4-ylmethyl)pyrimidin-2-amine
4-(2-naphthyl)-N-(pyridin-2-ylmethyl)pyrimidin-2-amine
4-({[4-(2-naphthyl)pyrimidin-2-yl]amino}methyl)benzenesulfonamide
4-(2-naphthyl)-N-(2-pyridin-3-ylethyl)pyrimidin-2-amine
4-(2-naphthyl)-N-(2-pyridin-4-ylethyl)pyrimidin-2-amine
tert-butyl (3S)-3-{[4-(2-naphthyl)pyrimidin-2-yl]amino}pyrrolidine-1-carboxylate
tert-butyl (3R)-3-{[4-(2-naphthyl)pyrimidin-2-yl]amino}pyrrolidine-1-carboxylate
4-(2-naphthyl)-N-[(3S)-pyrrolidin-3-yl]pyrimidin-2-amine
4-(2-naphthyl)-N-[(3R)-pyrrolidin-3-yl]pyrimidin-2-amine
tert-butyl (3R)-3-({[4-(2-naphthyl)pyrimidin-2-yl]amino}methyl)pyrrolidine-1-carboxylate
4-(2-naphthyl)-N-[(3S)-pyrrolidin-3-ylmethyl]pyrimidin-2-amine
4-(2-naphthyl)-N-(piperidin-4-ylmethyl)pyrimidin-2-amine
tert-butyl 4-({[4-(2-naphthyl)pyrimidin-2-yl]amino}methyl)piperidine-1-carboxylate
trans-N-[4-(2-naphthyl)pyrimidin-2-yl]cyclohexane-1,4-diamine
N-(4-methoxybenzyl)-4-(2-naphthyl)pyrimidin-2-amine
N-[2-(4-methylphenyl)ethyl]-4-(2-naphthyl)pyrimidin-2-amine
2-{methyl[4-(2-naphthyl)pyrimidin-2-yl]amino}ethanol
N-(2-methoxyethyl)-4-(2-naphthyl)pyrimidin-2-amine
2-{[4-(2-naphthyl)pyrimidin-2-yl]amino}ethanol
N-(2-methoxyethyl)-N-methyl-4-(2-naphthyl)pyrimidin-2-amine
N-methyl-4-(2-naphthyl)pyrimidin-2-amine
N,N-diethyl-4-(2-naphthyl)pyrimidin-2-amine
4-(2-naphthyl)-N-propylpyrimidin-2-amine
N-butyl-4-(2-naphthyl)pyrimidin-2-amine
N-isopropyl-4-(2-naphthyl)pyrimidin-2-amine
N-(sec-butyl)-4-(2-naphthyl)pyrimidin-2-amine
N-isobutyl-4-(2-naphthyl)pyrimidin-2-amine
N-(tert-butyl)-4-(2-naphthyl)pyrimidin-2-amine
N-benzyl-4-(2-naphthyl)pyrimidin-2-amine
4-(2-naphthyl)-N-(2-phenylethyl)pyrimidin-2-amine
N-cyclopentyl-4-(2-naphthyl)pyrimidin-2-amine
N-cyclohexyl-4-(2-naphthyl)pyrimidin-2-amine
tert-butyl 4-{[4-(2-naphthyl)pyrimidin-2-yl]amino}piperidine-1-carboxylate
4-(2-naphthyl)-N-piperidin-4-ylpyrimidin-2-amine
tert-butyl 4-{methyl[4-(2-naphthyl)pyrimidin-2-yl]amino}piperidine-1-carboxylate
N-methyl-4-(2-naphthyl)-N-piperidin-4-ylpyrimidin-2-amine
tert-butyl 4-(2-methoxy-1-{[4-(2-naphthyl)pyrimidin-2-yl]amino}-2-oxoethyl)piperidine-1-
carboxylate
4-{methyl[4-(2-naphthyl)pyrimidin-2-yl]amino}piperidine-1-carbaldehyde
N-(1-benzylpiperidin-4-yl)-N-methyl-4-(2-naphthyl)pyrimidin-2-amine
N-[(1-acetylazetidin-3-yl)methyl]-4-(2-naphthyl)pyrimidin-2-amine
4-(2-naphthyl)-N-{[1-(trifluoroacetyl)azetidin-3-yl]methyl}pyrimidin-2-amine
N-[(1-benzoylazetidin-3-yl)methyl]-4-(2-naphthyl)pyrimidin-2-amine
3-({[4-(2-naphthyl)pyrimidin-2-yl]amino}methyl)azetidine-1-carboxamide
N-ethyl-3-({[4-(2-naphthyl)pyrimidin-2-yl]amino}methyl)azetidine-1-carboxamide
N-{[1-(methylsulfonyl)azetidin-3-yl]methyl}-4-(2-naphthyl)pyrimidin-2-amine
N-({1-[(4-methylphenyl)sulfonyl]azetidin-3-yl}methyl)-4-(2-naphthyl)pyrimidin-2-amine
N,N-dimethyl-3-({[4-(2-naphthyl)pyrimidin-2-yl]amino}methyl)azetidine-1-sulfonamide
N-[(3S)-1-acetylpyrrolidin-3-yl]-4-(2-naphthyl)pyrimidin-2-amine
N-[(3S)-1-benzoylpyrrolidin-3-yl]-4-(2-naphthyl)pyrimidin-2-amine
methyl (3S)-3-{[4-(2-naphthyl)pyrimidin-2-yl]amino}pyrrolidine-1-carboxylate
(3S)—N,N-dimethyl-3-{[4-(2-naphthyl)pyrimidin-2-yl]amino}pyrrolidine-1-carboxamide
(3S)—N-ethyl-3-{[4-(2-naphthyl)pyrimidin-2-yl]amino}pyrrolidine-1-carboxamide
(3S)-3-{[4-(2-naphthyl)pyrimidin-2-yl]amino}pyrrolidine-1-carboxamide
(3S)—N,N-dimethyl-3-{[4-(2-naphthyl)pyrimidin-2-yl]amino}pyrrolidine-1-sulfonamide
N-[(3S)-1-(methylsulfonyl)pyrrolidin-3-yl]-4-(2-naphthyl)pyrimidin-2-amine
N-{(3S)-1-[(4-methylphenyl)sulfonyl]pyrrolidin-3-yl}-4-(2-naphthyl)pyrimidin-2-amine
(3S)-3-{[4-(2-naphthyl)pyrimidin-2-yl]amino}pyrrolidine-1-carbaldehyde
N-[(3S)-1-(morpholin-4-ylcarbonyl)pyrrolidin-3-yl]-4-(2-naphthyl)pyrimidin-2-amine
ethyl (3S)-3-{[4-(2-naphthyl)pyrimidin-2-yl]amino}pyrrolidine-1-carboxylate
N-[(3R)-1-acetylpyrrolidin-3-yl]-4-(2-naphthyl)pyrimidin-2-amine
N-[(3R)-1-benzoylpyrrolidin-3-yl]-4-(2-naphthyl)pyrimidin-2-amine
methyl (3R)-3-{[4-(2-naphthyl)pyrimidin-2-yl]amino}pyrrolidine-1-carboxylate
(3R)—N,N-dimethyl-3-{[4-(2-naphthyl)pyrimidin-2-yl]amino}pyrrolidine-1-carboxamide
(3R)—N-ethyl-3-{[4-(2-naphthyl)pyrimidin-2-yl]amino}pyrrolidine-1-carboxamide
(3R)-3-{[4-(2-naphthyl)pyrimidin-2-yl]amino}pyrrolidine-1-carboxamide
(3R)—N,N-dimethyl-3-{[4-(2-naphthyl)pyrimidin-2-yl]amino}pyrrolidine-1-sulfonamide
N-[(3R)-1-(methylsulfonyl)pyrrolidin-3-yl]-4-(2-naphthyl)pyrimidin-2-amine
N-{(3R)-1-[(4-methylphenyl)sulfonyl]pyrrolidin-3-yl}-4-(2-naphthyl)pyrimidin-2-amine
(3R)-3-{[4-(2-naphthyl)pyrimidin-2-yl]amino}pyrrolidine-1-carbaldehyde
N-[(3R)-1-(morpholin-4-ylcarbonyl)pyrrolidin-3-yl]-4-(2-naphthyl)pyrimidin-2-amine
ethyl (3R)-3-{[4-(2-naphthyl)pyrimidin-2-yl]amino}pyrrolidine-1-carboxylate
N-{[(3R)-1-acetylpyrrolidin-3-yl]methyl}-4-(2-naphthyl)pyrimidin-2-amine
methyl (3R)-3-({[4-(2-naphthyl)pyrimidin-2-yl]amino}methyl)pyrrolidine-1-carboxylate
(3R)-3-({[4-(2-naphthyl)pyrimidin-2-yl]amino}methyl)pyrrolidine-1-carbaldehyde
N-{[(3S)-1-acetylpyrrolidin-3-yl]methyl}-4-(2-naphthyl)pyrimidin-2-amine
methyl (3S)-3-({[4-(2-naphthyl)pyrimidin-2-yl]amino}methyl)pyrrolidine-1-carboxylate
(3S)-3-({[4-(2-naphthyl)pyrimidin-2-yl]amino}methyl)pyrrolidine-1-carbaldehyde
N-[(1-acetylpiperidin-4-yl)methyl]-4-(2-naphthyl)pyrimidin-2-amine
N,N-dimethyl-4-({[4-(2-naphthyl)pyrimidin-2-yl]amino}methyl)piperidine-1-carboxamide
N,N-diethyl-4-({[4-(2-naphthyl)pyrimidin-2-yl]amino}methyl)piperidine-1-carboxamide
4-(2-naphthyl)-N-{[1-(pyrrolidin-1-ylcarbonyl)piperidin-4-yl]methyl}pyrimidin-2-amine
N-{[1-(methylsulfonyl)piperidin-4-yl]methyl}-4-(2-naphthyl)pyrimidin-2-amine
N,N-dimethyl-4-({[4-(2-naphthyl)pyrimidin-2-yl]amino}methyl)piperidine-1-sulfonamide
4-({[4-(2-naphthyl)pyrimidin-2-yl]amino}methyl)piperidine-1-carboxamide
N-ethyl-4-({[4-(2-naphthyl)pyrimidin-2-yl]amino}methyl)piperidine-1-carboxamide
N-isopropyl-4-({[4-(2-naphthyl)pyrimidin-2-yl]amino}methyl)piperidine-1-carboxamide
N-cyclohexyl-4-({[4-(2-naphthyl)pyrimidin-2-yl]amino}methyl)piperidine-1-carboxamide
N-[(1-benzoylpiperidin-4-yl)methyl]-4-(2-naphthyl)pyrimidin-2-amine
N-[(1-ethylpiperidin-4-yl)methyl]-4-(2-naphthyl)pyrimidin-2-amine
N-[(1-benzylpiperidin-4-yl)methyl]-4-(2-naphthyl)pyrimidin-2-amine
4-(2-naphthyl)-N-{[1-(phenylacetyl)piperidin-4-yl]methyl}pyrimidin-2-amine
N-({1-[(4-methylphenyl)sulfonyl]piperidin-4-yl}methyl)-4-(2-naphthyl)pyrimidin-2-amine
N-(trans-4-{[4-(2-naphthyl)pyrimidin-2-yl]amino}cyclohexyl)acetamide
methyl (trans-4-{[4-(2-naphthyl)pyrimidin-2-yl]amino}cyclohexyl)carbamate
N,N-dimethyl-N′-(trans-4-{[4-(2-naphthyl)pyrimidin-2-yl]amino}cyclohexyl)urea
N-ethyl-N′-(trans-4-{[4-(2-naphthyl)pyrimidin-2-yl]amino}cyclohexyl)urea
N,N-dimethyl-N′-(trans-4-{[4-(2-naphthyl)pyrimidin-2-yl]amino}cyclohexyl)sulfamide
N-(trans-4-{[4-(2-naphthyl)pyrimidin-2-yl]amino}cyclohexyl)methanesulfonamide
4-methyl-N-(trans-4-{[4-(2-naphthyl)pyrimidin-2-yl]amino}cyclohexyl)benzenesulfonamide
5-(dimethylamino)-N-(trans-4-{[4-(2-naphthyl)pyrimidin-2-yl]amino}cyclohexyl)naphthalene-
1-sulfonamide
4-cyano-N-(3-{[4-(2-naphthyl)pyrimidin-2-yl]amino}cyclohexyl)benzamide
N-(1-acetylpiperidin-4-yl)-4-(2-naphthyl)pyrimidin-2-amine
N,N-dimethyl-4-{[4-(2-naphthyl)pyrimidin-2-yl]amino}piperidine-1-carboxamide
N,N-diethyl-4-{[4-(2-naphthyl)pyrimidin-2-yl]amino}piperidine-1-carboxamide
4-(2-naphthyl)-N-[1-(pyrrolidin-1-ylcarbonyl)piperidin-4-yl]pyrimidin-2-amine
N-[1-(morpholin-4-ylcarbonyl)piperidin-4-yl]-4-(2-naphthyl)pyrimidin-2-amine
N-[1-(methylsulfonyl)piperidin-4-yl]-4-(2-naphthyl)pyrimidin-2-amine
N,N-dimethyl-4-{[4-(2-naphthyl)pyrimidin-2-yl]amino}piperidine-1-sulfonamide
4-{[4-(2-naphthyl)pyrimidin-2-yl]amino}piperidine-1-carboxamide
N-ethyl-4-{[4-(2-naphthyl)pyrimidin-2-yl]amino}piperidine-1-carboxamide
N-isopropyl-4-{[4-(2-naphthyl)pyrimidin-2-yl]amino}piperidine-1-carboxamide
N-cyclohexyl-4-{[4-(2-naphthyl)pyrimidin-2-yl]amino}piperidine-1-carboxamide
N-(1-benzoylpiperidin-4-yl)-4-(2-naphthyl)pyrimidin-2-amine
N-(1-acetylpiperidin-4-yl)-N-methyl-4-(2-naphthyl)pyrimidin-2-amine
N-methyl-4-(2-naphthyl)-N-[1-(trifluoroacetyl)piperidin-4-yl]pyrimidin-2-amine
N-(1-benzoylpiperidin-4-yl)-N-methyl-4-(2-naphthyl)pyrimidin-2-amine
methyl 4-{methyl[4-(2-naphthyl)pyrimidin-2-yl]amino}piperidine-1-carboxylate
N,N-dimethyl-4-{methyl[4-(2-naphthyl)pyrimidin-2-yl]amino}piperidine-1-carboxamide
N-methyl-4-(2-naphthyl)-N-[1-(pyrrolidin-1-ylcarbonyl)piperidin-4-yl]pyrimidin-2-amine
N,N-diethyl-4-{methyl[4-(2-naphthyl)pyrimidin-2-yl]amino}piperidine-1-carboxamide
N-ethyl-4-{methyl[4-(2-naphthyl)pyrimidin-2-yl]amino}piperidine-1-carboxamide
4-{methyl[4-(2-naphthyl)pyrimidin-2-yl]amino}piperidine-1-carboxamide
N-methyl-N-[1-(methylsulfonyl)piperidin-4-yl]-4-(2-naphthyl)pyrimidin-2-amine
N-methyl-N-{1-[(4-methylphenyl)sulfonyl]piperidin-4-yl}-4-(2-naphthyl)pyrimidin-2-amine
N-methyl-4-(2-naphthyl)-N-(1-pyrimidin-2-ylpiperidin-4-yl)pyrimidin-2-amine
N-methyl-4-(2-naphthyl)-N-(1-propylpiperidin-4-yl)pyrimidin-2-amine
N-(1-benzylpiperidin-4-yl)-N-methyl-4-(2-naphthyl)pyrimidin-2-amine
2-(4-{methyl[4-(2-naphthyl)pyrimidin-2-yl]amino}piperidin-1-yl)acetamide
N-methyl-4-(2-naphthyl)-N-{1-[2-(trityloxy)ethyl]piperidin-4-yl}pyrimidin-2-amine
4-{methyl[4-(2-naphthyl)pyrimidin-2-yl]amino}piperidine-1-carboximidamide
tert-butyl (3-{[4-(2-naphthyl)pyrimidin-2-yl]amino}cyclohexyl)carbamate
4-(2-naphthyl)-2-[4-(pyrrolidin-1-ylmethyl)piperidin-1-yl]pyrimidine

### Methods for Using Naphthylpyrimidin, Naphthylpyrazine and Naphthylpyridazine Analogs

The naphthylpyrimidine analogs of the present invention exhibit agonism of the canonical Wnt-β-catenin cellular messaging system and, therefore, can be utilized in order to inhibit abnormal cell growth and/or encourage healthy cell regeneration or healthy cell growth. Thus, the naphthylpyrimidine analogs are effective in the treatment of disorders of the canonical Wnt-β-catenin cellular messaging system, including bone disorders. The naphthylpyrimidine analogs may also be effective to treat other disorders of the canonical Wnt-β-catenin cellular messaging system including, cancer and neurological conditions. In particular, the naphthylpyrimidine analogs of the present invention possess bone anabolic groth properties and have cancer cell growth inhibiting effects and are effective in treating cancers. Types of cancers that may be treated include, but are not limited to, solid cancers and malignant lymphomas, and also, leukemia, skin cancer, bladder cancer, breast cancer, uterus cancer, ovary cancer, prostate cancer, lung cancer, colon cancer, pancreas cancer, renal cancer, gastric cancer, brain tumor. Types of neurological conditions that may be treated include, but are not limited too, peripheral neuropathy, spinal cord injury, Parkinson's disease, memory loss, and Alzheimer's disease.

### Therapeutic Administration

When administered to an animal, the naphthylpyrimidine analogs or pharmaceutically acceptable salts of the naphthylpyrimidine analogs can be administered neat or as a component of a composition that comprises a physiologically acceptable carrier or vehicle. A composition of the invention can be prepared using a method comprising admixing the naphthylpyrimidine analogs or a pharmaceutically acceptable salt of the naphthylpyrimidine analogs and a physiologically acceptable carrier, excipient, or diluent. Admixing can be accomplished using methods well known for admixing a naphthylpyrimidine analog or a pharmaceutically acceptable salt of the naphthylpyrimidine analog and a physiologically acceptable carrier, exipient, or diluent.

The present compositions, comprising naphthylpyrimidine analogs or pharmaceutically acceptable salts of the naphthylpyrimidine analogs of the invention can be administered orally. The naphthylpyrimidine analogs or pharmaceutically acceptable salts of naphthylpyrimidine analogs of the invention can also be administered by any other convenient route, for example, by infusion or bolus injection, by absorption through epithelial or mucocutaneous linings (e.g., oral, rectal, vaginal, and intestinal mucosa) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravascular (e.g., intra-arterial or intravenous), subcutaneous, intranasal, epidural, oral, sublingual, intracerebral, intravaginal, transdermal, rectal, by inhalation, or topical, particularly to the ears, nose, eyes, or skin. In some instances, administration will result in release of the naphthylpyrimidine analog or a pharmaceutically acceptable salt of the naphthylpyrimidine analog into the bloodstream. A suitable mode of administration can be readily determined, and is left to the discretion of the practitioner.

In one embodiment, the naphthylpyrimidine analog or a pharmaceutically acceptable salt of the naphthylpyrimidine analog is administered orally.

In another embodiment, the naphthylpyrimidine analog or a pharmaceutically acceptable salt of the naphthylpyrimidine analog is administered intravenously.

In another embodiment, the naphthylpyrimidine analog or a pharmaceutically acceptable salt of the naphthylpyrimidine analog can be administered locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, the naphthylpyrimidine analog or a pharmaceutically acceptable salt of the naphthylpyrimidine analog can be introduced into the central nervous system, circulatory system or gastrointestinal tract by any suitable route, including intraventricular, intrathecal injection, paraspinal injection, epidural injection, enema, and by injection adjacent to the peripheral nerve. Intraventricular injection can be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the naphthylpyrimidine analog or a pharmaceutically acceptable salt of the naphthylpyrimidine analog can be formulated as a suppository, with traditional binders and excipients such as triglycerides.

In another embodiment, the naphthylpyrimidine analog or a pharmaceutically acceptable salt of the naphthylpyrimidine analog can be delivered in a vesicle, in particular a liposome (see Langer, Science 249:1527-1533 (1990) and Treat et al., Liposomes in the Therapy of Infectious Disease and Cancer pp. 317-327 and pp. 353-365 (1989)).

In yet another embodiment, the naphthylpyrimidine analog or a pharmaceutically acceptable salt of the naphthylpyrimidine analog can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment, a controlled- or sustained-release system can be placed in proximity of a target of the naphthylpyrimidine analog or a pharmaceutically acceptable salt of the naphthylpyrimidine analog, e.g., the reproductive organs, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment, the physiologically acceptable excipients are sterile when administered to an animal. The physiologically acceptable excipient should be stable under the conditions of manufacture and storage and should be preserved against the contaminating action of microorganisms. Water is a particularly useful excipient when the naphthylpyrimidine analog or a pharmaceutically acceptable salt of the naphthylpyrimidine analogs is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The naphthylpyrimidine analog or pharmaceutically acceptable salt of the naphthylpyrimidine analog of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose derivatives, including sodium carboxymethyl cellulose solution), alcohols (including monohydric alcohols and polyhydric alcohols, e.g., glycols) and their derivatives, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences pp. 1447-1676 (Alfonso R. Gennaro, ed., 19th ed. 1995, the disclosures of which are herein incorporated by reference).

In one embodiment, the naphthylpyrimidine analog or a pharmaceutically acceptable salt of the naphthylpyrimidine analog is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided naphthylpyrimidine analog or pharmaceutically acceptable salt of the naphthylpyrimidine analog. In tablets, the naphthylpyrimidine analog or pharmaceutically acceptable salt of the naphthylpyrimidine analog is mixed with a carrier having the necessary compression properties in suitable proportions and compacted in the shape and size desired. The powders and tablets can contain about 0.01% to 99% of the naphthylpyrimidine analog or pharmaceutically acceptable salt of the naphthylpyrimidine analog.

Capsules may contain mixtures of the naphthylpyrimidine analogs or pharmaceutically acceptable salts of the naphthylpyrimidine analogs with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the naphthylpyrimidine analog or a pharmaceutically acceptable salt of the naphthylpyrimidine analog can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampule or sachette indicating the quantity of active agent. Where the naphthylpyrimidine analog or a pharmaceutically acceptable salt of the naphthylpyrimidine analog is to be administered by infusion, it can be dispensed, for example, with an infusion bottle containing sterile pharmaceutical grade water or saline. Where the naphthylpyrimidine analog or a pharmaceutically acceptable salt of the naphthylpyrimidine analog is administered by injection, an ampule of sterile water for injection or saline can be provided so that the ingredients can be mixed prior to administration.

In another embodiment, the naphthylpyrimidine analog or pharmaceutically acceptable salt of the naphthylpyrimidine analog can be administered transdermally through the use of a transdermal patch. Transdermal administrations include administrations across the surface of the body and the inner linings of the bodily passages including epithelial and mucosal tissues. Such administrations can be carried out using the present naphthylpyrimidine analogs or pharmaceutically acceptable salts of the naphthylpyrimidine analogs, in lotions, creams, foams, patches, suspensions, solutions, and suppositories (e.g., rectal or vaginal).

Transdermal administration can be accomplished through the use of a transdermal patch containing the naphthylpyrimidine analog or pharmaceutically acceptable salt of the naphthylpyrimidine analog and a carrier that is inert to the naphthylpyrimidine analog or pharmaceutically acceptable salt of the naphthylpyrimidine analog, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the naphthylpyrimidine analog or pharmaceutically acceptable salt of the naphthylpyrimidine analog into the blood stream, such as a semi-permeable membrane covering a reservoir containing the naphthylpyrimidine analog or pharmaceutically acceptable salt of the naphthylpyrimidine analog with or without a carrier, or a matrix containing the active ingredient.

The naphthylpyrimidine analogs or pharmaceutically acceptable salts of the naphthylpyrimidine analogs of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The naphthylpyrimidine analog or a pharmaceutically acceptable salt of the naphthylpyrimidine analog can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the naphthylpyrimidine analog or a pharmaceutically acceptable salt of the naphthylpyrimidine analog, and can thus reduce the occurrence of adverse side effects.

Controlled- or sustained-release compositions can initially release an amount of the naphthylpyrimidine analog or a pharmaceutically acceptable salt of the naphthylpyrimidine analog that promptly produces the desired therapeutic or prophylactic effect, and gradually and continually release other amounts of the naphthylpyrimidine analog or a pharmaceutically acceptable salt of the naphthylpyrimidine analog to maintain this level of therapeutic or prophylactic effect over an extended period of time. To maintain a constant level of the naphthylpyrimidine analog or a pharmaceutically acceptable salt of the naphthylpyrimidine analog in the body, the naphthylpyrimidine analog or a pharmaceutically acceptable salt of the naphthylpyrimidine analog can be released from the dosage form at a rate that will replace the amount of the naphthylpyrimidine analog or a pharmaceutically acceptable salt of the naphthylpyrimidine analog being metabolized and excreted from the body. Controlled- or sustained-release of an active ingredient can be stimulated by various conditions, including but not limited to, changes in pH, changes in temperature, concentration or availability of enzymes, concentration or availability of water, or other physiological conditions.

In certain embodiments, the present invention is directed to prodrugs of the naphthylpyrimidine analogs or pharmaceutically acceptable salts of naphthylpyrimidine analogs of the present invention. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975). The amount of the naphthylpyrimidine analog or a pharmaceutically acceptable salt of the naphthylpyrimidine analog that is effective for treating or preventing a canonical Wnt-β-catenin cellular messaging system-related disorder can be determined using standard clinical techniques. In addition, in vitro or in vivo assays can optionally be employed to help identify suitable dosage ranges. The precise dose to be employed can also depend on the route of administration, the condition, the seriousness of the condition being treated, as well as various physical factors related to the individual being treated, and can be decided according to the judgment of an ordinarily skilled health-care practitioner. The typical dose will range from about 0.001 mg/kg to about 250 mg/kg of body weight per day, in one embodiment, from about 1 mg/kg to about 250 mg/kg body weight per day, in another embodiment, from about 1 mg/kg to about 50 mg/kg body weight per day, and in another embodiment, from about 1 mg/kg to about 20 mg/kg of body weight per day. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy can be readily determined according to the judgment of an ordinarily skilled health-care practitioner. The effective dosage amounts described herein refer to total amounts administered; that is, if more than one naphthylpyrimidine analog or a pharmaceutically acceptable salt of the naphthylpyrimidine analog is administered, the effective dosage amounts correspond to the total amount administered.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, pre-filled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The naphthylpyrimidine analog or a pharmaceutically acceptable salt of the naphthylpyrimidine analog can be assayed in vitro or in vivo for the desired therapeutic or prophylactic activity prior to use in humans. Animal model systems can be used to demonstrate safety and efficacy.

The present methods for treating or preventing a canonical Wnt-β-catenin cellular messaging system-related disorder, can further comprise administering another therapeutic agent to the animal being administered the naphthylpyrimidine analog or a pharmaceutically acceptable salt of the naphthylpyrimidine analog.

Effective amounts of the other therapeutic agents are well known to those skilled in the art. However, it is well within the skilled artisan's purview to determine the other therapeutic agent's optimal effective amount range. The naphthylpyrimidine analog or a pharmaceutically acceptable salt of the naphthylpyrimidine analog and the other therapeutic agent can act additively or, in one embodiment, synergistically. In one embodiment, of the invention, where another therapeutic agent is administered to an animal, the effective amount of the naphthylpyrimidine analog or a pharmaceutically acceptable salt of the naphthylpyrimidine analog is less than its effective amount would be where the other therapeutic agent is not administered. In this case, without being bound by theory, it is believed that the naphthylpyrimidine analog or a pharmaceutically acceptable salt of the naphthylpyrimidine analog and the other therapeutic agent act synergistically.

Suitable other therapeutic agents useful in the methods and compositions of the present invention include, but are not limited to cancer agents, Alzheimer's agents, bone disorder agents, osteoporosis agents, rheumatoid arthritis agents, osteoarthritis agents, and hormone replacement agents.

Suitable cancer agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, hexamethylmelamine, topotecan, tyrosine kinase inhibitors, tyrphostins herbimycin A, genistein, erbstatin, and lavendustin A.

Other therapeutic agents useful in the methods and compositions of the present invention include, but are not limited to hydroxyzine, glatiramer acetate, interferon beta-1a, interferon beta-1b, mitoxantrone, and natalizumab.

Suitable Alzheimer's agents useful in the methods and compositions of the present invention include, but are not limited to donepezil, galantamine, memantine, niacin, rivastigmine, and tacrine.

Suitable bone disorder and/or osteoporosis agents useful in the methods and compositions of the present invention include, but are not limited to alendronate, bazedoxifene, calcitonin, clomifene, lasofoxifene, ormeloxifene, raloxifene, tamoxifen, and toremifene.

Suitable rheumatoid arthritis agents useful in the methods and compositions of the present invention include, but are not limited to abatacept, acetaminophen adalimumab, aspirin, auranofin, azathioprine, celecoxib, cyclophosphamide, cyclosporine, diclofenac, etanercept, hydroxychloroquine, ibuprofen, indomethacin, infliximab, ketoprofen, leflunomide, methotrexate, minocycline, nabumetone, naproxen, rituximab, and sulfasalazine.

Suitable osteoarthritis agents useful in the methods and compositions of the present invention include, but are not limited to acetaminophen, aspirin, celecoxib, cortisone, hyaluronic acid, ibuprofen, nabumetone, naproxen, rofecoxib, and valdecoxib.

Suitable hormone replacement therapy agents useful in the methods and compositions of the present invention include, but are not limited to estrogen, estradiol, medroxyprogesterone, norethindrone, and progesterone.

In one embodiment, the naphthylpyrimidine analog or a pharmaceutically acceptable salt of the naphthylpyrimidine analog is administered concurrently with another therapeutic agent.

In one embodiment, a composition comprising an effective amount of the naphthylpyrimidine analog or a pharmaceutically acceptable salt of the naphthylpyrimidine analog and an effective amount of another therapeutic agent within the same composition can be administered.

In another embodiment, a composition comprising an effective amount of the naphthylpyrimidine analog or a pharmaceutically acceptable salt of the naphthylpyrimidine analog and a separate composition comprising an effective amount of another therapeutic agent can be concurrently administered.

In another embodiment, an effective amount of the naphthylpyrimidine analog or a pharmaceutically acceptable salt of the naphthylpyrimidine analog is administered prior to or subsequent to administration of an effective amount of another therapeutic agent. In this embodiment, the naphthylpyrimidine analog or a pharmaceutically acceptable salt of the naphthylpyrimidine analog is administered while the other therapeutic agent exerts its therapeutic effect, or the other therapeutic agent is administered while the naphthylpyrimidine analog or a pharmaceutically acceptable salt of the naphthylpyrimidine analog exerts its preventative or therapeutic effect for treating or preventing a canonical Wnt-β-catenin cellular messaging system-related disorder.

In another embodiment, the pharmaceutically acceptable carrier is suitable for oral administration and the composition comprises an oral dosage form.

The naphthylpyrimidine analogs and pharmaceutically acceptable salts of naphthylpyrimidine analogs can be prepared using a variety of methods starting from commercially available compounds, known compounds, or compounds prepared by known methods. General synthetic routes to many of the compounds of the invention are included in the following schemes. It is understood by those skilled in the art that protection and deprotection steps not shown in the Schemes may be required for these syntheses, and that the order of steps may be changed to accommodate functionality in the target molecule.

Methods useful for making the naphthylpyrimidine analogs are set forth in the Examples below and generalized in Schemes.

### Methods of Making Naphthylpyrimidine, Naphthylpyrazine and Naphthylpyridazine Analogs

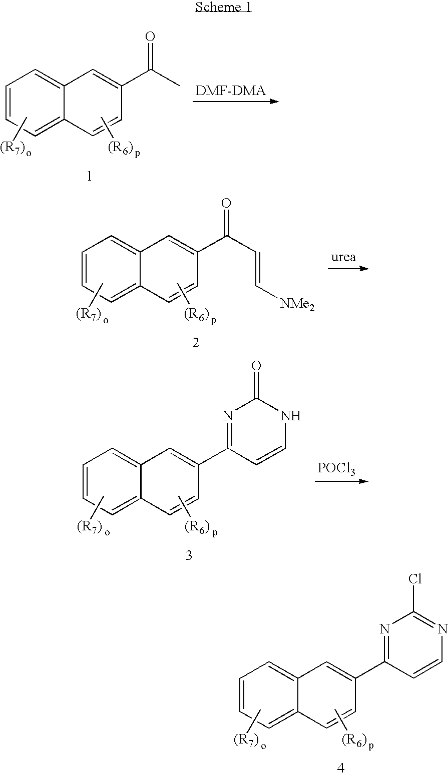

wherein R₆, R₇, o, and p are as defined above.

4-(2-naphthyl)-2-chloropyrimidine 4 can be prepared by treating a 2-acetylnaphthalene compound of formula 1 with DMF-dimethylacetyl to provide vinylogous amides of formula 2. Compounds of formula 2 can be treated with urea to form the pyrimidinone product of formula 3. Compounds of formula 3 can be converted to the chloride 4 by refluxing in phosphorous oxychloride for several hours.

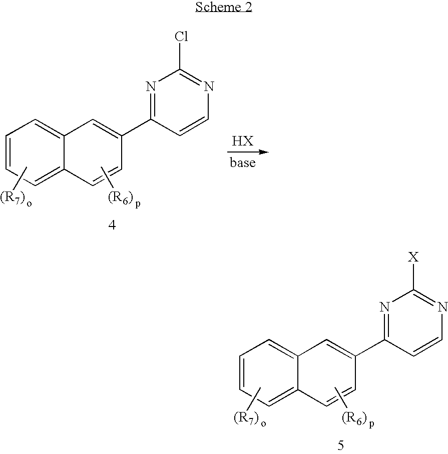

wherein R₆, R₇, o and p are as defined above and X is a primary alkyl or aryl amine, a secondary amine, a cyclicamine (e.g. a piperidine analog), an O(alkyl), O(aryl), an S(alkyl), or an S(aryl).

A pyrimidinyl chloride of formula 4 can be substituted in the 2 position by heating with a variety of nucleophiles including primary and secondary amines, oxygen nucleophiles, and sulfur nucleophiles, to provide compounds of formula 5.

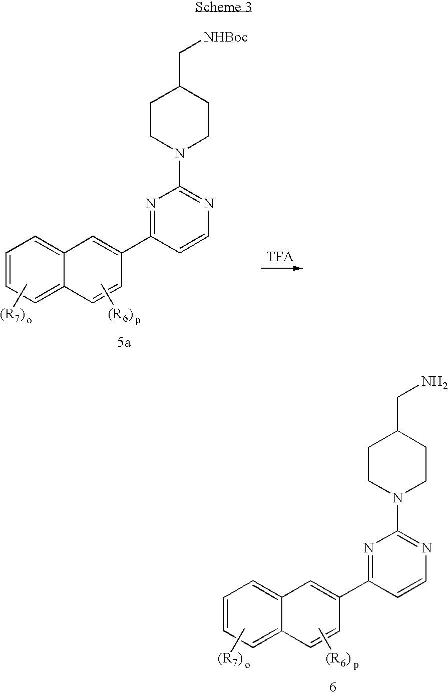

wherein R₆, R₇, o and p are as defined above in Formula I.

Additional manipulations of 5 include deprotection of typical groups such as the t-butyloxycarbonyl of 5a under acidic conditions to provide the amine compounds 6.

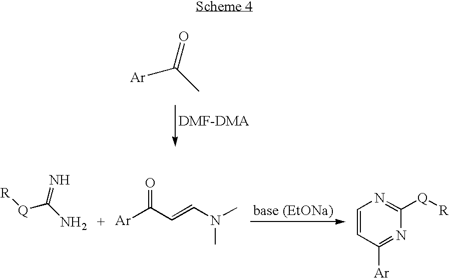

wherein Ar is

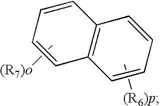

Q is N(CH₂)ᵣR₈ or CR₈R₉;
R is

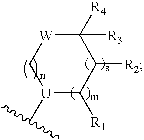

wherein U, R₁, R₂, R₃, R₄, W, R₆, R₇, R₈, R₉, m, n, o, p and s are as defined above in Formula A.

### EXAMPLES

The following general methods outline the synthesis of the naphthylpyrimidine analogs of the present invention.

### HPLC and LC/MS Methods Used for the Following Examples and Intermediates

Method A: Column; Xterra MS C18, 5 μ, 50×2.1 mm. Mobile phase: 90/10-5/95 water (0.1% formic acid)/acetonitrile (0.1% formic acid), 2 min, hold 1.5 min, 0.8 mL/min., 210-400 nm.

Method B: LC/MS: YMC CombiScreen ProC18 50×4.6 mm I.D. column, S-5 μm, 12 nm. Flow rate 1.0 mL/min. Gradient: 10/90 Acetonitrile/Water (0.1% TFA in both solvents) to 100% acetonitrile over 10 minutes. Hold 100% acetonitrile for 3 mins then back to 10/90 over 2 mins. MS detection using a ThermoFinnigan AQA mass spectrometer in ESI positive mode.

Method C: Column; Xterra RP18, 3.5 μ, 150×4.6 mm. Mobile phase: 85/15-5/95 Phosphate buffer (pH=2.1)/ACN+MeOH (1:1) for 10 min, hold 4 mins, 1.2 mL/min., 210-370 nm.

Method D: YMC CombiPrep ProC18 50×20 mm I.D. column, S-5 □m, 12 nm. Flow rate 20 mL/min. Gradient: 10/90 Acetonitrile/Water (0.1% TFA in both solvents) to 100% acetonitrile over 10 minutes then hold for three minutes at 100% acetonitrile and ramp back to 10/90 acetonitrile/water over two minutes.

Method E: Column: Waters Atlantis C18, 5 μ, 2×50 mm. Mobile phase: 95/5-5/95 water (10 mM ammonium acetate)/acetonitrile (10 mM ammonium acetate), 2.5 min., hold 1.5 min., 0.8 mL/min., 210-400 nm.

Method F: Column; Xterra RP18, 3.5 μ, 150×4.6 mm. Mobile phase: 85/15-5/95 Ammonium formate buffer (pH=3.5)/ACN+MeOH (1:1) for 10 min, hold 4 mins, 1.2 mL/min., 210-370 nm.

Method G: Column; Xterra RP18, 3.5 μ, 150×4.6 mm. Mobile phase: 85/15-5/95 Ammonium bicarbonate buffer (pH=9.5)/ACN+MeOH (1:1) for 10 min, hold 4 mins, 1.2 mL/min., 210-370 nm.

Method H: Column: Waters Atlantis C18, 5 μ, 4.6×150 mm. Mobile phase: 95/5-5/95 water (0.1% formic acid)/acetonitrile (0.1% formic acid), 6 min., hold 1.2 min., 1.5 mL/min., 210-400

Method I: Column: Sunfire prep C18, 5 μ, 19×50 mm. Flow rate 20 mL/min. Gradient: 10/90 Acetonitrile/Water to 100% acetonitrile over 10 minutes then hold for three minutes at 100% acetonitrile and ramp back to 10/90 acetonitrile/water over two minutes.

Method J: Waters Gemini C18 50×20 mm I.D. column, S-5 μm, 12 nm. Flow rate 20 mL/min. Gradient: 10/90 Acetonitrile/Water (0.05% ammonia in water) to 100% acetonitrile over 10 minutes then hold for three minutes at 100% acetonitrile and ramp back to 10/90 acetonitrile/water over two minutes.

### Example 1

### Preparation of 2-Chloro-4-(naphthalene-2-yl)pyrimidine

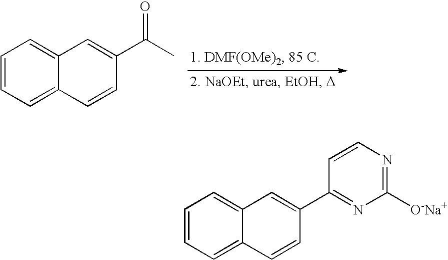

Sodium 4-(naphthalene-2-yl)pyrimidin-2-olate: 2-Acetyl naphthalene (15.0 g, 88.1 mmol) and DMF-dimethyl acetal (15.2 mL, 114.5 mmol) are combined and heated in an 85° C. bath overnight. The reaction is concentrated on a rotovap to a thick oil, and became a tan solid on standing under high vacuum. To the residue is added EtOH (anhydrous, 40 mL), urea (6.35 g, 105.7 mmol), and sodium ethoxide solution (21% weight solution in EtOH, 33 mL, 88.1 mmol) and the mixture is heated to gentle reflux overnight. The resulting mixture is cooled to room temperature, then filtered to collect a dark solid which is rinsed with EtOH. The solid is allowed to dry at room temperature for 1 h, then is suspended in H₂O and CH₂Cl₂ (1:1, ˜400 mL total). The resulting sticky material is collected by filtration and allowed to dry. An orange-pink powder (11.75 g, 54%) is obtained and carried on directly. LC/MS (Method A) rt=1.10 mins., calculated mass=222, [M−H]⁻=221.

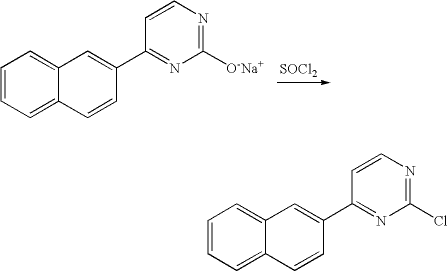

2-Chloro-4-(naphthalene-2-yl)pyrimidine: To thionyl chloride (56 mL, 0.77 mol) cooled in an ice bath is added the sodium salt (11.75 g, 48.1 mmol) in portions. To the mixture is added DMF (8 mL) and additional thionyl chloride (3 mL). The reaction is heated gradually in a 70° C. bath overnight. The solution is cooled to room temperature and concentrated in vacuuo. Benzene (˜15 mL) is added and the solution is concentrated. This is repeated to give an orange solid. The solid is cooled in an ice bath and H₂O and K₂CO₃ are added to neutralize any acid. The material is extracted with CH₂Cl₂. The organic extracts are washed with brine, dried (Na₂SO₄), filtered, and concentrated to provide a brown solid (13.4 g). The solid is adsorbed onto silica gel (˜200 mL) and the silica is placed on a fritted funnel and is washed with 25% EtOAc/hexane (800 mL), then 50% EtOAc/hexane (400 mL). The 50% filtrate is concentrated to afford 4.2 g of a tan powder which is pure by 1H NMR and LC/MS. The 25% filtrate is concentrated and the resulting solid is recrystallized from acetone to afford a first crop of 1.65 g and a second crop of 4 g of beige powder, which are pure by 1H NMR and LC/MS. LC/MS (Method A) rt=1.85 min., purity=96%, calculated mass=240, [M+H]⁺=241.

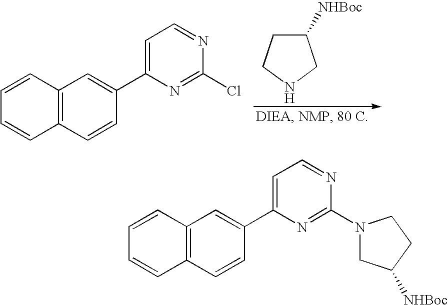

tert-butyl {(3S)-1-[4-(2-naphthyl)pyrimidin-2-yl]pyrrolidin-3-yl}carbamate. A solution of 2-Chloro-4-(naphthalene-2-yl)pyrimidine (0.25 g, 1.0 mmol), (3S)-(-)-3-(tert-butoxycarbonylamino)pyrrolidine (0.29 g, 1.5 mmol), and diisopropylethylamine (0.27 mL, 1.5 mmol) in N-methylpyrrolidine (2 mL) is heated in a vial in a sheker block at 80° C. for 14 h. The reaction is cooled to room temperature and EtOAc (200 mL) and water (25 mL) are used to transfer the contents of the vial to a separatory funnel. The layers are separated. The organic layer is washed with water (8×30 mL), and brine (30 mL), dried (Na₂SO₄), filtered, and concentrated. The crude material is purified by silica gel chromatography, eluting with 3% MeOH/CH₂Cl₂, to afford the title compound as an ivory powder (0.40 g, 99%). HPLC (Method C) purity 100%, rt=11.2 min; LC/MS (Method A), rt=1.78 mins., calculated mass=390, [M+H]⁺=391.

### Example 2

### Preparation of (3S)-1-[4-(2-naphthyl)pyrimidin-2-yl]pyrrolidin-3-amine

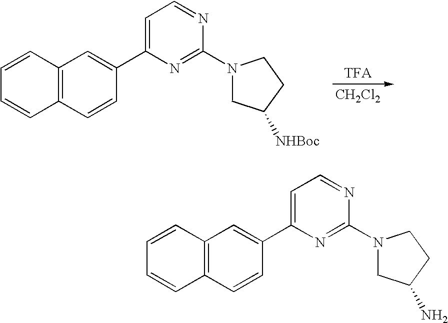

(3S)-1-[4-(2-naphthyl)pyrimidin-2-yl]pyrrolidin-3-amine. To a solution of tert-butyl {(3S)-1-[4-(2-naphthyl)pyrimidin-2-yl]pyrrolidin-3-yl}carbamate (0.35 g, 0.89 mmol) in CH₂Cl₂ (10 mL) is added trifluoroacetic acid (5 mL). The solution is stirred at room temperature for 14 h, then concentrated to a dark oil. To the residue is added a saturated K₂CO₃ solution (5 mL), followed by ethyl acetate (100 mL). The layers are separated and the aqueous layer is extracted with ethyl acetate (2×100 mL). The combined organic layers are washed with brine (25 mL), dried (Na₂SO₄), filtered, and concentrated to afford an orange oil (0.22 g, 85%). A sample is purified by RP HPLC (Method D) for analysis. HPLC (Method C) purity 99.7%, rt=7.5 min.; HRMS: calcd for C₁₈H₁₈N₄⁺H⁺, 291.16042; found ([M+H]⁺), 291.1617.

Additional examples prepared in a manner similar to examples 1 and 2 starting from 2-chloro-4-(2-naphthyl)pyrimidine and the appropriate amine or t-butyloxycarbonyl (Boc) protected diamine are listed in the following table:

| Example | | | Rt. | HPLC |
|---|---|---|---|---|
| no. | Compound | MS | (min.) | Method |
| 3 | 4-[4-(2-naphthyl)pyrimidin-2-yl]-1,4-diazepane-1- | 333 | 9.7 | C |
| | carbaldehyde | | | |
| 4 | 1-[4-(2-naphthyl)pyrimidin-2-yl]-1,4-diazepane | 305 | 8.0 | C |
| 5 | 1-[4-(2-naphthyl)pyrimidin-2-yl]azepan-4-amine | 319 | 8.4 | C |
| 6 | tert-butyl {1-[4-(2-naphthyl)pyrimidin-2-yl]azepan-4- | 419 | 11.7 | C |
| | yl}carbamate | | | |
| 7 | N-[4-(2-naphthyl)pyrimidin-2-yl]azepan-4-amine | 319 | 11.1 | C |
| 8 | tert-butyl 3-({[4-(2-naphthyl)pyrimidin-2- | 391 | 11.0 | C |
| | yl]amino}methyl)azetidine-1-carboxylate | | | |
| 9 | N-(azetidin-3-ylmethyl)-4-(2-naphthyl)pyrimidin-2-amine | 291 | 7.4 | C |
| 10 | tert-butyl ({1-[4-(2-naphthyl)pyrimidin-2-yl]azetidin-3- | 391 | 10.8 | C |
| | yl}methyl)carbamate | | | |
| 11 | 1-{1-[4-(2-naphthyl)pyrimidin-2-yl]azetidin-3- | 291 | 7.4 | C |
| | yl}methanamine | | | |
| 12 | tert-butyl ({1-[4-(2-naphthyl)pyrimidin-2-yl]pyrrolidin-3- | 405 | 11.4 | F |
| | yl}methyl)carbamate | | | |
| 13 | 1-{1-[4-(2-naphthyl)pyrimidin-2-yl]pyrrolidin-3- | 305 | 8.1 | F |
| | yl}methanamine | | | |
| 14 | tert-butyl (3S)-3-({[4-(2-naphthyl)pyrimidin-2- | 405 | 11.3 | G |
| | yl]amino}methyl)pyrrolidine-1-carboxylate | | | |
| 15 | 4-(2-naphthyl)-N-[(3R)-pyrrolidin-3-ylmethyl]pyrimidin-2- | 305 | 9.1 | G |
| | amine | | | |
| 16 | 4-(2-{4-[4-(2-naphthyl)pyrimidin-2-yl]piperazin-1- | 404 | 10.3 | G |
| | yl}ethyl)morpholine | | | |
| 17 | N-(2-morpholin-4-ylethyl)-4-(2-naphthyl)pyrimidin-2-amine | 335.3 | 2.1 | E |
| 18 | N-(3-morpholin-4-ylpropyl)-4-(2-naphthyl)pyrimidin-2-amine | 349.3 | 2.1 | E |
| 19 | 4-({4-[4-(2-naphthyl)pyrimidin-2-yl]piperazin-1- | 418.3 | 2.3 | E |
| | yl}acetyl)morpholine | | | |
| 20 | N,N-dimethyl-2-{4-[4-(2-naphthyl)pyrimidin-2-yl]piperazin-1- | 362.3 | 2.3 | E |
| | yl}ethanamine | | | |
| 21 | 2-(4-methylpiperazin-1-yl)-4-(2-naphthyl)pyrimidine | 305.3 | 2.2 | E |
| 22 | 4-(2-naphthyl)-2-(4-pyridin-2-ylpiperazin-1-yl)pyrimidine | 368.3 | 2.8 | E |
| 23 | 4-(2-naphthyl)-2-(4-pyrimidin-2-ylpiperazin-1-yl)pyrimidine | 369.3 | 2.7 | E |
| 24 | 1-{1-[4-(2-naphthyl)pyrimidin-2-yl]piperidin-4-yl}-1,3- | 422.3 | 2.6 | E |
| | dihydro-2H-benzimidazol-2-one | | | |
| 25 | 4-(2-naphthyl)-N-(pyridin-3-ylmethyl)pyrimidin-2-amine | 313 | 9.4 | F |
| 26 | 4-(2-naphthyl)-N-(pyridin-4-ylmethyl)pyrimidin-2-amine | 313 | 9.1 | F |
| 27 | 4-(2-naphthyl)-N-(pyridin-2-ylmethyl)pyrimidin-2-amine | 313 | 9.6 | F |
| 28 | 2-[4-(2-naphthyl)pyrimidin-2-yl]-1,2,3,4- | 338 | 12.3 | F |
| | tetrahydroisoquinoline | | | |
| 29 | 4-({[4-(2-naphthyl)pyrimidin-2- | 391 | 9.4 | F |
| | yl]amino}methyl)benzenesulfonamide | | | |
| 30 | (2-{4-[4-(2-naphthyl)pyrimidin-2-yl]piperazin-1- | 334 | 8.3 | F |
| | yl}ethyl)amine | | | |
| 31 | N,N-dimethyl-3-{4-[4-(2-naphthyl)pyrimidin-2-yl]piperazin-1- | 376 | 6.7 | F |
| | yl}propan-1-amine | | | |
| 32 | 4-(2-naphthyl)-2-(4-pyridin-4-ylpiperazin-1-yl)pyrimidine | 368 | 11.1 | G |
| 33 | 4-(3-{4-[4-(2-naphthyl)pyrimidin-2-yl]piperazin-1- | 418 | 6.9 | F |
| | yl}propyl)morpholine | | | |
| 34 | 2-[4-(2-furoyl)piperazin-1-yl]-4-(2-naphthyl)pyrimidine | 385 | 10.8 | F |
| 35 | 4-(2-naphthyl)-N-(2-pyridin-3-ylethyl)pyrimidin-2-amine | 327 | 4.3 | F |
| 36 | 4-(2-naphthyl)-N-(2-pyridin-4-ylethyl)pyrimidin-2-amine | 327 | 9.4 | F |
| 37 | tert-butyl {(3R)-1-[4-(2-naphthyl)pyrimidin-2-yl]pyrrolidin-3- | 391 | 11.2 | F |
| | yl}carbamate | | | |
| 38 | (3R)-1-[4-(2-naphthyl)pyrimidin-2-yl]pyrrolidin-3-amine | 291 | 7.5 | F |
| 39 | N,N-dimethyl-1-[4-(2-naphthyl)pyrimidin-2-yl]pyrrolidin-3- | 319 | 7.6 | F |
| | amine | | | |
| 40 | N,N-diethyl-1-[4-(2-naphthyl)pyrimidin-2-yl]pyrrolidin-3- | 347 | 8.0 | F |
| | amine | | | |
| 41 | tert-butyl (3S)-3-{[4-(2-naphthyl)pyrimidin-2- | 391 | 11.3 | F |
| | yl]amino}pyrrolidine-1-carboxylate | | | |
| 42 | tert-butyl (3R)-3-{[4-(2-naphthyl)pyrimidin-2- | 391 | 11.2 | F |
| | yl]amino}pyrrolidine-1-carboxylate | | | |
| 43 | 4-(2-naphthyl)-N-[(3S)-pyrrolidin-3-yl]pyrimidin-2-amine | 291 | 7.5 | F |
| 44 | 4-(2-naphthyl)-N-[(3R)-pyrrolidin-3-yl]pyrimidin-2-amine | 291 | 7.4 | F |
| 45 | (3R)—N-methyl-1-[4-(2-naphthyl)pyrimidin-2-yl]pyrrolidin-3- | 305 | 9.8 | G |
| | amine | | | |
| 46 | (3S)—N-methyl-1-[4-(2-naphthyl)pyrimidin-2-yl]pyrrolidin-3- | 305 | 9.8 | G |
| | amine | | | |
| 47 | tert-butyl (3R)-3-({[4-(2-naphthyl)pyrimidin-2- | 405 | 11.3 | F |
| | yl]amino}methyl)pyrrolidine-1-carboxylate | | | |
| 48 | 4-(2-naphthyl)-N-[(3S)-pyrrolidin-3-ylmethyl]pyrimidin-2- | 305 | 7.7 | F |
| | amine | | | |
| 49 | (3S)—N,N-dimethyl-1-[4-(2-naphthyl)pyrimidin-2-yl]pyrrolidin- | 319 | 7.7 | F |
| | 3-amine | | | |
| 50 | (3R)—N,N-dimethyl-1-[4-(2-naphthyl)pyrimidin-2- | 319 | 7.7 | F |
| | yl]pyrrolidin-3-amine | | | |
| 51 | N-methyl-N-{(3S)-1-[4-(2-naphthyl)pyrimidin-2-yl]pyrrolidin- | 347 | 9.8 | F |
| | 3-yl}acetamide | | | |
| 52 | 4-(2-naphthyl)-N-(piperidin-4-ylmethyl)pyrimidin-2-amine | 319.3 | 7.7 | F |
| 53 | tert-butyl 4-({[4-(2-naphthyl)pyrimidin-2- | 419.3 | 11.6 | F |
| | yl]amino}methyl)piperidine-1-carboxylate | | | |
| 54 | trans-N-[4-(2-naphthyl)pyrimidin-2-yl]cyclohexane-1,4- | 319.2 | 8.0 | F |
| | diamine | | | |
| 55 | {1-[4-(2-naphthyl)pyrimidin-2-yl]piperidin-4-yl}methanol | 320 | 10.4 | F |
| 56 | 2-{1-[4-(2-naphthyl)pyrimidin-2-yl]piperidin-4-yl}ethanol | 334 | 10.9 | F |
| 57 | N-(4-methoxybenzyl)-4-(2-naphthyl)pyrimidin-2-amine | 342 | 11.1 | F |
| 58 | ethyl 1-[4-(2-naphthyl)pyrimidin-2-yl]piperidine-4-carboxylate | 362 | 11.7 | F |
| 59 | ethyl {1-[4-(2-naphthyl)pyrimidin-2-yl]piperidin-4-yl}acetate | 376 | 11.8 | F |
| 60 | N-[2-(4-methylphenyl)ethyl]-4-(2-naphthyl)pyrimidin-2-amine | 339.9 | 11.9 | F |
| 61 | 2-{methyl[4-(2-naphthyl)pyrimidin-2-yl]amino}ethanol | 280.3 | 4.6 | H |
| 62 | {(2S)-1-[4-(2-naphthyl)pyrimidin-2-yl]pyrrolidin-2- | 306.3 | 4.9 | H |
| | yl}methanol | | | |
| 63 | 1-[4-(2-naphthyl)pyrimidin-2-yl]pyrrolidin-3-ol | 292.3 | 4.2 | H |
| 64 | 4-[4-(2-naphthyl)pyrimidin-2-yl]morpholine | 292.3 | 6.2 | H |
| 65 | {1-[4-(2-naphthyl)pyrimidin-2-yl]piperidin-2-yl}methanol | 320.3 | 5.6 | H |
| 66 | {1-[4-(2-naphthyl)pyrimidin-2-yl]piperidin-3-yl}methanol | 320.3 | 5.5 | H |
| 67 | 1-[4-(2-naphthyl)pyrimidin-2-yl]piperidin-4-ol | 306.3 | 5.1 | H |
| 68 | N-(2-methoxyethyl)-4-(2-naphthyl)pyrimidin-2-amine | 280.3 | 5.0 | H |
| 69 | 2-{[4-(2-naphthyl)pyrimidin-2-yl]amino}ethanol | 266.3 | 4.0 | H |
| 70 | 1-[4-(2-naphthyl)pyrimidin-2-yl]piperidin-3-ol | 306.3 | 5.3 | H |
| 71 | {(2R)-1-[4-(2-naphthyl)pyrimidin-2-yl]pyrrolidin-2- | 306.3 | 4.9 | H |
| | yl}methanol | | | |
| 72 | N-(2-methoxyethyl)-N-methyl-4-(2-naphthyl)pyrimidin-2- | 294.3 | 6.0 | H |
| | amine | | | |
| 73 | (1-(4-(naphthalen-2-yl)pyrimidin-2-yl)piperidin-4-yl)methanol | 320.3 | 5.3 | H |
| 74 | N-methyl-4-(2-naphthyl)pyrimidin-2-amine | 236.2 | 4.5 | H |
| 75 | N,N-diethyl-4-(2-naphthyl)pyrimidin-2-amine | 278.2 | 6.7 | H |
| 76 | 4-(2-naphthyl)-N-propylpyrimidin-2-amine | 264.2 | 5.4 | H |
| 77 | N-butyl-4-(2-naphthyl)pyrimidin-2-amine | 278.2 | 6.0 | H |
| 78 | N-isopropyl-4-(2-naphthyl)pyrimidin-2-amine | 264.2 | 5.5 | H |
| 79 | N-(sec-butyl)-4-(2-naphthyl)pyrimidin-2-amine | 278.2 | 5.9 | H |
| 80 | N-isobutyl-4-(2-naphthyl)pyrimidin-2-amine | 278.2 | 5.9 | H |
| 81 | N-(tert-butyl)-4-(2-naphthyl)pyrimidin-2-amine | 278.2 | 6.1 | H |
| 82 | N-benzyl-4-(2-naphthyl)pyrimidin-2-amine | 312.2 | 6.1 | H |
| 83 | 4-(2-naphthyl)-N-(2-phenylethyl)pyrimidin-2-amine | 326.2 | 6.3 | H |
| 84 | N-cyclopentyl-4-(2-naphthyl)pyrimidin-2-amine | 290.2 | 6.0 | H |
| 85 | N-cyclohexyl-4-(2-naphthyl)pyrimidin-2-amine | 304.2 | 6.5 | H |
| 86 | 4-(2-naphthyl)-2-pyrrolidin-1-ylpyrimidine | 276.2 | 5.4 | H |
| 87 | 4-(2-naphthyl)-2-piperidin-1-ylpyrimidine | 290.2 | 7.0 | H |
| 88 | 2-(4-methylpiperidin-1-yl)-4-(2-naphthyl)pyrimidine | 304.2 | 7.4 | H |
| 89 | 1-[4-(2-naphthyl)pyrimidin-2-yl]azepane | 304.2 | 7.1 | H |
| 90 | tert-butyl 4-{[4-(2-naphthyl)pyrimidin-2-yl]amino}piperidine- | 405 | 11.3 | F |
| | 1-carboxylate | | | |
| 91 | 4-(2-naphthyl)-N-piperidin-4-ylpyrimidin-2-amine | 305 | 7.6 | F |
| 92 | tert-butyl {1-[4-(2-naphthyl)pyrimidin-2-yl]piperidin-4- | 405 | 11.5 | F |
| | yl}carbamate | | | |
| 93 | tert-butyl 4-[4-(2-naphthyl)pyrimidin-2-yl]piperazine-1- | 391 | 11.8 | F |
| | carboxylate | | | |
| 94 | 1-[4-(2-naphthyl)pyrimidin-2-yl]piperidin-4-amine | 305 | 8.2 | F |
| 95 | 4-(2-naphthyl)-2-piperazin-1-ylpyrimidine | 291 | 7.8 | F |
| 96 | tert-butyl ({1-[4-(2-naphthyl)pyrimidin-2-yl]piperidin-3- | 419 | 11.7 | F |
| | yl}methyl)carbamate | | | |
| 97 | ({1-[4-(2-naphthyl)pyrimidin-2-yl]piperidin-3- | 319 | 8.5 | F |
| | yl}methyl)amine | | | |
| 98 | tert-butyl 4-{methyl[4-(2-naphthyl)pyrimidin-2- | 419 | 12.1 | F |
| | yl]amino}piperidine-1-carboxylate | | | |
| 99 | N-methyl-4-(2-naphthyl)-N-piperidin-4-ylpyrimidin-2-amine | 319 | 8.4 | F |
| 100 | (1R,5S,6s)-3-[4-(2-naphthyl)pyrimidin-2-yl]-3- | 303 | 8.0 | F |
| | azabicyclo[3.1.0]hexan-6-amine | | | |
| 101 | tert-butyl 4-(2-methoxy-1-{[4-(2-naphthyl)pyrimidin-2- | 477 | 11.3 | F |
| | yl]amino}-2-oxoethyl)piperidine-1-carboxylate | | | |
| 102 | ({1-[4-(2-naphthyl)pyrimidin-2-yl]piperidin-4- | 319 | 8.5 | F |
| | yl}methyl)amine | | | |
| 103 | 1-[4-(2-naphthyl)pyrimidin-2-yl]piperidine-4-carboxamide | 333 | 9.5 | F |
| 104 | 8-[4-(2-naphthyl)pyrimidin-2-yl]-1,4-dioxa-8- | 348 | 11.3 | F |
| | azaspiro[4.5]decane | | | |
| 105 | methyl 1-[4-(2-naphthyl)pyrimidin-2-yl]piperidine-4- | 348 | 11.3 | F |
| | carboxylate | | | |
| 106 | 1-[4-(2-naphthyl)pyrimidin-2-yl]piperidine-3-carboxamide | 332.8 | 9.8 | F |
| 107 | {(3S)-1-[4-(2-naphthyl)pyrimidin-2-yl]piperidin-3- | 320.1 | 10.5 | F |
| | yl}methanol | | | |
| 108 | {(3R)-1-[4-(2-naphthyl)pyrimidin-2-yl]piperidin-3- | 320.1 | 10.5 | F |
| | yl}methanol | | | |

### Example 109

### Preparation of N-{(3S)-1-[4-(2-naphthyl)pyrimidin-2-yl]pyrrolidin-3-yl}acetamide

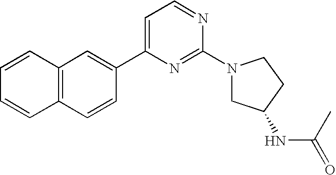

N-{(3S)-1-[4-(2-naphthyl)pyrimidin-2-yl]pyrrolidin-3-yl}acetamide. From a stock solution of 3S)-1-[4-(2-naphthyl)pyrimidin-2-yl]pyrrolidin-3-amine (0.197 g, 0.68 mmol) and diisopropylethylamine (0.145 mL, 1.36 mmol) in N-methylpyrrolidine (10 mL) is transferred to a vial a 0.5 mL aliquot. To the vial is added acetyl chloride (2.9 μL, 40.8 μmol) and the reaction is put on a shaker block for 14 h at room temperature. To the vial is added water (0.2 mL) and the solution is purified using RP HPLC (Method D) and concentrated on a speed vac to afford the title compound as an ivory powder (7.1 mg, 61%). LC/MS (Method HF gradient), rt=3.99 mins., purity=95%, calculated mass=332, [M+H]⁺=333.

### Example 110

### Preparation of 4-{Methyl[4-(2-naphthyl)pyrimidin-2-yl]amino}piperidine-1-carbaldehyde

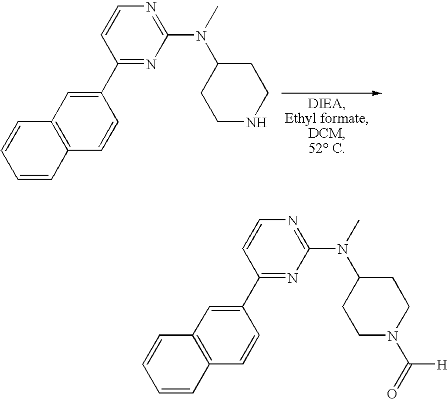

4-{Methyl[4-(2-naphthyl)pyrimidin-2-yl]amino}piperidine-1-carbaldehyde. To N-methyl-4-(2-naphthyl)-N-piperidin-4-ylpyrimidin-2-amine as the di-TFA salt 15.3 mg, 28 μMol) and diisopropylethylamine (19.4 μL, 112 μMol) is added ethyl formate (1 mL) and dichloromethane (1 mL). The reaction is shaken at 52° C. overnight. After cooling the crude is concentrated and diluted with methanol (0.5 mL) and water (0.3 mL) and purified by RP-HPLC (Method D, without TFA modifier) to yield (7.6 mg, 78%); HPLC (Method E): Purity=82%, Rt=2.5 mins. MS: (M+H)⁺=347.

### Example 111

### Preparation of N-({1-[4-(2-Naphthyl)pyrimidin-2-yl]piperidin-4-yl}methyl)guanidine

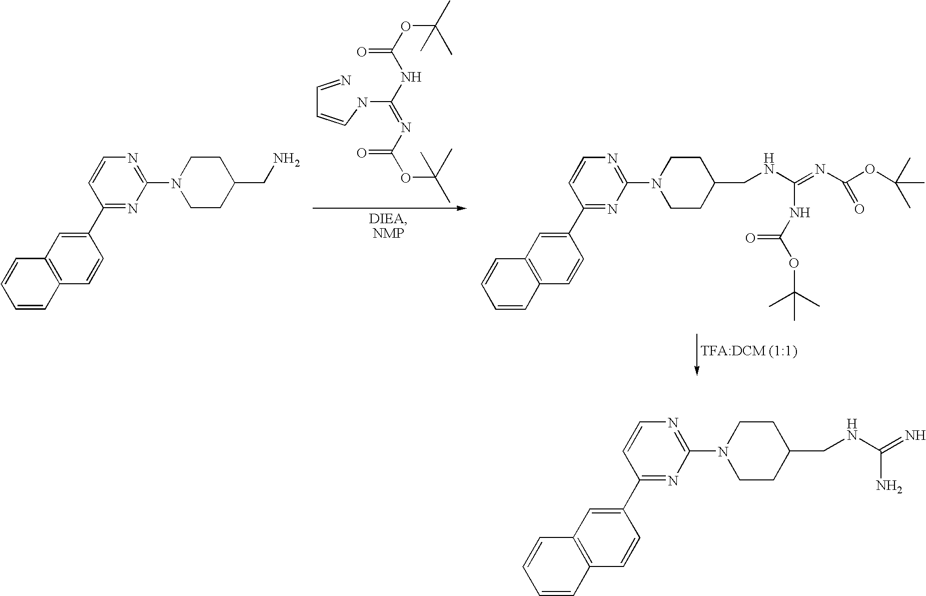

N-({1-[4-(2-Naphthyl)pyrimidin-2-yl]piperidin-4-yl}methyl)guanidine. To ({1-[4-(2-naphthyl)pyrimidin-2-yl]piperidin-4-yl}methyl)amine (12.1 mg, 38 μMol) and diisopropylethylamine (13.7 μL, 79 μMol) in NMP (1 mL) is added (tert-butoxycarbonylimino-pyrazol-1-yl-methyl)-carbamic acid tert-butyl ester (14.2 mg, 45 μMol). The reaction is stirred at room temperature overnight. The crude reaction is diluted with methanol (0.5 mL) and water (0.3 mL) and purified by RP-HPLC (Method D, without TFA modifier) to yield di-tert-butyl {(Z)-[({1-[4-(2-naphthyl)pyrimidin-2-yl]piperidin-4-yl}methyl)amino]methylylidene}biscarbamate (18.3 mg, 85.8%); HPLC (Method E): Purity=97%, Rt=3.2 mins. MS: (M+H)⁺=561.4. To (9.0 mg, 16.1 μMol) is added a solution of 50% trifluoroacetic acid in dichloromethane (1 mL) with stirring maintained overnight. The crude reaction is concentrated and diluted with methanol (0.5 mL) and water (0.3 mL) and purified by RP-HPLC (Method D, without TFA modifier) to yield N-({1-[4-(2-Naphthyl)pyrimidin-2-yl]piperidin-4-yl}methyl)guanidine as the di-TFA salt (8.4 mg, 89%); HPLC (Method E): Purity=91%, Rt=2.2 mins. MS: (M+H)⁺=361.3.

### Example 112

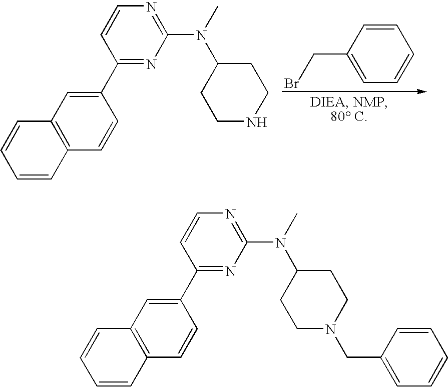

N-(1-Benzylpiperidin-4-yl)-N-methyl-4-(2-naphthyl)pyrimidin-2-amine. To a solution of N-methyl-4-(2-naphthyl)-N-piperidin-4-ylpyrimidin-2-amine as the di-TFA salt (15.3 mg, 28 μMol) and diisopropylethylamine (19.4 μL, 112 μMol) in NMP (1 mL) is added benzyl bromide (4.3 μL, 36.4 μMol). The reaction is shaken at 80° C. overnight. After cooling the crude is diluted with methanol (0.5 mL) and water (0.3 mL) and purified by RP-HPLC (Method D, without TFA modifier) to yield (6.4 mg, 56%); HPLC (Method E): Purity=98%, Rt=2.5 mins. MS: (M+H)⁺=409.

Using the methods for examples 109-112, the appropriate nucleophiles and electrophiles are reacted to form the following additional examples in the following table:

| Example | | | R.t. | HPLC |
|---|---|---|---|---|
| No | Compound | MS | (mins.) | method |
| 113 | N-ethyl-4-[4-(2-naphthyl)pyrimidin-2-yl]-1,4-diazepane-1- | 376 | 1.56 | A |
| | carboxamide | | | |
| 114 | methyl 4-[4-(2-naphthyl)pyrimidin-2-yl]-1,4-diazepane-1- | 363 | 1.80 | A |
| | carboxylate | | | |
| 115 | ethyl 4-[4-(2-naphthyl)pyrimidin-2-yl]-1,4-diazepane-1- | 377 | 1.91 | A |
| | carboxylate | | | |
| 116 | 1-acetyl-4-[4-(2-naphthyl)pyrimidin-2-yl]-1,4-diazepane | 347 | 1.54 | A |
| 117 | 1-[4-(2-naphthyl)pyrimidin-2-yl]-4-propionyl-1,4-diazepane | 361 | 1.69 | A |
| 118 | 1-[4-(2-naphthyl)pyrimidin-2-yl]-4-(trifluoroacetyl)-1,4- | 401 | 1.95 | A |
| | diazepane | | | |
| 119 | N,N-diethyl-4-[4-(2-naphthyl)pyrimidin-2-yl]-1,4-diazepane- | 404 | 1.88 | A |
| | 1-carboxamide | | | |
| 120 | 1-(methylsulfonyl)-4-[4-(2-naphthyl)pyrimidin-2-yl]-1,4- | 383 | 1.68 | A |
| | diazepane | | | |
| 121 | N,N-dimethyl-4-[4-(2-naphthyl)pyrimidin-2-yl]-1,4- | 412 | 1.82 | A |
| | diazepane-1-sulfonamide | | | |
| 122 | N,N-dimethyl-4-[4-(2-naphthyl)pyrimidin-2-yl]-1,4- | 376 | 1.67 | A |
| | diazepane-1-carboxamide | | | |
| 123 | 4-[4-(2-naphthyl)pyrimidin-2-yl]-1,4-diazepane-1- | 348 | 1.38 | A |
| | carboxamide | | | |
| 124 | 1-benzoyl-4-[4-(2-naphthyl)pyrimidin-2-yl]-1,4-diazepane | 409 | 1.82 | A |
| 125 | 1-[(4-methylphenyl)sulfonyl]-4-[4-(2-naphthyl)pyrimidin-2- | 459 | 2.06 | A |
| | yl]-1,4-diazepane | | | |
| 126 | N-{1-[4-(2-naphthyl)pyrimidin-2-yl]azepan-4-yl}acetamide | 361 | 1.50 | A |
| 127 | 2,2,2-trifluoro-N-{1-[4-(2-naphthyl)pyrimidin-2-yl]azepan- | 415 | 1.89 | A |
| | 4-yl}acetamide | | | |
| 128 | N,N-dimethyl-N′-{1-[4-(2-naphthyl)pyrimidin-2-yl]azepan- | 390 | 1.57 | A |
| | 4-yl}urea | | | |
| 129 | N-{1-[4-(2-naphthyl)pyrimidin-2-yl]azepan-4-yl}benzamide | 423 | 1.87 | A |
| 130 | N-{1-[4-(2-naphthyl)pyrimidin-2-yl]azepan-4-yl}urea | 361 | 1.43 | A |
| 131 | N-ethyl-N′-{1-[4-(2-naphthyl)pyrimidin-2-yl]azepan-4- | 390 | 1.54 | A |
| | yl}urea | | | |
| 132 | N-{1-[4-(2-naphthyl)pyrimidin-2-yl]azepan-4- | 397 | 1.58 | A |
| | yl}methanesulfonamide | | | |
| 133 | 4-methyl-N-{1-[4-(2-naphthyl)pyrimidin-2-yl]azepan-4- | 473 | 2.00 | A |
| | yl}benzenesulfonamide | | | |
| 134 | N,N-dimethyl-N′-{1-[4-(2-naphthyl)pyrimidin-2-yl]azepan- | 426 | 1.75 | A |
| | 4-yl}sulfamide | | | |
| 135 | N-[(1-acetylazetidin-3-yl)methyl]-4-(2-naphthyl)pyrimidin- | 333 | 1.30 | A |
| | 2-amine | | | |
| 136 | 4-(2-naphthyl)-N-{[1-(trifluoroacetyl)azetidin-3- | 387 | 1.73 | A |
| | yl]methyl}pyrimidin-2-amine | | | |
| 137 | N-[(1-benzoylazetidin-3-yl)methyl]-4-(2- | 395 | 1.63 | A |
| | naphthyl)pyrimidin-2-amine | | | |
| 138 | 3-({[4-(2-naphthyl)pyrimidin-2-yl]amino}methyl)azetidine- | 334 | 1.19 | A |
| | 1-carboxamide | | | |
| 139 | N-ethyl-3-({[4-(2-naphthyl)pyrimidin-2- | 362 | 1.12 | A |
| | yl]amino}methyl)azetidine-1-carboxamide | | | |
| 140 | N-{[1-(methylsulfonyl)azetidin-3-yl]methyl}-4-(2- | 369 | 1.46 | A |
| | naphthyl)pyrimidin-2-amine | | | |
| 141 | N-({1-[(4-methylphenyl)sulfonyl]azetidin-3-yl}methyl)-4- | 445 | 1.89 | A |
| | (2-naphthyl)pyrimidin-2-amine | | | |
| 142 | N,N-dimethyl-3-({[4-(2-naphthyl)pyrimidin-2- | 398 | 1.72 | A |
| | yl]amino}methyl)azetidine-1-sulfonamide | | | |
| 143 | N-({1-[4-(2-naphthyl)pyrimidin-2-yl]azetidin-3- | 333 | 1.31 | A |
| | yl}methyl)acetamide | | | |
| 144 | 2,2,2-trifluoro-N-({1-[4-(2-naphthyl)pyrimidin-2-yl]azetidin- | 387 | 1.69 | A |
| | 3-yl}methyl)acetamide | | | |
| 145 | N,N-dimethyl-N′-({1-[4-(2-naphthyl)pyrimidin-2-yl]azetidin- | 362 | 1.36 | A |
| | 3-yl}methyl)urea | | | |
| 146 | methyl ({1-[4-(2-naphthyl)pyrimidin-2-yl]azetidin-3- | 349 | 1.50 | A |
| | yl}methyl)carbamate | | | |
| 147 | N-({1-[4-(2-naphthyl)pyrimidin-2-yl]azetidin-3- | 334 | 1.22 | A |
| | yl}methyl)urea | | | |
| 148 | N-ethyl-N′-({1-[4-(2-naphthyl)pyrimidin-2-yl]azetidin-3- | 362 | 1.38 | A |
| | yl}methyl)urea | | | |
| 149 | N-({1-[4-(2-naphthyl)pyrimidin-2-yl]azetidin-3- | 369 | 1.41 | A |
| | yl}methyl)methanesulfonamide | | | |
| 150 | 4-methyl-N-({1-[4-(2-naphthyl)pyrimidin-2-yl]azetidin-3- | 445 | 1.85 | A |
| | yl}methyl)benzenesulfonamide | | | |
| 151 | {1-[4-(2-naphthyl)pyrimidin-2-yl]azepan-4-yl}formamide | 347 | 10.0 | F |
| 152 | N,N-dimethyl-N′-(2-{4-[4-(2-naphthyl)pyrimidin-2- | 404.9 | 2.2 | E |
| | yl]piperazin-1-yl}ethyl)urea | | | |
| 153 | N-(2-{4-[4-(2-naphthyl)pyrimidin-2-yl]piperazin-1- | 411.9 | 2.3 | E |
| | yl}ethyl)methanesulfonamide | | | |
| 154 | N-(2-{4-[4-(2-naphthyl)pyrimidin-2-yl]piperazin-1- | 419.9 | 2.1 | E |
| | yl}ethyl)dicarbonimidic diamide | | | |
| 155 | N-ethyl-N′-(2-{4-[4-(2-naphthyl)pyrimidin-2-yl]piperazin-1- | 404.9 | 2.2 | E |
| | yl}ethyl)urea | | | |
| 156 | N-isopropyl-N′-(2-{4-[4-(2-naphthyl)pyrimidin-2- | 418.9 | 2.3 | E |
| | yl]piperazin-1-yl}ethyl)urea | | | |
| 157 | diethyl (2-{4-[4-(2-naphthyl)pyrimidin-2-yl]piperazin-1- | 477.9 | 2.8 | E |
| | yl}ethyl)imidodicarbonate | | | |
| 158 | N-{(3R)-1-[4-(2-naphthyl)pyrimidin-2-yl]pyrrolidin-3- | 332.9 | 2.2 | E |
| | yl}acetamide | | | |
| 159 | 2,2,2-trifluoro-N-{(3R)-1-[4-(2-naphthyl)pyrimidin-2- | 386.9 | 2.5 | E |
| | yl]pyrrolidin-3-yl}acetamide | | | |
| 160 | methyl {(3R)-1-[4-(2-naphthyl)pyrimidin-2-yl]pyrrolidin-3- | 348.9 | 2.4 | E |
| | yl}carbamate | | | |
| 161 | N,N-dimethyl-N′-{(3R)-1-[4-(2-naphthyl)pyrimidin-2- | 361.9 | 2.3 | E |
| | yl]pyrrolidin-3-yl}urea | | | |
| 162 | N-{(3R)-1-[4-(2-naphthyl)pyrimidin-2-yl]pyrrolidin-3- | 387.9 | 2.4 | E |
| | yl}pyrrolidine-1-carboxamide | | | |
| 163 | N-{(3R)-1-[4-(2-naphthyl)pyrimidin-2-yl]pyrrolidin-3- | 333.9 | 2 | E |
| | yl}urea | | | |
| 164 | N,N-dimethyl-N′-{(3R)-1-[4-(2-naphthyl)pyrimidin-2- | 397.9 | 2.4 | E |
| | yl]pyrrolidin-3-yl}sulfamide | | | |
| 165 | N-{(3R)-1-[4-(2-naphthyl)pyrimidin-2-yl]pyrrolidin-3- | 368.9 | 2.2 | E |
| | yl}methanesulfonamide | | | |
| 166 | 4-methyl-N-{(3R)-1-[4-(2-naphthyl)pyrimidin-2- | 444.9 | 2.6 | E |
| | yl]pyrrolidin-3-yl}benzenesulfonamide | | | |
| 167 | N-{(3R)-1-[4-(2-naphthyl)pyrimidin-2-yl]pyrrolidin-3- | 403.9 | 2.2 | E |
| | yl}morpholine-4-carboxamide | | | |
| 168 | ethyl {(3R)-1-[4-(2-naphthyl)pyrimidin-2-yl]pyrrolidin-3- | 362.9 | 2.4 | E |
| | yl}carbamate | | | |
| 169 | N-isopropyl-N′-{(3R)-1-[4-(2-naphthyl)pyrimidin-2- | 375.9 | 2.3 | E |
| | yl]pyrrolidin-3-yl}urea | | | |
| 170 | N,N-diethyl-N′-{(3R)-1-[4-(2-naphthyl)pyrimidin-2- | 390 | 2.4 | E |
| | yl]pyrrolidin-3-yl}urea | | | |
| 171 | N-{(3R)-1-[4-(2-naphthyl)pyrimidin-2-yl]pyrrolidin-3- | 332.9 | 2 | E |
| | yl}guanidine | | | |
| 172 | 4-chloro-N-{(3R)-1-[4-(2-naphthyl)pyrimidin-2- | 428.9 | 2.6 | E |
| | yl]pyrrolidin-3-yl}benzamide | | | |
| 173 | 4-cyano-N-{(3R)-1-[4-(2-naphthyl)pyrimidin-2- | 419.9 | 2.5 | E |
| | yl]pyrrolidin-3-yl}benzamide | | | |
| 174 | N-{(3S)-1-[4-(2-naphthyl)pyrimidin-2-yl]pyrrolidin-3- | 333.2 | 2.1 | E |
| | yl}acetamide | | | |
| 175 | 2,2,2-trifluoro-N-{(3S)-1-[4-(2-naphthyl)pyrimidin-2- | 387.3 | 2.4 | E |
| | yl]pyrrolidin-3-yl}acetamide | | | |
| 176 | N-{(3S)-1-[4-(2-naphthyl)pyrimidin-2-yl]pyrrolidin-3- | 395.3 | 2.4 | E |
| | yl}benzamide | | | |
| 177 | methyl {(3S)-1-[4-(2-naphthyl)pyrimidin-2-yl]pyrrolidin-3- | 349.3 | 2.3 | E |
| | yl}carbamate | | | |
| 178 | N,N-dimethyl-N′-{(3S)-1-[4-(2-naphthyl)pyrimidin-2- | 362.3 | 2.2 | E |
| | yl]pyrrolidin-3-yl}urea | | | |
| 179 | N-{(3S)-1-[4-(2-naphthyl)pyrimidin-2-yl]pyrrolidin-3- | 388.3 | 2.3 | E |
| | yl}pyrrolidine-1-carboxamide | | | |
| 180 | N-ethyl-N′-{(3S)-1-[4-(2-naphthyl)pyrimidin-2- | 362.3 | 2.2 | E |
| | yl]pyrrolidin-3-yl}urea | | | |
| 181 | N-{(3S)-1-[4-(2-naphthyl)pyrimidin-2-yl]pyrrolidin-3- | 334.2 | 2 | E |
| | yl}urea | | | |
| 182 | N,N-dimethyl-N′-{(3S)-1-[4-(2-naphthyl)pyrimidin-2- | 398.3 | 2.3 | E |
| | yl]pyrrolidin-3-yl}sulfamide | | | |
| 183 | N-{(3S)-1-[4-(2-naphthyl)pyrimidin-2-yl]pyrrolidin-3- | 369.2 | 2.2 | E |
| | yl}methanesulfonamide | | | |
| 184 | 4-methyl-N-{(3S)-1-[4-(2-naphthyl)pyrimidin-2- | 445.2 | 2.6 | E |
| | yl]pyrrolidin-3-yl}benzenesulfonamide | | | |
| 185 | N-{(3S)-1-[4-(2-naphthyl)pyrimidin-2-yl]pyrrolidin-3- | 404.3 | 2.2 | E |
| | yl}morpholine-4-carboxamide | | | |
| 186 | ethyl {(3S)-1-[4-(2-naphthyl)pyrimidin-2-yl]pyrrolidin-3- | 363.3 | 2.4 | E |
| | yl}carbamate | | | |
| 187 | N-isopropyl-N′-{(3S)-1-[4-(2-naphthyl)pyrimidin-2- | 376.3 | 2.2 | E |
| | yl]pyrrolidin-3-yl}urea | | | |
| 188 | N,N-diethyl-N′-{(3S)-1-[4-(2-naphthyl)pyrimidin-2- | 390.3 | 2.4 | E |
| | yl]pyrrolidin-3-yl}urea | | | |
| 189 | 4-chloro-N-{(3S)-1-[4-(2-naphthyl)pyrimidin-2- | 429.2 | 2.6 | E |
| | yl]pyrrolidin-3-yl}benzamide | | | |
| 190 | 4-cyano-N-{(3S)-1-[4-(2-naphthyl)pyrimidin-2- | 420.3 | 2.4 | E |
| | yl]pyrrolidin-3-yl}benzamide | | | |
| 191 | N-[(3S)-1-acetylpyrrolidin-3-yl]-4-(2-naphthyl)pyrimidin-2- | 333 | 2.2 | E |
| | amine | | | |
| 192 | N-[(3S)-1-benzoylpyrrolidin-3-yl]-4-(2-naphthyl)pyrimidin- | 395 | 2.5 | E |
| | 2-amine | | | |
| 193 | methyl (3S)-3-{[4-(2-naphthyl)pyrimidin-2- | 349 | 2.4 | E |
| | yl]amino}pyrrolidine-1-carboxylate | | | |
| 194 | (3S)—N,N-dimethyl-3-{[4-(2-naphthyl)pyrimidin-2- | 362 | 2.3 | E |
| | yl]amino}pyrrolidine-1-carboxamide | | | |
| 195 | (3S)—N-ethyl-3-{[4-(2-naphthyl)pyrimidin-2- | 362 | 2.2 | E |
| | yl]amino}pyrrolidine-1-carboxamide | | | |
| 196 | (3S)-3-{[4-(2-naphthyl)pyrimidin-2-yl]amino}pyrrolidine-1- | 334 | 2.1 | E |
| | carboxamide | | | |
| 197 | (3S)—N,N-dimethyl-3-{[4-(2-naphthyl)pyrimidin-2- | 398 | 2.5 | E |
| | yl]amino}pyrrolidine-1-sulfonamide | | | |
| 198 | N-[(3S)-1-(methylsulfonyl)pyrrolidin-3-yl]-4-(2- | 369 | 2.3 | E |
| | naphthyl)pyrimidin-2-amine | | | |
| 199 | N-{(3S)-1-[(4-methylphenyl)sulfonyl]pyrrolidin-3-yl}-4-(2- | 445 | 2.7 | E |
| | naphthyl)pyrimidin-2-amine | | | |
| 200 | (3S)-3-{[4-(2-naphthyl)pyrimidin-2-yl]amino}pyrrolidine-1- | 319 | 2.2 | E |
| | carbaldehyde | | | |
| 201 | N-[(3S)-1-(morpholin-4-ylcarbonyl)pyrrolidin-3-yl]-4-(2- | 404 | 2.3 | E |
| | naphthyl)pyrimidin-2-amine | | | |
| 202 | ethyl (3S)-3-{[4-(2-naphthyl)pyrimidin-2- | 363 | 2.5 | E |
| | yl]amino}pyrrolidine-1-carboxylate | | | |
| 203 | N-[(3R)-1-acetylpyrrolidin-3-yl]-4-(2-naphthyl)pyrimidin-2- | 333 | 2.2 | E |
| | amine | | | |
| 204 | N-[(3R)-1-benzoylpyrrolidin-3-yl]-4-(2-naphthyl)pyrimidin- | 395 | 2.5 | E |
| | 2-amine | | | |
| 205 | methyl (3R)-3-{[4-(2-naphthyl)pyrimidin-2- | 349 | 2.4 | E |
| | yl]amino}pyrrolidine-1-carboxylate | | | |
| 206 | (3R)—N,N-dimethyl-3-{[4-(2-naphthyl)pyrimidin-2- | 362 | 2.3 | E |
| | yl]amino}pyrrolidine-1-carboxamide | | | |
| 207 | (3R)—N-ethyl-3-{[4-(2-naphthyl)pyrimidin-2- | 362 | 2.2 | E |
| | yl]amino}pyrrolidine-1-carboxamide | | | |
| 208 | (3R)-3-{[4-(2-naphthyl)pyrimidin-2-yl]amino}pyrrolidine-1- | 334 | 2.1 | E |
| | carboxamide | | | |
| 209 | (3R)—N,N-dimethyl-3-{[4-(2-naphthyl)pyrimidin-2- | 398 | 2.5 | E |
| | yl]amino}pyrrolidine-1-sulfonamide | | | |
| 210 | N-[(3R)-1-(methylsulfonyl)pyrrolidin-3-yl]-4-(2- | 369 | 2.3 | E |
| | naphthyl)pyrimidin-2-amine | | | |
| 211 | N-{(3R)-1-[(4-methylphenyl)sulfonyl]pyrrolidin-3-yl}-4-(2- | 445 | 2.7 | E |
| | naphthyl)pyrimidin-2-amine | | | |
| 212 | (3R)-3-{[4-(2-naphthyl)pyrimidin-2-yl]amino}pyrrolidine-1- | 319 | 2.2 | E |
| | carbaldehyde | | | |
| 213 | N-[(3R)-1-(morpholin-4-ylcarbonyl)pyrrolidin-3-yl]-4-(2- | 404 | 2.3 | E |
| | naphthyl)pyrimidin-2-amine | | | |
| 214 | ethyl (3R)-3-{[4-(2-naphthyl)pyrimidin-2- | 363 | 2.5 | E |
| | yl]amino}pyrrolidine-1-carboxylate | | | |
| 215 | N-{[(3R)-1-acetylpyrrolidin-3-yl]methyl}-4-(2- | 347 | 2.2 | E |
| | naphthyl)pyrimidin-2-amine | | | |
| 216 | methyl (3R)-3-({[4-(2-naphthyl)pyrimidin-2- | 363 | 2.4 | E |
| | yl]amino}methyl)pyrrolidine-1-carboxylate | | | |
| 217 | (3R)-3-({[4-(2-naphthyl)pyrimidin-2- | 333 | 2.2 | E |
| | yl]amino}methyl)pyrrolidine-1-carbaldehyde | | | |
| 218 | N-({1-[4-(2-naphthyl)pyrimidin-2-yl]pyrrolidin-3- | 347 | 2.3 | E |
| | yl}methyl)acetamide | | | |
| 219 | N-({1-[4-(2-naphthyl)pyrimidin-2-yl]pyrrolidin-3- | 409 | 2.6 | E |
| | yl}methyl)benzamide | | | |
| 220 | methyl ({1-[4-(2-naphthyl)pyrimidin-2-yl]pyrrolidin-3- | 363 | 2.5 | E |
| | yl}methyl)carbamate | | | |
| 221 | N,N-dimethyl-N′-({1-[4-(2-naphthyl)pyrimidin-2- | 376 | 2.4 | E |
| | yl]pyrrolidin-3-yl}methyl)urea | | | |
| 222 | N-ethyl-N′-({1-[4-(2-naphthyl)pyrimidin-2-yl]pyrrolidin-3- | 376 | 2.3 | E |
| | yl}methyl)urea | | | |
| 223 | N-({1-[4-(2-naphthyl)pyrimidin-2-yl]pyrrolidin-3- | 348 | 2.2 | E |
| | yl}methyl)urea | | | |
| 224 | N,N-dimethyl-N′-({1-[4-(2-naphthyl)pyrimidin-2- | 412 | 2.5 | E |
| | yl]pyrrolidin-3-yl}methyl)sulfamide | | | |
| 225 | N-({1-[4-(2-naphthyl)pyrimidin-2-yl]pyrrolidin-3- | 383 | 2.4 | E |
| | yl}methyl)methanesulfonamide | | | |
| 226 | N-({1-[4-(2-naphthyl)pyrimidin-2-yl]pyrrolidin-3- | 333 | 2.3 | E |
| | yl}methyl)formamide | | | |
| 227 | N-{[(3S)-1-acetylpyrrolidin-3-yl]methyl}-4-(2- | 347 | 2.2 | E |
| | naphthyl)pyrimidin-2-amine | | | |
| 228 | methyl (3S)-3-({[4-(2-naphthyl)pyrimidin-2- | 363 | 2.4 | E |
| | yl]amino}methyl)pyrrolidine-1-carboxylate | | | |
| 229 | (3S)-3-({[4-(2-naphthyl)pyrimidin-2- | 333 | 2.2 | E |
| | yl]amino}methyl)pyrrolidine-1-carbaldehyde | | | |
| 230 | N-[(1-acetylpiperidin-4-yl)methyl]-4-(2-naphthyl)pyrimidin- | 361 | 1.5 | A |
| | 2-amine | | | |
| 231 | N,N-dimethyl-4-({[4-(2-naphthyl)pyrimidin-2- | 390 | 1.6 | A |
| | yl]amino}methyl)piperidine-1-carboxamide | | | |
| 232 | N,N-diethyl-4-({[4-(2-naphthyl)pyrimidin-2- | 418 | 1.9 | A |
| | yl]amino}methyl)piperidine-1-carboxamide | | | |
| 233 | 4-(2-naphthyl)-N-{[1-(pyrrolidin-1-ylcarbonyl)piperidin-4- | 416 | 1.8 | A |
| | yl]methyl}pyrimidin-2-amine | | | |
| 234 | N-{[1-(methylsulfonyl)piperidin-4-yl]methyl}-4-(2- | 397 | 1.6 | A |
| | naphthyl)pyrimidin-2-amine | | | |
| 235 | N,N-dimethyl-4-({[4-(2-naphthyl)pyrimidin-2- | 426 | 1.8 | A |
| | yl]amino}methyl)piperidine-1-sulfonamide | | | |
| 236 | 4-({[4-(2-naphthyl)pyrimidin-2-yl]amino}methyl)piperidine- | 362 | 1.3 | A |
| | 1-carboxamide | | | |
| 237 | N-ethyl-4-({[4-(2-naphthyl)pyrimidin-2- | 390 | 1.5 | A |
| | yl]amino}methyl)piperidine-1-carboxamide | | | |
| 238 | N-isopropyl-4-({[4-(2-naphthyl)pyrimidin-2- | 404 | 1.6 | A |
| | yl]amino}methyl)piperidine-1-carboxamide | | | |
| 239 | N-cyclohexyl-4-({[4-(2-naphthyl)pyrimidin-2- | 444 | 1.9 | A |
| | yl]amino}methyl)piperidine-1-carboxamide | | | |
| 240 | N-[(1-benzoylpiperidin-4-yl)methyl]-4-(2- | 423 | 1.8 | A |
| | naphthyl)pyrimidin-2-amine | | | |
| 241 | N-[(1-ethylpiperidin-4-yl)methyl]-4-(2-naphthyl)pyrimidin- | 347 | 1.2 | A |
| | 2-amine | | | |
| 242 | N-[(1-benzylpiperidin-4-yl)methyl]-4-(2- | 409 | 1.6 | A |
| | naphthyl)pyrimidin-2-amine | | | |
| 243 | 4-(2-naphthyl)-N-{[1-(phenylacetyl)piperidin-4- | 437 | 1.8 | A |
| | yl]methyl}pyrimidin-2-amine | | | |
| 244 | N-({1-[(4-methylphenyl)sulfonyl]piperidin-4-yl}methyl)-4- | 473 | 2.1 | A |
| | (2-naphthyl)pyrimidin-2-amine | | | |
| 245 | N-(trans-4-{[4-(2-naphthyl)pyrimidin-2- | 361 | 2.2 | E |
| | yl]amino}cyclohexyl)acetamide | | | |
| 246 | methyl (trans-4-{[4-(2-naphthyl)pyrimidin-2- | 377 | 2.3 | E |
| | yl]amino}cyclohexyl)carbamate | | | |
| 247 | N,N-dimethyl-N′-(trans-4-{[4-(2-naphthyl)pyrimidin-2- | 390 | 2.3 | E |
| | yl]amino}cyclohexyl)urea | | | |
| 248 | N-ethyl-N′-(trans-4-{[4-(2-naphthyl)pyrimidin-2- | 390 | 2.2 | E |
| | yl]amino}cyclohexyl)urea | | | |
| 249 | N,N-dimethyl-N′-(trans-4-{[4-(2-naphthyl)pyrimidin-2- | 426 | 2.4 | E |
| | yl]amino}cyclohexyl)sulfamide | | | |
| 250 | N-(trans-4-{[4-(2-naphthyl)pyrimidin-2- | 397 | 2.2 | E |
| | yl]amino}cyclohexyl)methanesulfonamide | | | |
| 251 | 4-methyl-N-(trans-4-{[4-(2-naphthyl)pyrimidin-2- | 473 | 2.6 | E |
| | yl]amino}cyclohexyl)benzenesulfonamide | | | |
| 252 | 5-(dimethylamino)-N-(trans-4-{[4-(2-naphthyl)pyrimidin-2- | 551 | 2 | A |
| | yl]amino}cyclohexyl)naphthalene-1-sulfonamide | | | |
| 253 | N-(2-{1-[4-(2-naphthyl)pyrimidin-2-yl]piperidin-4- | 375 | 1.7 | E |
| | yl}ethyl)acetamide | | | |
| 254 | 2,2,2-trifluoro-N-(2-{1-[4-(2-naphthyl)pyrimidin-2- | 429 | 2 | E |
| | yl]piperidin-4-yl}ethyl)acetamide | | | |
| 255 | methyl (2-{1-[4-(2-naphthyl)pyrimidin-2-yl]piperidin-4- | 391 | 1.9 | E |
| | yl}ethyl)carbamate | | | |
| 256 | N,N-dimethyl-N′-(2-{1-[4-(2-naphthyl)pyrimidin-2- | 404 | 1.7 | E |
| | yl]piperidin-4-yl}ethyl)urea | | | |
| 257 | N,N-diethyl-N′-(2-{1-[4-(2-naphthyl)pyrimidin-2- | 432 | 1.9 | E |
| | yl]piperidin-4-yl}ethyl)urea | | | |
| 258 | N-ethyl-N′-(2-{1-[4-(2-naphthyl)pyrimidin-2-yl]piperidin-4- | 404 | 1.7 | E |
| | yl}ethyl)urea | | | |
| 259 | N-isopropyl-N′-(2-{1-[4-(2-naphthyl)pyrimidin-2- | 418 | 1.8 | E |
| | yl]piperidin-4-yl}ethyl)urea | | | |
| 260 | N-(2-{1-[4-(2-naphthyl)pyrimidin-2-yl]piperidin-4- | 376 | 1.5 | E |
| | yl}ethyl)urea | | | |
| 261 | N-cyclohexyl-N′-(2-{1-[4-(2-naphthyl)pyrimidin-2- | 458 | 2 | E |
| | yl]piperidin-4-yl}ethyl)urea | | | |
| 262 | N-(2-{1-[4-(2-naphthyl)pyrimidin-2-yl]piperidin-4- | 430 | 1.9 | E |
| | yl}ethyl)pyrrolidine-1-carboxamide | | | |
| 263 | N-(2-{1-[4-(2-naphthyl)pyrimidin-2-yl]piperidin-4- | 446 | 1.7 | E |
| | yl}ethyl)morpholine-4-carboxamide | | | |
| 264 | N,N-dimethyl-N′-(2-{1-[4-(2-naphthyl)pyrimidin-2- | 440 | 2.3 | E |
| | yl]piperidin-4-yl}ethyl)sulfamide | | | |
| 265 | N-(2-{1-[4-(2-naphthyl)pyrimidin-2-yl]piperidin-4- | 411 | 1.7 | E |
| | yl}ethyl)methanesulfonamide | | | |
| 266 | benzyl (2-{1-[4-(2-naphthyl)pyrimidin-2-yl]piperidin-4- | 467 | 2.5 | E |
| | yl}ethyl)carbamate | | | |
| 267 | N-(2-{1-[4-(2-naphthyl)pyrimidin-2-yl]piperidin-4- | 452 | 2.3 | E |
| | yl}ethyl)-N′-phenylurea | | | |
| 268 | N-(2-{1-[4-(2-naphthyl)pyrimidin-2-yl]piperidin-4- | 437 | 2.3 | E |
| | yl}ethyl)benzamide | | | |
| 269 | 4-cyano-N-(3-{[4-(2-naphthyl)pyrimidin-2- | 448 | 1.64 | A |
| | yl]amino}cyclohexyl)benzamide | | | |
| 270 | N-(1-acetylpiperidin-4-yl)-4-(2-naphthyl)pyrimidin-2-amine | 347 | 9.4 | F |
| 271 | N,N-dimethyl-4-{[4-(2-naphthyl)pyrimidin-2- | 376 | 1.6 | A |
| | yl]amino}piperidine-1-carboxamide | | | |
| 272 | N,N-diethyl-4-{[4-(2-naphthyl)pyrimidin-2- | 404 | 2 | A |
| | yl]amino}piperidine-1-carboxamide | | | |
| 273 | 4-(2-naphthyl)-N-[1-(pyrrolidin-1-ylcarbonyl)piperidin-4- | 402 | 1.8 | A |
| | yl]pyrimidin-2-amine | | | |
| 274 | N-[1-(morpholin-4-ylcarbonyl)piperidin-4-yl]-4-(2- | 418 | 1.6 | A |
| | naphthyl)pyrimidin-2-amine | | | |
| 275 | N-[1-(methylsulfonyl)piperidin-4-yl]-4-(2- | 383 | 1.6 | A |
| | naphthyl)pyrimidin-2-amine | | | |
| 276 | N,N-dimethyl-4-{[4-(2-naphthyl)pyrimidin-2- | 412 | 1.8 | A |
| | yl]amino}piperidine-1-sulfonamide | | | |
| 277 | 4-{[4-(2-naphthyl)pyrimidin-2-yl]amino}piperidine-1- | 348 | 8.9 | F |
| | carboxamide | | | |
| 278 | N-ethyl-4-{[4-(2-naphthyl)pyrimidin-2-yl]amino}piperidine- | 376 | 1.5 | A |
| | 1-carboxamide | | | |
| 279 | N-isopropyl-4-{[4-(2-naphthyl)pyrimidin-2- | 390 | 1.7 | A |
| | yl]amino}piperidine-1-carboxamide | | | |
| 280 | N-cyclohexyl-4-{[4-(2-naphthyl)pyrimidin-2- | 430 | 2 | A |
| | yl]amino}piperidine-1-carboxamide | | | |
| 281 | N-(1-benzoylpiperidin-4-yl)-4-(2-naphthyl)pyrimidin-2- | 409 | 1.8 | A |
| | amine | | | |
| 282 | N-(1-acetylpiperidin-4-yl)-N-methyl-4-(2- | 361 | 2.4 | E |
| | naphthyl)pyrimidin-2-amine | | | |
| 283 | N-methyl-4-(2-naphthyl)-N-[1-(trifluoroacetyl)piperidin-4- | 415 | 2.8 | E |
| | yl]pyrimidin-2-amine | | | |
| 284 | N-(1-benzoylpiperidin-4-yl)-N-methyl-4-(2- | 423 | 2.7 | E |
| | naphthyl)pyrimidin-2-amine | | | |
| 285 | methyl 4-{methyl[4-(2-naphthyl)pyrimidin-2- | 377 | 2.7 | E |
| | yl]amino}piperidine-1-carboxylate | | | |
| 286 | N,N-dimethyl-4-{methyl[4-(2-naphthyl)pyrimidin-2- | 390 | 2.6 | E |
| | yl]amino}piperidine-1-carboxamide | | | |
| 287 | N-methyl-4-(2-naphthyl)-N-[1-(pyrrolidin-1- | 416 | 2.4 | E |
| | ylcarbonyl)piperidin-4-yl]pyrimidin-2-amine | | | |
| 288 | N,N-diethyl-4-{methyl[4-(2-naphthyl)pyrimidin-2- | 418 | 2.3 | E |
| | yl]amino}piperidine-1-carboxamide | | | |
| 289 | N-ethyl-4-{methyl[4-(2-naphthyl)pyrimidin-2- | 390 | 2.4 | E |
| | yl]amino}piperidine-1-carboxamide | | | |
| 290 | 4-{methyl[4-(2-naphthyl)pyrimidin-2-yl]amino}piperidine- | 362 | 2.3 | E |
| | 1-carboxamide | | | |
| 291 | N-methyl-N-[1-(methylsulfonyl)piperidin-4-yl]-4-(2- | 397 | 2.5 | E |
| | naphthyl)pyrimidin-2-amine | | | |
| 292 | N-methyl-N-{1-[(4-methylphenyl)sulfonyl]piperidin-4-yl}- | 473 | 2.9 | E |
| | 4-(2-naphthyl)pyrimidin-2-amine | | | |
| 293 | N-methyl-4-(2-naphthyl)-N-(1-pyrimidin-2-ylpiperidin-4- | 397 | 3.1 | E |
| | yl)pyrimidin-2-amine | | | |
| 294 | N-methyl-4-(2-naphthyl)-N-(1-propylpiperidin-4- | 361 | 2.1 | E |
| | yl)pyrimidin-2-amine | | | |
| 295 | N-(1-benzylpiperidin-4-yl)-N-methyl-4-(2- | 409 | 2.5 | E |
| | naphthyl)pyrimidin-2-amine | | | |
| 296 | 2-(4-{methyl[4-(2-naphthyl)pyrimidin-2- | 376 | 2.2 | E |
| | yl]amino}piperidin-1-yl)acetamide | | | |
| 297 | N-methyl-4-(2-naphthyl)-N-{1-[2-(trityloxy)ethyl]piperidin- | 605 | 3.3 | E |
| | 4-yl}pyrimidin-2-amine | | | |
| 298 | 4-{methyl[4-(2-naphthyl)pyrimidin-2-yl]amino}piperidine- | 347 | 2.5 | E |
| | 1-carbaldehyde | | | |
| 299 | 4-{methyl[4-(2-naphthyl)pyrimidin-2-yl]amino}piperidine- | 361 | 2 | E |
| | 1-carboximidamide | | | |
| 300 | N-((1-(4-(naphthalen-2-yl)pyrimidin-2-yl)piperidin-4- | 361 | 10.2 | G |
| | yl)methyl)acetamide | | | |
| 301 | N-({1-[4-(2-naphthyl)pyrimidin-2-yl]piperidin-4- | 422.9 | 2.6 | E |
| | yl}methyl)benzamide | | | |
| 302 | methyl ({1-[4-(2-naphthyl)pyrimidin-2-yl]piperidin-4- | 377 | 2.5 | E |
| | yl}methyl)carbamate | | | |
| 303 | N,N-dimethyl-N′-({1-[4-(2-naphthyl)pyrimidin-2- | 390 | 2.4 | E |
| | yl]piperidin-4-yl}methyl)urea | | | |
| 304 | N-({1-[4-(2-naphthyl)pyrimidin-2-yl]piperidin-4- | 415.9 | 2.5 | E |
| | yl}methyl)pyrrolidine-1-carboxamide | | | |
| 305 | N-ethyl-N′-({1-[4-(2-naphthyl)pyrimidin-2-yl]piperidin-4- | 390 | 2.4 | E |
| | yl}methyl)urea | | | |
| 306 | N-({1-[4-(2-naphthyl)pyrimidin-2-yl]piperidin-4- | 361.9 | 2.2 | E |
| | yl}methyl)urea | | | |
| 307 | N,N-dimethyl-N′-({1-[4-(2-naphthyl)pyrimidin-2- | 425.9 | 2.6 | E |
| | yl]piperidin-4-yl}methyl)sulfamide | | | |
| 308 | N-({1-[4-(2-naphthyl)pyrimidin-2-yl]piperidin-4- | 396.9 | 2.4 | E |
| | yl}methyl)methanesulfonamide | | | |
| 309 | 4-methyl-N-({1-[4-(2-naphthyl)pyrimidin-2-yl]piperidin-4- | 472.9 | 2.8 | E |
| | yl}methyl)benzenesulfonamide | | | |
| 310 | N-({1-[4-(2-naphthyl)pyrimidin-2-yl]piperidin-4- | 396.9 | 2.7 | E |
| | yl}methyl)pyrimidin-2-amine | | | |
| 311 | di-tert-butyl {(Z)-[({1-[4-(2-naphthyl)pyrimidin-2- | 561.4 | 3.2 | E |
| | yl]piperidin-4- | | | |
| | yl}methyl)amino]methylylidene}biscarbamate | | | |
| 312 | di-tert-butyl ((E)-{4-[4-(2-naphthyl)pyrimidin-2- | 533 | 11.8 | F |
| | yl]piperazin-1-yl}methylylidene)biscarbamate | | | |
| 313 | 4-[4-(2-naphthyl)pyrimidin-2-yl]piperazine-1- | 333 | 8.6 | F |
| | carboximidamide | | | |

### Example 365

### Preparation of 1-{1-[2-(2-naphthyl)pyrimidin-4-yl]piperidin-4-yl}methanamine

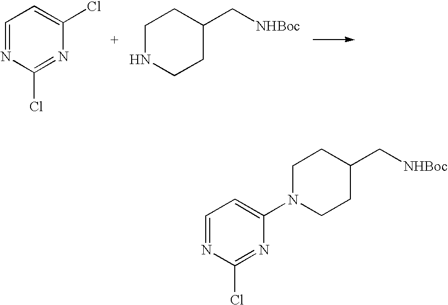

A solution of 2,4-dichloropyrimidine (6.3 g, 42 mmol) and diisopropylethylamine (6.1 g, 47 mmol, 8.4 ml) in DMSO (35 ml) was treated with tert-butyl piperidin-4-ylmethylcarbamate (10 g, 47 mmol) and stirred for an hour. The mixture was diluted with ethyl acetate (250 ml), washed with 1M sodium carbonate solution (100 mL), water (3×200 mL) and brine (100 mL). The organic layer was dried (MgSO₄) and evaporated. The crude product was purified by silica gel chromatography eluted with a gradient of 25-80% ethyl acetate in hexanes to leave the 4-substituted product (1.5 g, 11%) and the desired 2-substituted product (11.8 g, 86%). LC/MS (Method A) rt=1.63 mins., calculated mass=327, [M+H]⁺=328.

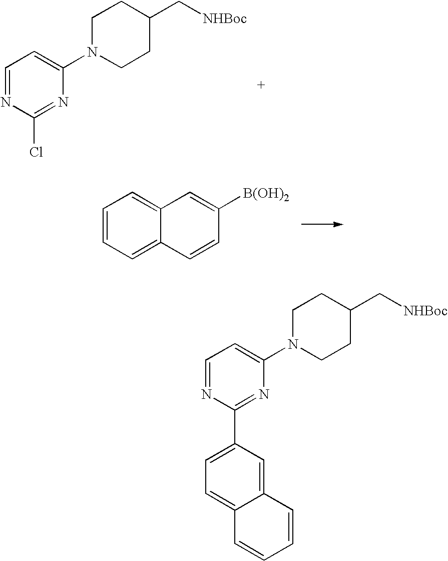

A mixture of tert-butyl (1-(2-chloropyrimidin-4-yl)piperidin-4-yl)methylcarbamate (10.9 g, 33.3 mmol), naphthalen-2-boronic acid (8.0 g, 46.6 mmol), Pd₂(dba)₃ (0.31 g, 0.33 mmol) and well-ground K₂CO₃ (12.1 g, 57 mmol) in anhydrous 1,4-dioxane was stirred and purged with nitrogen. 10% Tri(tert-butyl)phosphine in hexanes (1.7 mL, 0.83 mmol) was added and the mixture was stirred at 95 ° C. for 18 h. After cooling to room temperature the mixture was filtered through a plug of silica gel and eluted with ethyl acetate. The filtrate was evaporated in vacuo to approximately 100 mL and the residue was allowed to stand at −10° C. for 14 h. The crystalline product that formed was filtered, washed (ethyl acetate, 50 mL) and air dried to leave 10.5 g (75%). The filtrate was evaporated and purified by silica gel chromatagraphy eluted with a gradient of 25-75% ethyl acetate in hexanes to leave an additional 2.5 g (18%) of product. Total yield=13.0 g (93%). LC/MS (Method A) rt=1.46 mins., calculated mass=418, [M+H]⁺=419.

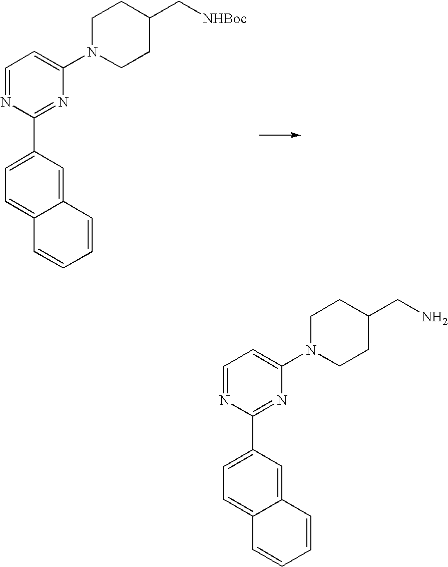

1-{1-[2-(2-naphthyl)pyrimidin-4-yl]piperidin-4-yl } methanamine: A solution of tert-butyl (1-(2-(naphthalen-2-yl)pyrimidin-4-yl)piperidin-4-yl)methylcarbamate (13 g, 31 mmol) in TFA (65 mL) and dichloromethane (65 mL) was stirred for 1 h then evaporated to dryness. The residue was partitioned between dichloromethane (200 mL) and 1 N NaOH solution (200 mL). The layers were separated and the aqueous layer was further extracted with dichloromethane (100 mL). The combined organic layers were dried (MgSO₄) and evaporated in vacuo to leave the product as a gum (9.9 g, 100%). The product was dissolved in MeOH (50 mL), treated with conc. HCl (2.4 mL, 31 mmol) and crystallized from a mixture of ethyl acetate and methanol. Left 9.4 g (86%) of monohydrochloride salt. HPLC (method F) rt=9.34 mins., purity>99.9%. ESMS [M+H]⁺=319.

### Example 366

### Preparation of 1-{1-[6-(2-naphthyl)pyrimidin-4-yl]piperidin-4-yl}methanamine

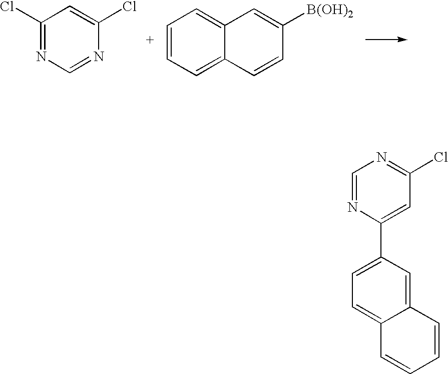

A mixture of 4,6-dichloropyrimidine (3.1 g, 21 mmol), naphthalene-2-boronic acid (1.8 g, 10 mmol), tetrakis(triphenylphosphine)palladium (0.58 g, 0.58 mmol) and tripotassium phosphate (8.5 g, 40 mmol) in anhydrous dioxane (30 mL) was purged with nitrogen and heated to 95° C. for 1.5 h. The dioxane was evaporated and the residue was dissolved in ethyl acetate (100 mL) and water (100 mL). The aqueous layer was separated and extracted with ethyl acetate (100 mL). The combined organic layers were washed with brine (50 mL), dried (MgSO₄) and evaporated in vacuo. The crude product was purified by silica gel chromatography eluted with a gradient of 5-50% ethyl acetate in hexanes to leave 1.7 g (71%) of a white solid that readily sublimes under high vacuum. LC/MS (Method A) rt=2.06 mins., calculated mass=240, [M+H]⁺=241.

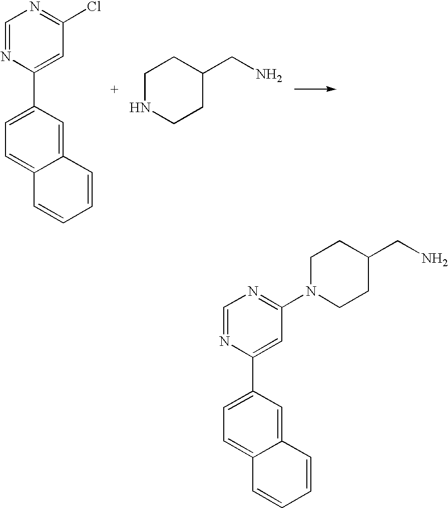

1-{1-[6-(2-naphthyl)pyrimidin-4-yl]piperidin-4-yl}methanamine: A solution of 4-chloro-6-(naphthalen-2-yl)pyrimidine (1.6 g, 6.6 mmol) and piperidin-4-ylmethanamine (1.5 g, 13 mmol) in DMSO (15 mL) was stirred and heated to 90° C. for 3 h. Water (3 mL) was added to the reaction mixture and the product was purified by reversed phase HPLC by direct injection of the reaction mixture without work-up. The product fractions were combined and lyophilized to leave the product as a bistrifluoroacetate salt (1.35 g, 37%). HPLC (method C) rt=5.71 mins., purity>99.9%. ESMS [M+H]⁺=319.

### Example 367

### Preparation of (1-(5-(Naphthalen-2-yl)pyrimidin-2-yl)piperidin-4-yl)methanamine

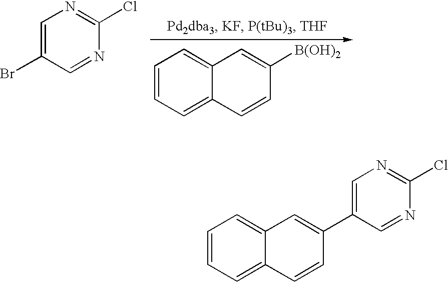

2-Chloro-5-(naphthalen-2-yl)pyrimidine. An oven-dried reaction flask containing a magnetic stir bar was charged with 5-bromo-2-chloropyrimidine (0.200 g, 1.03 mmol), 2-naphthaleneboronic acid (0.177 g, 1.03 mmol), tris(dibenzylideneacetone)dipalladium(0) (0.024 g, 0.026 mmol), and potassium fluoride (0.197 g, 3.40 mmol). The flask was purged with nitrogen, and anhydrous THF (2.5 mL) was added through a septum. A solution of 10% wt/wt tri-tert-butylphosphine in hexane (0.153 mL) was added by syringe while stirring. The reaction was stirred under nitrogen at rt for 6 h. The mixture was concentrated onto silica gel, and the mixture was flash chromatographed on silica gel eluting with 10% acetone/hexane to furnish the product (0.154 g, 62%) as a pale yellow solid. HPLC (method C): Rt=10.0 min. MS: [M+H]⁺=240.8

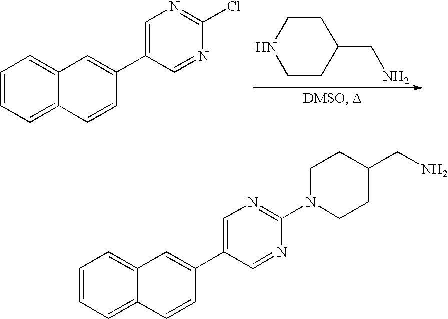

(1-(5-(Naphthalen-2-yl)pyrimidin-2-yl)piperidin-4-yl)methanamine. A solution of 2-chloro-5-(naphthalen-2-yl)pyrimidine (0.0125 g, 0.053 mmol) in DMSO (1 mL) was treated with 4-aminomethylpiperidine (0.0237 g, 0.212 mmol) and heated in an orbital vial shaker at 80° C. for 4h. The mixture was diluted with water (0.2 mL) and purified by reverse-phase HPLC (method I) to yield the product (7.2 mg, 44%) as a solid. HPLC (method H): Rt=3.43 min. MS: [M+H]⁺=319.1.

### Example 368

### PREPARATION OF (1-(6-(NAPHTHALEN-2-YL)PYRAZIN-2-YL)PIPERIDIN-4-YL)METHANAMIN

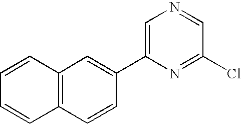

2-chloro-6-(naphthalen-2-yl)pyrazine; A mixture of 2,6-dichloropyrazine (215 mg, 1.4 mmol), 2-naphthaleneboronic acid (280 mg, 1.6 mmol), tetrakis(triphenylphosphine)palladium (81 mg, 70 μmol) and potassium carbonate (490 mg, 3.5 mmol) was purged with nitrogen, treated with anhydrous DMF (4 mL), stirred and heated to 100° C. for 18 h. The reaction mixture was cooled to room temperature, diluted with ethyl acetate (100 mL), washed with 1 M sodium carbonate (50 μmL) and water (3×100 mL), dried (MgSO₄) and evaporated. The residue was purified by silica gel chromatography eluted with a gradient of 10-40% ethyl acetate in hexanes to leave the product (148 mg, 44%). HPLC (method F) rt=10.9 mins., purity=99.1%. HR ESMS [M+H]⁺=241.0535.

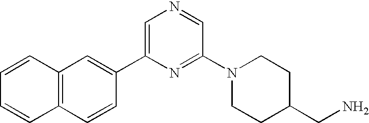

(1-(6-(naphthalen-2-yl)pyrazin-2-yl)piperidin-4-yl)methanamine; A solution of 2-chloro-6-(naphthalen-2-yl)pyrazine (100 mg, 0.42 mmol) and DIPEA (0.16 g, 1.2 mmol, 0.22 mL) in DMSO (4 mL) was treated with 4-(aminomethyl)piperidine (0.14 g, 1.2 mmol, 0.15 mL) and heated to 100° C. for 48 h. The reaction mixture was cooled to room temperature\, treated with water (1.5 mL) and the product was purified by reversed phase HPLC (method D). The product fractions were combined, neutralized with 1 M sodium carbonate solution and extracted with ethyl acetate (2×50 mL). The combined organic layers were washed with brine (50 mL), dried (MgSO₄) and evaporated to leave 82 mg (62%). HPLC (method G) rt=9.7 mins., purity=94.3%. HR ESMS [M+H]⁺=319.1909.

### Example 369

### Preparation of 1-{1-[5-(2-Naphthyl)pyridazin-3-yl]piperidin-4-yl}methanamine

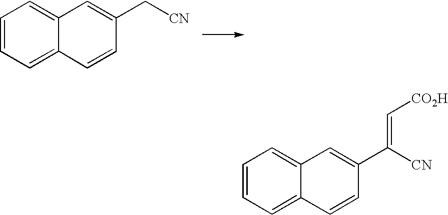

(2Z)-3-Cyano-3-(2-naphthyl)acrylic acid J. Org. Chem. 1993, 58, 7916. To cyanomethyl naphthalene (20 g, 0.12 mol) in MeOH (anh., 300 mL) was added an aqueous solution of glyoxilic acid (50% w/v, 27 mL, 0.18 mol, 1.5 equiv.). Solid K₂CO₃ (41.9 g, 0.30 mol, 2.54 equiv.) was added portionwise. The reaction was stirred and heated in a 60 C bath for 14 h. The reaction mixture was cooled to rt and filtered to collect a solid. The solid was stirred in water (300 mL) for 4 h at rt. The mixture was filtered to collect a solid, which was rinsed with DCM (25 mL). The solid was dried to afford the title compound (8.42 g, 27 % yield) as a white powder. HPLC (Method F) purity 98%, rt=7.7 min; MS (ESI+), calculated mass=222.23, [M+H]⁺=223.8.

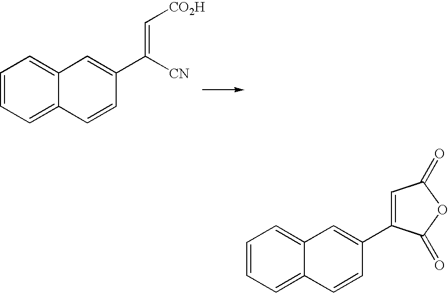

3-(Naphthalen-2-yl)furan-2,5-dione J. Org. Chem. 1993, 58, 7916. To a solution of (2Z)-3-cyano-3-(2-naphthyl)acrylic acid (31 g, 0.12 mol) in formic acid (96%, 200 mL) was added conc. H₂SO₄ (10 mL) dropwise. The mixture was heated in a 100 C bath for 14 h. The reaction was cooled to rt and poured into ice water. The resulting solid was collected by filtration and dried to afford the title compound (23.5 g, 88 % yield) as a shiny gold solid. LC/MS (Method A) rt=1.3 min; MS (ESI+), calculated mass=224.21, [M+H]⁺=225.

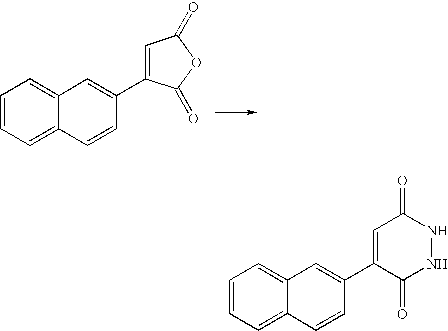

4-(Naphthalen-2-yl)-1,2-dihydropyridazine-3,6-dione J. Amer. Chem. Soc. 1954, 76, 2201. To a solution of hydrazine dihydrochloride (3.75 g, 0.0357 mol, 1 equiv.) in water (70 mL) was added 3-(naphthalen-2-yl)furan-2,5-dione (8.0 g, 0.0357 mol) and the reaction was heated in a 100 C bath for 14 h. The reaction was cooled to rt and THF (20 mL) was added. The reaction was heated in a 70 C bath for 24 h. The reaction was cooled to rt and concentrated to one-half the volume. The golden brown solid was collected by filtration and dried to afford the title compound (8.5 g, 100% yield). LC/MS (Method A) rt=1.1 min; MS (ESI+), calculated mass=238.24, [M+H]⁺=239.

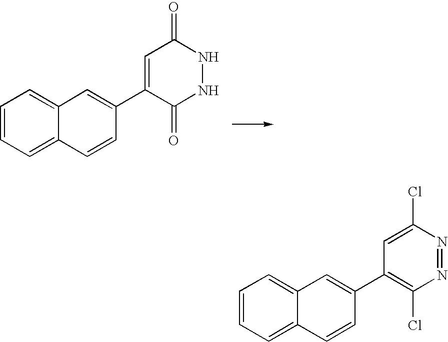

3,6-Dichloro-4-(2-naphthyl)pyridazine To 4-(naphthalen-2-yl)-1,2-dihydropyridazine-3,6-dione (8.4 g) in a flask fit with a drying tube was added phosphorous oxychloride (60 mL). The reaction was heated in an 80 C bath for 14 h. The reaction was cooled to rt and concentrated to afford a dark oil. The oil was dissolved in DCM (200 mL) and water (75 mL) was added. The mixture was cooled in an ice bath and solid NaHCO₃ was added in portions until it was of a neutral pH. The layers were separated and the aqueous layer was extracted with DCM (3×200 mL). The combined organic layers were washed with water (100 mL), dried (Na₂SO₄), filtered, and concentrated. The crude material was dissolved in phosphorous oxychloride (60 mL). The reaction was heated in a 95 C bath for 14 h. The reaction was cooled to rt and concentrated to afford a dark oil. The oil was dissolved in DCM (200 mL) and water (75 mL) was added. The mixture was cooled in an ice bath and solid NaHCO₃ was added in portions until it was of a neutral pH. The layers were separated and the aqueous layer was extracted with DCM (3×200 mL). The combined organic layers were washed with water (100 mL), dried (Na₂SO₄), filtered, and concentrated to afford the title compound (9.5 g, 97% yield) as a brown semi-solid. A 1 gram portion was purified using automated silica gel chromatography with an EtOAc/hexane (0 to 100%) gradient to afford an analytically pure sample. HPLC (Method F) purity 99%, rt=10.2 min; MS (ESI+), calculated mass=275.13, [M+H]⁺=274.8.

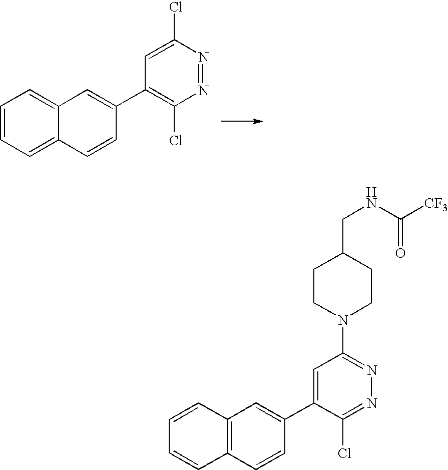

N-((1-(6-Chloro-5-(naphthalene-2-yl)pyridazin-3-yl)piperidin-4-yl)methyl-2,2,2-trifluoroacetamide To a solution of 3,6-dichloro-4-(2-naphthyl)pyridazine (0.114 g, 0.414 mmol) and 2,2,2-trifluoro-N-(piperidin-4-ylmethyl)acetamide (0.104 g, 0.497 mmol, 1.2 equiv.) in DMSO (1.5 mL) was added diisopropyl ethyl amine (0.16 mL, 0.91 mmol, 2.2 equiv.). The reaction was heated in a 60 C shaker block for 38 h. The reaction was cooled, water (0.1 mL) was added, and the reaction was purified by RP HPLC (method D) followed by automated silica gel chromatography with a gradient of 25% EtOAc/hexane to 100% EtOAc to afford the title compound (0.109 g, 58% yield) as a white powder.LC/MS (Method H) rt=5.92 min; MS (ESI+), calculated mass=448.87, [M+H]⁺=449.

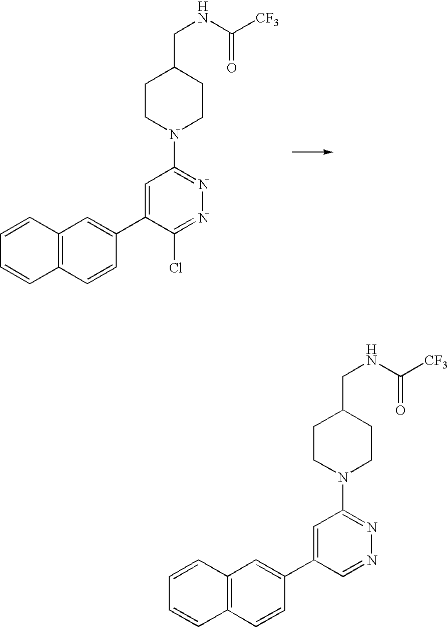

2,2,2-Trifluoro-N-((1-(5-(naphthalene-2-yl)pyridazin-3-yl)piperidin-4-yl)methyl)acetamide A solution of N-((1-(6-chloro-5-(naphthalene-2-yl)pyridazin-3-yl)piperidin-4-yl)methyl-2,2,2-trifluoroacetamide (0.109 g, 0.24 mmol) in EtOH (10 mL) and methoxyethanol (20 mL) was added to 10 % Pd/C, followed by triethyl amine (0.2 mL). The reaction was evacuated and a balloon of hydrogen was introduced. The reaction was stirred for 7 h, evacuated, and a fresh balloon of hydrogen was attached. The reaction was stirred for 14 h. The palladium was collected by filtration. The filtrate was concentrated and the residue was purified by automated silica gel chromatography with a gradient of 100% DCM to 15% MeOH/DCM. The title compound (59 mg, 59% yield) was isolated as a white powder. LC/MS (Method A) rt=1.45 min; MS (ESI+), calculated mass=414.45, [M+H]⁺=415.

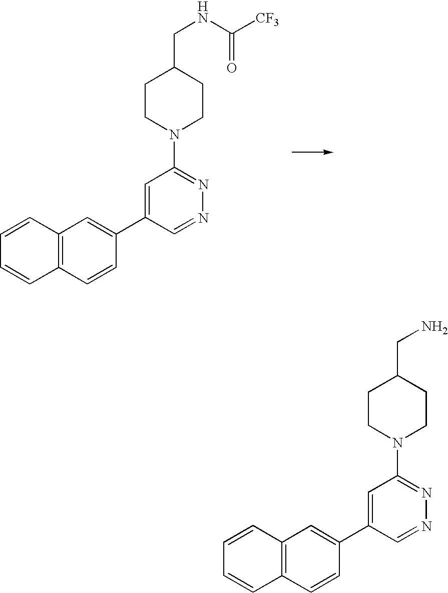

1-{1-[5-(2-Naphthyl)pyridazin-3-yl]piperidin-4-yl}methanamine The title compound was prepared from 2,2,2-trifluoro-N-((1-(5-(naphthalene-2-yl)pyridazin-3-yl)piperidin-4-yl)methyl)acetamide using the HPLC (Method F) purity 99%, rt=7.7 min; HRMS (ESI+), calculated mass=318.424, [M+H]⁺=319.1915.

### Example 370

### PREPARATION 1-{1-[4-(2-NAPHTHYL)PYRIMIDIN-2-YL]-1,2,3,4-TETRAHYDROQUINOLIN-4-YL}METHANAMINE

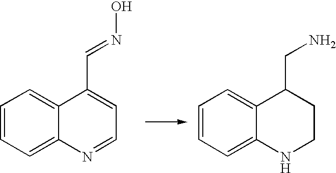

A solution of quinoline-4-carbaldehyde oxime (0.20 g, 1.2 mmol, prepared by the method of Barrow et. al., WO 01070229) in ethanol (8 mL), water (1 mL) and acetic acid (1 mL) was hydrogenated at 45 psi original pressure over 10% Pd/C (72 mg) for 140 h. The catalyst was filtered through diatomaceous earth, washed (MeOH, 10 mL) and evaporated in vacuo. The residue was dissolved in 1 M sodium carbonate solution (50 mL) and extracted with ethyl acetate (8×20 mL). The combined organic extracts were dried over MgSO₄ and evaporated to leave the product gum as a mixture of desired product and starting material (method A LC/MS indicated a product and starting material ratio of 7:1).

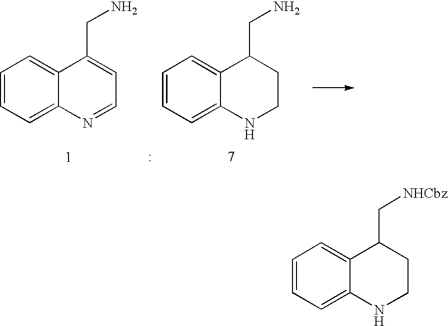

A solution of the mixture of (1,2,3,4-tetrahydroquinolin-4-yl)methanamine and quinolin-4-ylmethanamine prepared above (0.45 g, 2.8 mmol) and DIPEA in dichloromethane (30 mL) under nitrogen atmosphere and in an ice bath was treated with benzyl chloroformate (0.48 g, 2.8 mmol, 0.40 mL) and stirred 2 h. The reaction mixture was washed with 1 M sodium carbonate solution, dried (MgSO₄) and evaporated. The crude product was purified by silica chromatography eluted with 25-50% ethyl acetate in hexanes to leave 0.55 g (66%). LC/MS (Method A) rt=1.27 mins., calculated mass=296, [M+H]⁺=297.

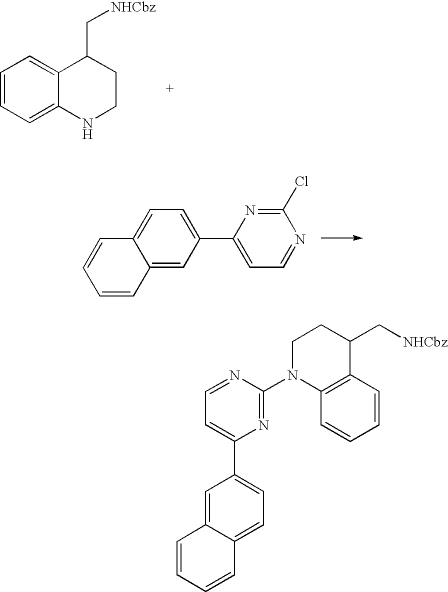

A solution of benzyl (1,2,3,4-tetrahydroquinolin-4-yl)methylcarbamate (50 mg, 0.17 mmol), 2-chloro-4-(naphthalen-2-yl)pyrimidine (41 mg, 0.17 mmol) and toluenesulfonic acid monohydrate (26 mg, 0.14 mmol) in 1,4-dioxane (1 mL) was stirred and heated to 100° C. for 18 h. The mixture was cooled to 20° C., diluted with ethyl acetate (25 mL) and 1 M sodium carbonate solution (25 mL). The aqueous layer was further extracted with ethyl acetate and the combined organic extracts were dried (MgSO₄) and evaporated. The crude product was purified by reversed phase HPLC (method D). LC/MS (Method A) rt=2.23 mins., calculated mass=500, [M+H]⁺=501.

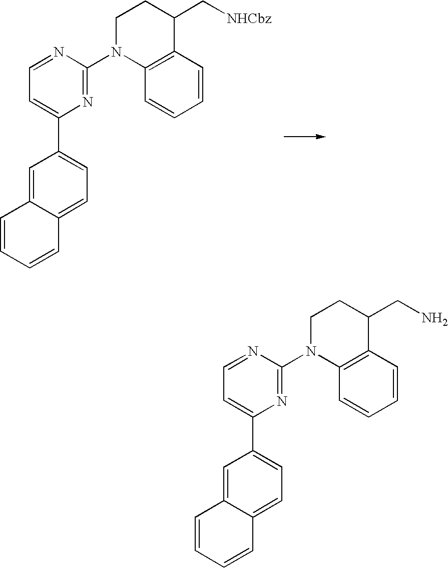

1-{1-[4-(2-naphthyl)pyrimidin-2-yl]-1,2,3,4-tetrahydroquinolin-4-yl}methanamine: A solution of benzyl (1-(4-(naphthalen-2-yl)pyrimidin-2-yl)-1,2,3,4-tetrahydroquinolin-4-yl)methylcarbamate (25 mg, 50 μmol) in aqueous conc. HBr (0.5 mL) was stirred at 55° C. for 1 h. The mixture was diluted with water (0.5 mL) and purified by reversed phase HPLC (method D) to leave the bistrifluoroacetate product as a gum (12 mg, 66%). HPLC (method C) rt=9.49 mins., purity >99.9%. ESMS [M+H]⁺=367.

### Examples 371 and 372

### Preparation of (S)-(1-(4-(naphthalen-2-yl)pyrimidin-2-yl)azepan-4-yl)methanamine and (R)-(1-(4-(naphthalen-2-yl)pyrimidin-2-yl)azepan-4-yl)methanamine

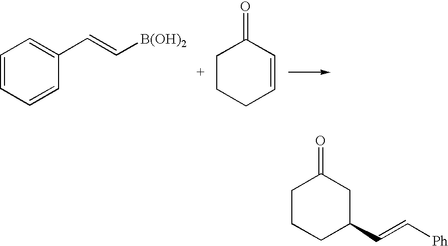

Prepared according to the procedure of Hayashi, J. Am. Chem. Soc. 1998, 120, 5579-5580; A solution of cyclohexenone (1.56 g, 16.3 mmol, 1.56 mL), (E)-styrylboronic acid (12 g, 81 mmol), acetoacetyldivinylrhodium (0.13 g, 0.49 mmol) and (S)-BINAP (0.31 g, 0.49 mmol) in water (4 mL) and 1,4-dioxane (40 mL) was purged with nitrogen and heated to 100° C. for 5 h. The reaction mixture was cooled to room temperature, diluted with ethyl acetate (200 mL) and washed with saturated sodium bicarbonate solution (2×100 mL), dried (MgSO₄) and evaporated in vacuo. Chromatographed on silica gel eluted with a gradient of 10-35% ethyl acetate in hexanes. Chromatography on silica gel was repeated eluting with a gradient of 50-100% dichloromethane in hexanes to leave the product as a white solid (2.1 g, 64%). HPLC (method C) rt=9.50 mins., purity=98.5%. ESMS [M+H]⁺=201. Enantiomeric excess=91% (chiral LC).

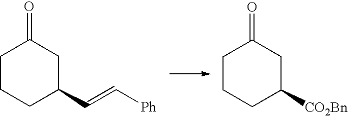

A mixture of (S,E)-3-styrylcyclohexanone (0.42 g, 2.1 mmol) and sodium periodate (1.8 g, 8.4 mmol) in carbon tetrachloride (4 mL), acetonitrile (4 mL) and water (6.5 mL) was rapidly stirred and treated with ruthenium(III) chloride (10 mg, 46 μmol). After 18 h the reaction mixture was diluted with water (50 mL) and dichloromethane (50 mL), separated and the aqueous layer was extracted with dichloromethane (3×50 mL). The combined organic extracts were dried (MgSO₄) and evaporated to leave a solid (0.36 g).

The solid residue was dissolved in dichloromethane (10 mL) and treated dropwise with a solution of diazotoluene (0.1 M in DCM, 85 mL, 8.5 mmol) prepared according to Synthesis, 1982, 419-421. The mixture stirred an additional 15 minutes then polystyrene bound carboxylic acid (0.70 mmol/g, 1.0 g, 0.70 mmol) was added and the reaction mixture was stirred for 1 h. The resin was filtered, washed (DCM, 2×25 mL) and the filtrate was evaporated. The crude product was purified by reversed phase HPLC (method D) to leave pure product (174 mg, 36% for 2 steps). LC/MS (Method A) rt=1.61 mins., calculated mass=232, [M+H]⁺=233.

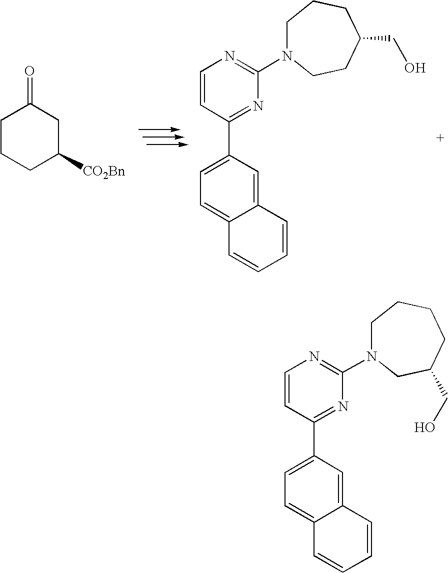

A mixture of (S)-benzyl 3-oxocyclohexanecarboxylate (0.63 g, 2.7 mmol) and sodium azide (0.53 g, 8.1 mmol) in chloroform (6 mL) was stirred rapidly under a nitrogen atmosphere and treated with methanesulfonic acid (2.6 g, 27 mmol, 1.7 mL) dropwise over 30 minutes. The reaction mixture was heated to reflux for 1 h. After cooling to room temperature the mixture was diluted with additional chloroform (50 mL) and 1 M sodium carbonate solution (50 mL), separated and the aqueous layer was further extracted with chloroform (3×25 mL). The combined organic extracts were washed with brine (100 mL), dried (MgSO₄) and evaporated. The crude product mixture was purified by silica gel chromatography eluted with 50-100% ethyl acetate in hexanes to leave a 2:1 mixture of regioisomeric lactams (NMR).

A solution of the lactam mixture (0.18 g, 0.73 mmol) in THF (4 mL) under nitrogen atmosphere was cooled in an ice bath and treated with a 1.0 M solution of lithium aluminum hydride in THF (2.2 mL, 2.2 mmol) dropwise over 5 mins. The mixture was refluxed for 3 h, cooled in an ice bath, cautiously treated with water (1 mL), then stirred rapidly at 50° C. for 1 h. The aluminum salts were filtered, washed with MeOH (2×5 mL) and evaporated.

The residue was dissolved in DMSO (3 mL), treated with DIPEA (0.19 g, 1.5 mmol, 0.27 mL) and 2-chloro-4-(naphthalen-2-yl)pyrimidine (0.24 g, 1.0 mmol), stirred and heated to 80° C. for 4 h. The mixture was cooled to room temperature, diluted with ethyl acetate (50 mL), washed with 1 M sodium carbonate solution (25 mL), water (2×25 mL) and brine (25 mL). The organic layer was dried (MgSO₄) and evaporated. The crude product mixture was purified by silica gel chromatography eluted with 35-80% ethyl acetate in hexanes to leave the minor product as a gum (48 mg, 5.3% for three steps). LC/MS (Method A) rt=1.90 mins., calculated mass=333, [M+H]⁺=334. The product, (S)-(1-(4-(naphthalen-2-yl)pyrimidin-2-yl)azepan-3-yl)methanol, was assigned by 2D NMR. The major product was isolated as a gum (120 mg, 13.3% for three steps). HPLC (method C) rt=11.90 mins., purity=98.9%. The product, (S)-(1-(4-(naphthalen-2-yl)pyrimidin-2-yl)azepan-4-yl)methanol, was assigned by 2D NMR.

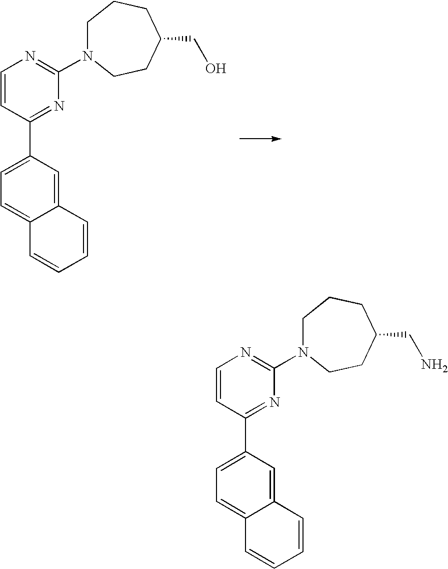

(S)-(1-(4-(naphthalen-2-yl)pyrimidin-2-yl)azepan-4-yl)methanamine. A mixture of (S)-(1-(4-(naphthalen-2-yl)pyrimidin-2-yl)azepan-4-yl)methanol (60 mg, 0.18 mmol), di-tert-butyl iminodicarbonate (0.17 g, 0.72 mmol) and triphenylphosphine on polystyrene resin (Argonaut Technologies, 1.24 mmol/g, 0.44 g, 0.54 mmol) in dichloromethane (4 mL) under nitrogen in an ice bath was treated with di-tert-butyl 1,2-azodicarboxylate (0.12 g, 0.54 mmol). The reaction mixture stirred for 1 h and the ice bath was removed. Stirring continued another 18 h. TFA (2 mL) was added and the mixture stirred for 1 h. The resin was filtered and washed with dichloromethane (2×5 mL) and MeOH (2×5 mL) and the combined filtrates were evaporated. The crude product was purified by reversed phase HPLC (method D) to leave the product as the bis TFA salt (37 mg, 37%). HPLC (method C) rt=9.0 mins., purity=98.0%. HR ESMS [M+H]⁺=333.2068.

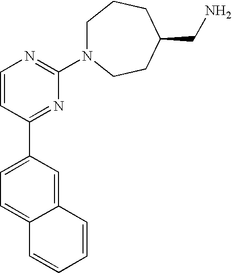

(R)-(1-(4-(naphthalen-2-yl)pyrimidin-2-yl)azepan-4-yl)methanamine was prepared in a manner similar to (S)-(1-(4-(naphthalen-2-yl)pyrimidin-2-yl)azepan-4-yl)methanamine in the example above. HPLC (method C) rt=9.8 mins., purity >99.9%. HR ESMS [M+H]⁺=333.2064.

### Example 373

### Preparation of 1-(4-(naphthalen-2-yl)pyrimidin-2-yl)azepan-4-amine

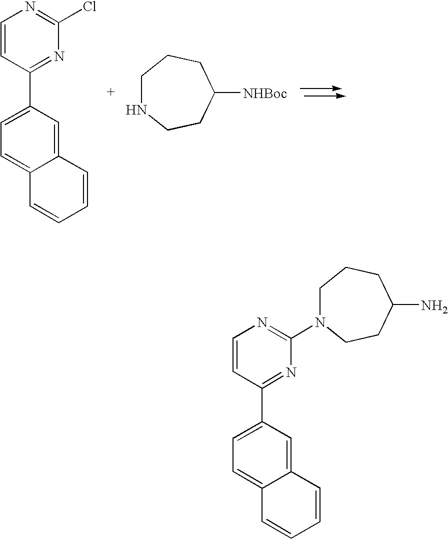

1-(4-(naphthalen-2-yl)pyrimidin-2-yl)azepan-4-amine; A solution of tert-butyl azepan-4-ylcarbamate (0.10 g, 0.47 mmol), DIPEA (0.12 g, 0.93 mmol, 0.17 mL) and 2-chloro-4-(naphthalen-2-yl)pyrimidine (90 mg, 0.37 mmol) in DMSO (1.0 mL) was heated to 80° C. for 18 h. After cooling to room temperature the mixture was diluted with ethyl acetate (25 mL) and washed with 1 M sodium carbonate (20 mL), water (2×20 mL) and brine (20 mL). The organic layer was dried (MgSO₄) and evaporated. The crude product was purified by silica gel chromatography eluting with 0-50% ethyl acetate in hexanes to leave the product (0.15 g, 76%) which was dissolved in 1:1 TFA-DCM (1.6 mL) and stirred for 1 h. The reaction mixture was diluted with DCM (25 mL) and washed with 1 M sodium carbonate. The aqueous layer was extracted with DCM (3×25 mL) and the combined organic extracts were dried (MgSO₄) and evaporated to leave 95 mg (68% over two steps) of product. HPLC (method C) rt=8.4 mins., purity=97.4%. HR ESMS [M+H]⁺=319.1928.

### Example 374

### Preparation of N-(1-(4-(naphthalen-2-yl)pyrimidin-2-yl)azepan-4-yl)acetamide

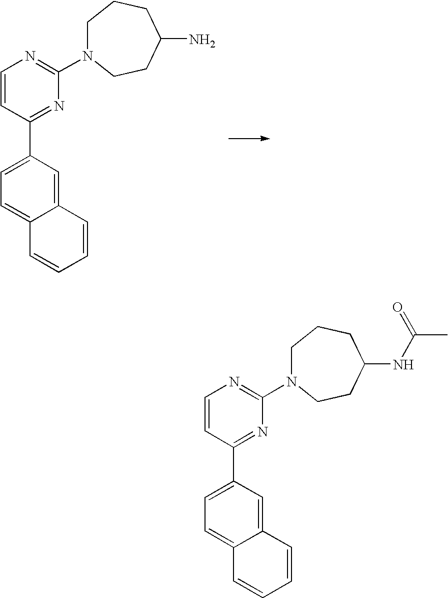

N-(1-(4-(naphthalen-2-yl)pyrimidin-2-yl)azepan-4-yl)acetamide; A solution of 1-(4-(naphthalen-2-yl)pyrimidin-2-yl)azepan-4-amine (3.2 mg, 12 μmol) and DIPEA (4.5 mg, 35 μmol, 6.3 μL) in DCM (0.1 mL) was treated with acetic anhydride solution (0.2 M, 117 uL, 23 μmol) in DCM. The mixture was shaken 18 h then diluted with MeOH (1 mL) and water (0.2 mL) and purified by RPHPLC (method C). Product fractions were evaporated to leave 3.0 mg of the TFA salt (63%). LC/MS (Method A) rt=1.50 mins., calculated mass=360, [M+H]⁺=361.

The same method was used to prepare the compounds in the following table using the procedure for the example above and the same aminoazepine starting material. Reagents and MS results are indicated.

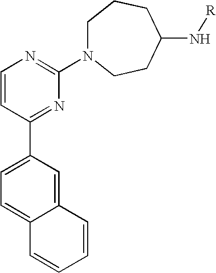

| Example | | | | HPLC |
|---|---|---|---|---|
| No. | Compound | MS | Rt | Method |
| 375 | 2,2,2-trifluoro-N-{1-[4-(2-naphthyl)pyrimidin-2-yl]azepan-4- | 414.9 | 2.70 | E |
| | yl}acetamide | | | |
| 376 | N,N-dimethyl-N′-{1-[4-(2-naphthyl)pyrimidin-2-yl]azepan-4- | 390.0 | 2.40 | E |
| | yl}urea | | | |
| 377 | N-{1-[4-(2-naphthyl)pyrimidin-2-yl]azepan-4-yl}benzamide | 422.9 | 2.60 | E |
| 378 | N-{1-[4-(2-naphthyl)pyrimidin-2-yl]azepan-4-yl}urea | 361.9 | 2.20 | E |
| 379 | N-ethyl-N′-{1-[4-(2-naphthyl)pyrimidin-2-yl]azepan-4-yl}urea | 390.0 | 2.40 | E |
| 380 | N-{1-[4-(2-naphthyl)pyrimidin-2-yl]azepan-4- | 396.9 | 2.40 | E |
| | yl}methanesulfonamide | | | |
| 381 | 4-methyl-N-{1-[4-(2-naphthyl)pyrimidin-2-yl]azepan-4- | 472.9 | 2.70 | E |
| | yl}benzenesulfonamide | | | |
| 382 | N,N-dimethyl-N′-{1-[4-(2-naphthyl)pyrimidin-2-yl]azepan-4- | 425.9 | 2.60 | E |
| | yl}sulfamide | | | |

### Example 383 and 383a

### Preparation of ((1R,4S)-2-(4-(naphthalen-2-yl)pyrimidin-2-yl)-2-azabicyclo[2.2.1]heptane-5,5-diyl)dimethanamine and ((1S,4R)-2-(4-(naphthalen-2-yl)pyrimidin-2-yl)-2-azabicyclo[2.2.1]heptane-5,5-diyl)dimethanamine

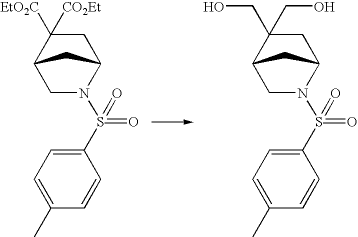

(1R,4S)-2-tosyl-2-azabicyclo[2.2.1 ]heptane-5,5-diyl)dimethanol—A solution of (1R,4S)-diethy 2-tosyl-2-azabicyclo[2.2.1]heptane-5,5-dicarboxylate (1.0 g, 2.5 mmol), prepared by the method of Portoghese (J. Heter. Chem. 1971, 8, 993-998), in anhydrous THF (20 mL) under nitrogen atmosphere was cooled in an ice bath and treated dropwise with a solution of lithium aluminum hydride in THF (2.5 mL, 5.0 mmol). The mixture warmed to room temperature over 2 h and it was then recooled in an ice bath, and cautiously treated with water (3 mL). The mixture stirred 14 h and warmed to room temperature. The aluminum salts were filtered through diatomaceous earth, washed with MeOH (25 mL) and evaporated in vaccuo. The residue was dissolved in dichloromethane (100 mL), washed with 1 N sodium hydroxide solution (50 mL), dried (MgSO₄) and evaporated to a white solid (0.45 g, 58%). HPLC (method C) rt=6.5 mins., purity=97.9%. HR ESMS [M+H]⁺ calc'd.=312.1264; found=312.1266.

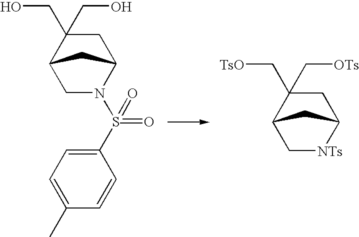

((1R,4S)-2-tosyl-2-azabicyclo[2.2.1]heptane-5,5-diyl)bis(methylene)bis(4-methylbenzenesulfonate); A solution of ((1R,4S)-2-tosyl-2-azabicyclo[2.2.1]heptane-5,5-diyl)dimethanol (0.21 g, 0.68 mmol), triethylamine (0.27 g, 2.7 mmol) and DMAP (0.17 g, 1.4 mmol) in DCM (8 mL) under nitrogen atmosphere was cooled in an ice bath and treated with p-toluenesulfonyl chloride (0.52 g, 2.7 mmol). The mixture warmed to room temperature over 2 h and stirred an additional 96 h. The reaction mixture was diluted with DCM to 50 mL, washed with 1 N HCl solution (50 mL), water (50 mL), 1 M sodium carbonate solution (50 mL) and water (50 mL). The organic layer was dried (MgSO₄) and evaporated. The residue was chromatographed on silica gel eluted with a gradient of 25-50% ethyl acetate in hexanes to leave the pure product as a white solid (360 mg, 86%). LC/MS (Method A) rt=3.68 mins., calculated mass=619, [M+H]⁺=620.

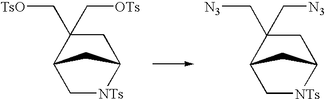

(1R,4S)-5,5-bis(azidomethyl)-2-tosyl-2-azabicyclo[2.2.1]heptane; A solution of ((1R,4S)-2-tosyl-2-azabicyclo[2.2.1]heptane-5,5-diyl)bis(methylene)bis(4-methylbenzenesulfonate) (0.35 g, 0.57 mmol) and sodium azide (0.37 g, 5.7 mmol) in DMSO (3 mL) was stirred at 60° C. for 96 h. The reaction mixture was diluted with ethyl acetate (50 mL), washed with water (2×30 mL), brine (30 mL), dried (MgSO4) and evaporated in vaccuo. The crude product was purified on silica gel eluted with a gradient of 20-50% ethyl acetate in hexanes to leave a colorless gum (183 mg, 89%). LC/MS (Method A) rt=3.29 mins., calculated mass=361, [M+H]⁺=362.

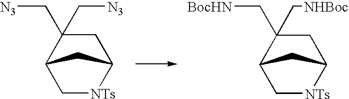

tert-butyl ((1R,4S)-2-tosyl-2-azabicyclo[2.2.1]heptane-5,5-diyl)bis(methylene)dicarbamate: A solution of (1R,4S)-5,5-bis(azidomethyl)-2-tosyl-2-azabicyclo[2.2.1]heptane (170 mg, 0.47 mmol) in MeOH (4 mL) was hydrogenated over 10% Pd/C (20 mg) at 1 atmosphere hydrogen pressure for 3 h. Di-tert-butyldicarbonate (300 mg, 1.4 mmol) was added and the mixture stirred 14 h. The reaction mixture was filtered over diatomaceous earth, washed with MeOH (2×5 mL) and the combined filtrates were evaporated. The crude product was purified by silica gel chromatography eluted with 25-50% ethyl acetate in hexanes to leave 219 mg of product (92%). LC/MS (Method A) rt=3.63 mins., calculated mass=509, [M+H]⁺=510.

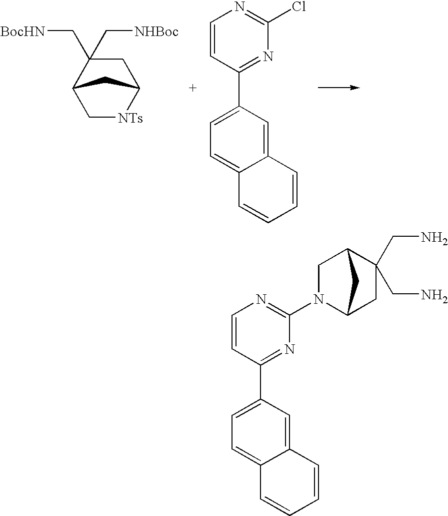

((1R,4S)-2-(4-(naphthalen-2-yl)pyrimidin-2-yl)-2-azabicyclo[2.2.1]heptane-5,5-diyl)dimethanamine; A solution of tert-butyl ((1R,4S)-2-tosyl-2-azabicyclo[2.2. 1]heptane-5,5-diyl)bis(methylene)dicarbamate (210 mg, 0.41 mmol) in MeOH (4 mL) was treated with Mg turnings and stirred rapidly for 18 h. The reaction mixture was diluted with 1 N HCl (25 mL) and washed with ethyl acetate (25 mL). The aqueous layer was neutralized with solid sodium carbonate (pH=10) and extracted with ethyl acetate (2×25 mL). The combined organic aextracts were dried (Na₂SO₄) and evaporated to leave a tan gum (32 mg, 22%). A sample of the amine (16 mg, 45 μmol) was dissolved in DMSO (0.5 mL) and treated with DIPEA (13 mg, 100 μmol, 18 μL) and 2-chloro-4-(naphthalen-2-yl)pyrimidine (54 μmol) and stirred at 70° C. for 18 h. The reaction mixture was diluted with water (0.1 mL) and MeOH (0.2 mL) and purified by reversed phase HPLC (method method J). Left 15 mg of the product as a tan gum (60%). This sample was dissolved in 1:1 dichloromethane-TFA (0.5 mL) and stirred for 1 h. The solvents were evaporated in vacuo to leave the product as a tris TFA salt (19 mg, 100%). HPLC (method C) rt=6.8 mins., purity=98.5%. HR ESMS [M+H]⁺ calc'd=360.2183, obs'd=360.2172.

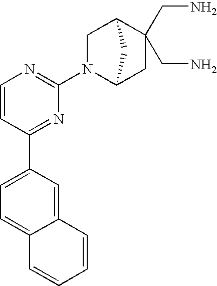

((1S,4R)-2-(4-(naphthalen-2-yl)pyrimidin-2-yl)-2-azabicyclo[2.2.1]heptane-5,5-diyl)dimethanamine was prepared using methods similar to those used for the example above starting from (1S,4R)-diethyl 2-tosyl-2-azabicyclo[2.2.1]heptane-5,5-dicarboxylate. LC/MS (Method A) rt=1.38 mins., calculated mass =359, [M+H]⁺=360.

### Example 384 and 385

### Preparation of ((1R,4R,5S)-2-(4-(naphthalen-2-yl)pyrimidin-2-yl)-2-azabicyclo[2.2.1]heptan-5-yl)methanamine and ((1R,4R,5R)-2-(4-(naphthalen-2-yl)pyrimidin-2-yl)-2-azabicyclo[2.2.1]heptan-5-yl)methanamine

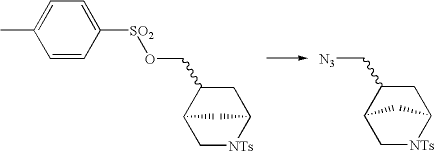

(1R,4R)-5-(azidomethyl)-2-tosyl-2-azabicyclo[2.2.1]heptane; A solution of ((1S,4R)-2-tosyl-2-azabicyclo[2.2.1]heptan-5-yl)methyl 4-methylbenzenesulfonate (0.79 g, 1.8 mmol), prepared by the method of Jordis (J. Heter. Chem. 1991, 28, 2045-2047), in DMSO (5 mL) was treated with sodium azide (0.59 g, 9.1 mmol) and stirred at 60° C. for 6 h. The reaction mixture was cooled to room temperature, diluted with ethyl acetate (50 mL), washed with water (2×50 mL) then brine (50 mL) and dried (MgSO₄). The solvent was evaporated to leave product as a 3:1 mixture of diastereomers (NMR). LC/MS (Method A) rt=2.95 mins., calculated mass=306, [M+H]⁺=307.

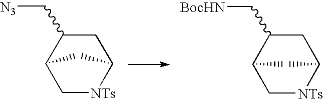

tert-Butyl ((1R,4R)-2-tosyl-2-azabicyclo[2.2.1]heptan-5-yl)methylcarbamate; A solution of (1R,4R)-5-(azidomethyl)-2-tosyl-2-azabicyclo[2.2.1]heptane (0.54 g, 1.8 mmol) in MeOH (20 mL) was hydrogenated over 10% Pd/C at 1 atmosphere hydrogen pressure for 3 h. Di-tert-butyldicarbonate (0.46 g, 2.2 mmol) was added and the mixture stirred for 16 h. The catalyst was filtered through diatomaceous earth and washed with MeOH (2×10 mL). The combined filtrates were evaporated and the crude product was purified by silica gel chromatography eluted with a gradient of 30-60% ethyl acetate in hexanes. Left 0.60 g (88%) of tan gum. LC/MS (Method A) rt=3.00 mins., calculated mass=380, [M+H]⁺=381.

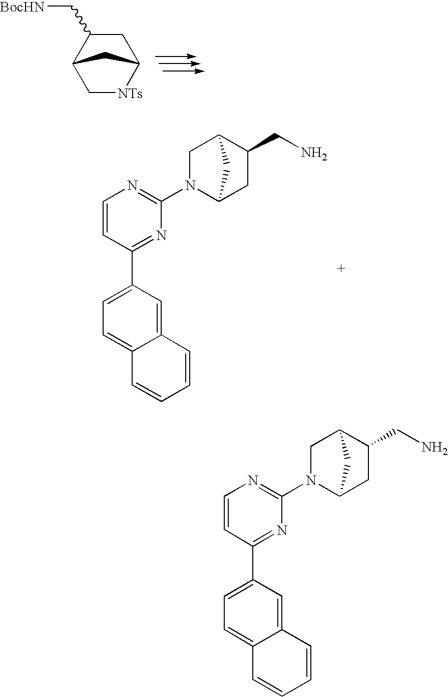

((1R,4R,5S)-2-(4-(naphthalen-2-yl)pyrimidin-2-yl)-2-azabicyclo[2.2.1]heptan-5-yl)methanamine and ((1R,4R,5R)-2-(4-(naphthalen-2-yl)pyrimidin-2-yl)-2-azabicyclo[2.2.1]heptan-5-yl)methyanamine; A solution of tert-butyl ((1R,4R)-2-tosyl-2-azabicyclo[2.2.1]heptan-5-yl)methylcarbamate (0.59 g, 1.6 mmol) in MeOH (15 mL) was treated with Mg turnings (0.37 g, 16 mmol) and stirred 16 h. The reaction mixture was diluted with 1 N HCl (50 mL), washed with ethyl acetate (25 mL), neutralized with solid sodium carbonate (pH=10) and extracted with ethyl acetate (3×25 mL). The combined organic extracts were dried (Na₂SO₄) and evaporated to leave a tan gum (0.12 g, 33%). A sample of this amine (30 mg, 0.13 mmol) and DIPEA (21 mg, 0.16 mmol, 29 μL) in DMSO (1 mL) was treated with 2-chloro-4-(naphthalen-2-yl)pyrimidine (38 mg, 0.16 mmol), stirred and heated to 70° C. for 18 h. Water (0.15 mL) was added to the reaction mixture after cooling to room temperature and the product was purified by reversed phase HPLC (method D) to leave the diastereomeric product mixture as a mono TFA salt (36 mg, 79%). The diastereomers were separated by SFC on a PERPSFC1 instrument with a Kromasil CN column (20×250 mm, 92:8 CO₂/MeOH w/0.2% dimethylethylamine, 50 mL/min., 220 nm, 35° C). The products weighed 7.0 mg and 21 mg respectively. Stereochemical assignments were made by 2D NMR techniques. The minor diastereomer is tert-butyl ((1R,4R,5S)-2-(4-(naphthalen-2-yl)pyrimidin-2-yl)-2-azabicyclo[2.2.1]heptan-5-yl)methylcarbamate and the major isomer is tert-butyl ((1R,4R,5R)-2-(4-(naphthalen-2-yl)pyrimidin-2-yl)-2-azabicyclo[2.2.1]heptan-5-yl)methylcarbamate. Both compounds were dissolved in 1:1 DCM-TFA (1 mL), stirred for 1 h then purified by reversed phase HPLC (method J) to leave 5 mg (98%) of the minor isomer ((1R,4R,5S)-2-(4-(naphthalen-2-yl)pyrimidin-2-yl)-2-azabicyclo[2.2.1]heptan-5-yl)methanamine. LC/MS (Method A) rt=1.73 mins., calculated mass=330, [M+H]⁺=331. The major isomer, ((1R,4R,5R)-2-(4-(naphthalen-2-yl)pyrimidin-2-yl)-2-azabicyclo[2.2.1]heptan-5-yl)methanamine weighed 15 mg (93%). LC/MS (Method A) rt=1.35 mins., calculated mass=330, [M+H]⁺=331.

### Examples 386 and 387

### Preparation of ((1S,4S,5S)-2-(4-(naphthalen-2-yl)pyrimidin-2-yl)-2-azabicyclo[2.2.1]heptan-5-yl)methanamine and ((1S,4S,5R)-2-(4-(naphthalen-2-yl)pyrimidin-2-yl)-2-azabicyclo[2.2.1]heptan-5-yl)methanamine

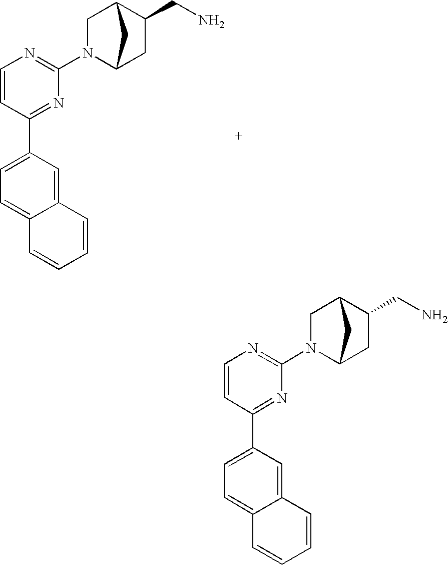

((1S,4S,5S)-2-(4-(naphthalen-2-yl)pyrimidin-2-yl)-2-azabicyclo[2.2.1]heptan-5-yl)methanamine and ((1S,4S,5R)-2-(4-(naphthalen-2-yl)pyrimidin-2-yl)-2-azabicyclo[2.2.1]heptan-5-methanamine were prepared in an identical manner to the examples above. The SSS isomer was prepared on a 6 mg scale. HPLC (method C) rt=8.5 mins., purity >99.9%. HR ESMS [M+H]⁺ calc'd=331.1917, obs'd=331.1920. The SSR isomer weighed 0.7 mg. HPLC (method C) rt=8.4 mins., purity>99.9%. HR ESMS [M+H]⁺ calc'd=331.1917, obs'd=331.1926.

### Examples 388 and 389

### Preparation of ((1R,3r,5S)-8-(4-(naphthalen-2-yl)pyrimidin-2-yl)-8-azabicyclo[3.2.1]octan-3-yl)methanamine and ((1R,3s,5S)-8-(4-(naphthalen-2-yl)pyrimidin-2-yl)-8-azabicyclo[3.2.1]octan-3-yl)methanamine

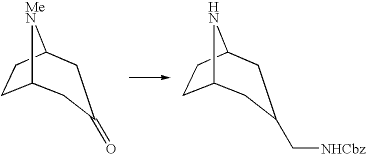

Benzyl 8-azabicyclo[3.2.1 ]octan-3-ylmethylcarbamate; A mixture of 8-methyl-8-azabicyclo[3.2.1]octan-3-one (5.0 g, 36 mmol), tert-butyl diethylphosphonoacetate (13.6 g, 54 mmol, 12.7 mL) and lithium chloride (2.3 g, 54 mmol) in acetonitrile (150 mL) was treated with DBU (8.2 g, 54 mmol, 8.0 mL) and stirred for 6 days. The solvent was evaporated and the residue was dissloved in ethyl acetate (200 mL) and 1 M sodium carbonate solution (200 mL). The layers were separated and the organic layer was washed with water (200 mL) and brine (100 mL), dried (MgSO₄) and evaporated to leave 13 g. The crude product was purified by reversed phase HPLC (method D) to leave 5.2 g of colorless oil (61%). A sample of this product (4.0 g, 17 mmol) was hydrogenated over 5% Pd/C (0.70 g) in MeOH (50 mL) at 1 atmosphere hydrogen pressure for 48 h. The catalyst was filtered, washed with MeOH (2×25 mL) and evaporated to leave a colorless gum (3.7 g, 91%). ¹H-NMR indicated a 2:1 mixture of isomers. The product (3.7 g, 15 mmol) was stirred in 1:1 TFA-DCM (80 mL) for 1 h. The solvents were evaporated to leave the product as a TFA salt (4.6 g, 100%).

A 5.6 g (19 mmol) sample of the acid prepared above was dissolved in toluene (100 mL) and treated with DIPEA (4.9 g, 38 mmol, 6.8 mL) under nitrogen. To this solution was added diphenylphosphorylazide (9.3 g, 34 mmol, 7.2 mL) and the reaction mixture was heated to reflux for 1.5 h. Benzyl alcohol (10.3 g, 95 mmol, 9.9 mL) was added and reflux continued for an additional 2 h. The mixture was cooled to 20° C., diluted with ethyl acetate (100 mL), extracted with 1 N HCl (3×100 mL) and the combined aqueous layers were washed with ethyl acetate (100 mL), neutralized with solid sodium carbonate (pH=13) and extracted with ethyl acetate (3×150 mL). The combined organic extracts were dried (MgSO₄) and evaporated. The crude product was purified by reversed phase HPLC (method J) and the product fractions were lyophilized to leave 0.18 g (3.3%). The amine was dissolved in 1,2-dichloroethane (6 mL) and treated with DIPEA (98 mg, 0.76 mmol, 0.14 mL) and 1′-chloroethyl chloroformate (0.18 g, 1.3 mmol, 0.14 mL) and heated to reflux under nitrogen for 2 h. The solvents were evaporated and the residue was dissolved in MeOH (12 mL) and heated to reflux for 2 h. The solvent was evaporated and the residue was dissolved in ethyl acetate (50 mL), washed with 1 M sodium carbonate (50 mL), dried (MgSO₄) and evaporated in vacuo. Left 151 mg (85%). LC/MS (Method A) rt=1.22 mins., calculated mass=274, [M+H]⁺=275.

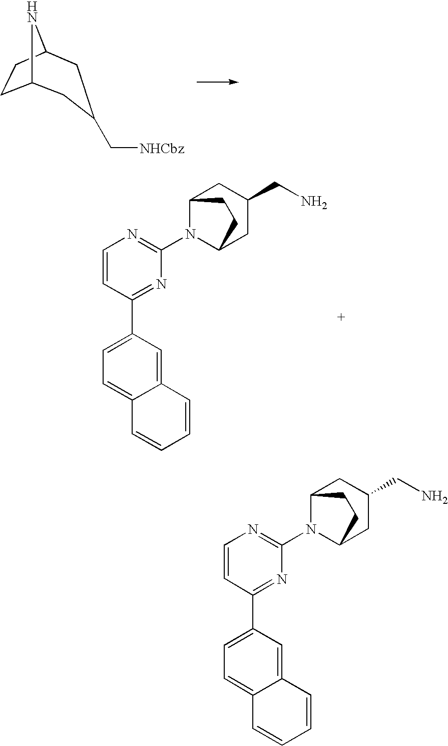

((1R,3r,5S)-8-(4-(naphthalen-2-yl)pyrimidin-2-yl)-8-azabicyclo[3.2.1]octan-3yl)methanamine and ((1R,3s,5S)-8-(4-(naphthalen-2-yl)pyrimidin-2-yl)-8-azabicyclo[3.2.1]octan-3-yl)methanamine; A solution of benzyl 8-azabicyclo[3.2.1]octan-3-ylmethylcarbamate (47 mg, 0.17 mmol), DIPEA (27 mg, 0.21 mmol) and 2-chloro-4-(naphthalen-2-yl)pyrimidine (49 mg, 0.21 mmol) in DMSO (1 mL) was heated to 80° C. for 3 h. The reaction mixture was diluted with ethyl acetate (25 mL), washed with 1 M sodium carbonate (20 mL), water (2×20 mL) and brine (20 mL). The organic layer was dried (MgSO₄) and evaporated. The residue was purified by silica gel chromatography eluted with 25-75% ethyl acetate in hexanes (47 mg, 64%). The diastereomers were separated by SFC on an ethylpyridine column (83:17 CO₂/MeOH w/0.2% dimethylethylamine). Peak 1 weighed 25 mg (31%) and peak 2 was 17 mg (21%). 2D NMR confirmed peak 1 as ((1R,3r,5S)-8-(4-(naphthalen-2-yl)pyrimidin-2-yl)-8-azabicyclo[3.2.1]octan-3-yl)methanamine. LC/MS (Method A) rt=3.66 mins., calculated mass=478, [M+H]⁺=479. 2D NMR confirmed peak 2 as ((1R,3s,5S)-8-(4-(naphthalen-2-yl)pyrimidin-2-yl)-8-azabicyclo[3.2.1]octan-3-yl)methanamine. LC/MS (Method A) rt=3.71 mins., calculated mass=478, [M+H]⁺=479.

### Example 390

### Preparation of 2-(4-(naphthalen-2-yl)pyrimidin-2-yl)-2,8-diazaspiro[4.5]decane

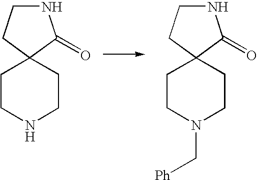

8-Benzyl-2,8-diazaspiro[4.5]decan-1-one; A solution of 2,8-diazaspiro[4.5]decan-1-one (0.75 g, 3.9 mmol) and benzaldehyde (0.63 g, 5.9 mmol, 0.60 mL) in NMP (20 mL) was treated with sodium triacetoxyborohydride (1.3 g, 5.9 mmol) and stirred for 3 h. The reaction mixture was diluted with ethyl acetate to 200 mL and washed with 1 M sodium bicarbonate solution (100 mL). The organic layer was dried (MgSO₄), filtered and treated with sulfonic acid on polystyrene (1.4 mmol/g, 7.0 g, 9.8 mmol) and allowed to stand for 18 h. The resin was filtered, washed with dichloromethane (3×50 mL) and treated with 10% triethylamine in MeOH (5×50 mL) and the combined filtrate was evaporated to leave a tan solid (372 mg, 39%). LC/MS (Method H) rt=2.14 mins., calculated mass=244, [M+H]⁺=245.

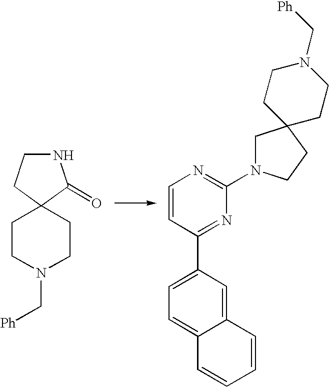

8-Benzyl-2-(4-(naphthalen-2-yl)pyrimidin-2-yl)-2,8-diazaspiro[4.5]decane; A solution of 8-benzyl-2,8-diazaspiro[4.5]decan-1-one (0.16 g, 0.66 mmol) in THF (3 mL) under nitrogen atmosphere and cooled in an ice bath was treated with a 2.0 M solution of lithium aluminum hydride in THF (0.65 mL, 1.3 mmol). The ice bath was removed and the reaction mixture was heated to 60° C. for 2 h. Cooled to room temperature, treated with water (0.5 mL) and stirred for 18 h. The mixture was filtered, washed with MeOH (2×10 mL) and evaporated to dryness to leave a tan oil (127 mg, 84%). A sample of this oil (30 mg, 0.13 mmol), DIPEA (20 mg, 0.16 mmol) and 2-chloro-4-(naphthalen-2-yl)pyrimidine (38 mg, 0.16 mmol) in DMSO (1 mL) was heated to 100° C. for 8 h. The reaction mixture was diluted with ethyl acetate (50 mL), washed with 1 M sodium carbonate solution (25 mL), water (2×25 mL) and brine (25 mL). The organic layer was dried (MgSO₄) and evaporated. The crude product was purified on silica gel eluted with a gradient of 50-100% ethyl acetate in hexanes to leave 32 mg (63%). HPLC (method C) rt=9.7 mins., purity >95.0%. HR ESMS [M+H]⁺ calc'd =435.2543, obs'd=435.2546.

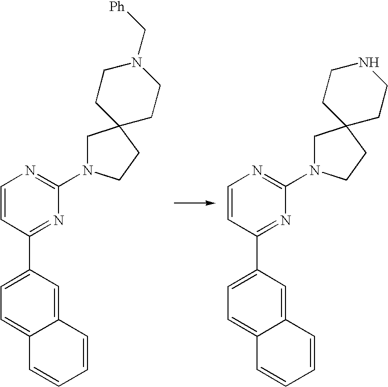

2-(4-(naphthalen-2-yl)pyrimidin-2-yl)-2,8-diazaspiro[4.5]decane; A solution of 8-benzyl-2-(4-(naphthalen-2-yl)pyrimidin-2-yl)-2,8-diazaspiro[4.5]decane (30 mg, 69 μmol) and DIPEA (22 mg, 168 μmol, 30 μL) in 1,2-dichloroethane (2 mL) was treated with 1-chloroethyl chloroformate (20 mg, 140 μmol, 15 μL) and stirred 15 at room temperature then 2 h at reflux. The solvents were evaporated and the residue was treated with MeOH (2 mL) and heated to reflux for 2 h. The solvent was evaporated and the crude product was purified by reversed phase HPLC (method D) to leave 12 mg (43%). HPLC (method C) rt=8.6 mins., purity>97.1%. HR ESMS [M+H]⁺ calc'd=345.2074, obs'd=345.2079.

### Example 391

### Preparation of (R)—N1,N1,N2-trimethyl-N2-(1-(4-(naphthalen-2-yl)pyrimidin-2-yl)pyrrolidin-3-yl)ethane-1,2-diamine

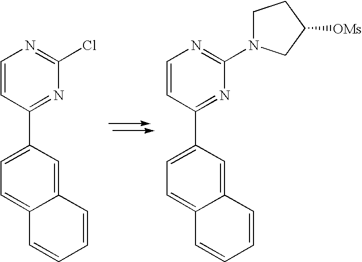

(S)-1-(4-(naphthalen-2-yl)pyrimidin-2-yl)pyrrolidin-3-yl methanesulfonate; A solution of 2-chloro-4-(naphthalen-2-yl)pyrimidine (4.8 g, 20 mmol), (S)-3-pyrrolidinol (2.6 g, 30 mmol) and DIPEA (7.8 g, 60 mmol) in DMSO (20 mL) was stirred and heated to 80° C. for 5 h. The reaction mixture was cooled to room temperature, diluted with ethyl acetate (200 mL), washed with water (2×200 mL) and brine (100 mL), dried (MgSO₄) and evaporated to leave 5.1 g (87%). A sample of the alcohol (4.7 g, 16 mmol) in dichloromethane (50 mL) was treated with DIPEA (8.4 g, 65 mmol) and DMAP (0.44 g, 3.6 mmol). Methanesulfonyl chloride (5.5 g, 48 mmol) was added dropwise over 5 minutes under nitrogen atmosphere and the reaction mixture was stirred for 3 h. The solution was diluted with dichloromethane (100 mL), washed with water (2×100 mL), 0.1 N HCl (100 mL) and water (100 mL). The organic layer was dried (MgSO₄) and evaporated and the residue was crystallized form ethyl acetate and ether. Left 5.2 g (87%) of solid.

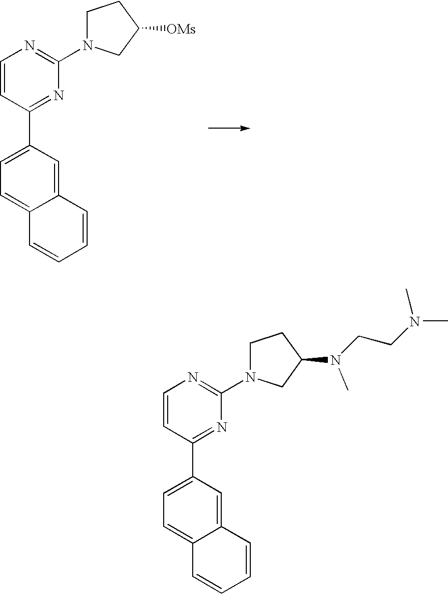

(R)—N1,N1,N2-trimethyl-N2-(1-(4-(naphthalen-2-yl)pyrimidin-2-yl)pyrrolidin-3-yl)ethane-1,2-diamine; A mixture of (S)-1-(4-(naphthalen-2-yl)pyrimidin-2-yl)pyrrolidin-3-yl methanesulfonate (11.7 g, 31.7 mmol) in DMSO (32 mL) was treated with N1,N1,N2-trimethylethylenediamine (32 g, 317 mmol, 41 mL) and stirred at 90° C. for 6 h. The reaction mixture was cooled to room temperature, diluted with ethyl acetate (300 mL), washed with water (3×200 mL) and brine (200 mL), dried (Na₂SO₄) and evaporated. Purified by reversed phase chromatography (method I with 0.1% formic acid buffer). Left 7.0 g (59%) which was dissolved in THF (100 mL) and treated with 1.0 equivalent of conc. HCl. The precipatate that formed was treated with ethyl acetate (100 mL), filtered, washed with 1:1 THF-ethyl acetate (50 mL) and dried to leave a beige solid (5.9 g, 45%). HPLC (method C) rt=8.9 mins., purity=98.8%. HR ESMS [M+H]⁺ calc'd=376.2496, obs'd=376.2498.

The same method was used to prepare the compounds in the following table using the procedure for the example above.

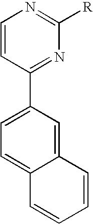

| Example | | | | HPLC |
|---|---|---|---|---|
| No. | Compound | MS | Rt | Method |
| 392 | (3S,3′R)-1′-[4-(2-naphthyl)pyrimidin-2-yl]-1,3′-bipyrrolidin-3-ol | 317 | 7.80 | E |
| 393 | 5-(4-naphthalen-2-ylpyrimidin-2-yl)octahydropyrrolo[3,4-b]pyrrole | 317 | 8.80 | E |
| 394 | 1-({1-[4-(2-naphthyl)pyrimidin-2-yl]pyrrolidin-3- | 389 | 8.10 | E |
| | yl}methyl)piperidin-4-ol | | | |
| 395 | (3S)-1-({1-[4-(2-naphthyl)pyrimidin-2-yl]pyrrolidin-3- | 389 | 8.20 | E |
| | yl}methyl)piperidin-3-ol | | | |
| 396 | (3R)-1-({1-[4-(2-naphthyl)pyrimidin-2-yl]pyrrolidin-3- | 389 | 8.20 | E |
| | yl}methyl)piperidin-3-ol | | | |
| 397 | (3S)-1-({1-[4-(2-naphthyl)pyrimidin-2-yl]pyrrolidin-3- | 375 | 8.10 | E |
| | yl}methyl)pyrrolidin-3-ol | | | |
| 398 | (3R)-1-({1-[4-(2-naphthyl)pyrimidin-2-yl]pyrrolidin-3- | 375 | 8.10 | E |
| | yl}methyl)pyrrolidin-3-ol | | | |
| 399 | (3S,3′R)-1′-[4-(2-naphthyl)pyrimidin-2-yl]-1,3′-bipyrrolidin-3-ol | 361 | 7.70 | E |
| 400 | 2-{(3R)-1-[4-(2-naphthyl)pyrimidin-2-yl]pyrrolidin-3- | 414 | 8.80 | E |
| | yl}octahydro-2H-pyrido[1,2-a]pyrazine | | | |
| 401 | (3S,3′R)-1′-[4-(2-naphthyl)pyrimidin-2-yl]-1,3′-bipyrrolidin-3- | 360 | 7.90 | E |
| | amine | | | |
| 402 | 3-(methyl{(3R)-1-[4-(2-naphthyl)pyrimidin-2-yl]pyrrolidin-3- | 361 | 7.70 | E |
| | yl}amino)propan-1-ol | | | |
| 403 | N,N-dimethyl-N′-{(3R)-1-[4-(2-naphthyl)pyrimidin-2- | 360 | 7.60 | E |
| | yl]pyrrolidin-3-yl}ethane-1,2-diamine | | | |
| 404 | N,N′-dimethyl-N-{(3R)-1-[4-(2-naphthyl)pyrimidin-2- | 362 | 8.60 | E |
| | yl]pyrrolidin-3-yl}ethane-1,2-diamine | | | |
| 405 | (3R,3′R)-1′-[4-(2-naphthyl)pyrimidin-2-yl]-1,3′-bipyrrolidin-3-ol | 386 | 7.70 | E |
| 406 | (3R,3′R)-1′-[4-(2-naphthyl)pyrimidin-2-yl]-1,3′-bipyrrolidin-3- | 362 | 7.90 | E |
| | amine | | | |
| 407 | (1S,4S)-2-methyl-5-{(3R)-1-[4-(2-naphthyl)pyrimidin-2- | 386 | 8.20 | E |
| | yl]pyrrolidin-3-yl}-2,5-diazabicyclo[2.2.1]heptane | | | |
| 408 | 5-{(3R)-1-[4-(2-naphthyl)pyrimidin-2-yl]pyrrolidin-3 - | 386 | 8.80 | E |
| | yl}octahydropyrrolo[3,4-b]pyrrole | | | |
| 409 | N,N,N′-trimethyl-N′-{(3R)-1-[4-(2-naphthyl)pyrimidin-2- | 390 | 6.60 | E |
| | yl]pyrrolidin-3-yl}propane-1,3-diamine | | | |
| 410 | (3R,3′S)-1′-[4-(2-naphthyl)pyrimidin-2-yl]-1,3′-bipyrrolidin-3-ol | 361 | 7.70 | E |
| 411 | (3S,3′S)-1′-[4-(2-naphthyl)pyrimidin-2-yl]-1,3′-bipyrrolidin-3-ol | 361 | 7.70 | E |
| 412 | N,N-dimethyl-N′-{(3S)-1-[4-(2-naphthyl)pyrimidin-2- | 362 | 7.50 | E |
| | yl]pyrrolidin-3-yl}ethane-1,2-diamine | | | |
| 413 | N,N,N′-trimethyl-N′-{(3S)-1-[4-(2-naphthyl)pyrimidin-2- | 376 | 8.50 | E |
| | yl]pyrrolidin-3-yl}ethane-1,2-diamine | | | |
| 414 | 3-(methyl{(3S)-1-[4-(2-naphthyl)pyrimidin-2-yl]pyrrolidin-3- | 363 | 7.30 | E |
| | yl}amino)propan-1-ol | | | |
| 415 | 5-{(3S)-1-[4-(2-naphthyl)pyrimidin-2-yl]pyrrolidin-3- | 386 | 8.80 | E |
| | yl}octahydropyrrolo[3,4-b]pyrrole | | | |
| 416 | N,N,N′-trimethyl-N′-{(3S)-1-[4-(2-naphthyl)pyrimidin-2- | 390 | 6.60 | E |
| | yl]pyrrolidin-3-yl}propane-1,3-diamine | | | |

### Example 417

### Preparation of 2-(Ethyl{(3S)-1-[4-(2-naphthyl)pyrimidin-2-yl]pyrrolidin-3-yl}amino)ethanol

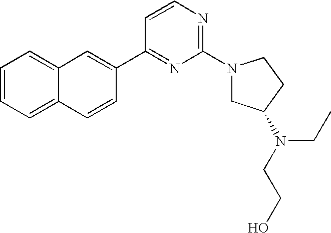

2-(Ethyl{(3S)-1-[4-(2-naphthyl)pyrimidin-2-yl]pyrrolidin-3-yl}amino)ethanol To a vial was added a solution of (R)-1-(4-(naphthalen-2-yl)pyrimidin-2-yl)pyrrolidin-3-yl methanesulfonate in NMP (1 mL of a 0.0812 mM solution, 0.0812 mmol) followed by 2-(ethylamino)ethanol (0.0792 mL, 0.812 mmol, 10 equiv.). The sealed vial was heated in an 80 C shaker block for 14 h. The reaction was cooled to rt and water (0.1 mL) was added. The reaction was purified by reverse phase HPLC (method D without TFA) to provide the title compound as a tan solid (12 mg, 41%). LC/MS (method E) rt=3.6 min; calculated mass =362, [M+H]⁺=363.

The same method was used to prepare the compounds in the following table using the procedure for the example above.

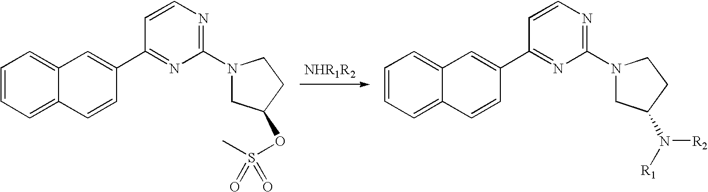

| Example | | | | HPLC |
|---|---|---|---|---|
| No. | Compounds | MS | Rt | Method |
| 418 | 2-[(3S)-3-(1H-imidazol-1-yl)pyrrolidin-1-yl]-4-(2-naphthyl)pyrimidine | 342 | 4.00 | A |
| 419 | (3S)-N-cyclohexyl-N-methyl-1-[4-(2-naphthyl)pyrimidin-2- | 387 | 3.10 | A |
| | yl]pyrrolidin-3-amine | | | |
| 420 | (3S)-N-(tert-butyl)-1-[4-(2-naphthyl)pyrimidin-2-yl]pyrrolidin-3-amine | 347 | 2.80 | A |
| 421 | 4-(2-naphthyl)-2-[(3S)-3-piperazin-1-ylpyrrolidin-1-yl]pyrimidine | 360 | 2.70 | A |
| 422 | 4-{(3S)-1-[4-(2-naphthyl)pyrimidin-2-yl]pyrrolidin-3-yl}morpholine | 361 | 4.70 | A |
| 423 | 4-{(3S)-1-[4-(2-naphthyl)pyrimidin-2-yl]pyrrolidin-3-yl}piperazin-2- | 374 | 3.60 | A |
| | one | | | |
| 424 | 1-{(3S)-1-[4-(2-naphthyl)pyrimidin-2-yl]pyrrolidin-3-yl}azepane | 374 | 2.50 | A |
| 425 | (3′S)-1′-[4-(2-naphthyl)pyrimidin-2-yl]-1,3′-bipyrrolidine | 345 | 2.90 | A |
| 426 | 4-(2-naphthyl)-2-[(3S)-3-piperidin-1-ylpyrrolidin-1-yl]pyrimidine | 359 | 3.00 | A |
| 427 | 2-[(3S)-3-(4-methylpiperazin-1-yl)pyrrolidin-1-yl]-4-(2- | 374 | 2.80 | A |
| | naphthyl)pyrimidine | | | |
| 428 | (1-{(3S)-1-[4-(2-naphthyl)pyrimidin-2-yl]pyrrolidin-3-yl}piperidin-3- | 389 | 8.00 | F |
| | yl)methanol | | | |
| 429 | (1-{(3S)-1-[4-(2-naphthyl)pyrimidin-2-yl]pyrrolidin-3-yl}piperidin-4- | 389 | 7.90 | F |
| | yl)methanol | | | |
| 430 | 1-{(3S)-1-[4-(2-naphthyl)pyrimidin-2-yl]pyrrolidin-3-yl}piperidin-3-ol | 375 | 8.00 | F |
| 431 | (3S)-N-(2-morpholin-4-ylethyl)-1-[4-(2-naphthyl)pyrimidin-2- | 404 | 8.10 | F |
| | yl]pyrrolidin-3-amine | | | |
| 432 | 1-{(3S)-1-[4-(2-naphthyl)pyrimidin-2-yl]pyrrolidin-3-yl}piperidin-4-ol | 375 | 7.70 | F |
| 433 | (3R,3′S)-N,N-dimethyl-1′-[4-(2-naphthyl)pyrimidin-2-yl]-1,3′- | 388 | 8.40 | F |
| | bipyrrolidin-3-amine | | | |
| 434 | (3S,3′S)-N,N-dimethyl-1′-[4-(2-naphthyl)pyrimidin-2-yl]-1,3′- | 388 | 8.40 | F |
| | bipyrrolidin-3-amine | | | |
| 435 | (3R)-1-{(3S)-1-[4-(2-naphthyl)pyrimidin-2-yl]pyrrolidin-3- | 375 | 7.80 | F |
| | yl}piperidin-3-ol | | | |
| 436 | (3S)-1-{(3S)-1-[4-(2-naphthyl)pyrimidin-2-yl]pyrrolidin-3- | 375 | 7.80 | F |
| | yl}piperidin-3-ol | | | |

The following compounds were prepared using the above method but starting from (S)-1-(4-(naphthalen-2-yl)pyrimidin-2-yl)pyrrolidin-3-yl methanesulfonate prepared from (3S)-i-[4-(2-naphthyl)pyrimidin-2-yl]pyrrolidin-3-ol as in the example above

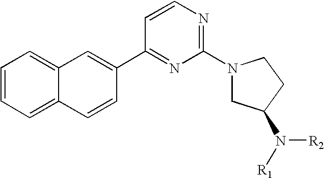

| Example | | | Rt | HPLC |
|---|---|---|---|---|
| No. | Compound | MS | (min.) | Method |
| 437 | 2-(ethyl{(3R)-1-[4-(2-naphthyl)pyrimidin-2-yl]pyrrolidin-3- | 363 | 4.20 | A |
| | yl}amino)ethanol | | | |
| 438 | 2-[(3R)-3-(1H-imidazol-1-yl)pyrrolidin-l-yl]-4-(2-naphthyl)pyrimidine | 342 | 4.00 | A |
| 439 | (3R)-N-cyclohexyl-N-methyl-1-[4-(2-naphthyl)pyrimidin-2- | 387 | 3.20 | A |
| | yl]pyrrolidin-3-amine | | | |
| 440 | (3R)-N-(tert-butyl)-1-[4-(2-naphthyl)pyrimidin-2-yl]pyrrolidin-3- | 347 | 2.90 | A |
| | amine | | | |
| 441 | 4-(2-naphthyl)-2-[(3R)-3-piperazin-1-ylpyrrolidin-1-yl]pyrimidine | 360 | 2.60 | A |
| 442 | 4-{(3R)-1-[4-(2-naphthyl)pyrimidin-2-yl]pyrrolidin-3-yl}morpholine | 361 | 4.70 | A |
| 443 | 4-{(3R)-1-[4-(2-naphthyl)pyrimidin-2-yl]pyrrolidin-3-yl}piperazin-2- | 374 | 3.60 | A |
| | one | | | |
| 444 | 1-{(3R)-1-[4-(2-naphthyl)pyrimidin-2-yl]pyrrolidin-3-yl}azepane | 374 | 2.40 | A |
| 445 | (3′R)-1′-[4-(2-naphthyl)pyrimidin-2-yl]-1,3′-bipyrrolidine | 345 | 2.80 | A |
| 446 | 4-(2-naphthyl)-2-[(3R)-3-piperidin-1-ylpyrrolidin-1-yl]pyrimidine | 359 | 2.90 | A |
| 447 | 2-[(3R)-3-(4-methylpiperazin-1-yl)pyrrolidin-1-yl]-4-(2- | 374 | 2.80 | A |
| | naphthyl)pyrimidine | | | |
| 448 | (1-{(3R)-1-[4-(2-naphthyl)pyrimidin-2-yl]pyrrolidin-3-yl}piperidin-3- | 389 | 8.00 | F |
| | yl)methanol | | | |
| 449 | (1-{(3R)-1-[4-(2-naphthyl)pyrimidin-2-yl]pyrrolidin-3-yl}piperidin-4- | 389 | 7.90 | F |
| | yl)methanol | | | |
| 450 | 1-{(3R)-1-[4-(2-naphthyl)pyrimidin-2-yl]pyrrolidin-3-yl}piperidin-3-ol | 375 | 7.90 | F |
| 451 | (3R)-N-(2-morpholin-4-ylethyl)-1-[4-(2-naphthyl)pyrimidin-2- | 404 | 8.10 | F |
| | yl]pyrrolidin-3-amine | | | |
| 452 | 1-{(3R)-1-[4-(2-naphthyl)pyrimidin-2-yl]pyrrolidin-3-yl}piperidin-4-ol | 375 | 7.70 | F |
| 453 | (3R,3′R)-N,N-dimethyl-1′-[4-(2-naphthyl)pyrimidin-2-yl]-1,3′- | 388 | 8.40 | F |
| | bipyrrolidin-3-amine | | | |
| 454 | (3S,3′R)-N,N-dimethyl-1′-[4-(2-naphthyl)pyrimidin-2-yl]-1,3′- | 388 | 8.40 | F |
| | bipyrrolidin-3-amine | | | |
| 455 | (3R)-1-{(3R)-1-[4-(2-naphthyl)pyrimidin-2-yl]pyrrolidin-3- | 375 | 7.80 | F |
| | yl}piperidin-3-ol | | | |
| 456 | (3S)-1-{(3R)-1-[4-(2-naphthyl)pyrimidin-2-yl]pyrrolidin-3- | 375 | 7.85 | F |
| | yl}piperidin-3-ol | | | |

### Example 457

### Preparation of (1-(4-(naphthalene-2-yl)pyrimidin-2-yl)pyrrolidin-3-yl)methanol

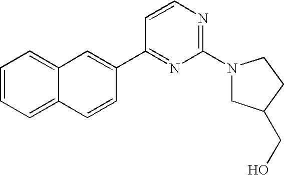

(1-(4-(naphthalene-2-yl)pyrimidin-2-yl)pyrrolidin-3-yl)methanol A solution of pyrrolidin-3-ylmethanol (240 mg, 2.4 mmol), DIPEA (6 mmol, 1.04 mL) and 2-chloro-4-(naphthalen-2-yl)pyrimidine (481 mg, 2 mmol) in DMSO (3 mL) was heated to 80° C. for 18 h. After cooling to room temperature the mixture was diluted with ethyl acetate (50 mL) and washed with 1 M sodium carbonate (50 mL), water (2×50 mL) and brine (50 mL). The organic layer was dried (MgSO₄) and evaporated. The crude product was purified by silica gel chromatography eluting with 0-100% ethyl acetate in hexanes to give the product (464 mg, 76%). HPLC (method C) rt=9.7 mins., purity=100%. HR ESMS [M+H]⁺=306.1582.

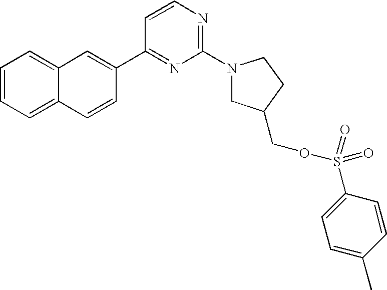

(1-(4-(naphthalen-2-yl)pyrimidin-2-yl)pyrrolidin-3-yl)methyl 4-methyl benzenesulfonate To a solution of (1-(4-(naphthalene-2-yl)pyrimidin-2-yl)pyrrolidin-3-yl)methanol (156 mg, 0.51 mmol) in dichloromethane (8 mL) were added DMAP (catalytic quantity), triethyl amine (1 mL), and 4-methylbenzenesulfonyl chloride (195 mg, 1.02 mmol). The reaction was stirred at rt for 5 h. To the reaction was added water (25 mL), and the layers were separated. The organic layer was washed with sat. NaHCO₃ (2×25 mL), 0.5 M HCl (20 mL), and brine (20 mL). The DCM layer was dried (Na₂SO₄), filtered, and concentrated. The crude material was purified by silica gel chromatography, eluting with a gradient of 5% EtOAc/hexane to 60% EtOAc, to afford the title compound (217 mg, 93%). HPLC (method C) rt=11.5 mins., purity=100%. HR ESMS [M+H]⁺=460.1696.

### Example 458

### Preparation of 1-((1-(4-(naphthalen-2-yl)pyrimidin-2-yl)pyrrolidin-3-yl)methyl)azepane

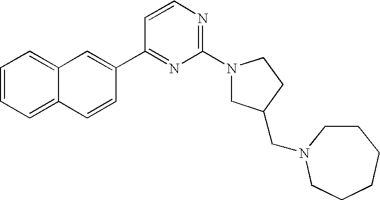

1-((1-(4-(naphthalen-2-yl)pyrimidin-2-yl)pyrrolidin-3-yl)methyl)azepane To a vial was added a solution of (1-(4-(naphthalen-2-yl)pyrimidin-2-yl)pyrrolidin-3-yl)methyl 4-methyl benzenesulfonate in NMP (0.5 mL of a 0.086 mM solution, 0.043 mmol) followed by hexamethyleneimine (0.024 mL, 0.217 mmol, 5 equiv.). The sealed vial was heated in an 80 C shaker block for 14 h. The reaction was cooled to rt and water (0.1 mL) was added. The reaction was purified by reverse phase HPLC (method D without TFA) to provide the title compound (10 mg, 61%). LC/MS (method E) rt=1.0 min; calculated mass=386, [M+H]⁺=387.

The same method was used to prepare the compounds in the following table using the procedure for the example above.

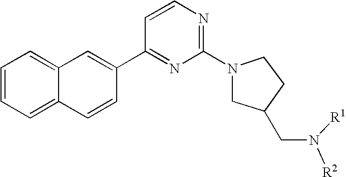

| Example | | | Rt | HPLC |
|---|---|---|---|---|
| No. | Compound | MS | (min.) | method |
| 459 | 4-(2-naphthyl)-2-[3-(piperidin-1-ylmethyl)pyrrolidin-1- | 373 | 1.10 | E |
| | yl]pyrimidine | | | |
| 460 | 4-(2-naphthyl)-2-[3-(pyrrolidin-1-ylmethyl)pyrrolidin-1- | 359 | 1.10 | E |
| | yl]pyrimidine | | | |
| 461 | 2-[3-(azetidin-1-ylmethyl)pyrrolidin-1-yl]-4-(2- | 345 | 1.10 | E |
| | naphthyl)pyrimidine | | | |
| 462 | 4-({1-[4-(2-naphthyl)pyrimidin-2-yl]pyrrolidin-3- | 375 | 1.10 | E |
| | yl}methyl)morpholine | | | |
| 463 | 4-({1-[4-(2-naphthyl)pyrimidin-2-yl]pyrrolidin-3- | 391 | 1.10 | E |
| | yl}methyl)thiomorpholine | | | |
| 464 | N-ethyl-N-({1-[4-(2-naphthyl)pyrimidin-2-yl]pyrrolidin-3- | 361 | 1.20 | E |
| | yl}methyl)ethanamine | | | |
| 465 | 2-methyl-N-({1-[4-(2-naphthyl)pyrimidin-2-yl]pyrrolidin-3- | 361 | 1.20 | E |
| | yl}methyl)propan-2-amine | | | |
| 466 | 2-[ethyl({1-[4-(2-naphthyl)pyrimidin-2-yl]pyrrolidin-3- | 377 | 1.10 | E |
| | yl}methyl)amino]ethanol | | | |
| 467 | 2-{3-[(4-methylpiperazin-1-yl)methyl]pyrrolidin-1-yl}-4-(2- | 388 | 1.10 | E |
| | naphthyl)pyrimidine | | | |
| 468 | 4-({1-[4-(2-naphthyl)pyrimidin-2-yl]pyrrolidin-3- | 388 | 1.10 | E |
| | yl}methyl)piperazin-2-one | | | |
| 469 | 2-[3-(1H-imidazol-1-ylmethyl)pyrrolidin-1-yl]-4-(2- | 356 | 1.10 | E |
| | naphthyl)pyrimidine | | | |
| 470 | 2-[3-(chloromethyl)pyrrolidin-1-yl]-4-(2-naphthyl)pyrimidine | 324 | 1.90 | E |

The following compounds were prepared using the above method but starting from (R)-(1-(4-(naphthalen-2-yl)pyrimidin-2-yl)pyrrolidin-3-yl)methyl 4-methylbenzenesulfonate prepared from (R)-(1-(4-(naphthalen-2-yl)pyrimidin-2-yl)pyrrolidin-3-yl)methanol as in the above example.

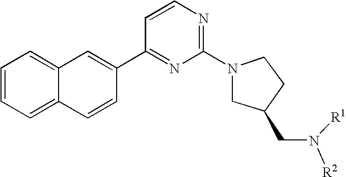

| Example | Chemical | | Rt | HPLC |
|---|---|---|---|---|
| No. | Name | MS | (min.) | method |
| 471 | N,N-dimethyl-1-{(3S)-1-[4-(2-naphthyl)pyrimidin-2-yl]pyrrolidin- | 333 | 3.27 | B |
| | 3-yl}methanamine | | | |
| 472 | 4-(2-naphthyl)-2-[(3S)-3-(piperidin-1-ylmethyl)pyrrolidin-1- | 373 | 3.44 | B |
| | yl]pyrimidine | | | |
| 473 | 4-(2-naphthyl)-2-[(3S)-3-(pyrrolidin-1-ylmethyl)pyrrolidin-1- | 359 | 3.36 | B |
| | yl]pyrimidine | | | |
| 474 | N-methyl-1-{(3S)-1-[4-(2-naphthyl)pyrimidin-2-yl]pyrrolidin-3- | 319 | 3.20 | B |
| | yl}methanmine | | | |
| 475 | N-methyl-N-({(3S)-1-[4-(2-naphthyl)pyrimidin-2-yl]pyrrolidin-3- | 401 | 3.76 | B |
| | yl}methyl)cyclohexanamine | | | |
| 476 | 1-({(3R)-1-[4-(2-naphthyl)pyrimidin-2-yl]pyrrolidin-3- | 387 | 4.73 | B |
| | yl}methyl)piperidin-2-one | | | |
| 477 | tert-butyl 4-({(3S)-1-[4-(2-naphthyl)pyrimidin-2-yl]pyrrolidin-3- | 474 | 3.84 | B |
| | yl}methyl)piperazine-1-carboxylate | | | |
| 478 | 2-methyl-N-({(3S)-1-[4-(2-naphthyl)pyrimidin-2-yl]pyrrolidin-3- | 361 | 3.46 | B |
| | yl}methyl)propan-2-amine | | | |
| 479 | 2-[ethyl({(3S)-1-[4-(2-naphthyl)pyrimidin-2-yl]pyrrolidin-3- | 377 | 3.29 | B |
| | yl}methyl)amino]ethanol | | | |
| 480 | 2-{(3S)-3-[(4-methylpiperazin-1-yl)methyl]pyrrolidin-1-yl}-4-(2- | 388 | 3.14 | B |
| | naphthyl)pyrimidine | | | |
| 481 | (3S)-N,N-dimethyl-1-({(3S)-1-[4-(2-naphthyl)pyrimidin-2- | 402 | 2.80 | B |
| | yl]pyrrolidin-3-yl}methyl)pyrrolidin-3-amine | | | |
| 482 | 2-[(3R)-3-(1H-imidazol-1-ylmethyl)pyrrolidin-1-yl]-4-(2- | 356 | 3.31 | B |
| | naphthyl)pyrimidine | | | |
| 483 | (3R)-N,N-dimethyl-1-({(3S)-1-[4-(2-naphthyl)pyrimidin-2- | 402 | 2.78 | B |
| | yl]pyrrolidin-3-yl}methyl)pyrrolidin-3-amine | | | |

The following compounds were prepared using the above method but starting from (S)-(1-(4-(naphthalen-2-yl)pyrimidin-2-yl)pyrrolidin-3-yl)methyl 4-methylbenzenesulfonate prepared from (S)-(1-(4-(naphthalen-2-yl)pyrimidin-2-yl)pyrrolidin-3-yl)methanol as in the above example.

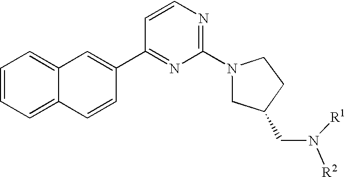

| Example | | | Rt | HPLC |
|---|---|---|---|---|
| No. | Compound | MS | (mim) | method |
| 484 | N,N-dimethyl-1-{(3R)-1-[4-(2-naphthyl)pyrimidin-2-yl]pyrrolidin- | 333 | 3.30 | B |
| | 3-yl}methanamine | | | |
| 485 | 4-(2-naphthyl)-2-[(3R)-3-(piperidin-1-ylmethyl)pyrrolidin-1- | 373 | 3.52 | B |
| | yl]pyrimidine | | | |
| 486 | 4-(2-naphthyl)-2-[(3R)-3-(pyrrolidin-1-ylmethyl)pyrrolidin-1- | 359 | 3.42 | B |
| | yl]pyrimidine | | | |
| 487 | N-methyl-1-{(3R)-1-[4-(2-naphthyl)pyrimidin-2-yl]pyrrolidin-3- | 319 | 3.23 | B |
| | yl}methanamine | | | |
| 488 | N-methyl-N-({(3R)-1-[4-(2-naphthyl)pyrimidin-2-yl]pyrrolidin-3- | 401 | 3.82 | B |
| | yl}methyl)cyclohexanamine | | | |
| 489 | 1-({(3S)-1-[4-(2-naphthyl)pyrimidin-2-yl]pyrrolidin-3- | 387 | 4.78 | B |
| | yl}methyl)piperidin-2-one | | | |
| 490 | tert-butyl 4-({(3R)-1-[4-(2-naphthyl)pyrimidin-2-yl]pyrrolidin-3- | 474 | 3.88 | B |
| | yl}methyl)piperazine-1-carboxylate | | | |
| 491 | 2-methyl-N-({(3R)-1-[4-(2-naphthyl)pyrimidin-2-yl]pyrrolidin-3- | 361 | 3.54 | B |
| | yl}methyl)propan-2-amine | | | |
| 492 | 2-[ethyl({(3R)-1-[4-(2-naphthyl)pyrimidin-2-yl]pyrrolidin-3- | 377 | 3.33 | B |
| | yl}methyl)amino]ethanol | | | |
| 493 | 2-{(3R)-3-[(4-methylpiperazin-1-yl)methyl]pyrrolidin-1-yl}-4-(2- | 388 | 3.25 | B |
| | naphthyl)pyrimidine | | | |
| 494 | (3S)-N,N-dimethyl-1-({(3R)-1-[4-(2-naphthyl)pyrimidin-2- | 402 | 3.06 | B |
| | yl]pyrrolidin-3-yl}methyl)pyrrolidin-3-amine | | | |
| 495 | 2-[(3S)-3-( 1H-imidazol-1-ylmethyl)pyrrolidin-1-yl]-4-(2- | 356 | 3.37 | B |
| | naphthyl)pyrimidine | | | |
| 496 | (3R)-N,N-dimethyl-1-({(3R)-1-[4-(2-naphthyl)pyrimidin-2- | 402 | 2.90 | B |
| | yl]pyrrolidin-3-yl}methyl)pyrrolidin-3-amine | | | |

### Example 497

### Preparation of (R)—N,N-dimethyl-1-(4-(naphthalen-2-yl)pyrimidin-2-yl)pyrrolidine-2-carboxamide

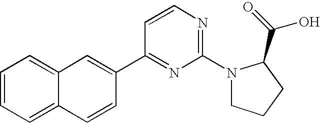

(R)-1-(4-(naphthalen-2-yl)pyrimidin-2-yl)pyrrolidine-2-carboxylic acid A solution of L-proline (429 mg, 3.73 mmol), DIPEA (7.47 mmol, 1.30 mL) and 2-chloro-4-(naphthalen-2-yl)pyrimidine (600 mg, 2.49 mmol) in DMSO (5 mL) was heated to 80° C. for 18 h. After cooling to room temperature the mixture, it was added water (50 mL) and the PH brought to 3-4. The precipitated product was filtered and washed with cold water, then dried to leave the product (520 mg, 65%). HPLC (method C) rt=9.7 mins., purity=98.9%.

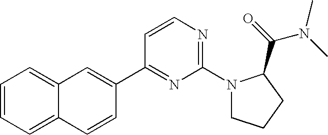

(R)—N,N-dimethyl-1-(4-(naphthalen-2-yl)pyrimidin-2-yl)pyrrolidine-2-carboxamide (To a vial was added a solution of (R)-1-(4-(naphthalen-2-yl)pyrimidin-2-yl)pyrrolidine-2-carboxylic acid (25 mg, 0.078 mmol) in DMF (0.5 mL) followed by HATU ( mg, 0.156 mmol, 2 equiv.) and dimethylamine (solution 2 M in THF, 0.19 mL, 0.391 mmol, 5 equiv.). The sealed vial was stirred in a shaker block at room temperature for 30 min. It was added water (0.1 mL). The reaction was purified by reverse phase HPLC (method D without TFA) to provide the title compound (5.3 mg, 20%). LC/MS (method E) rt=1.47 min; calculated mass=346, [M+H]⁺=347.

The same method was used to prepare the compounds in the following table using the procedure for the example above.

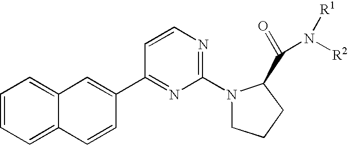

| Example | | | | HPLC |
|---|---|---|---|---|
| No. | Compound | MS | Rt | method |
| 498 | 4-(2-naphthyl)-2-[(2S)-2-(piperidin-1-ylcarbonyl)pyrrolidin-1- | 387 | 1.69 | A |
| | yl]pyrimidine | | | |
| 499 | 4-(2-naphthyl)-2-[(2S)-2-(pyrrolidin-1-ylcarbonyl)pyrrolidin-1- | 373 | 1.57 | A |
| | yl]pyrimidine | | | |
| 500 | N-tert-butyl-1-[4-(2-naphthyl)pyrimidin-2-yl]-L-prolinamide | 375 | 1.73 | A |
| 501 | N-ethyl-N-(2-hydroxyethyl)-1-[4-(2-naphthyl)pyrimidin-2-yl]-L- | 391 | 1.38 | A |
| | prolinamide | | | |
| 502 | 2-{(2S)-2-[(4-methylpiperazin-1-yl)carbonyl]pyrrolidin-1-yl}-4-(2- | 402 | 1.20 | A |
| | naphthyl)pyrimidine | | | |
| 503 | (3S)-N,N-dimethyl-1-{1-[4-(2-naphthyl)pyrimidin-2-yl]-L- | 416 | 1.15 | A |
| | prolyl}pyrrolidin-3-amine | | | |
| 504 | (3R)-N,N-dimethyl-1-{1-[4-(2-naphthyl)pyrimidin-2-yl]-L- | 416 | 1.19 | A |
| | prolyl}pyrrolidin-3-amine | | | |

The following compounds were prepared using the above method but starting from (S)-1-(4-(naphthalen-2-yl)pyrimidin-2-yl)pyrrolidine-2-carboxylic acid that was prepared as in the example above.

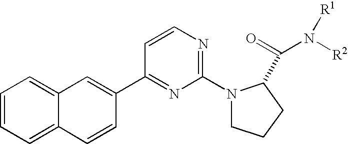

| Example | | | | HPLC |
|---|---|---|---|---|
| No. | Compound | MS | Rt | method |
| 505 | N,N-dimethyl-1-[4-(2-naphthyl)pyrimidin-2-yl]-D-prolinamide | 347 | 1.43 | A |
| 506 | 4-(2-naphthyl)-2-[(2R)-2-(piperidin-1-ylcarbonyl)pyrrolidin-1- | 387 | 1.24 | A |
| | yl]pyrimidine | | | |
| 507 | 4-(2-naphthyl)-2-[(2R)-2-(pyrrolidin-1-ylcarbonyl)pyrrolidin-1- | 373 | 1.22 | A |
| | yl]pyrimidine | | | |
| 508 | N-methyl-1-[4-(2-naphthyl)pyrimidin-2-yl]-D-prolinamide | 333 | 1.42 | A |
| 509 | N-tert-butyl-1-[4-(2-naphthyl)pyrimidin-2-yl]-D-prolinamide | 375 | 1.72 | A |
| 510 | 2-{(2R)-2-[(4-methylpiperazin-1-yl)carbonyl]pyrrolidin-1-yl}-4- | 402 | 1.19 | A |
| | (2-naphthyl)pyrimidine | | | |
| 511 | (3S)-N,N-dimethyl-1-{1-[4-(2-naphthyl)pyrimidin-2-yl]-D- | 416 | 1.13 | A |
| | prolyl}pyrrolidin-3-amine | | | |
| 512 | (3R)-N,N-dimethyl-1-{1-[4-(2-naphthyl)pyrimidin-2-yl]-D- | 416 | 1.15 | A |
| | prolyl}pyrrolidin-3-amine | | | |

### Example 513

### Preparation of (3′S)-1′-[4-(2-Naphthyl)pyrimidin-2-yl]-1,3′-bipyrrolidin-2-one

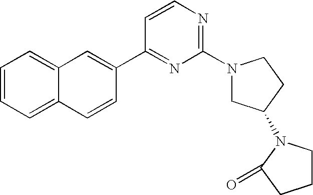

(3′S)-1′-[4-(2-Naphthyl)pyrimidin-2-yl]-1,3′-bipyrrolidin-2-one To a vial containing NaH (60% dispersion, 0.043 g, 1.08 mmol, 10 equiv.) was added dropwise a solution of pyrrolidinone (0.083 mL, 1.08 mmol, 10 equiv.) in NMP (1 mL) and the mixture was stirred for 1 h. To the reaction was added a solution of (R)-1-(4-(naphthalen-2-yl)pyrimidin-2-yl)pyrrolidin-3-yl methanesulfonate in NMP (1 mL of a 0.0812 mM solution, 0.0812 mmol) and the reaction was heated in an 80 C shaker block for 14 h. The reaction was cooled to rt, and water (10 mL) and EtOAc (50 mL) were added. The EtOAc layer was washed with water (3×20 mL) and brine (20 mL), dried (Na₂SO₄), filtered, and concentrated. The residue was purified by reverse phase HPLC (method D without TFA) to provide the title compound as a tan solid (9 mg, 31%). LC/MS (method A) rt=3.7 min; calculated mass=358, [M+H]⁺=359.

### Example 514

### Preparation of (3′R)-1′-[4-(2-Naphthyl)pyrimidin-2-yl]-1,3′-bipyrrolidin-2-one

(3′R)-1′-[4-(2-Naphthyl)pyrimidin-2-yl]-1,3′-bipyrrolidin-2-one was prepared in a similar method starting from (S)-1-(4-(naphthalen-2-yl)pyrimidin-2-yl)pyrrolidin-3-yl methanesulfonate. LC/MS (method A) rt=3.7 min; calculated mass=358, [M+H]⁺=359.

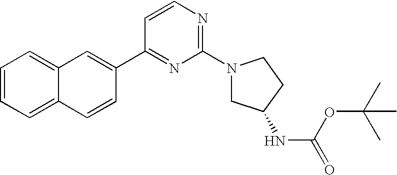

### Example 515

### Preparation of tert-Butyl {(3S)-1-[4-(2-naphthyl)pyrimidin-2-yl]pyrrolidin-3-yl }carbamate

tert-Butyl {(3S)-1-[4-(2-naphthyl)pyrimidin-2-yl]pyrrolidin-3-yl}carbamate A solution of 2-chloro-4-(naphthalene-2-yl)pyrimidine (0.25 g, 1.0 mmol), (3S)-(-)-3-(tert-butoxycarbonylamino)pyrrolidine (0.29 g, 1.5 mmol), and diisopropylethylamine (0.27 mL, 1.5 mmol) in N-methylpyrrolidine (2 mL) was heated in a vial in a shaker block at 80° C. for 14 h. The reaction was cooled to room temperature and EtOAc (200 mL) and water (25 mL) were used to transfer the contents of the vial to a separatory funnel. The layers were separated. The organic layer was washed with water (8×30 mL), and brine (30 mL), dried (Na₂SO₄), filtered, and concentrated. The crude material was purified by silica gel chromatography, eluting with 3% MeOH/CH₂Cl₂, to afford the title compound as an ivory powder (0.40 g, 99%). HPLC (Method C) purity 100%, rt=11.2 min; LC/MS (Method A), rt=1.78 mins., calculated mass=390, [M+H]⁺=391.

The following compounds were prepared in a manner similar to the example above from 2-chloro-4-(naphthalene-2-yl)pyrimidine and the appropriate amine.

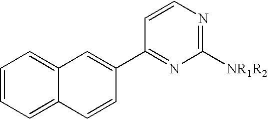

| Example | | | Rt | HPLC |
|---|---|---|---|---|
| No. | Compound | MS | (min.) | Method |
| 516 | 2-[(3R)-3-methoxypyrrolidin-1-yl]-4-(2-naphthyl)pyrimidine | 306 | 10.5 | F |
| 517 | 2-[(3S)-3-methoxypyrrolidin-1-yl]-4-(2-naphthyl)pyrimidine | 306 | 10.5 | F |
| 518 | 2,2,2-trifluoro-N-(2-{(2S)-1-[4-(2-naphthyl)pyrimidin-2-yl]pyrrolidin-2- | 415 | 11.5 | F |
| | yl}ethyl) acetamide | | | |
| 519 | 2,2,2-trifluoro-N-(2-{(2R)-1-[4-(2-naphthyl)pyrimidin-2-yl]pyrrolidin-2- | 415 | 11.5 | F |
| | yl}ethyl) acetamide | | | |
| 520 | (3R)-N-methyl-1-[4-(2-naphthyl)pyrimidin-2-yl]pyrrolidin-3-amine | 305 | 9.8 | G |
| 521 | (3S)-N-methyl-1-[4-(2-naphthyl)pyrimidin-2-yl]pyrrolidin-3-amine | 305 | 9.8 | G |
| 522 | (3R)-N,N-dimethyl-1-[4-(2-naphthyl)pyrimidin-2-yl]pyrrolidin-3-amine | 319 | 7.7 | F |

### Example 523

### Preparation of N-{(3S)-1-[4-(2-Naphthyl)pyrimidin-2-yl]pyrrolidin-3-yl}-N-(2,2,2-trifluoroethyl)acetamide

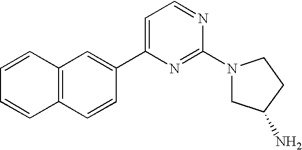

(3S)-1-[4-(2-naphthyl)pyrimidin-2-yl]pyrrolidin-3-amine. To a solution of tert-butyl {(3S)-1-[4-(2-naphthyl)pyrimidin-2-yl]pyrrolidin-3-yl}carbamate (0.35 g, 0.89 mmol) in CH₂Cl₂ (10 mL) was trifluoroacetic acid (5 mL). The solution was stirred at room temperature for 14 h, then concentrated to a dark oil. To the residue was added a saturated K₂CO₃ solution (5 mL), followed by ethyl acetate (100 mL). The layers were separated and the aqueous layer was extracted with ethyl acetate (2×100 mL). The combined organic layers were washed with brine (25 mL), dried (Na₂SO₄), filtered, and concentrated to afford an orange oil (0.22 g, 85%). A sample was purified by RP HPLC (Method D) for analysis. HPLC (Method C) purity 99.7%, rt=7.5 min.; HRMS: calcd for C₁₈H₁₈N₄⁺H⁺, 291.16042; found ([M+H]⁺), 291.1617.

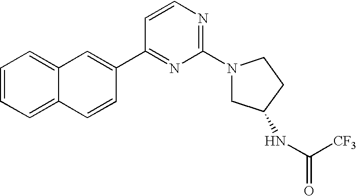

(S)-2,2,2-Trifluoro-N-(1-(4-(□aphthalene-2-yl)pyrimidin-2-yl)pyrrolidin-3-yl)acetamide To a solution of (3S)-1-[4-(2-naphthyl)pyrimidin-2-yl]pyrrolidin-3-amine (0.30 g, 1.03 mmol)in MeOH (anhydrous, 6 mL) was added ethyl trifluoroacetate (0.141 mL, 1.18 mmol, 1.15 equiv.) followed by triethyl amine (0.166 mL, 1.18 mmol, 1.15 equiv.). The reaction was stirred at rt for 1.5 h then concentrated. The residue was dissolved in EtOAc (125 mL), washed with water (75 mL) and brine (75 mL). The EtOAc solution was dried (Na₂SO₄), filtered, and concentrated to afford a light yellow powder (0.44 g, 95%) which was used without further purification. LC/MS (Method A) rt=2.4 min; calculated mass=386.37, [M+H]⁺=387.3.

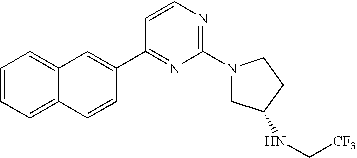

(3S)-1-[4-(2-Naphthyl)pyrimidin-2-yl]-N-(2,2,2-trifluoroethyl)pyrrolidin-3-amine To a solution of (S)-2,2,2-trifluoro-N-(1-(4-(□aphthalene-2-yl)pyrimidin-2-yl)pyrrolidin-3-yl)acetamide (0.256 g, 0.66 mmol) in THF (anhydrous, 5 mL) was added dropwise a solution of borane in THF (1.0 M, 2.8 mL, 2.8 mmol, 4.2 equiv.). The reaction was heated in a 60 C bath for 5.5 h then cooled to rt. The reaction was cooled in an ice bath and quenched with MeOH (2 mL). The solution was concentrated and redissolved in MeOH (20 mL). To the solution was added a solution of HCl (4 M in dioxane, 1.25 mL) and stirred at rt for 0.5 h, then concentrated. The residue was taken up in CH₂Cl₂ (150 mL) and washed with sat. K₂CO₃ solution (2×75 mL) and brine (75 mL). The CH₂Cl₂ layer was dried (Na₂SO₄), filtered, and concentrated. The crude material was purified by automated flash chromatography using a gradient of 10% EtOAc/hexane to 100% EtOAc to provide the title compound as a colorless oil which solidified on standing (0.164 g, 66%). HPLC (Method F) purity 98.9%, rt=11 min.; HRMS: calcd for C₂₀H₁₉F₃N₄, 372.16; found ([M+H]⁺), 373.1671.

### Example 524

### Preparation of N-{(3S)-1-[4-(2-Naphthyl)pyrimidin-2-yl]pyrrolidin-3-yl}-N-(2,2,2-trifluoroethyl)acetamide

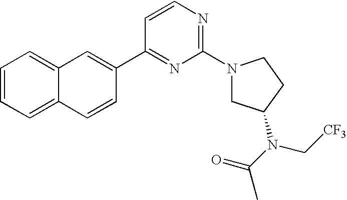

N-{(3S)-1-[4-(2-Naphthyl)pyrimidin-2-yl]pyrrolidin-3-yl}-N-(2,2,2-trifluoroethyl)acetamide To a solution of (3S)-1-[4-(2-naphthyl) pyrimidin-2-yl]-N-(2,2,2-trifluoroethyl)pyrrolidin-3-amine (45.1 mg, 0.121 mmol) in acetic anhydride (3 mL) was added a catalytic amount of DMAP. The reaction was heated in a 60 C bath for 1 h, then cooled to rt. The reaction was concentrated and the residue was dissolved in EtOAc (75 mL) and washed with sat. Na₂CO₃ solution (2×40 mL), water (40 mL), and brine (40 mL).). The EtOAc layer was dried (Na₂SO₄), filtered, and concentrated. The crude material was purified by automated flash chromatography using a gradient of 50% EtOAc/hexane to 100% EtOAc to provide the title compound as a glassy solid (44 mg, 88%). HPLC (Method F) purity 98.2%, rt=10.7 min.; HRMS: calcd for C₂₂H₂₁F₃N₄O, 414.431; found ([M+H]⁺), 415.1732.

### Example 525

### Preparation of 2-{(2R)-1-[4-(2-Naphthyl)pyrimidin-2-yl]pyrrolidin-2-yl}ethanamine Preparation of Amines

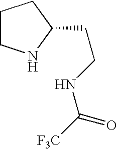

R-2,2,2-Trifluoro-N-(2-(pyrrolidin-2-yl)ethyl)acetamide To a solution of R-tert-butyl 2-(2-aminoethyl)pyrrolidine-1-carboxylate (0.770 g, 3.59 mmol) in MeOH (anhydrous, 10 mL) was added ethyl trifluoroacetate (0.53 mL, 4.49 mmol, 1.25 equiv.) followed by triethyl amine (0.75 mL, 5.38 mmol, 1.5 equiv.). The reaction was stirred at rt for 14 h. The reaction was concentrated and the resulting oil was dissolved in EtOAc (150 mL) and washed with 10% citric acid solution (50 mL), water (2×50 mL), and brine (50 mL).). The EtOAc layer was dried (Na₂SO₄), filtered, and concentrated. To a solution of the golden oil in CH₂Cl₂ (20 mL) was added trifluoroacetic acid (5 mL). The reaction was stirred at rt for 4 h then concentrated. The residue was dissolved in 10% MeOH/CH₂Cl₂ (100 mL) and MP-carbonate resin was added. The mixture was placed on a shaker block for 1 h then filtered to collect the resin. The filtrate was concentrated and used directly for reactions.

(S)-2,2,2-Trifluoro-N-(2-(pyrrolidin-2-yl)ethyl)acetamide was prepared according to the procedure above starting from (S)-tert-butyl 2-(2-aminoethyl)pyrrolidine-1-carboxylate.

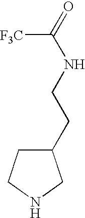

2,2,2-Trifluoro-N-(2-(pyrrolidin-3-yl)ethyl)acetamide To a solution of benzyl 3-(2-aminoethyl)pyrrolidine-1-carboxylate (0.46 g, 1.85 mmol) in MeOH (anhydrous, 20 mL) was added ethyl trifluoroacetate (0.26 mL, 2.2 mmol, 1.3 equiv.) followed by triethyl amine (0.30 mL, 2.4 mmol, 1.3 equiv.). The reaction was stirred at rt for 14 h. The reaction was concentrated and the resulting oil was dissolved in EtOAc (150 mL) and washed with 10% citric acid solution (50 mL), water (2×50 mL), and brine (50 mL). ). The EtOAc layer was dried (Na₂SO₄), filtered, and concentrated. The resulting oil was dissolved in EtOH (20 mL) and stirred over 10% Pd/C (catalytic amount) under a hydrogen balloon for 14 h. The reaction was filtered to remove the palladium. The filtrate was concentrated to afford the title compound (0.32 g, 82%) as a colorless oil which was used directly in reactions.

Trifluoroacetamide deprotections:

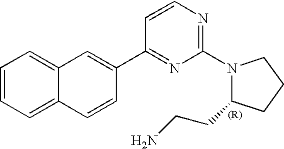

2-{(2R)-1-[4-(2-Naphthyl)pyrimidin-2-yl]pyrrolidin-2-yl}ethanamine To a solution of 2,2,2-trifluoro-N-(2-{(2R)-1-[4-(2-naphthyl)pyrimidin-2-yl]pyrrolidin-2-yl}ethyl)acetamide (100 mg, 0.24 mmol) in MeOH (anhydrous, 3 mL) was added K₂CO₃ (130 mg, 0.96 mmol, 4 equiv.). The reaction was heated in a 50 C shaker block for 14 h. The cooled reaction was filtered through a plug of cotton and rinsed with MeOH (15 mL). The filtrate was concentrated to dryness and water (2 mL) was added. The suspension was heated in a 50 C shaker block for 1 h and the solution was decanted. The residual oil was dried in vacuuo to afford the title compound (74.8 mg, 98%) as a beige powder. HPLC (Method F) purity 97.9%, rt=9.9 min.; HRMS: calcd for C₂₀H₂₂N₄, 318.424; found ([M+H]⁺), 319.1923.

### Example 526

### Preparation of 2-{(2S)-1-[4-(2-Naphthyl)pyrimidin-2-yl]pyrrolidin-2-yl}ethanamine

2-{(2S)-1-[4-(2-Naphthyl)pyrimidin-2-yl]pyrrolidin-2-yl}ethanamine was prepared in the same manner as above starting from 2,2,2-trifluoro-N-(2-{(2S)-1-[4-(2-naphthyl)pyrimidin-2-yl]pyrrolidin-2-yl}ethyl)acetamide. HPLC (Method F) purity 96.8%, rt=9.9 min.; HRMS: calcd for C₂₀H₂₂N₄, 318.424; found ([M+H]⁺), 319.1925.

### Example 527

### Preparation of 2-{1-[4-(2-Naphthyl)pyrimidin-2-yl]pyrrolidin-3-yl}ethanamine

2-{1-[4-(2-Naphthyl)pyrimidin-2-yl]pyrrolidin-3-yl}ethanamine was prepared in the same manner as above starting from 2,2,2-trifluoro-N-(2-{(2S)-1-[4-(2-naphthyl)pyrimidin-2-yl]pyrrolidin-2-yl}ethyl)acetamide. HPLC (Method F) purity 96.9%, rt=8.5 min.; HRMS: calcd for C₂₀H₂₂N₄, 318.428; found ([M+H]⁺), 319.1908.

### Example 528

### Preparation of N-Methyl-N-{(3R)-1-[4-(2-naphthyl)pyrimidin-2-yl]pyrrolidin-3-yl}acetamide

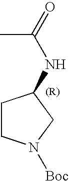

3R-tert-Butyl-3-Acetamidopyrrolidine-1-carboxylate To a solution of R-tert-butyl-3-aminopyrrolidine-1-carboxylate (0.526 g, 2.84 mmol) and diisopropyl ethylamine (0.52 mL, 3.8 mmol, 1.35 equiv.) in THF (anhydrous, 30 mL) was added dropwise acetyl chloride (0.249 mL, 3.53 mmol, 1.25 equiv.). The reaction was stirred at rt for 14 h. The mixture was concentrated and the residue taken up in EtOAc (150 mL). The organic layer was washed with 10% KHSO₄ solution (75 mL), sat. NaHCO₃ (75 mL), and brine (75 mL). The EtOAc layer was dried (Na₂SO₄), filtered, and concentrated to afford the title compound (0.633 g, 98%) as a golden oil which was used without further purification.

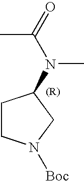

3R-tert-Butyl-3-(N-Methylacetamido)pyrrolidine-l-carboxylate To a suspension of NaH (60% dispersion, 0.166 g, 4.16 mmol, 1.5 equiv.) in THF (10 mL) cooled in an ice bath was added dropwise a solution of ®-tert-butyl-3-acetamidopyrrolidine-1-carboxylate (0.633 g, 2.77 mmol, 1 equiv.) in THF (7 mL), followed by DMSO (1 mL). The reaction was stirred for 10 min., and methyl iodide (0.52 mL, 8.32 mmol, 3 equiv.) was added. The reaction was allowed to gradually come to rt over 14 h. To the mixture was added EtOAc (200 mL) and water (50 mL), and the layers were separated. The organic layer was washed with brine (50 mL), dried (Na₂SO₄), filtered, and concentrated. The crude material was purified by automated silica gel chromatography using a gradient of 100% CH₂Cl₂ to 10% MeOH/CH₂Cl₂ to afford the product (0.145 g, 21.5%) as a colorless oil along with recovered impure material.

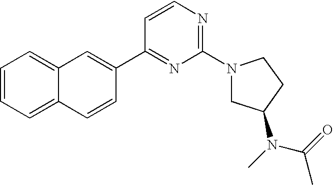

N-Methyl-N-{(3R)-1-[4-(2-naphthyl)pyrimidin-2-yl]pyrrolidin-3-yl}acetamide To a solution of 3R-tert-butyl-3-(N-methylacetamido)pyrrolidine-1-carboxylate (0.073 g, 0.30 mmol, 2 equiv.) in CH₂Cl₂ (10 mL)was added trifluoroacetic acid (1 mL) and the reaction was stirred at rt for 5 h. The solution was concentrated and the residue was dissolved in 10% MeOH/CH₂Cl₂ (40 mL). The solution was placed on a shaker block with MP-carbonate resin for 1 h, then filtered to collect the resin. The filtrate was concentrated and dissolved in NMP (1 mL). The NMP solution was added to a vial containing 2-chloro-4-(naphthalene-2-yl)pyrimidine (36.2 mg, 0.150 mmol, 1 equiv.) and diisoprpropyl ethyl amine (0.10 mL, 0.6 mmol, 4 equiv.) was added. The reaction was heated at 60 C for 14 h. The reaction was cooled to rt and water (0.1 mL) was added. The crude solution was purified by RP HPLC (Method D without TFA) to afford the title compound (42 mg, 80%) as a beige powder. HPLC (Method F) purity 100%, rt=9.9 min.; HRMS: calcd for C₂₁H₂₂N₄O, 346.434; found ([M+H]⁺), 347.1867.

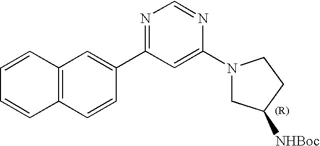

3R-tert-butyl-1-(6-(Naphthalen-2-yl)pyrimidin-4-yl)pyrrolidin-3-yl carbamate A solution of (R)-tert-butyl pyrrolidin-3-yl carbamate (52.3 mg, 0.28 mmol, 1.5 equiv.), 4-chloro-6-(2-naphthyl)pyrimidine (45 mg, 0.18 mmol, 1 equiv.) and diisopropyl ethyl amine (48.8 μL, 0.28 mmol, 1.5 equiv.) in DMSO (1 mL) was heated in an 80 C shaker block for 48 h. The reaction was cooled and EtOAc (100 mL) was added. The solution was washed with water (3×40 mL) and brine (40 mL), dried (Na₂SO₄), filtered, and concentrated. The crude material was purified by automated silica gel chromatography using a gradient of 100% hexane to 100% EtOAc to afford the product (62 m, 85%) as an oil. HPLC (Method H), rt=3.8 min; ([M+H]⁺), 391.

The following examples were prepared in a similar manner:

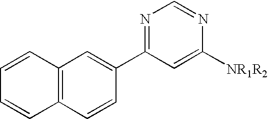

| | | Rt | HPLC |
|---|---|---|---|
| Compound | MS | (min.) | Method |
| 2,2,2-trifluoro-N-(2-{1-[6-(2-naphthyl)pyrimidin-4-yl] | 415 | 9.5 | F |
| pyrrolidin-3-yl}ethyl)acetamide | | | |
| 2,2,2-trifluoro-N-(2-{(2R)-1-[6-(2-naphthyl)pyrimidin-4-yl] | 415 | 10 | F |
| pyrrolidin-2-yl}ethyl) acetamide | | | |
| 2,2,2-trifluoro-N-(2-{(2S)-1-[6-(2-naphthyl)pyrimidin-4-yl] | 415 | 10 | F |
| pyrrolidin-2-yl}ethyl) acetamide | | | |

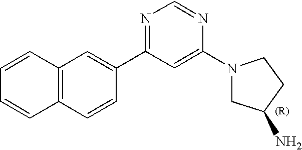

(3R)-1-[6-(2-naphthyl)pyrimidin-4-yl]pyrrolidin-3-amine To a solution of (R)-tert-butyl-1-(6-(naphthalen-2-yl)pyrimidin-4-yl)pyrrolidin-3-yl carbamate (62 mg, 0.158 mmol) in DCM (15 mL) was added trifluoroacetic acid (2 mL). The reaction was placed on a shaker block at rt for 14 h then concentrated. The residue was dissolved in EtOAc (50 mL) and washed with saturated K₂CO₃ solution (3×20 mL). The EtOAc layer was dried (Na₂SO₄), filtered, and concentrated. Lyophilization of the residue provided the title compound as its trifluoroacetic acid salt (50 mg, 99%) as indicated by 19F NMR. HPLC (Method F) purity 96.1%, rt=5.8 min.; HRMS: calcd for C₁₈Hl₈N₄, 290.37; found ([M+H]⁺), 291.1609.

The following examples were prepared by the above methods:

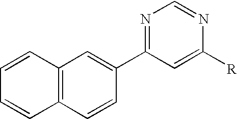

| | | | HPLC |
|---|---|---|---|
| Compound | MS | Rt | Method |
| 2-{(2R)-1-[6-(2-naphthyl)pyrimidin- | 319 | 7.6 | F |
| 4-yl]pyrrolidin-2-yl}ethanamine | | | |
| 2-{1-[6-(2-naphthyl)pyrimidin-4- | 319 | 5.8 | F |
| yl]pyrrolidin-3-yl}ethanamine | | | |
| 2-{(2S)-1-[6-(2-naphthyl)pyrimidin- | 320 | 7.6 | F |
| 4-yl]pyrrolidin-2-yl}ethanamine | | | |
| (3S)-1-[6-(2-naphthyl)pyrimidin- | 291 | 5.8 | F |
| 4-yl]pyrrolidin-3-amine | | | |
| 1-{(3S)-1-[6-(2-naphthyl)pyrimidin- | 305 | 8.5 | G |
| 4-yl]pyrrolidin-3-yl}methanamine | | | |
| 1-{(3R)-1-[6-(2-naphthyl)pyrimidin- | 305 | 8.5 | G |
| 4-yl]pyrrolidin-3-yl}methanamine | | | |

### Example 529

### Preparation of (R)—N1-ethyl-N1,N2-dimethyl-N2-(1-(4-(naphthalen-2-yl)pyrimidin-2-yl)pyrrolidin-3-yl)ethane-1,2-diamine

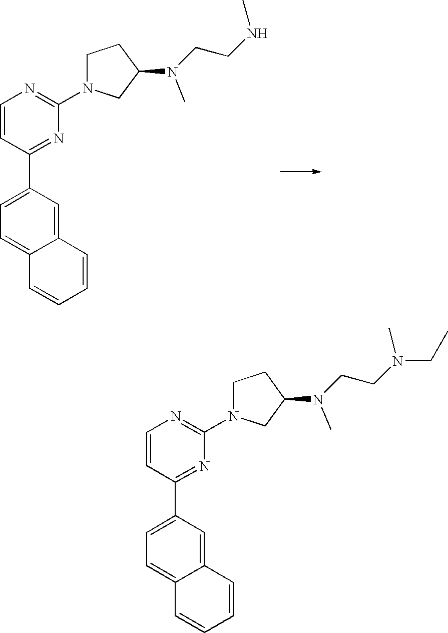

(R)—N1-ethyl-N1,N2-dimethyl-N2-(1-(4-(naphthalen-2-yl)pyrimidin-2-yl)pyrrolidin-3-yl)ethane-1,2-diamine; A solution of (R)—N1,N2-dimethyl-N1-(1-(4-(naphthalen-2-yl)pyrimidin-2-yl)pyrrolidin-3-yl)ethane-1,2-diamine (15 mg, 41 μmol) and sodium triacetoxyborohydride (26 mg, 123 μmol) in dichloromethane (0.4 mL) was treated with acetaldehyde (5.4 mg, 123 μmol, 7 μL) and stirred 18 h. The reaction mixture was diluted with MeOH (1 mL) and water (0.5 mL) and purified by reversed phase HPLC (method D) to leave 20 mg (67%) of the product as a trisTFA salt. HPLC (method C) rt=9.0 mins., purity=98.7%. HR ESMS [M+H]⁺ calc'd=390.2652, obs'd=390.2649.

The same method was used to prepare the compounds in the following table using the procedure for the example above. Starting material, reagents and MS results are indicated.

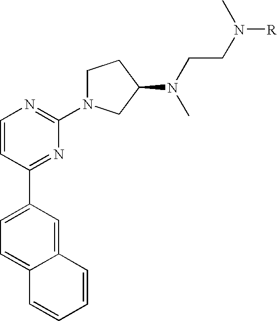

| Example | | | | HPLC |
|---|---|---|---|---|
| No. | Compound | MS | Rt | method |
| 530 | N,N′-dimethyl-N-{(3R)-1-[4-(2-naphthyl)pyrimidin-2-yl]pyrrolidin- | 404 | 9.4 | E |
| | 3-yl}-N′-propylethane-1,2-diamine | | | |
| 531 | N-isopropyl-N,N′-dimethyl-N′-{(3R)-1-[4-(2-naphthyl)pyrimidin-2- | 404 | 9.2 | E |
| | yl]pyrrolidin-3-yl}ethane-1,2-diamine | | | |
| 532 | N-benzyl-N,N′-dimethyl-N′-{(3R)-1-[4-(2-naphthyl)pyrimidin-2- | 452 | 10.0 | E |
| | yl]pyrrolidin-3-yl}ethane-1,2-diamine | | | |

### Example 533

### Preparation of (R)—N-methyl-N-(2-(methyl(1-(4-(naphthalen-2-yl)pyrimidin-2-yl)pyrrolidin-3-yl)amino)ethyl)acetamide

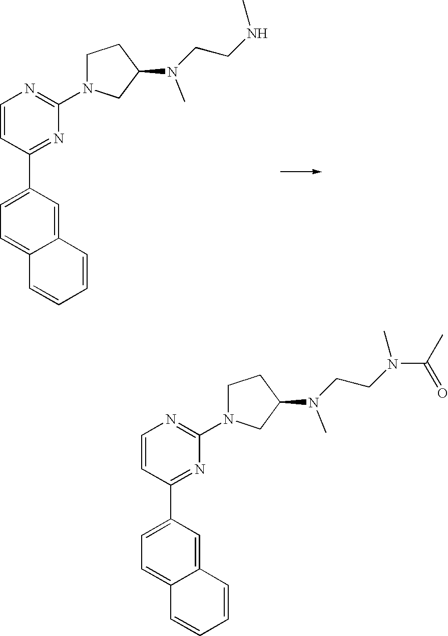

(R)—N-methyl-N-(2-(methyl(1-(4-(naphthalen-2-yl)pyrimidin-2-yl)pyrrolidin-3-yl)amino)ethyl)acetamide; A solution of (R)—N1,N2-dimethyl-N1-(1-(4-(naphthalen-2-yl)pyrimidin-2-yl)pyrrolidin-3-yl)ethane-1,2-diamine (10 mg, 28 μmol) and DIPEA (7.2 mg, 56 μmol, 10 μL) in dichlorormethane (0.3 mL) was treated with acetic anhydride (3.4 mg, 34 μmol, 3.1 μL) and shaken for 18 h. The reaction mixture was diluted with MeOH (1 mL) and water (0.4 mL) and purified by reversed phase HPLC (method D) to leave the product as a bisTFA salt (15 mg, 83%). HPLC (method C) rt=8.1 mins., purity=96.8%. HR ESMS [M+H]⁺ calc'd=404.2445, obs'd=404.2448.

The same method was used to prepare the compounds in the following table using the procedure for the example above.

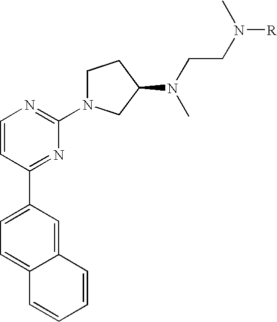

| Example | | | | HPLC |
|---|---|---|---|---|
| No. | Compound | MS | Rt | method |
| 534 | 2,2,2-trifluoro-N-methyl-N-[2-(methyl{(3R)-1-[4-(2- | 458 | 10.3 | E |
| | naphthyl)pyrimidin-2-yl]pyrrolidin-3-yl}amino)ethyl]acetamide | | | |
| 535 | N-methyl-N-[2-(methyl{(3R)-1-[4-(2-naphthyl)pyrimidin-2- | 418 | 8.6 | E |
| | yl]pyrrolidin-3-yl}amino)ethyl]propanamide | | | |
| 536 | N,2-dimethyl-N-[2-(methyl{(3R)-1-[4-(2-naphthyl)pyrimidin-2- | 432 | 9.0 | E |
| | yl]pyrrolidin-3-yl}amino)ethyl]propanamide | | | |
| 537 | N-methyl-N-[2-(methyl{(3R)-1-[4-(2-naphthyl)pyrimidin-2- | 440 | 8.5 | E |
| | yl]pyrrolidin-3-yl}amino)ethyl]methanesulfonamide | | | |
| 538 | 1,1-diethyl-3-methyl-3-[2-(methyl{(3R)-1-[4-(2-naphthyl)pyrimidin- | 461 | 9.3 | E |
| | 2-yl]pyrrolidin-3-yl}amino)ethyl]urea | | | |
| 539 | methyl methyl[2-(methyl{(3R)-1-[4-(2-naphthyl)pyrimidin-2- | 420 | 8.6 | E |
| | yl]pyrrolidin-3-yl}amino)ethyl]carbamate | | | |
| 540 | N-methyl-N-[2-(methyl{(3R)-1-[4-(2-naphthyl)pyrimidin-2- | 454 | 8.9 | E |
| | yl]pyrrolidin-3-yl}amino)ethyl]ethanesulfonamide | | | |
| 541 | N,N,N′-trimethyl-N′-[2-(methyl{(3R)-1-[4-(2-naphthyl)pyrimidin-2- | 469 | 9.1 | E |
| | yl]pyrrolidin-3-yl}amino)ethyl]sulfamide | | | |
| 542 | N-methyl-N-[2-(methyl{(3R)-1-[4-(2-naphthyl)pyrimidin-2- | 466 | 9.5 | E |
| | yl]pyrrolidin-3-yl}amino)ethyl]benzamide | | | |
| 543 | N-methyl-N-[2-(methyl{(3R)-1-[4-(2-naphthyl)pyrimidin-2- | 502 | 10.0 | E |
| | yl]pyrrolidin-3-yl}amino)ethyl]benzenesulfonamide | | | |

### Example 544

### Preparation of tert-Butyl (1-(4-(naphthalen-2-yl)pyrimidin-2-yl)azetidin-3-yl)methylcarbamate

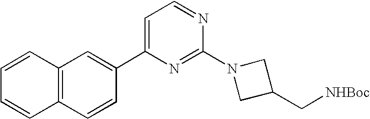

tert-Butyl (1-(4-(naphthalen-2-yl)pyrimidin-2-yl)azetidin-3-yl)methylcarbamate; A solution of tert-butyl azetidin-3-ylmethylcarbamate (300 mg, 1.6 mmol), 2-chloro-4-(naphthalen-2-yl)pyrimidine (310 mg, 1.3 mmol) and DIPEA (416 mg, 3.2 mmol, 0.58 mL) in DMSO (10 mL) was heated to 80° C. for 18 h. The reaction mixture was cooled to room temperature, diluted with ethyl acetate (100 mL), washed with 1 M sodium carbonate (50 mL), water (2×50 mL) and brine (50 mL), dried (MgSO₄) and evaporated. The crude product was purified by silica gel chromatography eluted with a gradient of 50-100% ethyl acetate in hexanes to leave 390 mg (78%) of pure product. HPLC (method C) rt=10.8 mins., purity=99.9%. HR ESMS [M+H]⁺ obs'd=391.213.

### Example 545

### Preparation of (1-(4-(naphthalen-2-yl)pyrimidin-2-yl)azetidin-3-yl)methanamine

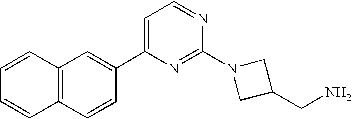

(1-(4-(naphthalen-2-yl)pyrimidin-2-yl)azetidin-3-yl)methanamine; A solution of tert-butyl (1-(4-(naphthalen-2-yl)pyrimidin-2-yl)azetidin-3-yl)methylcarbamate (350 mg, 0.90 mmol) in 1:1 TFA-DCM (5 mL) was stirred for 1.5 h then evaporated and dried under hi-vac for 24 h. The product was left as a bistrifluoroacetate salt (0.46 g, 100%). HPLC (method C) rt=4.8 mins., purity=97.0%. HR ESMS [M+H]⁺ obs'd=291.1611.

### Example 546

### Preparation of methyl ({1-[4-(2-naphthyl)pyrimidin-2-yl]azetidin-3-yl}methyl)carbamate

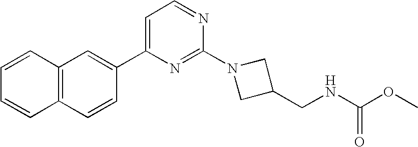

A solution of (1-(4-(naphthalen-2-yl)pyrimidin-2-yl)azetidin-3-yl)methanamine (5.8 mg, 20 μmol) and DIPEA (26 mg, 0.20 mmol, 36 μL) in DCM (0.2 mL) was treated with a 0.20 M solution of methyl chloroformate in DMF (0.20 mL, 40 μmol) and shaken 18 h. The reaction mixture was diluted with MeOH (1.0 mL) and water (0.5 mL) and purified by reversed phase HPLC (method D) to leave the product as a trifluoroacetate salt (4.6 mg, 66%). LC/MS (Method A) rt=1.50 mins., calculated mass=348, [M+H]⁺=349.

The same method was used to prepare the compounds in the following table using the procedure for the example above.

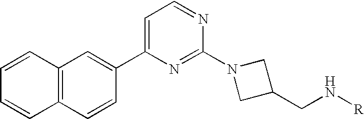

| Example | | | Rt | HPLC |
|---|---|---|---|---|
| No. | Compound | MS | (min.) | Method |
| 547 | 2,2,2-trifluoro-N-({1-[4-(2-naphthyl)pyrimidin-2-yl]azetidin-3- | 387 | 2.5 | E |
| | yl}methyl) acetamide | | | |
| 548 | N,N-dimethyl-N′-({1-[4-(2-naphthyl)pyrimidin-2-yl]azetidin-3- | 362 | 2.2 | E |
| | yl}methyl)urea | | | |
| 549 | N-({1-[4-(2-naphthyl)pyrimidin-2-yl]azetidin-3-yl}methyl)urea | 334 | 2.0 | E |
| 550 | N-ethyl-N′-({1-[4-(2-naphthyl)pyrimidin-2-yl]azetidin-3- | 362 | 2.2 | E |
| | yl}methyl)urea | | | |
| 551 | N-({1-[4-(2-naphthyl)pyrimidin-2-yl]azetidin-3- | 369 | 2.3 | E |
| | yl}methyl)methanesulfonamide | | | |
| 552 | 4-methyl-N-({1-[4-(2-naphthyl)pyrimidin-2-yl]azetidin-3- | 445 | 2.7 | E |
| | yl}methyl)benzenesulfonamide | | | |

### Example 553

### Preparation of): tert-Butyl {2-[1-(4-naphthalen-2-ylpyrimidin-2-yl)azetidin-3-yl]ethyl}carbamate

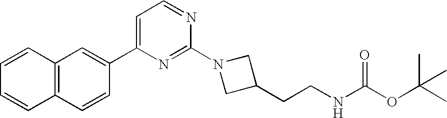

tert-Butyl {2-[1-(4-naphthalen-2-ylpyrimidin-2-yl)azetidin-3-yl]ethyl }carbamate was prepared using the procedure above for the preparation of tert-butyl (1-(4-(naphthalen-2-yl)pyrimidin-2-yl)azetidin-3-yl)methylcarbamate. Left 25 mg (81%). HPLC (method F) rt=11.5 mins., purity=98.9%. HR ESMS [M+H]⁺ calc'd=405.2285, obs'd=405.2288.

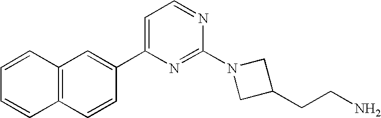

### Example 554

### Preparation of 2-[1-(4-naphthalen-2-ylpyrimidin-2-yl)azetidin-3-yl]ethanamine

2-[1-(4-naphthalen-2-ylpyrimidin-2-yl)azetidin-3-yl]ethanamine was prepared using the procedure above for the preparation of (1-(4-(naphthalen-2-yl)pyrimidin-2-yl)azetidin-3-yl)methanamine. Left 45 mg (100%). HPLC (method F) rt=9.3 mins., purity=99.0%. HR ESMS [M+H]⁺ calc'd=305.1761, obs'd=305.1763.

### Example 555

### Preparation of tert-Butyl [(trans-4-{[4-(2-naphthyl)pyrimidin-2-yl]oxy}cyclohexyl)methyl]carbamate

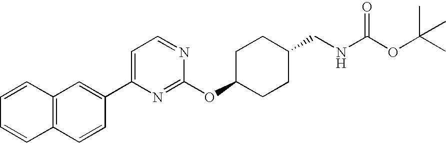

tert-Butyl [(trans-4-{[4-(2-naphthyl)pyrimidin-2-yl]oxy}cyclohexyl)methyl]carbamate; A solution of trans-4-(bocaminomethyl)cyclohexanol (100 mg, 0.437 mmol) in DMF (2 mL) was treated with sodium hydride (60% in mineral oil, 21 mg, 0.52 mmol) and shaken for 30 minutes. 2-Chloro-4-(naphthalen-2-yl)pyrimidine (126 mg, 0.52 mmol) was added the mixture was stirred for 18 h. More sodium hydride (21 mg, 0.52 mmol) was added and the mixture stirred an additional 24 h. Water (0.5 mL) was added and the mixture stirred 30 minutes. Ethyl acetate (50 mL) was added and the mixture was washed with water (2×50 mL) and brine (50 mL), dried (MgSO₄) and evaporated. The crude product was purified by reversed phase HPLC (method D). The product fractions were combined, neutralized with 1 M sodium carbonate solution and extracted with ethyl acetate (2×50 mL). The combined organic layers were washed with brine (50 mL), dried (MgSO₄) and evaporated to leave 151 mg (68%). HPLC (method F) rt=11.7 mins., purity=89.0%. HR ESMS [M+H]⁺ calc'd=434.2438, obs'd=434.2437.

### Example 556

### Preparation of 1-(trans-4-{[4-(2-naphthyl)pyrimidin-2-yl]oxy}cyclohexyl)methanamine

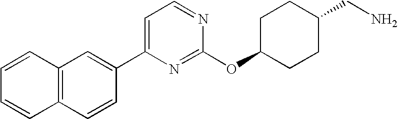

1-(trans-4-{[4-(2-naphthyl)pyrimidin-2-yl]oxy}cyclohexyl)methanamine; A solution of tert-butyl [(trans-4-{[4-(2-naphthyl)pyrimidin-2-yl]oxy}cyclohexyl)methyl]carbamate (87 mg, 0.20 mmol) in TFA (1 mL) and DCM (1 mL) was stirred for 2 h then evaporated to dryness and held under hi-vacuum for 18 h. HPLC (method F) rt=8.5 mins., purity=83.5%. HR ESMS [M+H]⁺ calc'd=334.1914, obs'd=334.1913.

### Example 557

### Preparation of tert-Butyl ({cis-4-[(4-naphthalen-2-ylpyrimidin-2-yl)oxy]cyclohexyl}methyl)carbamate

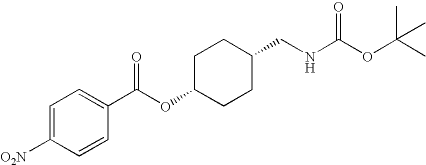

(1s,4s)-4-((tert-butoxycarbonylamino)methyl)cyclohexyl 4-nitrobenzoate; A mixture of trans-4-(bocaminomethyl)cyclohexanol (1.0 g, 4.4 mmol), 4-nitrobenzoic acid (1.4 g, 8.8 mmol) and polystyrene resin bound triphenylphosphine (3.0 mmol/g, 5.0 g, 15 mmol) in DCM (50 mL) was stirred under nitrogen atmosphere, cooled in an ice bath and treated di-tert-butylazodicarboxylate (2.4 g, 11 mmol). The mixture slowly warmed to room temperature and stirred for 18 h. The resin was filtered and washed (DCM) and the combined filtrates were evaporated. The crude product was purified by silica gel chromatography eluted with a gradient of 25-60% ethyl acetate in hexanes. Addiotional silica gel chromatography eluted with a gradient of 3-15% ethyl acetate in DCM left pure product (0.27 g, 17%). LC/MS (Method A) rt=3.35 mins.

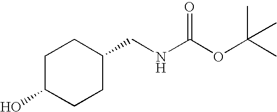

tert-Butyl ((1s,4s)-4-hydroxycyclohexyl)methylcarbamate; A solution of (1s,4s)-4-((tert-butoxycarbonylamino)methyl)cyclohexyl 4-nitrobenzoate (0.26 g, 0.71 mmol) and lithium hydroxide monohydrate (0.15 g, 3.6 mmol) in THF (10 mL) and water (10 mL) was stirred for 18 h then diluted with ethyl acetate (50 mL), washed with water (50 mL) and brine (50 mL) then dried (MgSO₄) and evaporated to leave a colorless gum (160 mg, 100%).

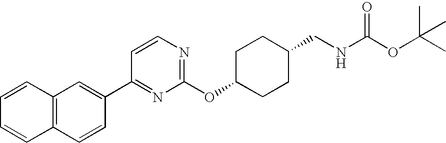

tert-Butyl ({cis-4-[(4-naphthalen-2-ylpyrimidin-2-yl)oxy]cyclohexyl}methyl)carbamate; Sample was prepared on 35 mg scale under identical conditions as the example above. HPLC (method F) rt=12.0 mins., purity=99.9%. HR ESMS [M+H]⁺ calc'd=434.2438, obs'd=434.2443.

### Example 558

### Preparation of 1-{cis-4-[(4-naphthalen-2-ylpyrimidin-2-yl)oxy]cyclohexyl}methanamine

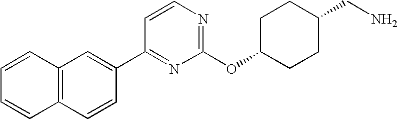

1-{cis-4-[(4-naphthalen-2-ylpyrimidin-2-yl)oxy]cyclohexyl }methanamine; Prepared on 29 mg scale under conditions identical to the example above. HPLC (method F) rt=9.2 mins., purity=95.8%. HR ESMS [M+H]⁺ calc'd=334.1914, obs'd=334.1920.

### Example 559

### Preparation of N-((1r,4r)-4-(Aminomethyl)cyclohexyl)-N-methyl-4-(naphthalen-2-yl)pyrimidin-2-amine

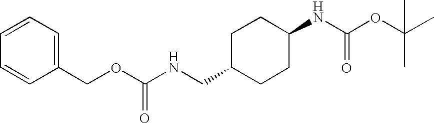

A solution of tert-butyl (1r,4r)-4-(aminomethyl)cyclohexylcarbamate (750 mg, 3.3 mmol) and DIPEA (0.47 g, 3.6 mmol, 0.65 mL) in DCM (30 mL) was cooled in an ice bath and treated with benzyl chloroformate (0.62 g, 3.6 mmol, 0.51 mL). The reaction mixture was allowed to warm slowly to room temperature and stir for 18 h. The solvent was evaporated and the residue was dissolved in ethyl acetate (50 mL), washed with 1 N HCl (30 mL), water (30 mL) and brine (30 mL). The organic layer was dried (MgSO₄) and evaporated to leave 1.1 g (95%).

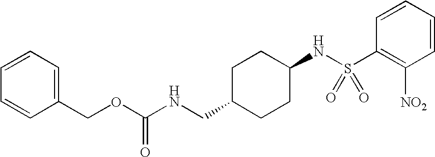

Benzyl ((1r,4r)-4-(2-nitrophenylsulfonamido)cyclohexyl)methylcarbamate; A solution of the Boc protected amine prepared above (1.1 g, 3.0 mmol) was dissolved in 1:1 TFA-DCM (50 mL) and stirred for 1 h. The solvents were evaporated and the residue was dissolved in ethyl acetate (100 mL) and washed with 1 M sodium carbonate solution (50 mL) and brine (50 mL). The organic layer was dried (MgSO₄) and evaporated and the residue was dissolved in DCM (30 mL), treated with DIPEA (0.43 g, 3.3 mmol, 0.6 mL) and cooled in an ice bath. The reaction mixture was treated with 2-nitrobenzenesulfonyl chloride (0.74 g, 3.3 mmol) and stirred for 15 minutes. The ice bath was removed and stirring continued for 1 h. The solvents were evaporated and the residue was dissolved in ethyl acetate (100 mL), washed with 1 N HCl (100 mL), water (100 mL) and brine (100 mL), dried (MgSO₄) and evaporated. The crude product was purified on silica gel eluted with a gradient of 25-70% ethyl acetate in hexanes to leave 0.77g (57%). LC/MS (Method A) rt=2.90 mins., calculated mass=447, [M+H]⁺=448.

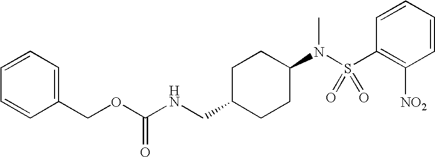

Benzyl ((1r,4r)-4-(N-methyl-2-nitrophenylsulfonamido)cyclohexyl)methylcarbamate; A mixture of benzyl ((1r,4r)-4-(2-nitrophenylsulfonamido)cyclohexyl)methylcarbamate (0.76 g, 1.7 mmol), polystyrene resin supported triphenylphosphine (3.0 mmol/g, 1.4 g, 4.3 mmol) and anhydrous MeOH (0.11 g, 3.4 mmol, 0.14 mL) in DCM (20 mL) under nitrogen atmosphere was cooled in an ice bath and treated with di-tert-butylazodicarboxylate (DBAD, 0.74 g, 3.4 mmol) The reaction mixture stirred 18 h and slowly warmed to room temperature. The mixture was cooled in ice and treated with additional resin bound triphenylphosphine (1.4 g, 4.3 mmol), MeOH (0.11 g, 3.4 mmol, 0.14 mL) and DBAD (0.74 g, 3.4 mmol) and stirred for 1 h. TFA (10 mL) was added and stirring continued for 1 h. The reaction mixture was filtered through diatomaceous earth, the residue was washed with DCM (50 mL) and the filtrate was evaporated. The crude product was dissolved in ethyl acetate (100 mL), washed with saturated sodium bicarbonate solution (100 mL), water (100 mL) and brine (100 mL). The organic layer was dried (MgSO₄) and evaporated. Purification on silica gel eluted with a gradient of 25-70% ethyl acetate in hexanes netted 0.71 g (91%) of pure product. LC/MS (Method A) rt=3.10 mins., calculated mass=461, [M+H]⁺=462.

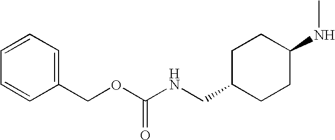

Benzyl ((1r,4r)-4-(methylamino)cyclohexyl)methylcarbamate; A solution of benzyl ((1r,4r)-4-(N-methyl-2-nitrophenylsulfonamido)cyclohexyl)methylcarbamate (0.70 g, 1.5 mmol) and thioacetic acid (0.28 g, 3.0 mmol, 0.21 mL) in DMF (12 mL) was treated with lithium hydroxide monohydrate (0.25 g, 6.0 mmol) and stirred for 2 h. More thioacetic acid (0.28 g, 3.0 mmol, 0.21 mL) and lithium hydroxide monohydrate (0.25 g, 6.0 mmol) and stirring continued for 1 h. The reaction mixture was diluted with saturated sodium bicarbonate solution (50 mL) and ethyl acetate (50 mL). The organic layer was separated and washed with water (2×50 mL). The product was extracted with 1 N HCl (3×50 mL) and the combined acidic layers were neutralized with solid sodium carbonate (pH=11). The product was extracted with ethyl acetate (2×50 mL), the organic extracts combined, dried (MgSO₄) and evaporated to leave a colorless gum (124 mg, 30%). LC/MS (Method A) rt=1.27 mins., calculated mass=276, [M+H]⁺=277.

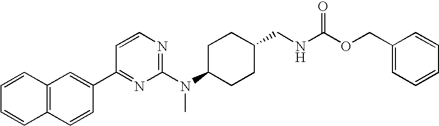

Benzyl ((1r,4r)-4-(methyl(4-(naphthalen-2-yl)pyrimidin-2-yl)amino)cyclohexyl)methylcarbamate; A solution of benzyl ((1r,4r)-4-(methylamino)cyclohexyl)methylcarbamate (26 mg, 94 μmol) and DIPEA (16 mg, 0.12 mmol, 22 μL) in DMSO (1 mL) was treated with 2-chloro-4-(naphthalen-2-yl)pyrimidine (29 mg, 0.12 mmol), stirred and heated to 80° C. for 48 h. The reaction mixture was cooled to room temperature, treated with water (0.5 mL) and MeOH (1 mL) and purified by reversed phase HPLC (method D) to leave the product as a monoTFA salt (22 mg, 39%). HPLC (method F) rt=12.5 mins., purity=99.9%.

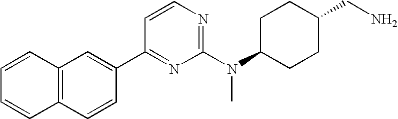

N-((1r,4r)-4-(Aminomethyl)cyclohexyl)-N-methyl-4-(naphthalen-2-yl)pyrimidin-2-amine; A solution of benzyl ((1r,4r)-4-(methyl(4-(naphthalen-2-yl)pyrimidin-2-yl)amino)cyclohexyl)methylcarbamate (16 mg, 27 μmol) in conc. aq. HBr (1 mL) was stirred and heated to 60° C. for 3 h. The solvent was evaporated and the crude product was purified by reversed phase HPLC (method D) to leave 9.2 mg (59%) of the bisTFA salt. HPLC (method F) rt=11.1 mins., purity=99.9%. HR ESMS [M+H]⁺ calc'd=347.2230, obs'd=347.2233.

### Example 560

### Preparation of Benzyl ((1s,4s)-4-(methyl(4-(naphthalen-2-yl)pyrimidin-2-yl)amino)cyclohexyl)methylcarbamate

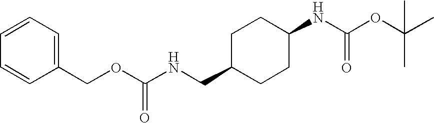

Prepared under identical conditions and scale to the trans isomer above. Left 1.2 g (100%).

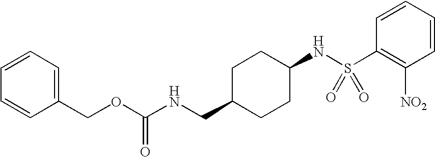

Benzyl ((1s,4s)-4-(2-nitrophenylsulfonamido)cyclohexyl)methylcarbamate: Prepared under identical conditions and scale to the trans isomer above. Left 1.0 g (74%). LC/MS (Method A) rt=2.96 mins., calculated mass=447, [M+H]⁺=448.

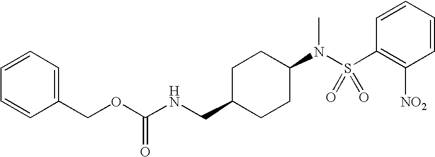

Benzyl ((1s,4s)-4-(N-methyl-2-nitrophenylsulfonamido)cyclohexyl)methylcarbamate; Prepared under identical conditions to the trans isomer above on 0.98 g (2.2 mmol) scale. Left 0.91 g (90%). LC/MS (Method A) rt=3.09 mins., calculated mass=461, [M+H]⁺=462.

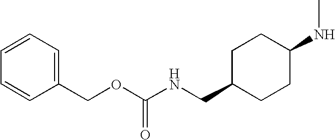

Benzyl ((1s,4s)-4-(methylamino)cyclohexyl)methylcarbamate: Prepared under identical conditions to the trans isomer above on 0.90 g (2.0 mmol) scale. Left 242 mg (44%). LC/MS (Method A) rt=1.27 mins., calculated mass=276, [M+H]⁺=277.

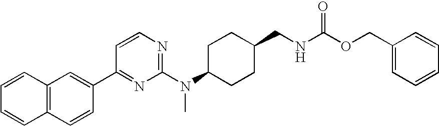

Benzyl ((1s,4s)-4-(methyl(4-(naphthalen-2-yl)pyrimidin-2-yl)amino)cyclohexyl)methylcarbamate; Prepared under identical conditions to the trans isomer above on 50 mg (0.18 mmol) scale. Left 60 mg (69%). HPLC (method F) rt=12.5 mins., purity=99.7%.

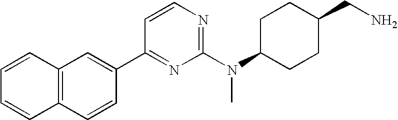

### Example 561

### Preparation of N-[cis-4-(Aminomethyl)cyclohexyl]-N-methyl-4-(2-naphthyl)pyrimidin-2-amin

N-[cis-4-(Aminomethyl)cyclohexyl]-N-methyl-4-(2-naphthyl)pyrimidin-2-amine; Prepared under identical conditions to the trans isomer above on 40 mg (67 μmol) scale. Left 16 mg (39%). HPLC (method F) rt=11.0 mins., purity=99.9%. HR ESMS [M+H]⁺ calc'd=347.2230, obs'd=347.2234.

### Example 562

### Preparation of 1-{4-[4-(2-naphthyl)pyrimidin-2-yl]phenyl}methanamine

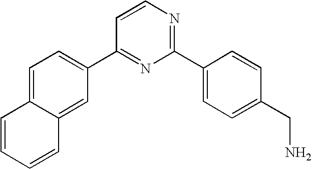

1-{4-[4-(2-naphthyl)pyrimidin-2-yl]phenyl }methanamine; A mixture of benzylamine-4-boronic acid (75 mg, 0.40 mmol), 2-chloro-4-(naphthalen-2-yl)pyrimidine (72 mg, 0.30 mmol), potassium bicarbonate (110 mg, 1.1 mmol), palladium(II) acetate (7 mg, 30 μmol) and triphenylphosphine 22 mg, 90 μmol) was purged with nitrogen and charged with a 3:1 mixture of dimethoxyethane and water (1.5 mL). The mixture was heated to 150° C. for 1 h in a microwave, cooled to room temperature, diluted with ethyl acetate (50 mL) and water (50 mL), filtered through diatomaceous earth, washed with ethyl acetate (25 mL), and the filtrate was separated. The organic layer was washed with brine (50 mL), dried (MgSO₄) and evaporated. The crude reaction mixture was purified by reversed phase HPLC (method D) to leave the product as a bisTFA salt (9.3 mg, 6%). HPLC (method F) rt=8.9 mins., purity=98.4%. HR ESMS [M+H]⁺ calc'd=312.1495, obs'd=312.1497.

### Example 563

### Preparation of 1-[trans-4-(4-naphthalen-2-ylpyrimidin-2-yl)cyclohexyl]methanamine

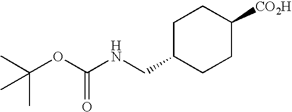

(1r,4r)-4-((tert-Butoxycarbonylamino)methyl)cyclohexanecarboxylic acid; A solution of (1r,4r)-4-(aminomethyl)cyclohexanecarboxylic acid (4.4 g, 28 mmol) and sodium carbonate (3.0 g, 14 mmol) in 1,4-dioxane (100 mL) and water (100 mL) was treated with di-tert-butyldicarbonate (7.2 g, 34 mmol) and stirred for 18 h. The mixture was treated with water (300 mL) and washed with ethyl acetate (2×300 mL). The aqueous layer was neutralized (pH=2) with conc. HCl and the product was extracted with ethyl acetate (2×200 mL). The combined extracts were washed with brine (100 mL), dried (MgSO₄) and evaporated to a white solid (7.0 g, 97%).

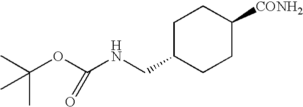

tert-butyl ((1r,4r)-4-carbamoylcyclohexyl)methylcarbamate: A solution of (1r,4r)-4-((tert-butoxycarbonylamino)methyl)cyclohexanecarboxylic acid (7.0 g, 37 mmol) and DIPEA (3.9 g, 30 mmol, 5.4 mL) in THF (100 mL) under nitrogen atmosphere was cooled in an ice bath and treated with a 1.0 M solution of isopropylchloroformate in toluene (30 mL, 30 mmol) and stirred for 30 minutes. Conc. ammonia solution (3.6 mL, 54 mmol) was added and the mixture warmed to room temperature and stirred an additional 18 h. The reaction mixture was cooled in ice and water (100 mL) was added. The precipitate that formed was filtered, washed with water (3×25 mL) and dried under vacuum. Left a white solid (6.4 g, 93%).

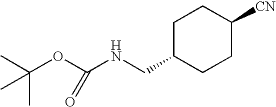

tert-Butyl ((1r,4r)-4-cyanocyclohexyl)methylcarbamate; A solution of DIPEA (12.9 g, 100 mmol, 18 mL) in DMF (200 mL) under nitrogen atmosphere was cooled in an ice bath and treated dropwise with thionyl chloride (11.9 g, 100 mmol, 7.3 mL) over 15 minutes. The deep reddish brown solution stirred an additional 30 minutes then tert-butyl ((1r,4r)-4-carbamoylcyclohexyl)methylcarbamate (6.4 g, 25 mmol) was added to the solution as a solid all at once. After stirring for 1 h the mixture was diluted with ethyl acetate (500 mL), washed with water (3×500 mL), brine (500 mL), dried (MgSO₄) and evaporated. The residue was purified on silica gel eluted with a gradient of 15-60% ethyl acetate in hexanes (fractions stained with ninhydrin following tlc development) to leave the product as a reddish-brown solid (4.4 g, 74%).

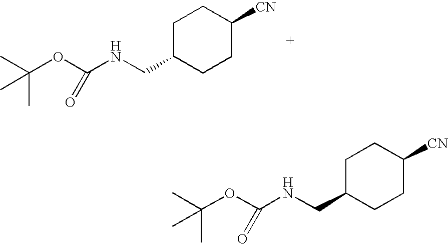

tert-Butyl ((1r,4r)-4-cyanocyclohexyl)methylcarbamate and tert-butyl ((1s,4s)-4-cyanocyclohexyl)methylcarbamate (3:2 mixture). A solution of tert-Butyl ((1r,4r)-4-cyanocyclohexyl)methylcarbamate (2.0 g, 8.4 mmol) in THF (80 mL) under nitrogen atmosphere was cooled in an ice bath and treated with potassium tert-butoxide (4.0 g, 36 mmol) and stirred 3 h. The reaction mixture was treated with 1 N HCl (80 mL), stirred for 5 minutes then diluted with water (300 mL) and ethyl acetate (300 mL). The aqueous layer was separated and extracted with ethyl acetate (300 mL). The extracts were combined, washed with brine (200 mL), dried (MgSO₄) and evaporated to leave 1.9 g (95%). NMR indicates a 3:2 mixture of trans:cis isomers.

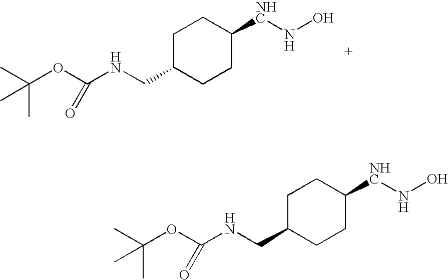

tert-Butyl ((1r,4r)-4-(N-hydroxycarbamimidoyl)cyclohexyl)methylcarbamate and tert-Butyl ((1s,4s)-4-(N-hydroxycarbamimidoyl)cyclohexyl)methylcarbamate: A solution of tert-butyl ((1r,4r)-4-cyanocyclohexyl)methylcarbamate and tert-butyl ((1s,4s)-4-cyanocyclohexyl)methylcarbamate (3:2 mixture, 1.9 g, 8.0 mmol) in ethanol (45 mL) was treated with sodium carbonate (3.4 g, 32 mmol) and hydroxylamine hydrochloride (2.2 g, 32 mmol), stirred and refluxed for 6 h. Additional sodium carbonate (3.4 g, 32 mmol) and hydroxylamine hydrochloride (2.2 g, 32 mmol) were added and the mixture refluxed for 18 h. The reaction mixture was cooled to room temperature and diluted with ethyl acetate (100 mL) and water (100 mL). The aqueous layer was extracted with ethyl acetate (2×100 mL) and the combined organic extracts were washed with brine (100 mL), dried (Na₂SO₄) and evaporated to leave a semi-crystalline solid (2.0 g, 92%).

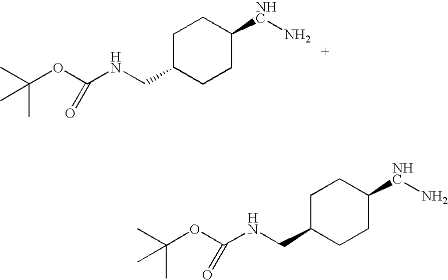

tert-Butyl ((1r,4r)-4-carbamimidoylcyclohexyl)methylcarbamate and tert-Butyl ((1s,4s)-4-carbamimidoylcyclohexyl)methylcarbamate (3:2 mixture); A solution of tert-butyl ((1r,4r)-4-(N-hydroxycarbamimidoyl)cyclohexyl)methylcarbamate and tert-Butyl ((1s,4s)-4-(N-hydroxycarbamimidoyl)cyclohexyl)methylcarbamate (2.0 g, 3.6 mmol) in acetic anhydride (2 mL) and acetic acid (18 mL) was hydrogenated at 45 psi hydrogen pressure over 5% Pd/C for 18 h. The catalyst was filtered and washed (MeOH) and the filtrate was evaporated. The residue was lyophilized to leave a hygroscopic solid.

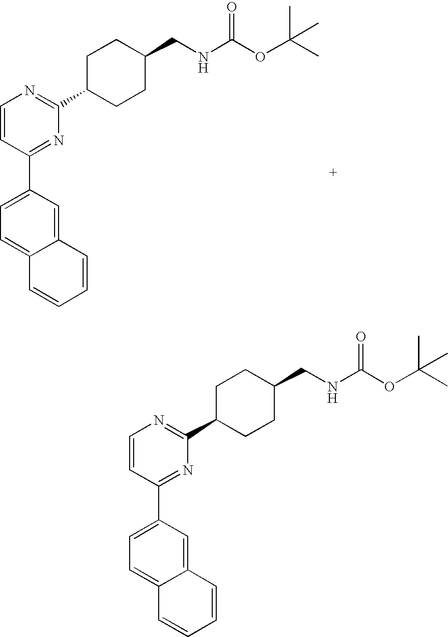

tert-Butyl ((1r,4r)-4-(4-(naphthalen-2-yl)pyrimidin-2-yl)cyclohexyl)methylcarbamate and tert-butyl ((1s,4s)-4-(4-(naphthalen-2-yl)pyrimidin-2-yl)cyclohexyl)methylcarbamate; To a 0.63 M solution of sodium ethoxide (6 mL) was added a 3:2 mixture of tert-butyl ((1r,4r)-4-carbamimidoylcyclohexyl)methylcarbamate acetate and tert-butyl ((1s,4s)-4-carbamimidoylcyclohexyl)methylcarbamate acetate (0.38 g, 1.5 mmol) and the mixture stirred until solution was attained (30 minutes). To this reaction mixture was added (E)-3-(dimethylamino)-1-(naphthalen-2-yl)prop-2-en-1-one (0.23 g, 1.0 mmol) and the mixture stirred at 60° C. for 18 h for 48 h. The reaction mixture was diluted with water (50 mL) and ethyl acetate (50 mL), separated and the organic layer was washed with water (30 mL) and brine (30 mL). The organic layer was dried (MgSO₄) and evaporated. The crude product mixture was purified by silica gel chromatography eluting with a gradient of 25-70% ethyl acetate in hexanes. The first product fractions left the cis isomer (22 mg, 5%) while the second fractions contained the trans isomer (152 mg, 36%). Cis isomer LC/MS (Method A) rt=3.89 mins., calculated mass=417, [M+H]⁺=418. Trans isomer HPLC (method F) rt=11.9 mins., purity=98.1%. HR ESMS [M+H]⁺ calc'd=418.2489, obs'd=418.2487.

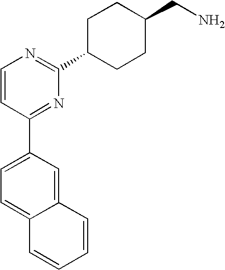

1-[trans-4-(4-naphthalen-2-ylpyrimidin-2-yl)cyclohexyl]methanamine; A solution of tert-Butyl ((1r,4r)-4-(4-(naphthalen-2-yl)pyrimidin-2-yl)cyclohexyl)methylcarbamate (137 mg, 0.33 mmol) was stirred in 1:1 TFA-DCM (2 mL) for 30 minutes. The solvent was evaporated and the residue was purified by reversed phase HPLC (method D) to leave the product as a bistrifluoroacetate salt (179 mg, 100%). HPLC (method F) rt=8.8 mins., purity=98.8%. HR ESMS [M+H]⁺ calc'd=318.1965, obs'd=318.1972.

### Example 564

### Preparation of 1-{cis-4-[4-(2-naphthyl)pyrimidin-2-yl]cyclohexyl}methanamine

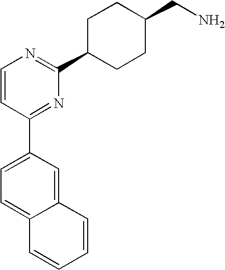

1-{cis-4-[4-(2-naphthyl)pyrimidin-2-yl]cyclohexyl}methanamine; A solution of tert-butyl ((1s, 4s)-4-(4-(napthalen-2-yl)pyrimidin-2-yl)cyclohexyl)methylcarbamate (7.5 mg, 17 μmol) was stirred in 1:1 TFA-DCM (1 mL) for 30 minutes. The solvent was evaporated to leave the product as a bistrifluoroacetate salt (10 mg, 100%). HPLC (method F) rt=8.6 mins., purity>99.9%. ESMS [M+H]⁺ calc'd=318, obs'd=318.

### Example 565

### Preparation of (1-(4-(6-methoxynaphthalen-2-yl)pyrimidin-2-yl)piperidin-4-yl)methanamine

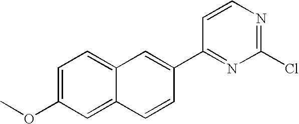

2-chloro-4-(6-methoxynaphthalen-2-yl)pyrimidine 6-Methoxy-2-naphthyl boronic acid (1.68 g, 8.3 mmol), 2,4-dichloropyrimidine (1.24 g, 8.3 mmol), potassium phosphate (5.3 g, 25 mmol) and tetrakis palladium (960 mg, 0.83 mmol) were dissolved in dioxane (30 mL) under nitrogen. This reaction mixture was allowed to stir for 16 h at 100° C. The solvent was removed under vacuum and the crude was partitioned between ethyl acetate and brine solution. The ethyl acetate was washed twice with brine and the combined ethyl acetate fractions were dried over MgSO₄. This crude was purified on silica in a gradient proceeding from 5% to 70% ethyl acetate/hexane to yield the title product (1.30 g (58%), 4.8 mmol). MS (ES) m/z 271.8 [M+H]⁺. Open access: about 95% at 254 nm RT=2.83 min.

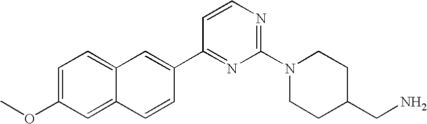

(1-(4-(6-methoxynaphthalen-2-yl)pyrimidin-2-yl)piperidin-4-yl)methanamine 2-Chloro-4-(6-methoxynaphthalen-2-yl)pyrimidine (480 mg, 1.78 mmol) was dissolved in DMSO (100 ml) and the solution was charged with piperidin-4-ylmethanamine (1 ml). This reaction mixture was allowed to stir for 16 h at 1 00° C. The solvent was partitioned between ethyl acetate and brine solution, and a precipitate was observed and filtered, yielding the title product. The ethyl acetate was washed twice with brine and the combined ethyl acetate fractions were dried over MgSO₄, yielding more title product (310 mg (50%), 0.89 mmol). MS (ES) m/z 349.12 [M−H]⁻. Open access(HF): about 95% at 254 nm RT=3.34 min.

The following compounds were prepared in a similar manner as the example above:

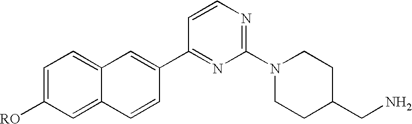

| Examples | | | | HPLC |
|---|---|---|---|---|
| No. | Compound | MS | Rt | method |
| 566 | (1-(4-(6-propoxynaphthalen-2-yl)pyrimidin-2- | 377 | 3.94 | HF |
| | yl)piperidin-4-yl)methanamine | | | |
| 567 | (1-(4-(6-isobutoxynaphthalen-2-yl)pyrimidin-2- | 391 | 4.17 | HF |
| | yl)piperidin-4-yl)methanamine | | | |
| 568 | 6-(2-(4-(aminomethyl)piperidin-1-yl)pyrimidin-4- | 377 | 3.56 | HF |
| | yl)naphthalen-2-yl acetate | | | |

### Example 569

### Preparation of 6-(2-(4-(aminomethyl)piperidin-1-yl)pyrimidin-4-yl)naphthalen-2-ol

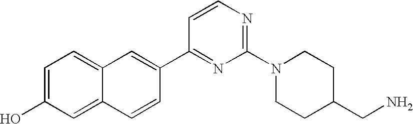

6-(2-(4-(aminomethyl)piperidin-1-yl)pyrimidin-4-yl)naphthalen-2-ol 2,2,2-trifluoro-N-((1-(4-(6-hydroxynaphthalen-2-yl)pyrimidin-2-yl)piperidin-4-yl)methyl)acetamide (30 mg, 0.069 mmol) taken up in MeOH (2 ml), treated with a saturated potassium carbonate/water solution (0.5 ml) and heated to 60° C. for 16 h. The solvent was removed under vacuum and the crude was partitioned between ethyl acetate and brine solution. The ethyl acetate was washed twice with brine and the combined ethyl acetate fractions were dried over MgSO₄ to yield the title product. MS (ES) m/z 335.10 [M+H]⁺. Open access(HF): 100% at 254 nm RT=2.70 min.

### Example 570

### Preparation of 2,2,2-trifluoro-N-((1-(4-(6-hydroxynaphthalen-2-yl)pyrimidin-2-yl)piperidin-4-yl)methyl)acetamide

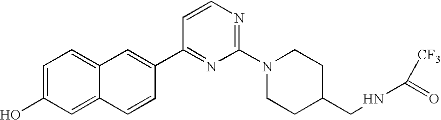

2,2,2-trifluoro-N-((1-(4-(6-hydroxynaphthalen-2-yl)pyrimidin-2-yl)piperidin-4-yl)methyl)acetamide 2,2,2-trifluoro-N-((1-(4-(6-methoxynaphthalen-2-yl)pyrimidin-2-yl)piperidin-4-yl)methyl)acetamide mg, 0.17 mmol) was dissolved in DCM (4 mL) and cooled to 0° C. under nitrogen. BBr₃ solution (0.4 mL of a 1M solution in DCM, 0.4 mmol) was added dropwise, and the solution was allowed to warm to room temperature. The reaction was allowed to stir for 16 h. The solvent was removed under vacuum and the crude was partitioned between ethyl acetate and brine solution. The ethyl acetate was washed twice with brine and the combined ethyl acetate fractions were dried over MgSO₄, yielding the title product as a yellow solid (53 mg, (73%).12 mmol). MS (ES) m/z 431.09 [M+H]⁺. Open access(HF): about 90% at 254 nm RT=4.64 min.

### Example 571

### Preparation of 2,2,2-trifluoro-N-((1-(4-(6-methoxynaphthalen-2-yl)pyrimidin-2-yl)piperidin-4-yl)methyl)acetamide

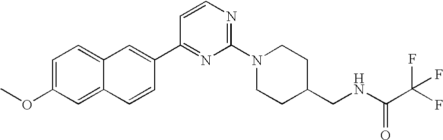

2,2,2-trifluoro-N-((1-(4-(6-methoxynaphthalen-2-yl)pyrimidin-2-yl)piperidin-4-yl)methyl)acetamide (4-(4-(6-Methoxynaphthalen-2-yl)pyrimidin-2-yl)cyclohexyl)methanamine (500 mg, 1.4 mmol) was dissolved in MeOH (50 mL), DCM (50 mL) and ethyl trifluoroacetate (2 ml). This reaction mixture was allowed to stir for 16 h. The solvent was removed and the crude was purified on silica in a gradient proceeding from 0% to 100% ethyl acetate/hexane to yield the title product (520 mg (87%), 1.2 mmol). MS (ES) m/z 445.61 [M+H]⁺. Open access(HF): about 85% at 254 nm RT=5.84 min.

### Example 572

### 6-(2-(4-((2,2,2-trifluoroacetamido)methyl)piperidin-1-yl)pyrimidin-4-yl) aphthalene-2-yl trifluoromethanesulfonate

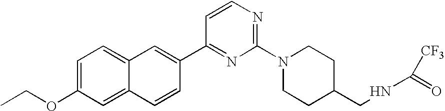

N-((1-(4-(6-ethoxynaphthalen-2-yl)pyrimidin-2-yl)piperidin-4-yl)methyl)-2,2,2-trifluoroacetamide 2,2,2-trifluoro-N-((1-(4-(6-hydroxynaphthalen-2-yl)pyrimidin-2-yl)piperidin-4-yl)methyl)acetamide mg, 0.05 mmol) was dissolved in DMF (2ml) and charged with cesium carbonate (136 mg, 0.20 mmol) and bromoethane (104 mg, 0.95 mmol). The reaction mixture was stirred for 16 h. The solvent was partitioned between ethyl acetate and brine solution. The ethyl acetate was washed twice with brine and the combined ethyl acetate fractions were dried over MgSO₄. This crude was purified on silica in a gradient proceeding from 0% to 100% ethyl acetate/hexane to yield the protected product. This product taken up in MeOH (2 ml), treated with a saturated potassium carbonate/water solution (0.5 ml) and heated to 60° C. for 16 h. The solvent was removed under vacuum and the crude was partitioned between ethyl acetate and brine solution. The ethyl acetate was washed twice with brine and the combined ethyl acetate fractions were dried over MgSO₄ to yield the title product (12 mg (52%), .027 mmol). MS (ES) m/z 363.16 [M+H]⁺. Open access (HF): about 90% at 254 nm RT=3.60 min.

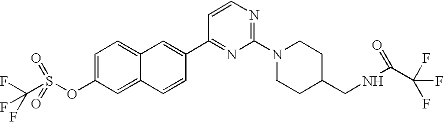

6-(2-(4-((2,2,2-trifluoroacetamido)methyl)piperidin-1-yl)pyrimidin-4-yl)□aphthalene-2-yl trifluoromethanesulfonate 2,2,2-trifluoro-N-((1-(4-(6-hydroxynaphthalen-2-yl)pyrimidin-2-yl)piperidin-4-yl)methyl)acetamide (35 mg, 0.08 mmol) was dissolved in THF (4 mL) under nitrogen. N-Phenylbis(trifluoromethanesulfonimide) (64 mg, 0.18 mmol) was added with sodium carbonate (48 mg, 0.48 mmol). The reaction was heated overnight at 60° C. to stir for 16 h. The solvent was removed under vacuum and the crude was partitioned between ethyl acetate and brine solution. The ethyl acetate was washed twice with brine and the combined ethyl acetate fractions were dried over MgSO₄, yielding the title product (25 mg, (50%)0.044 mmol). MS (ES) m/z 562.95 [M+H]⁺. Open access (normal gradient): about 90% at 254 nm RT=2.19 min.

### Example 578

### Preparation of (1-(4-(6-methylnaphthalen-2-yl)pyrimidin-2-yl)piperidin-4-yl)methanamine

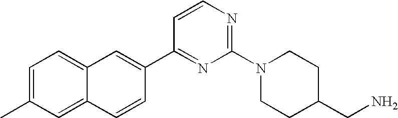

(1-(4-(6-methylnaphthalen-2-yl)pyrimidin-2-yl)piperidin-4-yl)methanamine 6-(2-(4-((2,2,2-trifluoroacetamido)methyl)piperidin-1-yl)pyrimidin-4-yl)naphthalen-2-yl trifluoromethanesulfonate (100 mg, 0.18 mmol) was dissolved in dioxane (2ml) and charged with dimethylzinc solution (0.18 ml of a 2.0 M solution in toluene, 0.36 mmol) and Pd(dppf)Cl₂ (4.4 mg, 0.0054 mmol). The reaction mixture was stirred at 100° C. for 16 h. The solvent was removed and the intermediate was purified on silica in a gradient proceeding from 0% to 100% ethyl acetate/hexane to yield the protected product. This product was taken up in MeOH (2 ml), treated with a saturated sodium carbonate/water solution (0.5 ml) and heated to 60° C. for 16 h. The solvent was removed under vacuum and the crude was partitioned between ethyl acetate and brine solution. The ethyl acetate was washed twice with brine and the combined ethyl acetate fractions were dried over MgSO₄ to yield the title product (12 mg (15%), 0.028 mmol). MS (ES) m/z 333.20 [M+H]⁺. Open access (HF): about 97% at 254 nm RT=3.49 min.

### Example 575

### Preparation of (1-(4-(6-phenylnaphthalen-2-yl)pyrimidin-2-yl)piperidin-4-yl)methanamine

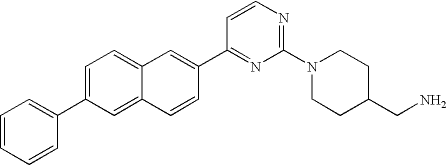

(1-(4-(6-phenylnaphthalen-2-yl)pyrimidin-2-yl)piperidin-4-yl)methanamine 6-(2-(4-((2,2,2-trifluoroacetamido)methyl)piperidin-1-yl)pyrimidin-4-yl)naphthalen-2-yl trifluoromethanesulfonate (50 mg, 0.09 mmol) was dissolved in DMF (0.5 ml) and charged with phenylboronic acid (17 mg, 0.14 mmol), lithium chloride (11 mg, 0.27 mmol), sodium carbonate (29 mg, 0.27 mmol) and Pd(PPh3)₂Cl₂ (2 mg, 0.003 mmol). The reaction mixture was stirred at 100° C. for 16 h. The solvent was removed and the intermediate was purified on silica in a gradient proceeding from 10% to 100% ethyl acetate/hexane to yield the protected product. This product was taken up in MeOH (2 ml), treated with a saturated sodium carbonate/water solution (0.5 ml) and heated to 60° C. for 16 h. The solvent was removed under vacuum and the crude was partitioned between ethyl acetate and brine solution. The ethyl acetate was washed twice with brine and the combined ethyl acetate fractions were dried over MgSO₄ to yield the title product (16 mg (36%), 0032 mmol). MS (ES) m/z 395.19 [M+H]⁺. Open access (HF): about 95% at 254 nm RT=4.14 min.

### Example 576

### Preparation of 2,2,2-trifluoro-N-((1-(4-(6-formylnaphthalen-2-yl)pyrimidin-2-yl)piperidin-4-yl)methyl)acetamide

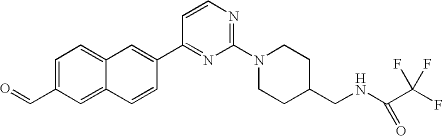

2,2,2-trifluoro-N-((1-(4-(6-formylnaphthalen-2-yl)pyrimidin-2-yl)piperidin-4-yl)methyl)acetamide (1-(4-(6-vinylnaphthalen-2-yl)pyrimidin-2-yl)piperidin-4-yl)methanamine (22 mg, 0.05 mmol) was dissolved in dioxane (0.5 ml) and water (0.75 ml). The reaction mixture was charged with NaIO₄ (64 mg, 0.3 mmol) and osmium tetroxide (0.33 ml of a 2.5% solution). The reaction mixture was stirred at RT for 16 h. The solvent was removed and the intermediate was purified on silica in a gradient proceeding from 10% to 100% ethyl acetate/hexane to yield the product (15 mg (68%), 0.03 mmol). MS (ES) m/z 442.96 [M+H]⁺. Open access (normal): about 90% at 254 nm RT=2.05 min.

### Example 577

### Preparation of (1-(4-(6-vinylnaphthalen-2-yl)pyrimidin-2-yl)piperidin-4-yl)methanamine

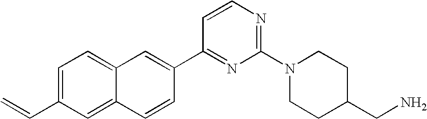

(1-(4-(6-vinylnaphthalen-2-yl)pyrimidin-2-yl)piperidin-4-yl)methanamine 6-(2-(4-((2,2,2-trifluoroacetamido)methyl)piperidin-1-yl)pyrimidin-4-yl)naphthalen-2-yl trifluoromethanesulfonate (500 mg, 0.89 mmol) was dissolved in dioxane (80ml) and charged with tributylvinyl tin (338 mg, 1.1 mmol), lithium chloride (113 mg, 2.7 mmol) and palladium tetrakis (21 mg, 0.018 mmol). The reaction mixture was stirred at 100° C. for 16 h. The solvent was removed and the intermediate was purified on silica in a gradient proceeding from 10% to 100% ethyl acetate/hexane to yield the protected product. This product was taken up in MeOH (2 ml), treated with a saturated sodium carbonate/water solution (0.5 ml) and heated to 60° C. for 16 h. The solvent was removed under vacuum and the crude was partitioned between ethyl acetate and brine solution. The ethyl acetate was washed twice with brine and the combined ethyl acetate fractions were dried over MgSO₄ to yield the title product (110 mg (31%), .28 mmol). MS (ES) m/z 345.20 [M+H]⁺. Open access (HF): about 98% at 254 nm RT=3.60 min.

A similar method was used to prepare the compounds shown below:

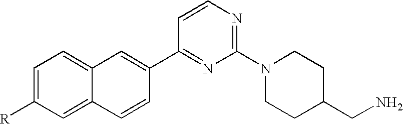

| | | | | HPLC |
|---|---|---|---|---|
| Example | Chemical Name | MS | Rt | method |
| 572 | (1-(4-(6-(thiophen-2-yl)naphthalen-2-yl)pyrimidin-2- | 401 | 3.90 | HF |
| | yl)piperidin-4-yl)methanamine | | | |
| 573 | (1-(4-(6-(2-methoxyphenyl)naphthalen-2-yl)pyrimidin-2- | 425 | 4.53 | HF |
| | yl)piperidin-4-yl)methanamine | | | |
| 574 | (1-(4-(6-(4-methoxyphenyl)naphthalen-2-yl)pyrimidin-2- | 425 | 3.93 | HF |
| | yl)piperidin-4-yl)methanamine | | | |

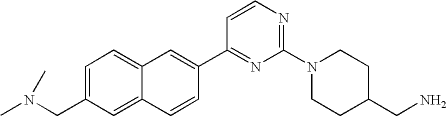

1-(6-(2-(4-(aminomethyl)piperidin-1-yl)pyrimidin-4-yl)naphthalen-2-yl)-N,N-dimethylmethanamine, 2,2,2-trifluoro-N-((1-(4-(6-formylnaphthalen-2-yl)pyrimidin-2-yl)piperidin-4-yl)methyl)acetamide (40 mg, 0.09 mmol) was dissolved in THF (3 ml) and charged with dimethylamine (0.135 ml of a 2M THF solution), sodium triacetoxyborohydride (58 mg, 0.27 mmol), and a drop of acetic acid. The reaction mixture was stirred at RT for 16 h. The solvent was removed and the intermediate was purified on silica in a gradient proceeding from 10% to 100% ethyl acetate/hexane to yield the protected product. This product was taken up in MeOH (2 ml), treated with a saturated sodium carbonate/water solution (0.5 ml) and heated to 60° C. for 16 h. The solvent was removed under vacuum and the crude was partitioned between ethyl acetate and brine solution. The ethyl acetate was washed twice with brine and the combined ethyl acetate fractions were dried over MgSO₄ to yield the title product (10 mg (22%), 0.026 mmol). MS (ES) m/z 376.26[M+H]⁺. Open access (HF): about 99% at 254 nm RT=2.26 min.

Additional compounds were prepared by a similar method:

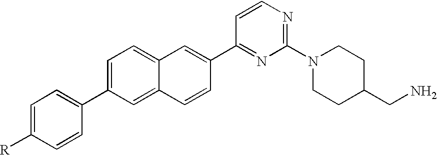

| Example | | | | HPLC |
|---|---|---|---|---|
| No. | Compound | MS | Rt | method |
| 579 | (1-(4-(6-(4-(piperidin-1-ylmethyl)phenyl)naphthalen-2-yl)pyrimidin- | 416 | 2.54 | HF |
| | 2-yl)piperidin-4-yl)methanamine | | | |
| 580 | (1-(4-(6-(4-(morpholinomethyl)phenyl)naphthalen-2-yl)pyrimidin-2- | 418 | 2.30 | HF |
| | yl)piperidin-4-yl)methanamine | | | |

### Example 314

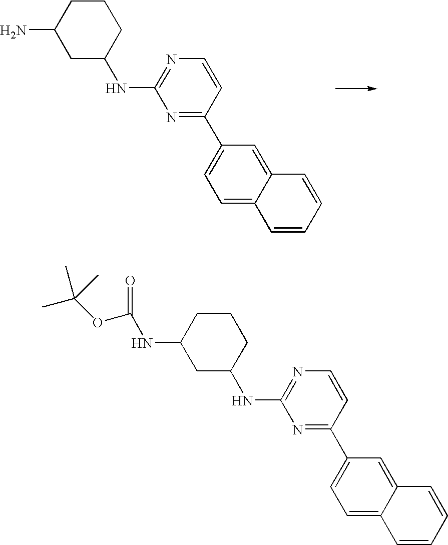

[3-(4-Naphthalen-2-yl-pyrimidin-2-ylamino)-cyclohexyl]-carbamic acid tert-butyl ester. N-(4-Naphthalen-2-yl-pyrimidin-2-yl)-cyclohexane-1,3-diamine (35 mg, 0.11 mmol) is dissolved in THF (1 ml) with BOC-anhydride (24 mg, 0.11 mmol). The solution is allowed to stir for 16 h. It is purified by HPLC method D to yield the title product (2.5 mg (5%), 0.006 mmol). ES POS: [M+H]+419 ; Retention time 2.00 min (Method C).

### Example 315

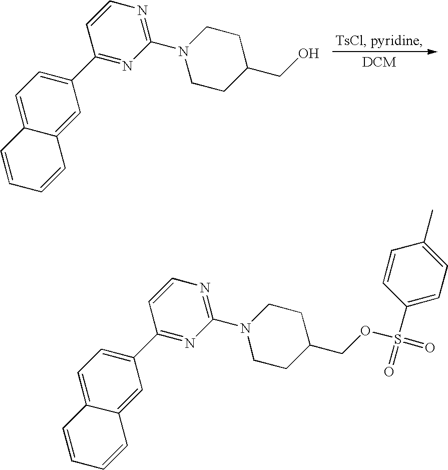

{1-[4-(2-naphthyl)pyrimidin-2-yl]piperidin-4-yl}methyl 4-methylbenzenesulfonate. To a solution of the alcohol (627 mg, 1.97mmol) in DCM is added pyridine (478 μL, 5.91 mmol) and tosyl chloride (751 mg, 3.94 mmol). The reaction is stirred at room temperature for 16 h, then tosyl chloride (375 mg, 1.97 mmol) is added to drive the reaction to completion. The reaction is stirred at room temperature for an additional 16 h, and then partitioned between DCM and water. The aqueous layer is extracted with DCM (1×) and the combined organic layers are dried (MgSO₄), filtered, concentrated, and chromatographed on silica gel (30% ethyl acetate:hexane as the eluent) to yield the tosylate (228 mg 24%). HPLC (method F): Rt=11.9 mins. MS: (M+H)⁺=474.

### Example 316 indicates a mesylate prepared in a similar manner as the above tosylate using methanesulfonyl chloride and 4-(2-naphthyl)-2-(4-(1′-hydroxyeth-2-yl)piperidin-1-yl)pyrimidine.

| Example | | | HPLC R.t | HPLC |
|---|---|---|---|---|
| no. | Compound | MS | (mins.) | Method |
| 316 | 2-{1-[4-(2-naphthyl)pyrimidin- | 412 | 11.1 | F |
| | 2-yl]piperidin-4-yl}ethyl | | | |
| | methanesulfonate | | | |

### Example 317

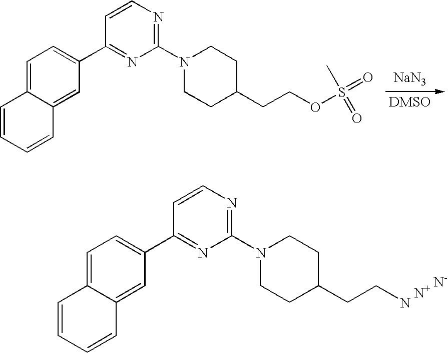

2-[4-(2-azidoethyl)piperidin-1-yl]-4-(2-naphthyl)pyrimidine. To a solution of mesylate (676 mg, 1.64 mmol) in DMSO (10 mL) is added NaN₃ (128 mg, 1.97 mmol). The reaction is heated to 45° C. for 16 h, then partitioned between ethyl acetate and water. The layers are separated, and the aqueous layer is extracted with ethyl acetate (2×). The combined organic layers are washed with water (3×), saturated sodium bicarbonate (1×), and brine (1×). The organic layer is dried (MgSO₄), filtered, concentrated, and chromatographed on silica gel (eluted with 80% ethyl acetate: hexane) to provide the product (560 mg, 95%). HPLC (method F): Rt=11.2 mins. MS: (M+H)⁺=358.

### Example 318

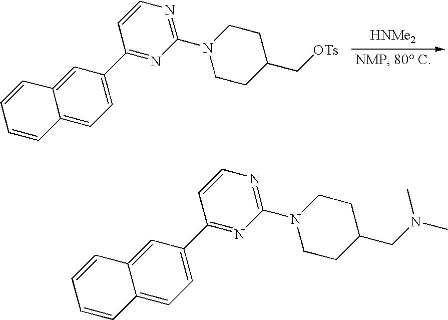

### General Procedure for Tertiary Amines.

N,N-dimethyl-1-{1-[4-(2-naphthyl)pyrimidin-2-yl]piperidin-4-yl }methanamine. Dimethyl amine (233 μL, 0.47 mmol) is added to a solution of tosylate (22 mg, 0.047 mmol) in NMP (0.7 mL). The reaction is heated at 80° C. for 16 h. After cooling to room temperature the mixture is diluted with water (0.10 mL) and triethylamine (0.10 mL) and purified by direct HPLC injection (method D) to leave (8 mg, 49%) of the dimethyl product. HPLC (method E): Rt=2.2 mins. MS: (M+H)⁺=347.

The following examples were prepared using the method of Example 318.

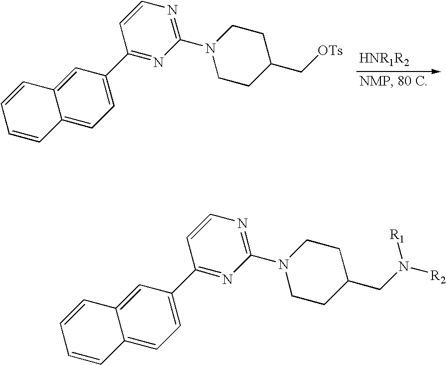

| Example | | | Rt | HPLC |
|---|---|---|---|---|
| No. | Compound | MS | (min.) | Method |
| 581 | 2-{4-[(4-methylpiperazin-1-yl)methyl]piperidin-1-yl}-4-(2- | 402 | 3.36 | H |
| | naphthyl)pyrimidine | | | |
| 582 | N-methyl-N-({1-[4-(2-naphthyl)pyrimidin-2-yl]piperidin-4- | 415 | 3.97 | H |
| | yl}methyl)cyclohexanamine | | | |
| 583 | 1-({1-[4-(2-naphthyl)pyrimidin-2-yl]piperidin-4-yl}methyl)azepane | 402 | 2.84 | H |
| 584 | 2-[ethyl({1-[4-(2-naphthyl)pyrimidin-2-yl]piperidin-4- | 391 | 3.53 | H |
| | yl}methyl)amino]ethanol | | | |
| 585 | 2-methyl-N-({1-[4-(2-naphthyl)pyrimidin-2-yl]piperidin-4- | 375 | 3.69 | H |
| | yl}methyl)propan-2-amine | | | |
| 586 | 1-({1-[4-(2-naphthyl)pyrimidin-2-yl]piperidin-4-yl}methyl)pyrrolidin- | 387 | 5.6 | H |
| | 2-one | | | |
| 587 | 1-({1-[4-(2-naphthyl)pyrimidin-2-yl]piperidin-4-yl}methyl)piperidin-2- | 401 | 5.88 | H |
| | one | | | |
| 588 | 4-(2-naphthyl)-2-[4-(piperazin-1-ylmethyl)piperidin-1-yl]pyrimidine | 388 | 4.00 | H |
| 589 | 4-({1-[4-(2-naphthyl)pyrimidin-2-yl]piperidin-4-yl}methyl)morpholine | 389 | 3.51 | H |
| 590 | 4-({1-[4-(2-naphthyl)pyrimidin-2-yl]piperidin-4-yl}methyl)piperazin-2- | 402 | 3.53 | H |
| | one | | | |

### Example 319

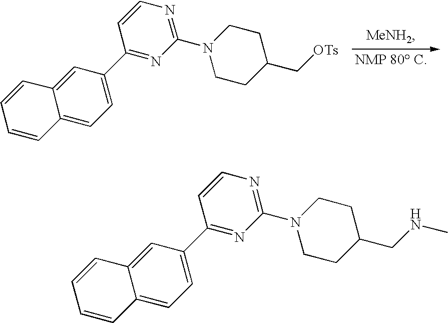

### General Procedure for Secondary Amines.

N-methyl-1-{1-[4-(2-naphthyl)pyrimidin-2-yl]piperidin-4-yl}methanamine. Methylamine (233 μL, 0.47 mmol) is added to a solution of tosylate (22 mg, 0.047 mmol) in NMP (0.7 mL). The reaction is heated at 80° C. for 16 h. The reaction is cooled to room temperature, DCM (1mL) and TsOH resin (429 mg, 0.60 mmol) are added, and the mixture is stirred 16 h at room temperature. The reaction mixture is filtered. The resin is washed with DMF (5×), methanol (5×), DCM (5×), and methanol (1×). To the resin is added 2.0 M ammonia in methanol (3 mL). The mixture is stirred at room temperature for 2 h, then filtered. The resin is washed with methanol (3×). The combined filtrate and washings are concentrated to afford 3 mg (19%) of the desired product. HPLC (method E): Rt=2.0 mins. MS: (M+H)⁺=332.

The following additional examples were prepared using the method described in example 319.

| Example | | | R.t | HPLC |
|---|---|---|---|---|
| no. | Compound | MS | (mins.) | Method |
| 321 | 2-[4-(1H-imidazol-1-ylmethyl)piperidin-1-yl]-4-(2- | 370 | 2.2 | E |
| | naphthyl)pyrimidine | | | |
| 322 | 4-(2-naphthyl)-2-[4-(pyrrolidin-1-ylmethyl)piperidin- | 373 | 2.3 | E |
| | 1-yl]pyrimidine | | | |
| 323 | N-ethyl-N-({1-[4-(2-naphthyl)pyrimidin-2- | 375 | 2.4 | E |
| | yl]piperidin-4-yl}methyl)ethanamine | | | |
| 324 | 4-(2-naphthyl)-2-[4-(piperidin-1-ylmethyl)piperidin- | 387 | 2.4 | E |
| | 1-yl]pyrimidine | | | |
| 325 | N-methyl-1-{1-[4-(2-naphthyl)pyrimidin-2- | 333 | 2 | E |
| | yl]piperidin-4-yl}methanamine | | | |
| 326 | N-({1-[4-(2-naphthyl)pyrimidin-2-yl]piperidin-4- | 347 | 2 | E |
| | yl}methyl)ethanamine | | | |
| 327 | 2-methyl-N-({1-[4-(2-naphthyl)pyrimidin-2- | 375 | 2.3 | E |
| | yl]piperidin-4-yl}methyl)propan-1-amine | | | |

### Example 328

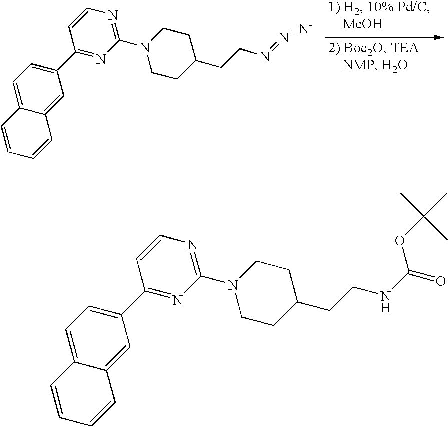

tert-butyl (2-{1-[4-(2-naphthyl)pyrimidin-2-yl]piperidin-4-yl}ethyl)carbamate. A solution of the azide (762 mg, 2.13 mmol) in methanol is flushed with nitrogen (3×), and 10% palladium on carbon (75 mg) is added. The mixture is flushed with nitrogen (3×), fitted with a hydrogen balloon, and flushed with hydrogen (3×). The reaction is stirred at room temperature under a hydrogen atmosphere for 18 h. The reaction is 50% complete and the balloon is deflated. After the addition of more 10% palladium on carbon to the reaction, the balloon is refilled with hydrogen, and the reaction is stirred under a hydrogen atmosphere for 2 h. The reaction is filtered through celite. The crude naphthyl amine (300 mg, 0.904) is dissolved in NMP (5 mL). Di-tert-butyldicarboxylate (197 mg, 0.904 mmol) and triethylamine (189 μL, 1.36 mmol) are added and the reaction is stirred at room temperature for 1 h. The reaction is partitioned between ethyl acetate and water. The layers are separated. The aqueous layer is extracted with ethyl acetate (1×). The combined organinc layers are washed with water (2×), saturated sodium bicarbonate (1×), and brine (1×). The organic layer is dried (MgSO₄), filtered, concentrated, and chromatographed on silica gel (eluted with 20% ethyl acetate: hexane) to provide the product (221 mg). HPLC (method F): Rt=12.0 mins. MS: (M+H)⁺=433

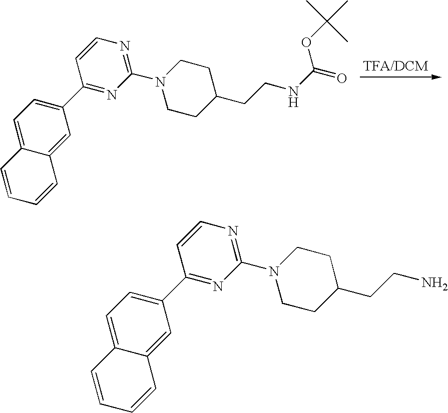

2-{1-[4-(2-naphthyl)pyrimidin-2-yl]piperidin-4-yl}ethanamine. A solution of the protected amine (204 mg, 0.471 mmol), DCM (2.5 mL), and TFA (2.5 mL) is stirred at room temperature for 1 h. The reaction is concentrated. Toluene (10 mL) is added, and the resulting mixture is concentrated. The toluene wash is repeated (3×), and the resulting oil is freeze-dried to yield pure amine as the di-TFA salt (259 mg, 98%). HPLC (method F): Rt=9.0 mins. MS: (M+H)⁺=333.

### Example 329

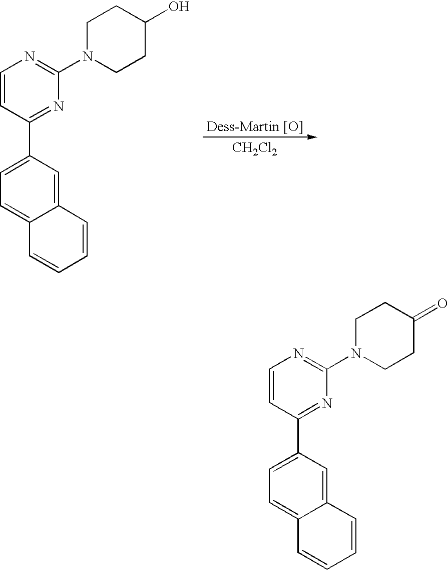

1-[4-(2-naphthyl)pyrimidin-2-yl]piperidin-4-one. 1-[4-(2-naphthyl)pyrimidin-2-yl]piperidin-4-ol (0.1156 g, 0.378 mmol) is dissolved in dichloromethane (3 mL), treated with Dess-Martin periodinane (0.376 g, 0.886 mmol) and stirred at room temperature for 2h. When the reaction is nearly complete by TLC, the mixture is concentrated to a residue and purified by semi-preparative reversed-phase HPLC (Method D). The pure fractions are concentrated in a Genevac evaporator to give 0.0498 g (37%) of the product. HPLC (Method G) rt=10.4 min, calculated mass 303.1, [M+H]⁺=304.1.

### Examples 330 and 331

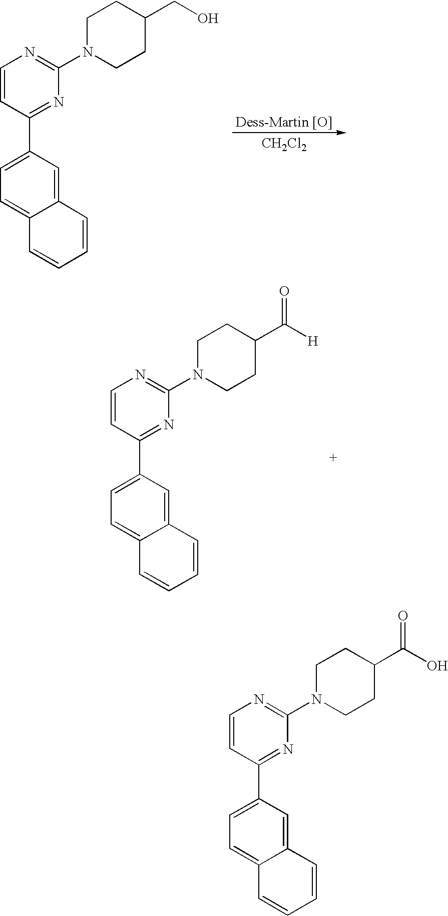

1-[4-(2-naphthyl)pyrimidin-2-yl]piperidine-4-carbaldehyde. { 1-[4-(2-naphthyl)pyrimidin-2-yl]piperidin-4-yl}methanol (0.0808 g, 0.253 mmol) is dissolved in dichloromethane (3 mL) and stirred at room temperature for 2 h. When TLC showed near complete consumption of starting material, the mixture is concentrated to a residue and purified by semi-preparative reversed-phase HPLC (method D). The pure fractions are concentrated in a Genevac evaporator to give 0.0192 g (24%) of the aldehyde product. HPLC (Method G) rt=10.7 min, calculated mass 317.1, [M+H]⁺=318.2. Additionally, the corresponding carboxylic acid (see below) is isolated as a side product.

1-[4-(2-naphthyl)pyrimidin-2-yl]piperidine-4-carboxylic acid The oxidation reaction described above for {1-[4-(2-naphthyl)pyrimidin-2-yl]piperidin-4-yl}methanol also furnished 0.0226 g (27%) of 1-[4-(2-naphthyl)pyrimidin-2-yl]piperidine-4-carboxylic acid as additional product. HPLC (method F) rt=10.6 min, calculated mass 333.1, [M+H]⁺=334.2.

### Example 332

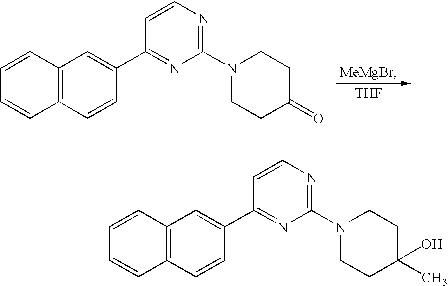

4-methyl-1-[4-(2-naphthyl)pyrimidin-2-yl]piperidin-4-ol. To a solution of ketone (100 mg, 0.33 mmol) in THF (5 mL) at 0° C. is added MeMgBr (0.550 mL, 1.65 mmol). The reaction is allowed to warm to room temperature and stirred for 16 h. The reaction is partitioned between ethyl acetate and saturated ammonium chloride, and the layers are separated. The aqueous layer is extracted with ethyl acetate (2×). The combined organic layers are dried (MgSO₄), filtered, concentrated, and chromatographed on silica gel (eluted with 50% ethyl acetate: hexane) to provide the product (78 mg, 74%). HPLC (method F): Rt=10.6 mins. MS: (M+H)⁺=320.

### Example 333

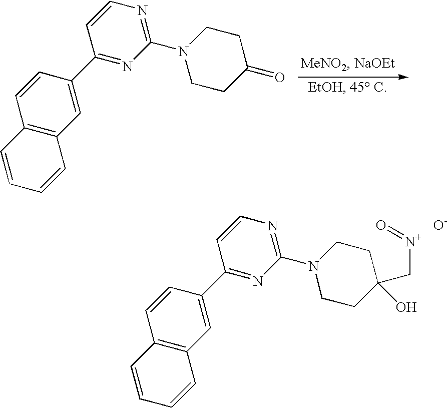

1-[4-(2-naphthyl)pyrimidin-2-yl]-4-(nitromethyl)piperidin-4-ol: To a solution of ketone (600 mg, 2.0 mmol) and nitromethane (0.360 mL, 6.0 mmol) in ethanol (30 mL) is added sodium ethoxide (21% wt, 1.48 mL 4.0 mmol). The reaction is heated at 45° C. for 16 h, then concentrated and partitioned between ethyl acetate and saturated ammonium chloride. The layers are separated. The aqueous layer is extracted with ethyl acetate (3×). The combined organic extracts are washed with brine (1×), dried (MgSO₄), filtered, concentrated, and chromatographed on silica gel (eluted with 30% ethyl acetate: hexane) to provide the product (670 mg, 92%) HPLC (method F): Rt=10.5 mins. MS: (M+H)⁺=365.

### Example 334

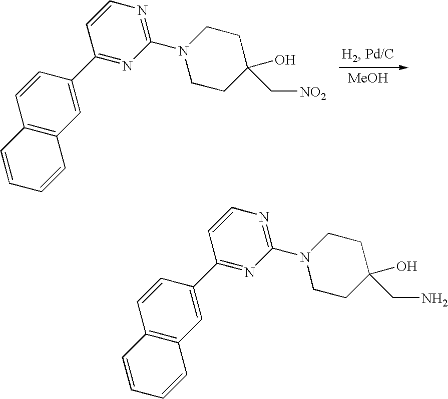

4-(aminomethyl)-1-[4-(2-naphthyl)pyrimidin-2-yl]piperidin-4-ol: The nitro alcohol (147 mg, 0.403 mmol) is placed in methanol (5 mL). After flushing with nitrogen, 10% Pd/C (catalytic amount) is added. The reaction is flushed with nitrogen, then with hydrogen. A hydrogen balloon is affixed to the reaction vessel, and the reaction is stirred at room temperature. After 3 days, the reaction is filtered through celite, washed with methanol, and concentrated. The resulting residue is chromatographed on silica gel (eluted with 100% ethyl acetate followed by 100% methanol) to provide the product (35 mg, 25%). HPLC (method F): Rt=8.2 mins. MS: (M+H)⁺=335.

### Example 335

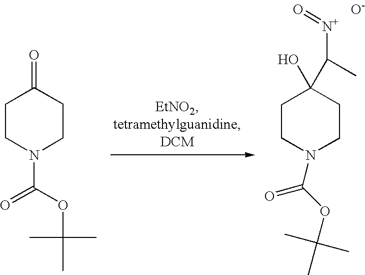

N-Boc-4-(1-Nitro-ethyl)-piperidin-4-ol. To a solution of the ketone (3 g, 15.1 mmol) in DCM (20 mL) is added nitroethane (3.3 mL, 45.3 mmol) and tetramethylguanidine (1.2 mL, 15.1 mmol). The reaction is stirred at room temperature for 4 days, partitioned between DCM and water, and the layers are separated. The aqueous layer is extracted with DCM (1×). The combined organic layers are dried (MgSO₄), filtered, concentrated, and chromatographed on silica gel (eluted with 30% ethyl acetate: hexane) to provide the product (2.0 g, 48%). HPLC (method C): Rt=7.6 mins. MS: (M+H)⁺=275.

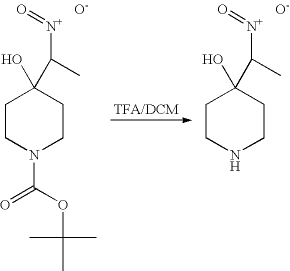

4-(1-Nitro-ethyl)-piperidin-4-ol. A solution of protected piperidine (300 mg, 1.09 mmol), DCM (5 mL) and TFA (5 mL) is stirred at room temperature for 1 h., then the reaction is concentrated. Toluene (10 mL) is added, and the resulting mixture is concentrated. The toluene wash is repeated (3×), and the resulting oil is freeze-dried to yield pure amine as the TFA salt (310 mg, 99%). HPLC (method A): Rt=0.30 mins. MS: (M+H)⁺=175.

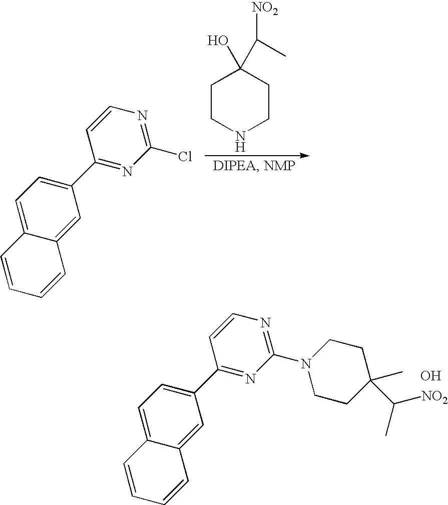

1-[4-(2-naphthyl)pyrimidin-2-yl]-4-(1-nitroethyl)piperidin-4-ol. To a solution of naphthylpyrimidine (162 mg, 0.672 mmol) in NMP (4.5 mL) is added the nitro-piperinyl alcohol (291 mg, 1.01 mmol) and diisopropylethylamine (258 μL, 1.48 mmol). The reaction is heated at 80° C. for 1 h, then partitioned between ethyl acetate and water. The aqueous layer is extracted with ethyl acetate (2×) and the combined organic layers are washed with water (4×), and brine (1×). The organic layers are dried (MgSO₄), filtered, concentrated, and chromatographed on silica gel (40% ethyl acetate:hexane as the eluent) to yield the aminopyrimidine (100 mg 40%). HPLC (method F): Rt=10.8 mins. MS: (M+H)⁺=379.

### Example 336

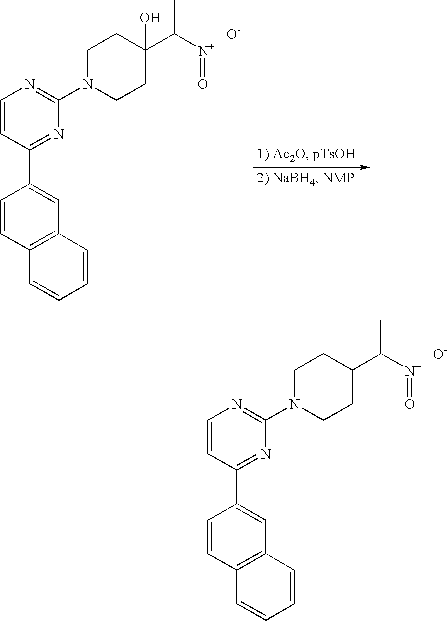

4-(2-naphthyl)-2-[4-(1-nitroethyl)piperidin-1-yl]pyrimidine. Prepared according to the procedure found in Tetrahedron 50, 33; 9961-9974, 1994. To a solution of the alcohol (110 mg, 0.291 mmol) in acetic anhydride (2 mL) is added pTsOH-monohydrate (55 mg, 0.291 mmol). The reaction is stirred at room temperature for 18 h. The reaction is poured into ice-cold water and stirred for 5 min. Ethyl acetate is added, and the layers are separated. The organic layer is washed with water (1×), saturated sodium bicarbonate (3×), and brine (1×). The organic layer is dried (MgSO₄), filtered, concentrated to yield the crude acetate that is used without further purification. The crude acetate (90 mg, 0.214 mmol) is dissolved in ethanol (1.5 mL), and sodium borohydride (11 mg, 0.279 mmol) is added. After 3 h of stirring at room temperature, more sodium hydride is added (11 mg, 0.279 mmol). The reaction is stirred at room temperature for 18 h. Water and ethyl acetate are added. The layers are separated, and brine is added to the aqueous layer. The aqueous layer is extracted with ethyl acetate (5×). The combined organic layers are washed with water (4×) and brine (2×). The organic layer is dried (MgSO₄), filtered, concentrated, and chromatographed on silica gel (eluted with 20% ethyl acetate: hexane) to provide the product (55 mg, 52%). HPLC (method F): Rt=11.6 mins. MS: (M+H)⁺=363.

Example 337 was prepared according to example 336.

| Example | | | R.t | HPLC |
|---|---|---|---|---|
| no. | Compound | MS | (mins.) | Method |
| 337 | 4-(naphthalen-2-yl)-2-(4- | 349 | 11.3 | F |
| | (nitromethyl)piperidin-1- | | | |
| | yl)pyrimidine | | | |

### Examples 338 and 339

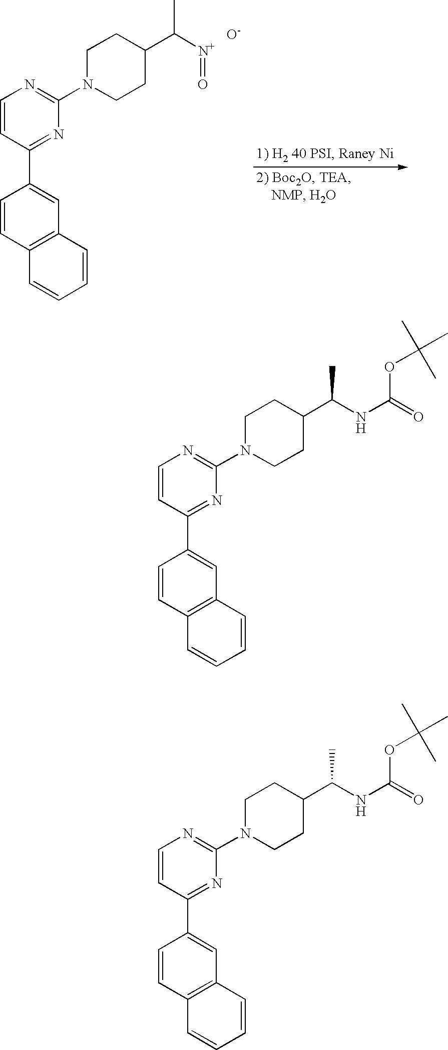

tert-butyl (1-{1-[4-(2-naphthyl)pyrimidin-2-yl]piperidin-4-yl}ethyl)carbamate. The nitro compound (150 mg, 0.414 mmol) and methanol (250 mL) are combined in a Parr flask and flushed with nitrogen. Raney Ni (1 mL) is added and the flask is placed on a Parr hydrogenator at 40 PSI for 16 h. The reaction is filtered through celite, and the resulting amine is used crude in the next step. The crude amine is dissolved in NMP (12 mL) and water (1.2 mL). Di-tert-butyldicarboxylate (135 mg, 0.621 mmol) and triethylamine (0.115 mL, 0.828 mmol) are added, and the reaction is stirred at room temperature. After 3 h, the reaction is partitioned between ethyl acetate and water. The layers are separated. The aqueous layer is extracted with ethyl acetate (1×). The combined organinc layers are washed with water (3×), saturated sodium bicarbonate (1×), and brine (1×). The organic layer is dried (MgSO₄), filtered, concentrated, and chromatographed on silica gel (eluted with 20% ethyl acetate: hexane) to provide the product (121 mg, 68%). HPLC (method F): Rt=11.9 mins. MS: (M+H)⁺=433. Approximately 120 mg of racemic mixture, CAT 1427003, is dissolved in 17.5 mL of CH₂Cl₂/methanol/acetonitrile (0.3:1:1). 400 μL of the resulting solution is repetitively injected onto the Supercritical Fluid Chromatography instrument, and the baseline resolved enantiomers are separately collected using the conditions described below. The chiral purity of each enantiomer is determined under the same Supercritical Fluid Chromatography conditions using a Chiralpak AD-H 5μm, 250 mm×4.6 mm ID column at 2.0 mL/min flow rate using Analytical Supercritical Fluid Chromatography (Berger Instruments, Inc. Newark, Del.). Both enantiomers are found to be >99.9% enantiomerically pure.

### Example 340

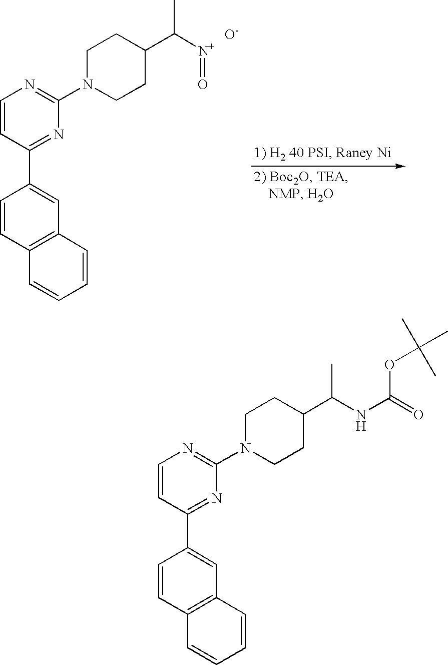

tert-butyl (1-{1-[4-(2-naphthyl)pyrimidin-2-yl]piperidin-4-yl}ethyl)carbamate. The nitro compound (150 mg, 0.414 mmol) and methanol (250 mL) are combined in a Parr flask and flushed with nitrogen. Raney Ni (1 mL) is added and the flask is placed on a Parr hydrogenator at 40 PSI for 16 h. The reaction is filtered through celite, and the resulting amine is used crude in the next step. The crude amine is dissolved in NMP (12 mL) and water (1.2 mL). Di-tert-butyldicarboxylate (135 mg, 0.621 mmol) and triethylamine (0.115 mL, 0.828 mmol) are added, and the reaction is stirred at room temperature. After 3 h, the reaction is partitioned between ethyl acetate and water. The layers are separated. The aqueous layer is extracted with ethyl acetate (1×). The combined organinc layers are washed with water (3×), saturated sodium bicarbonate (1×), and brine (1×). The organic layer is dried (MgSO₄), filtered, concentrated, and chromatographed on silica gel (eluted with 20% ethyl acetate: hexane) to provide the product (121 mg, 68%). HPLC (method F): Rt=11.9 mins. MS: (M+H)⁺=433.

### Example 341

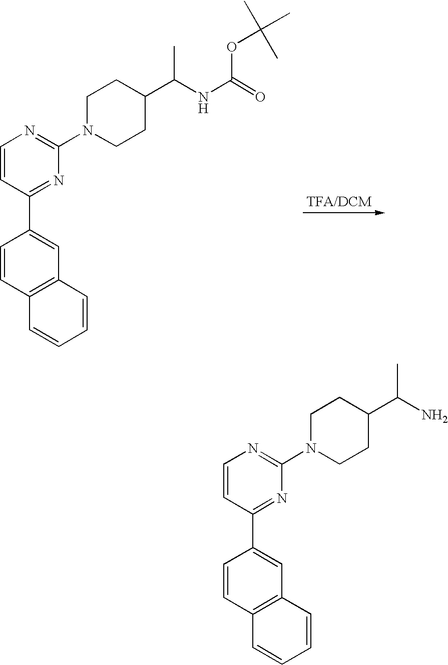

1-{1-[4-(2-naphthyl)pyrimidin-2-yl]piperidin-4-yl}ethanamine. A solution of the protected amine (29 mg, 0.067 mmol), DCM (1 mL), and TFA (1 mL) is stirred at room temperature for 1 h., then the reaction is concentrated. Toluene (10 mL) is added, and the resulting mixture is concentrated. The toluene wash is repeated (3×), and the resulting oil is freeze-dried to yield pure amine as the di-TFA salt (36 mg, 96%). HPLC (method G): Rt=10.9 mins. MS: (M+H)⁺=333

Enantiomer examples 342-343 were prepared according to the deprotection method for example 341 above.

| | | | R.t | HPLC |
|---|---|---|---|---|
| Example | Compound | MS | (mins.) | Method |
| 342 | (1R)-1-{1-[4-(2- | 333 | 8.9 | F |
| | naphthyl)pyrimidin-2-yl]piperidin- | | | |
| | 4-yl}ethanamine | | | |
| 343 | (1S)-1-{1-[4-(2- | 333 | 8.9 | F |
| | naphthyl)pyrimidin-2-yl]piperidin- | | | |
| | 4-yl}ethanamine | | | |

### Example 344

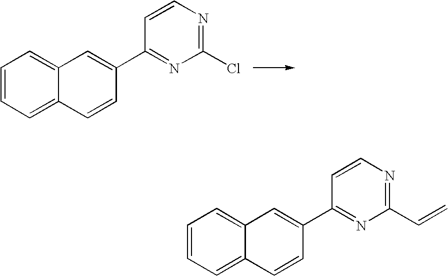

4-Naphthalen-2-yl-2-vinyl-pyrimidine. 2-Chloro-4-naphthalen-2-yl-pyrimidine (360 mg, 1.5 mmol) is dissolved in DMF (9 ml) with K₂CO₃ (621 mg, 4.5 mmol) and tetraethylammonium chloride (747 mg, 4.5 mmol). Tributylvinyltin (0.43 g, 4.5 mmol) is added, followed by dichloro(bistriphenylphosphine)palladium (105 mg, 0.15 mmol). The reaction mixture is allowed to stir for 16 h at 100° C. under a nitrogen atmosphere. The DMF is partitioned between ethyl acetate and brine. The brine is washed an additional time with ethyl acetate, and the combined ethyl acetate fractions are washed three additional times with brine. The solution is dried over magnesium sulfate, and the solvents are removed by vacuum distillation. This crude is purified by column chromatography (100% hexane to 100% ethyl acetate gradient) to yield the title product (301 mg (86%), 1.3 mmol). ES POS: [M+H]+233; Retention time 10.5 min (Method F).

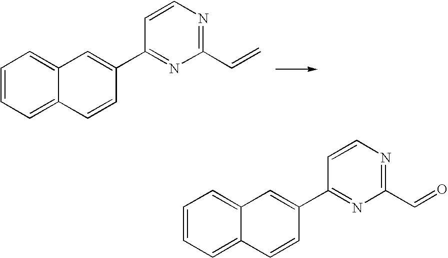

4-Naphthalen-2-yl-pyrimidine-2-carbaldehyde (L33458-9-3): 4-Naphthalen-2-yl-2-vinyl-pyrimidine (380 mg, 1.6 mmol) is dissolved in dioxane (8 ml). NaIO₄ (1.03 g 4.8 mmol) is dissolved in water (12 ml), and the two solutions are combined. OsO₄ (1 ml to a 2.5% solution in t-BuOH, 0.5 mmol) is added and the reaction is allowed to stir for 16 hours. The reaction mixture is partitioned between ethyl acetate and brine. The brine is washed an additional time with ethyl acetate, and the combined ethyl acetate fractions are washed three additional times with brine. The solution is dried over magnesium sulfate, and the solvents are removed by vacuum distillation. The crude is purified using column chromatography (0-50% ethyl acetate/hexane) to yield the title product (110 mg (29%), 0.47 mmol). ES POS: [M+H]+235; Retention time 1.14 min (Method A).

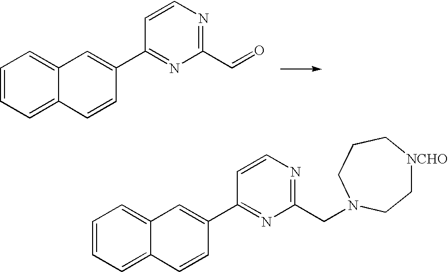

4-(4-Naphthalen-2-yl-pyrimidin-2-yl)-[1,4]diazepane-1-carbaldehyde. 4-Naphthalen-2-yl-pyrimidine-2-carbaldehyde (14 mg, 0.075 mmol) is dissolved in DCM (2 ml) with [1,4]Diazepane-1-carbaldehyde (13mg, 0.1 mmol). Sodium triacetoxyborohydride (42 mg, 0.2 mmol) is added and stirred for 48 hours. Solvent is removed under vacuum, and the crude is purified by HPLC method D to yield the title product (10 mg (40%), 0.03 mmol). ES POS: [M+H]+347; Retention time 2.92 min (Method H).

### Example 345 was prepared according to the method for example 344.

| Example | | | R.t | HPLC |
|---|---|---|---|---|
| no. | Compound | MS | (mins.) | Method |
| 345 | 1-(1-{[4-(2-naphthyl)pyrimidin-2- | 333 | 2.52 | H |
| | yl]methyl}piperidin- | | | |
| | 4-yl)methanamine | | | |

### Example 346

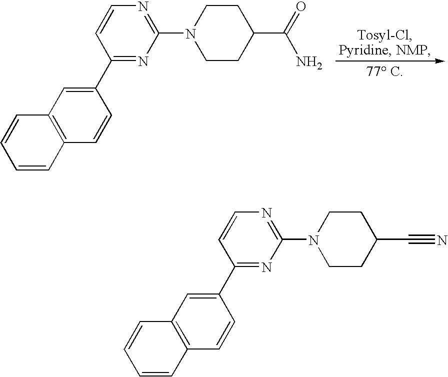

1-(4-Naphthalen-2-yl-pyrimidin-2-yl)-piperidine-4-carbonitrile. To a solution of amide (200 mg, 0.6 mMol), pyridine (7 mL) and NMP (2 mL) is added tosyl chloride (230 mg, 1.2 mMol) and the reaction is heated overnight at 77° C. After this time the crude reaction is concentrated and partitioned between ethyl acetate (100 mL) and water (100 mL). The organic phase is washed with water (3×) followed by brine (1×), then dried over MgSO₄ and concentrated. The crude material is purified by silica gel chromatography, eluting with 3% MeOH/CH₂Cl₂ (R_{f}=0.7), to afford the title compound as an off-white solid (140 mg, 74.3%). HPLC (Method F) purity 100%, rt=10.9 min; HRMS: calcd for C₂₀H₁₈N₄+H+, 315.16042; found (ESI, [M+H]+), 315.1604.

### Example 347

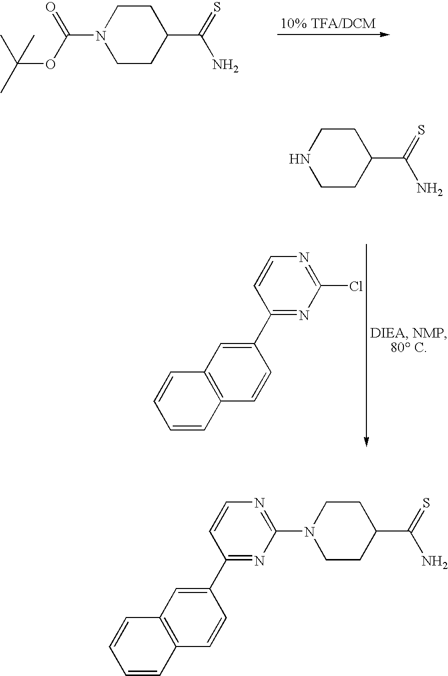

1-[4-(2-Naphthyl)pyrimidin-2-yl]piperidine-4-carbothioamide. A solution of 10% trifluoroacetic acid in dichloromethane (10 mL) is added to tert-butyl-4-(aminocarbothioyl)tetrahydropyridine-1(2H)-carboxylate (340 mg, 1.4 mmol). After 3 h the reaction is concentrated to dryness and the amine is used without purification. A portion of the resulting amine (464 μMol), 2-Chloro-4-naphthalen-2-yl-pyrimidine (29 mg, 120 μMol) and diisopropylethylamine (244 μL, 1.4 mMol) are combined in NMP (1 mL) and heated at 80° C. overnight. After cooling the crude is diluted with methanol (0.5 mL) and water (0.3 mL) and purified by RP-HPLC (Method D, without TFA modifier) to yield (5.6 mg, 13.4%); HPLC (Method E): Purity=94%, Rt=2.4 mins. MS: (M+H)⁺=349.

### Example 348

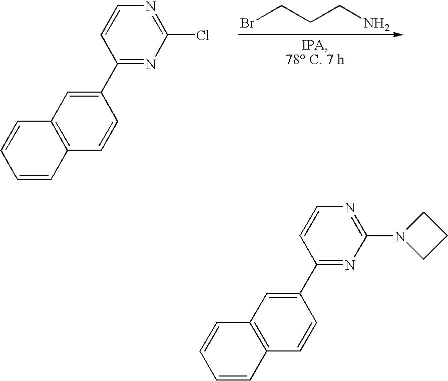

2-Azetidin-1-yl-4-(2-naphthyl)pyrimidine. A mixture of 2-chloro-4-naphthalen-2-yl-pyrimidine (72.2 mg, 300 μMol) and 3-bromopropylamine hydrobromide (65.7 mg, 300 μMol) in isopropanol (4 mL) is heated to 60° C. Diisopropylethylamine (157 μL, 900 μMol) is added and the reaction is heated at 78° C. for 7 hours. After cooling to room temperature the crude is concentrated and partitioned between ethyl acetate (50 mL) and water (50 mL). The organic phase is dried over MgSO₄ and concentrated. The crude material is purified by silica gel chromatography, eluting with 5% MeOH/CH₂Cl₂ (R_{f}=0.45), to afford the title compound as a white solid (30 mg, 38.3%). HPLC (Method F) purity 99.3%, rt=10.6 min; HRMS: calcd for C17H15N3+H+, 262.13387; found (ESI, [M+H]+), 262.1351.

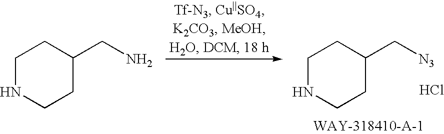

4-(Azidomethyl)piperidine. Prepared utilizing the method of Wong (Tetrahedron Lett. 1996, 37, 6029-6032) for carbohydrates with an customized work-up to isolate the HCl salt. Triflyl azide preparation: A solution of sodium azide (5.75 g, 88.5 mMol) is dissolved in distilled H₂O (13.5 mL) with CH₂Cl₂ (2×11.25 mL) and cooled on an ice bath. Triflyl anhydride (5.0 g, 17.7 mmol) is added slowly over 5 min with stirring continued for 2 h. The mixture is placed in a separatory funnel and the CH₂Cl₂ phase removed. The aqueous portion is extracted with CH₂Cl₂ (2×11.25 mL). The organic fractions, containing the triflyl azide, are pooled and washed once with saturated Na₂CO₃ and used without further purification. Target preparation: 4-(aminomethyl)piperidine (2.12 mL, 4.19 mMol) is combined with K₂CO₃ (3.66 g, 26.5 mMol) and Cu^{II}SO₄ pentahydrate (44 mg, 176 μmol) distilled H₂O (27 mL) and CH₃OH (54 mL). The triflyl azide in CH₂Cl₂ (45 mL) is added and the mixture is stirred at ambient temperature and pressure for 18 h. Subsequently, the organic solvents are removed under reduced pressure and the aqueous slurry is diluted with saturated Na₂CO₃ (50 mL) and extracted with ethyl acetate (3×100 mL). The ethyl acetate fractions are pooled and washed with water (3×100 mL). The ethyl acetate is then treated with 1N HCl (100 mL). After extraction the aqueous phase is washed with CH₂Cl₂ (3×100 mL). The aqueous phase is concentrated and freeze-dried to provide 360 mg (11.5%) of the title compound as a tan solid. HRMS: calcd for C₆H₁₂N₄+H⁺, 141.11347; found (ESI, [M+H]+), 141.1243. IR 2100 cm−1.

### Example 349

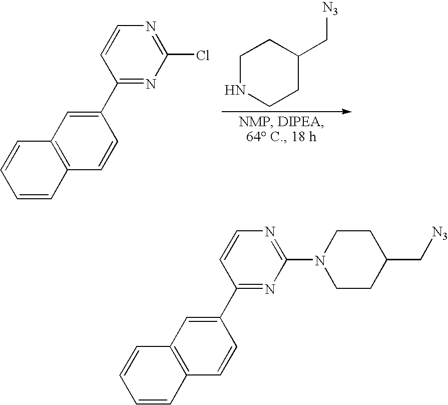

2-[4-(Azidomethyl)piperidin-1-yl]-4-(2-naphthyl)pyrimidine. A solution of 2-Chloro-4-(naphthalene-2-yl)pyrimidine (30 mg, 124.6 μMol), 4-(azidomethyl)piperidine (28.5 mg, 162 μMol), and diisopropylethylamine (65 μL, 374 μMol) in N-methylpyrrolidine (1 mL) is heated in a vial in a sheker block at 64° C. for 18 h. The reaction is cooled to room temperature and triethylamine (100 μL), water (300 μL) and methanol (500 μL) are added and the crude material is purified by RP-HPLC (Method D, without TFA modifier) to yield the tan solid (25.4 mg, 59.2%) after freeze-drying; HPLC (Method F): Purity 100%, Rt=12 min. HRMS: calcd for C₂₀H₂₀N₆+H+, 345.18222; found (ESI, [M+H]+), 345.1808.

### Example 350

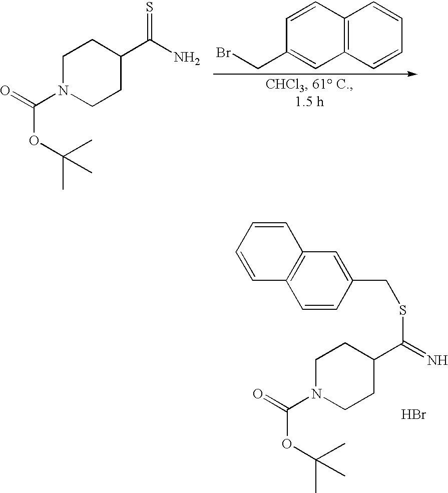

tert-Butyl 4-{imino[(2-naphthylmethyl)thio]methyl}piperidine-1-carboxylate. To solution of 4-thiocarbamoyl-piperidine-1-carboxylic acid tert-butyl ester (489 mg, 2 mMol) in chloroform (4 mL) is added 2-bromomethyl-naphthalene (442 mg, 2 mMol). The reaction is heated for 1.5 h at 64° C. following a literature procedure (Tetrahedron Lett. 1997, 38, 179-182). The solvent is evaporated and diethyl ether (30 mL) is added. The title compound precipitated as a white solid and is isolated by filtration (716 mg, 76.9%); HPLC (Method F): Purity 94.5%, Rt=10.1 min. HRMS: calcd for C₂₂H₂₈N₂O₂S+H+, 385.19442; found (ESI, [M+H]+), 385.1934.

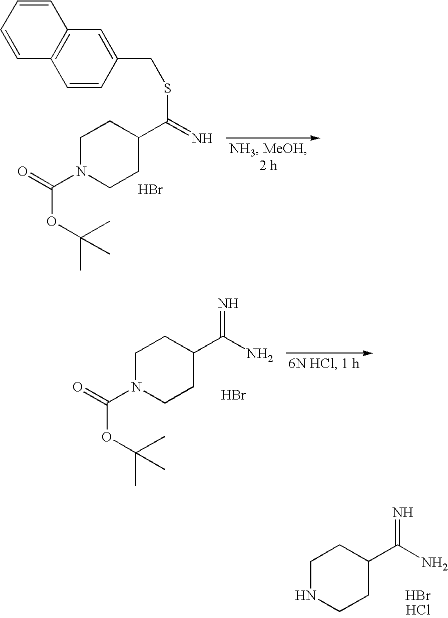

Piperidine-4-carboxamidine. A solution of tert-Butyl 4-{imino[(2-naphthylmethyl)thio]methyl}piperidine-1-carboxylate (93 mg, 200 μMol) in methanol (1 mL) at 0° C. is added ammonia (200 μL, 2 M solution in methanol, 400 μMol). After warming to room temperature over 2 h the reaction is concentrated. The crude is partitioned between water (30 mL) and diethyl ether (30 mL). The water layer is washed with diethyl ether (30 mL). The aqueous portion is concentrated and freeze-dried to provide the light pink solid (tert-butyl 4-[amino(imino)methyl]piperidine-1-carboxylate, (40 mg, 64.9%). HRMS: calcd for C₁₁H₂₁N₃O₂+H+, 228.17065; found (ESI, [M+H]+), 228.1714. tert-Butyl 4-[amino(imino)methyl]piperidine-1-carboxylate (190 mg, 617 μMol) is treated with 6N HCl (10 mL) for 1 h. The reaction is concentrated and dried under vacuum to provide piperidine-4-carboxamidine HBr HCl (150 mg, 100%), which is used without further purification.

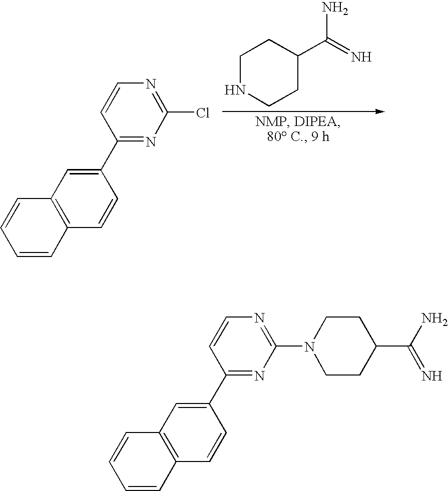

1-[4-(2-Naphthyl)pyrimidin-2-yl]piperidine-4-carboximidamide. A solution of 2-Chloro-4-(naphthalene-2-yl)pyrimidine (104 mg, 432 microMol), piperidine-4-carboxamidine HBr HCl (150 mg, 612 μMol), and diisopropylethylamine (522 μL, 3 mMol) in N-methylpyrrolidine (3 mL) is heated in a vial in a sheker block at 80° C. for 18 h. The reaction is cooled and TFA (500 μL) is added to eventually obtain one salt form at the amidine. The crude is diluted with saturated Na₂CO₃ (50 mL) and ethyl acetate (50 mL) and separated. The aqueous phase is re-extracted with ethyl acetate (2×50 mL). The combined organic extracts are washed with brine and dried over MgSO₄ and concentrated. The crude is diluted with water (300 μL) and methanol (500 μL) and is purified by RP-HPLC (Method D, without TFA modifier) to yield the white solid from clean fractions (12 mg, 6.2%) after freeze-drying (mono TFA salt by F¹⁹ NMR); HPLC (Method F): Purity 98.2%, Rt=8.6 min. HRMS: calcd for C₂₀H₂₁N₅+H+, 332.18697; found (ESI, [M+H]+), 332.1877

### Example 351

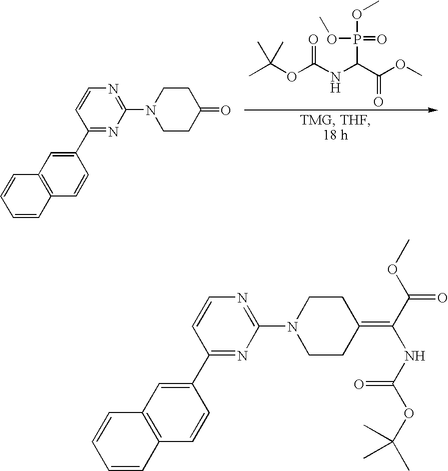

Methyl [(tert-butoxycarbonyl)amino]{1-[4-(2-naphthyl)pyrimidin-2-yl]piperidin-4-ylidene}acetate. To tert-butoxycarbonylamino-(dimethoxy-phosphoryl)-acetic acid methyl ester (102 mg, 343 μMol) in THF (1 mL) under nitrogen is added tetramethylguanidine (56 μL, 448 μMol). Stirring is continued for 15 min. at which time the ketone (448 μMol) in THF (1 mL) is added by syringe. After stirring for 18 h the reaction is concentrated. Crystallization in methanol afforded the title compound as an off-white solid (69.5 mg, 42.7%). HPLC (Method F) purity two peaks 47.8% and 52.2% (100% overall), rt=11.4 min and 11.5 min; HRMS: calcd for C27H30N404+H+, 475.23398; found (ESI, [M+H]+), 475.2344.

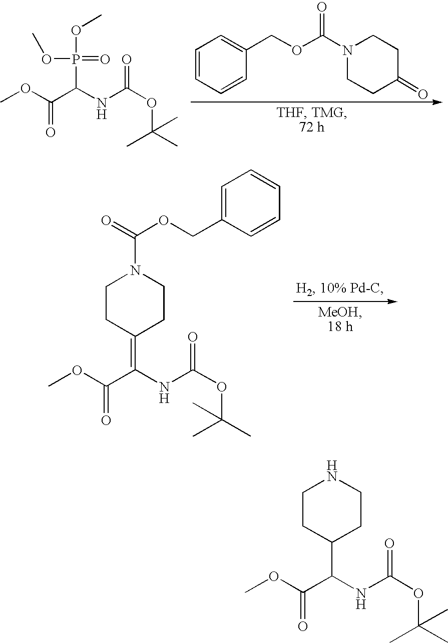

Methyl [(tert-butoxycarbonyl)amino](piperidin-4-yl)acetate. Prepared according to the literature procedure (Bioorganic & Medicinal Chemistry Letters, 1998, 8, 3409-3414). To a solution of tert-butoxycarbonylamino-(dimethoxy-phosphoryl)-acetic acid methyl ester (1.78 g, 6.0 mMol) in THF (4 mL) under nitrogen is added tetramethylguanidine (980 μL, 7.8 mMol). Stirring is continued for 15 min. at which time 4-oxo-piperidine-1-carboxylic acid benzyl ester (2.8 g, 12 mMol) in THF (3 mL) is added by syringe. After stirring for 72 h the reaction is concentrated. Recrystallization in ethyl acetate:hexane (1:4, 5 mL) provided 1.05 g (43.4%) of the white solid (benzyl 4-{1-[(tert-butoxycarbonyl)amino]-2-methoxy-2-oxoethylidene}piperidine-1-carboxylate). HPLC (Method F): Purity 100%, Rt=9.4 min. HRMS: calcd for C₂₁H₂₈N₂O₆+H+, 405.20201; found (ESI, [M+H]⁺), 405.2024. Benzyl 4-{1-[tert-butoxycarbonyl)amino]-2-methoxy-2-oxoethylidene}piperidine-1-carboxylate (669 mg, 1.65 mMol) is dissolved in methanol (10 mL). 10% Palladium on carbon (100 mg) is added under nitrogen and a hydrogen balloon is placed onto a 3-way stopcock over the reaction vessel. The reaction is purged under vacuum and filled with hydrogen. The purger-fill procedure is repeated two more times and the reaction is stirred under hydrogen overnight. The reaction is filtered through Celite and rinsed with methanol (3×30 mL). The combined filtrate is concentrated to give 450 mg (100%) of the pale oil, methyl [(tert-butoxycarbonyl)amino](piperidin-4-yl)acetate. GC/MS [M+H]⁺=272, Purity=100%, Rt=3.9 min.; HRMS: calcd for C₁₃H₂₄N₂O₄, 272.17361; found (EI, M⁺.), 272.1749.

### Example 352

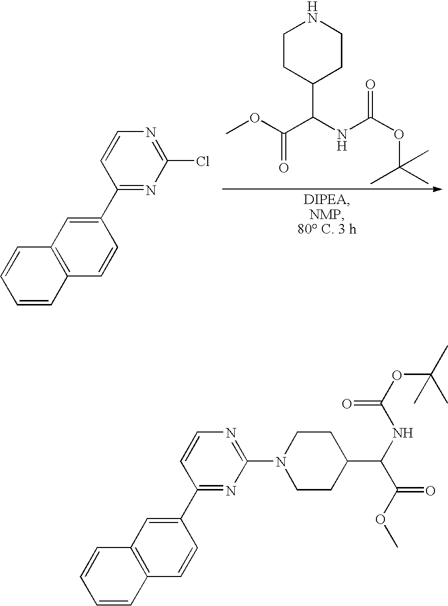

Methyl [(tert-butoxycarbonyl)amino]{-[4-(2-naphthyl)pyrimidin-2-yl]piperidin-4-yl}acetate. A mixture of 2-chloro-4-naphthalen-2-yl-pyrimidine (330 mg, 1.37 mMol) and [(tert-butoxycarbonyl)amino](piperidin-4-yl)acetate (410 mg, 1.5 mMol) in NMP (3 mL) with diisopropylethylamine (715 μL, 4.11 mMol) is heated at 80° C. for 3 hours. After cooling to room temperature the crude is partitioned between ethyl acetate (50 mL) and saturated NaHCO₃ (50 mL). The organic phase is washed with water (3×) and dried over MgSO₄ and concentrated. The crude material is purified by silica gel chromatography, eluting with 5% MeOH/CH₂Cl₂ (R_{f}=0.4), to afford the title compound as an off-white solid (465 mg, 71.2%). HPLC (Method F) purity 100%, rt=11.6 min; HRMS: calcd for C₂₇H₃₂N₄O₄+H⁺, 477.24963; found (ESI, [M+H]+), 477.2474. The racemate is separated by preparative chiral SFC described below to give the two enaniomers, methyl (2R)-[(tert-butoxycarbonyl)amino]{1-[4-(2-naphthyl)pyrimidin-2-yl]piperidin-4-yl}acetate, (peak 1, Rt=7.70 min.) and methyl (2S)-[(tert-butoxycarbonyl)amino]{1-[4-(2-naphthyl)pyrimidin-2-yl]piperidin-4-yl}acetate, (peak 2, Rt=9.59 min.).

Approximately 168 mg of racemic mixture, is dissolved in 10 mL of methanol/acetonitrile. 500 μL of the resulting solution is repetitively injected onto the Supercritical Fluid Chromatography instrument, and the baseline resolved enantiomers are separately collected using the conditions described below. The chiral purity of each enantiomer is determined under the same Supercritical Fluid Chromatography conditions using a Chiralpak AD-H 5 μm, 250 mm×4.6 mm ID column at 2.0 mL/min flow rate using Analytical Supercritical Fluid Chromatography (Berger Instruments, Inc. Newark, Del.). Both enantiomers are found to be >99.9% enantiomerically pure.

Enantiomer examples 353-354 were prepared from the chiral separation of example 352.

| Example | | | R.t | HPLC |
|---|---|---|---|---|
| no. | Compound | MS | (mins.) | Method |
| 353 | methyl (2R)-[(tert- | 477 | 11.6 | F |
| | butoxycarbonyl)amino]{1-[4-(2- | | | |
| | naphthyl)pyrimidin-2-yl]piperidin- | | | |
| | 4-yl}acetate | | | |
| 354 | methyl (2S)-[(tert- | 477 | 11.6 | F |
| | butoxycarbonyl)amino]{1-[4-(2- | | | |
| | naphthyl)pyrimidin-2-yl]piperidin- | | | |
| | 4-yl}acetate | | | |

### Example 355

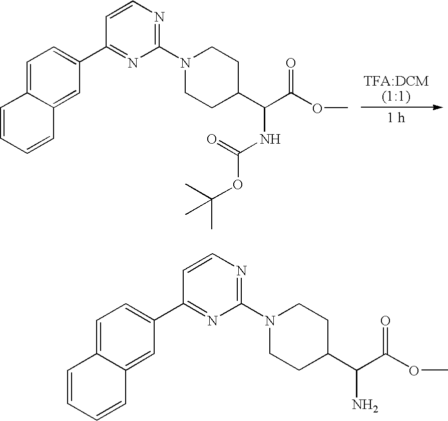

Methyl amino{1-[4-(2-naphthyl)pyrimidin-2-yl]piperidin-4-yl}acetate. Methyl [(tert-butoxycarbonyl)amino]{1-[4-(2-naphthyl)pyrimidin-2-yl]piperidin-4-yl}acetate (12 mg, 25.1 μMol) is dissolved in TFA:DCM (1:1, 5 mL) and stirred at room temperature for 1 h. The reaction is concentrated on a rotary evaporator at 60° C. Toluene is added (2 mL) and the evaporated on a rotary evaporator at 60° C. for 20 min. This is repeated (2×) and the resulting solid is freeze-dried for 2 days to provide the di-TFA salt as a light yellow solid (16 mg, 100%); HPLC (Method F): Purity 100%, Rt=8.9 min. HRMS: calcd for C₂₂H₂₄N₄O₂+H+, 377.19720; found (ESI, [M+H]+), 377.1971.

Additional enantiomer examples were prepared from examples 353 and 354 using the method for example 355.

| Example | | | R.t | HPLC |
|---|---|---|---|---|
| no. | Compound | MS | (mins.) | Method |
| 356 | methyl (2R)-amino{1-[4-(2- | 377 | 9 | F |
| | naphthyl)pyrimidin-2- | | | |
| | yl]piperidin-4-yl}acetate | | | |
| 357 | methyl (2S)-amino{1-[4-(2- | 377 | 8.9 | F |
| | naphthyl)pyrimidin-2- | | | |
| | yl]piperidin-4-yl}acetate | | | |

### Example 358

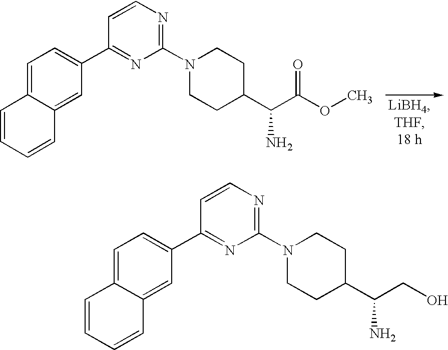

(2R)-2-Amino-2-{1-[4-(2-naphthyl)pyrimidin-2-yl]piperidin-4-yl}ethanol. To a solution of the free based methyl (2R)-amino{1-[4-(2-naphthyl)pyrimidin-2-yl]piperidin-4-yl}acetate (43 mg, 114 μMol) in THF (3 mL) is added LiBH₄ (114 μL, 2 M solution in THF, 228 μMol) at room temperature under nitrogen for 18 h. The crude is diluted with acetonitrile (2 mL), methanol (1 mL) and water (1 mL) and is purified by RP-HPLC (Method D, without TFA modifier) to yield the off-white solid (20 mg, 50.5%) after freeze-drying; HPLC (Method F): Purity 98.6%, Rt=8.3 min. HRMS: calcd for C₂₁H₂₄N₄O⁺H⁺, 349.20229; found (ESI, [M+H]⁺), 349.2009.

### Example 359

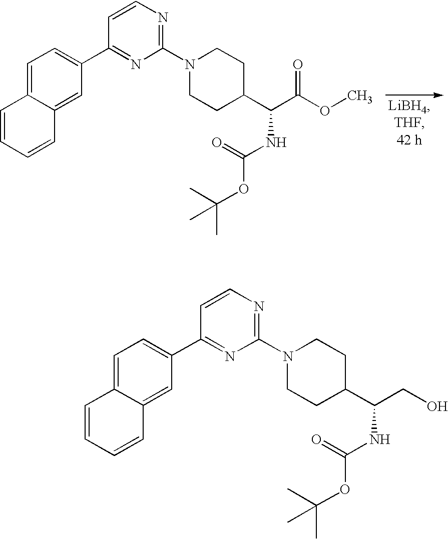

tert-Butyl ((1R)-2-hydroxy-1-{1-[4-(2-naphthyl)pyrimidin-2-yl]piperidin-4-yl}ethyl)carbamate. To a solution of methyl (2R)-[(tert-butoxycarbonyl)amino]{1-[4-(2-naphthyl)pyrimidin-2-yl]piperidin-4-yl}acetate (25.3 mg, 53 μMol) in THF (2 mL) is added LiBH₄ (53 μL, 2 M solution in THF, 106 μMol) at room temperature under nitrogen for 18 h. The reaction is ⅔ complete by LC/MS and another equivalent of LiBH₄ is added (3 equiv. total) with stirring continued overnight (42 h total). The reaction is diluted with water (3 mL) and concentrated. The crude is partitioned between ethyl acetate (25 mL) and saturated NaHCO₃ (25 mL). The organic phase is washed with brine and dried over MgSO₄ and concentrated. The crude material is analyzed by thin layer chromatography, eluting with 5% MeOH/CH₂Cl₂ (R_{f}=0.2), and concentrated without purification to afford the title compound as a white solid (23.2 mg, 97.5%); HPLC (Method F): Purity 100%, Rt=11 min. HRMS: calcd for C₂₆H₃₂N₄O₃+H+, 449.25472; found (ESI, [M+H]+), 449.2553.

### Example 360 was prepared according to the method for example 359.

| Example | | | R.t | HPLC |
|---|---|---|---|---|
| no. | Compound | MS | (mins.) | Method |
| 360 | tert-butyl ((1S)-2-hydroxy-1-{1-[4- | 449 | 11 | F |
| | (2-naphthyl)pyrimidin-2- | | | |
| | yl]piperidin-4-yl}ethyl)carbamate | | | |

### Example 361

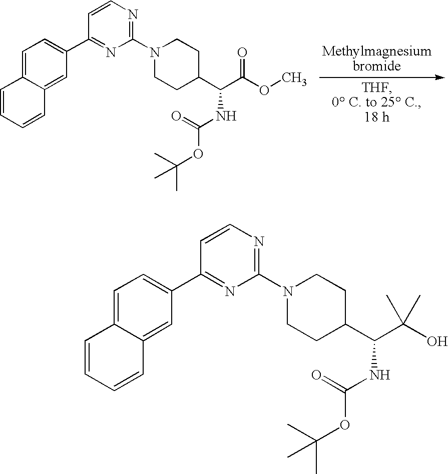

tert-Butyl ((1R)-2-hydroxy-2-methyl-1-{1-[4-(2-naphthyl)pyrimidin-2-yl]piperidin-4-yl}propyl)carbamate. To a solution of methyl (2R)-[(tert-butoxycarbonyl)amino]{1-[4-(2-naphthyl)pyrimidin-2-yl]piperidin-4-yl}acetate (180 mg, 378 μMol) in THF (10 mL) at 0° C. under nitrogen is added methylmagnesium bromide (630 μL, 3 M in diethyl ether, 1.89 mMol). The reaction is warmed to room temperature overnight and poured into saturated ammonium chloride (50 mL). Ethyl acetate is added and the layers are separated. The organic layer is washed with brine (70 mL), dried over magnesium sulfate and concentrated. The crude is diluted with methanol (3 mL) and water (1 mL) and is purified by RP-HPLC (Method D, without TFA modifier) to yield the white solid (112.1 mg, 62.3%) after freeze-drying; HPLC (Method F): Purity 100%, Rt=11.4 min. HRMS: calcd for C₂₈H₃₆N₄O₃+H⁺, 477.28602; found (ESI, [M+H]+), 477.286.

### Example 362

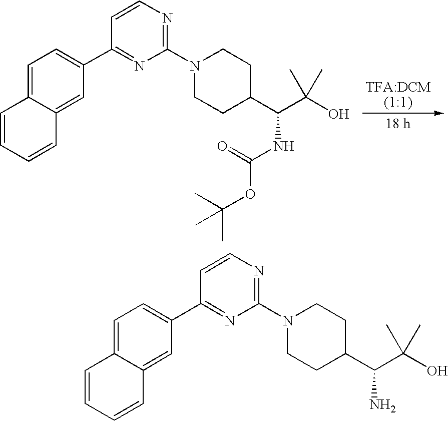

(1R)-1-amino-2-methyl-1-{1-[4-(2-naphthyl)pyrimidin-2-yl]piperidin-4-yl}propan-2-ol. tert-Butyl ((1R)-2-hydroxy-2-methyl-1-{1-[4-(2-naphthyl)pyrimidin-2-yl]piperidin-4-yl}propyl)carbamate (28 mg, 58.7 μMol) is dissolved in TFA:DCM (1:1, 3 mL) and stirred at room temperature for 18 h. The reaction is concentrated on a rotary evaporator at 60° C. Toluene is added (2 mL) and the evaporated on a rotary evaporator at 60° C. for 20 min. This is repeated (2×) and the resulting solid is freeze-dried for 2 days to provide the di-TFA salt as a tan solid (35.2 mg, 99.2%); HPLC (Method F): Purity 96%, Rt=8.8 min. HRMS: calcd for C₂₃H₂₈N₄O⁺H⁺, 377.23359; found (ESI, [M+H]+), 377.2339.

### Example 363

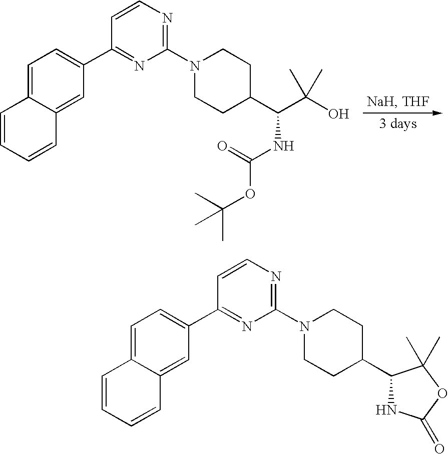

(4R)-5,5-dimethyl-4-{1-[4-(2-naphthyl)pyrimidin-2-yl]piperidin-4-yl}-1,3-oxazolidin-2-one. Following a literature procedure (Synthetic Communications, 2003, 33, 2907-2916), tert-butyl ((1R)-2-hydroxy-2-methyl-1-{1-[4-(2-naphthyl)pyrimidin-2-yl]piperidin-4-yl}propyl)carbamate (48 mg, 100 μMol) is dissolved in THF (1.3 mL) under nitrogen. Sodium hydride (7.0 mg, 60% dispersion in mineral oil, 100 μMol) is added and the reaction is stirred for 3 days under nitrogen. The reaction is diluted with DCM (0.5 mL) and concentrated. The crude is taken up in half-saturated brine (20 mL) and DCM (30 mL) and the layers are separated. The organic layer is dried over magnesium sulfate and concentrated to provide as an off-white solid (34.7 mg, 86.2%); HPLC (Method F): Purity 100%, Rt=10.6 min. [M+H]⁺403.1.

Example 364 was prepared according to the method for example 363.

| Example | | | R.t | HPLC |
|---|---|---|---|---|
| no. | Compound | MS | (mins.) | Method |
| 364 | (4R)-4-{1-[4-(2- | 375 | 10 | F |
| | naphthyl)pyrimidin-2-yl]piperidin- | | | |
| | 4-yl}-1,3-oxazolidin-2-one | | | |

### Example 591

### Preparation of (4-(Aminomethyl)piperidin-1-yl)(4-(naphthalene-2-yl)pyrimidin-2-yl)methanone

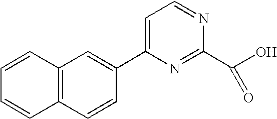

4-(Naphthalen-2-yl)pyrimidine-2-carboxylic acid: 4-(Naphthalen-2-yl)pyrimidine-2-carbaldehyde (500 mg, 2.16 mmol) was dissolved in MeOH (10 mL) and treated with a NaOCl/water solution (5 ml of 5% NaOCl/water). This reaction mixture was allowed to stir for 16 h. The solvent was removed under vacuum and the crude was partitioned between ethyl acetate and bicarbonate solution. The ethyl acetate was extracted twice with bicarbonate, and the combined bicarbonate fractions were washed one additional time with fresh ethyl acetate. The combine bicarbonate fractions were acidified with conc. HCl and extracted three times with ethyl acetate. The combined ethyl acetate fractions were dried over MgSO₄ to yield the title product (80 mg (14%), 0.31 mmol). MS (ES) m/z 249.2 ([M−H]⁻. HPLC 92.4% at 210-370 nm, RT=4.6 min.

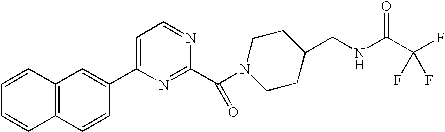

2,2,2-Trifluoro-N-((1-(4-(naphthalene-2-yl)pyrimidine-2-carbonyl)piperidin-4-yl)methyl)acetamide: 4-(Naphthalen-2-yl)pyrimidine-2-carboxylic acid (40 mg, 0.16 mmol) was dissolved in NMP (2 ml). DIEA (41 mg, 0.32 mmol) was added, followed by HATU (122 mg, 0.32 mmol). The reaction mixture was allowed to stir for 16 h at room temperature. The reaction mix was partitioned between ethyl acetate and brine. The ethyl acetate was washed three times with brine and the solvent was removed under vacuum onto Celite™ and chromatographed on silica with an gradient elution of 10% to 100% ethyl acetate/hexane to yield the title product (24 mg (35%), 0.054 mmol). MS (ES) m/z 443.1 ([M+H]⁺. Open access: about 95% at 254 nm RT=1.74 min.

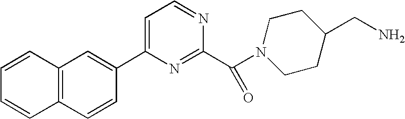

(4-(Aminomethyl)piperidin-1-yl)(4-(naphthalene-2-yl)pyrimidin-2-yl)methanone: 2,2,2-Trifluoro-N-((1-(4-(naphthalene-2-yl)pyrimidine-2-carbonyl)piperidin-4-yl)methyl)acetamide (16 mg, 0.036 mmol) was dissolved in MeOH (5 ml). Sodium carbonate (300 mg, 2.83 mmol) dissolved in water (1 ml) was added. The reaction mixture was heated to 45° C. for sixteen hours. The mixture was partitioned between ethyl acetate and water. The ethyl acetate was washed two times with water, one time with brine and the combined ethyl acetate fractions were dried over MgSO₄ to yield the title product (8 mg (64%), 0.023 mmol). MS (ES) m/z 347.1 ([M+H]⁺. Open access: about 95% at 254 nm RT=1.53 min.

### Example 592

### Preparation of (3aR,7aS)-5-[4-(2-Naphthyl)pyrimidin-2-yl]octahydro-1H-pyrrolo[3,4-c]pyridine

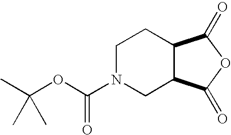

(cis)-tert-Butyl 1,3-dioxohexahydrofuro[3,4-c]pyridine-5(1H)-carboxylate. Following the literature procedure (C-B. Xue et al. Bioorganic Medicinal Chemistry Letters 14 (2004) 4453-4459) reduction of 3,4-pyridinedicarboxylic acid was performed by hydrogenation in 1 N HCl using platinum oxide as the catalyst. Treatment with di-tert-butyl dicarbonate provided N-Boc-cis-3,4-piperidinedicarboxylic acid. This dicarboxylic acid was treated with acetic anhydride in THF at 60° C. and subsequently concentrated under reduced pressure providing the titled compound as a mixture of the cis-enantiomers, which was used without further purification.

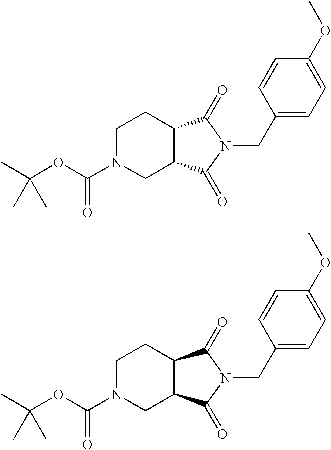

tert-Butyl (cis)-2-(4-methoxybenzyl)-1,3-dioxooctahydro-5H-pyrrolo[3,4-c]pyridine-5-carboxylate: To (cis)-tert-butyl 1,3-dioxohexahydrofuro[3,4-c]pyridine-5(1H)-carboxylate (6.23 g, 24.4 mMol) was added 4-methoxy-benzylamine (12.7 mL, 97.6 mMol) followed by toluene (15 mL) under nitrogen. After heating at 80° C. for 24 h the reaction was concentrated and purified by flash chromatography eluting with ethyl acetate: hexane (1:1) to yield the titled compound, a white solid (4.10 g, 45%). MS (ESI) m/z 274.9 ([M+H-tBoc]⁺); HPLC (Method F): Purity=100%, Rt=9.6 mins.

Chiral Separation: Approximately 3.58 g of racemic mixture was dissolved in 30 mL of methanol/acetonitrile. 100 μL of the resulting solution was repetitively injected onto the Supercritical Fluid Chromatography instrument, and the baseline resolved enantiomers were separately collected using the conditions described below. The chiral purity of each enantiomer was determined under the same Supercritical Fluid Chromatography conditions using a Chiralpak AD-H 5 μm, 250 mm×4.6 mm ID column at 2.0 mL/min flow rate using Analytical Supercritical Fluid Chromatography (Berger Instruments, Inc. Newark, Del.). Enantiomer 1 (t_{R}=12.5 min) was found to be >99.9% enantiomerically pure. Enantiomer 2 (t_{R}=13.8 min) was found to be >98.1% enantiomerically pure.

| | |
|---|---|
| SFC Instrument: | Berger MultiGram Prep SFC (Berger Instruments, |
| | Inc. Newark, DE) |
| Column: | Chiralpak AD-H; 5 μm; 250 mm L × 20 mm ID |
| | (Chiral Technologies, Inc, Exton, PA) |
| Column temperature: | 35° C. |
| SFC Modifier: | 10% MeOH/90% CO2 |
| Flow rate: | 50 mL/min |
| Outlet Pressure: | 100 bar |

tert-Butyl (3aS,7aS)-2-(4-methoxybenzyl)-1,3-dioxooctahydro-5H-pyrrolo[3,4-c]pyridine-5-carboxylate Concentration of the pooled fractions (enantiomer 1) from chiral SFC provided the titled compound (1.65 g) as a tan solid. MS: ([M+H-tBoc]⁺)=275.2. HPLC (Method F): Purity=96.9%, Rt=8.7 mins.

tert-Butyl (3aR,7aR)-2-(4-methoxybenzyl)-1,3-dioxooctahydro-5H-pyrrolo[3,4-c]pyridine-5-carboxylate Concentration of the pooled fractions (enantiomer 2) from chiral SFC provided the titled compound (1.63 g) as a tan solid. MS: ([M+H-tBoc]⁺)=275.1. HPLC (Method F): Purity=98.1%, Rt=8.7 mins.

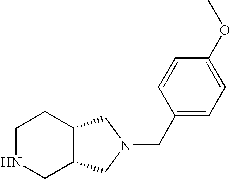

(3aS,7aS)-2-(4-Methoxybenzyl)octahydro-1H-pyrrolo[3,4-c]pyridine A solution of tert-butyl (3aS,7aS)-2-(4-methoxybenzyl)-1,3-dioxooctahydro-5H-pyrrolo[3,4-c]pyridine-5-carboxylate (1.08 g, 2.87 mMol) in dry THF (100 mL) under nitrogen was cooled at 0° C. with stirring. A solution of borane in THF (1 M solution, 28.7 mL, 28.7 mMol) was added slowly over 10 min. After stirring for 15 min at 0° C. the reaction temperature was gradually raised to 65° C. and stirred at this temperature for 18 h. The reaction was then cooled to 0° C. and slowly added to a 1000 ML flask filled halfway with crushed ice. After 2 h the THF was removed under reduced pressure and the aqueous solution was extracted with ethyl acetate (200 mL). The ethyl acetate layer was washed with brine, dried over MgSO₄, filtered and concentrated. The resulting crude oil was treated with 6 N HCl (10 mL) and heated at 70° C. for 1 h. The solvent was removed and the resulting product was lyophilized for 2 days providing the titled compound, a pale oil as the di-hydrochloride salt (1.09 g), which was used without further purification. MS (ESI) m/z 247.2 ([M+H]⁺); HPLC (Method G): Purity=96.7%, Rt=7.3 mins.; HRMS: calcd for C₁₅H₂₂N₂O+H+, 247.1805; found (ESI, [M+H]⁺), 247.1792.

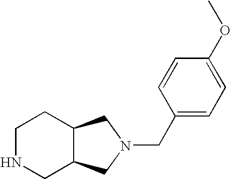

(3aR,7aR)-2-(4-Methoxybenzyl)octahydro-1H-pyrrolo[3,4-c]pyridine In an analogous manner used to prepare (3aS,7aS)-2-(4-methoxybenzyl)octahydro-1H-pyrrolo[3,4-c]pyridine, the titled compound was prepared from tert-butyl (3aR,7aR)-2-(4-methoxybenzyl)-1,3-dioxooctahydro-5H-pyrrolo[3,4-c]pyridine-5-carboxylate, as a light yellow solid. MS (ESI) m/z 247.2 ([M+H]⁺); HPLC (Method F): Purity=100%, Rt=1.9 mins.

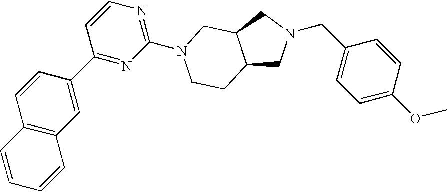

(3aR,7aS)-2-(4-Methoxybenzyl)-5-[4-(2-naphthyl)pyrimidin-2-yl]octahydro-1H-pyrrolo[3,4-c]pyridine A solution of (3aS,7aS)-2-(4-methoxybenzyl)octahydro-1H-pyrrolo[3,4-c]pyridine di-hydrochloride (86 mg, 270 μMol), 2-chloro-4-naphthalen-2-yl-pyrimidine (65 mg, 270 μMol) and diisopropylethylamine (235 μL, 1.34 mMol) in NMP (1 mL) was heated at 80° C. for 3 days. After cooling the crude was diluted with methanol (0.5 mL) and water (0.3 mL) and purified by RP-HPLC (Method D, without TFA modifier) to yield the titled compound as a tan solid (34.7 mg, 28.7%); HPLC (Method F): Purity=99.1%, Rt=9.8 mins. MS (ESI) m/z 451.2 ([M+H]⁺); HRMS: calcd for C₂₉H₃₀N₄O+H+, 451.2492; found (ESI, [M+H]⁺), 451.2502.

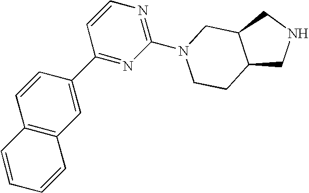

(3aR,7aS)-5-[4-(2-Naphthyl)pyrimidin-2-yl]octahydro-1H-pyrrolo[3,4-c]pyridine (3aR,7aS)-2-(4-Methoxybenzyl)-5-[4-(2-naphthyl)pyrimidin-2-yl]octahydro-1H-pyrrolo[3,4-c]pyridine (70 mg, 155 μMol) was dissolved in CH₂Cl₂ (2 mL) and stirred at 0° C. under nitrogen. 1-Chloroethyl chloroformate (22 μL, 201 μMol) was added and after 20 min. the solvent was removed under reduced pressure. The crude material was dissolved in methanol (5 mL) and heated to 65° C. for 15 min. and then concentrated under reduced pressure. The crude was diluted with acetonitrile (0.5 mL), methanol (1.5 mL) DMSO (0.3 mL) and water (0.1 mL) and purified by RP-HPLC (Method D, without TFA modifier) to yield the desired tan product as the hydrochloride salt (11.0 mg, 19.4%); HPLC (Method G): Purity=90%, Rt=10.1 mins. MS (ESI) m/z 331.1 ([M+H]⁺); HRMS: calcd for C₂₁H₂₂N₄+H+, 331.1918; found (ESI, [M+H]⁺), 331.1914.

### Biological Evaluation—Functional Dkk1-LRP5-TCF-Luciferase Assay in U2OS Cells

Frozen U2OS-Dkk1-HTS Reporter cells generation: U2OS Human Bone derived cells (Osteosarcoma) are grown in McCoy's 5A Medium (Modified), with L -glutamine (GIBCO Cat No. 16600-082)+1% Pen-Strep+5% FBS) plated at 1×107 cells/T175 cm flask. The next day, the cells are co-transfected overnight with the following plasmids: (a) Test reporter (16×TCF-TK-FireFly-Luci), (b) Internal Control Reporter (TK-Renilla-Luci), (c) Wnt3a and (d) Dkk1. GIBCO's Lipofectamine 2000 and OptiMEM are used for the transfection. After a minimum of 4 hr of transfection at 37° C., the plasmid-transfected cells are trypsinized, counted, and suspended in freezing medium (95% FBS+5% DMSO). The reporter cells are frozen at 1×107/ml concentrations, aliquoted into 0.5 ml or 2.5 ml/tube and stored at 70° C.

The following day, test compounds are added under HTS setup by Plate Track into 384 well plates (white, TC treated, Falcon plate) such that the final concentration of the compounds in 20 μL/well cell will be 5 μg/ml (final concentration of DMSO=0.25% and final compound concentration=20 μM). Vials of frozen reporter cells are thawed by warming the vial in a 37° C. water bath for 60-120 seconds with some shaking until the cells formed a suspension. The thawed cells are transferred into a cold 50 ml (or larger) tube and mixed well by gentle pipetting. The appropriate amount of cold Phenol Red Free RPMI medium-1640 (GIBCO, Cat #11835-030) with L-glutamine is added, both with ˜5% FBS (GIBCO-BRL, Cat. #16000-044), so that 20 μl of the final cell suspension will contain ˜5,000 cells. The cell dilution is done such that the final concentration of FBS is ˜5%.

Diluted cells (20 μl) are added into each well in a 384 well plate. The plate is incubated at 37° C. under 5% CO₂ for ˜20 h. Bright-Glo substrate, 2.5 μl/well is added, and the Fire Fly Luciferase is measured using VLUX (60 second exposure) immediately after the substrate is added. Test compounds are dissolved in DMSO (100%) and added to specified wells. Raw luciferase signal data obtained as relative luminescence units (RLUs) for the test compounds are normalized to the signal of the mean of the sample reporter cell plate with DMSO.

Active compounds have TCF-luciferase ratios of 2.5 fold or greater over DMSO. All compounds show a signal increase of at least 10% compared to a signal with only DMSO added. Results from the above biologic procedures of example compounds are shown in the following table:

| | EC50 | | | |
|---|---|---|---|---|
| Compound | (μm) | A | B | C |
| N-(2-morpholin-4-ylethyl)-4-(2-naphthyl)pyrimidin-2-amine | 5.700 | 1.853 | 2.110 | 2.445 |
| N-(1-acetylpiperidin-4-yl)-4-(2-naphthyl)pyrimidin-2-amine | 5.500 | 2.428 | 1.093 | 2.527 |
| N,N-dimethyl-4-{[4-(2-naphthyl)pyrimidin-2-yl]amino}piperidine-1-carboxamide | 0.640 | 1.810 | 1.133 | 1.583 |
| N,N-diethyl-4-{[4-(2-naphthyl)pyrimidin-2-yl]amino}piperidine-1-carboxamide | | | 1.650 | |
| 4-(2-naphthyl)-N-[1-(pyrrolidin-1-ylcarbonyl)piperidin-4-yl]pyrimidin-2-amine | | | 1.060 | 0.960 |
| N-[1-(morpholin-4-ylcarbonyl)piperidin-4-yl]-4-(2-naphthyl)pyrimidin-2-amine | | | 1.030 | 1.070 |
| N-[1-(methylsulfonyl)piperidin-4-yl]-4-(2-naphthyl)pyrimidin-2-amine | 0.060 | 1.730 | 2.240 | 1.540 |
| N,N-dimethyl-4-{[4-(2-naphthyl)pyrimidin-2-yl]amino}piperidine-1-sulfonamide | 11.960 | 3.210 | 0.970 | 1.750 |
| 4-{[4-(2-naphthyl)pyrimidin-2-yl]amino}piperidine-1-carboxamide | 5.283 | 3.552 | 1.313 | 3.173 |
| N-ethyl-4-{[4-(2-naphthyl)pyrimidin-2-yl]amino}piperidine-1-carboxamide | 18.220 | 2.280 | 1.140 | 1.600 |
| N-isopropyl-4-{[4-(2-naphthyl)pyrimidin-2-yl]amino}piperidine-1-carboxamide | | | 1.070 | 1.370 |
| N-cyclohexyl-4-{[4-(2-naphthyl)pyrimidin-2-yl]amino}piperidine-1-carboxamide | | | | 1.450 |
| N-(1-benzoylpiperidin-4-yl)-4-(2-naphthyl)pyrimidin-2-amine | | 1.490 | 1.950 | 0.010 |
| N-[(1-acetylpiperidin-4-yl)methyl]-4-(2-naphthyl)pyrimidin-2-amine | | | 1.020 | 1.327 |
| N,N-dimethyl-4-({[4-(2-naphthyl)pyrimidin-2-yl]amino}methyl)piperidine-1-carboxamide | | | 0.870 | 1.237 |
| N,N-diethyl-4-({[4-(2-naphthyl)pyrimidin-2-yl]amino}methyl)piperidine-1-carboxamide | | | 1.220 | 1.040 |
| 4-(2-naphthyl)-N-{[1-(pyrrolidin-1-ylcarbonyl)piperidin-4-yl]methyl}pyrimidin-2-amine | | | 1.080 | 0.907 |
| N-{[1-(methylsulfonyl)piperidin-4-yl]methyl}-4-(2-naphthyl)pyrimidin-2-amine | | | 1.540 | 1.350 |
| N,N-dimethyl-4-({[4-(2-naphthyl)pyrimidin-2-yl]amino}methyl)piperidine-1-sulfonamide | | | | 1.210 |
| 4-({[4-(2-naphthyl)pyrimidin-2-yl]amino}methyl)piperidine-1-carboxamide | | | 0.880 | 1.583 |
| N-ethyl-4-({[4-(2-naphthyl)pyrimidin-2-yl]amino}methyl)piperidine-1-carboxamide | | | 1.350 | 1.303 |
| N-isopropyl-4-({[4-(2-naphthyl)pyrimidin-2-yl]amino}methyl)piperidine-1-carboxamide | | | 1.315 | 0.540 |
| N-cyclohexyl-4-({[4-(2-naphthyl)pyrimidin-2-yl]amino}methyl)piperidine-1-carboxamide | | 1.230 | 1.810 | 0.067 |
| N-[(1-benzoylpiperidin-4-yl)methyl]-4-(2-naphthyl)pyrimidin-2-amine | | | 1.440 | 0.627 |
| N-[(1-ethylpiperidin-4-yl)methyl]-4-(2-naphthyl)pyrimidin-2-amine | 3.940 | 3.450 | 1.500 | 0.908 |
| N-[(1-benzylpiperidin-4-yl)methyl]-4-(2-naphthyl)pyrimidin-2-amine | | | | 1.490 |
| 4-(2-naphthyl)-N-{[1-(phenylacetyl)piperidin-4-yl]methyl}pyrimidin-2-amine | | | 1.030 | 1.870 |
| N-({1-[(4-methylphenyl)sulfonyl]piperidin-4-yl}methyl)-4-(2-naphthyl)pyrimidin-2-amine | | | 1.170 | 0.920 |
| N-ethyl-4-[4-(2-naphthyl)pyrimidin-2-yl]-1,4-diazepane-1-carboxamide | 12.700 | 1.640 | | 2.580 |
| methyl 4-[4-(2-naphthyl)pyrimidin-2-yl]-1,4-diazepane-1-carboxylate | 30.000 | 1.930 | | 1.880 |
| ethyl 4-[4-(2-naphthyl)pyrimidin-2-yl]-1,4-diazepane-1-carboxylate | 30.000 | 1.790 | | 2.490 |
| 1-acetyl-4-[4-(2-naphthyl)pyrimidin-2-yl]-1,4-diazepane | 7.300 | 1.757 | 1.460 | 3.205 |
| 1-[4-(2-naphthyl)pyrimidin-2-yl]-4-propionyl-1,4-diazepane | 28.500 | 2.260 | | 2.230 |
| 1-[4-(2-naphthyl)pyrimidin-2-yl]-4-(trifluoroacetyl)-1,4-diazepane | | | | 1.235 |
| N,N-diethyl-4-[4-(2-naphthyl)pyrimidin-2-yl]-1,4-diazepane-1-carboxamide | 30.000 | 3.070 | | 1.490 |
| 1-(methylsulfonyl)-4-[4-(2-naphthyl)pyrimidin-2-yl]-1,4-diazepane | 5.610 | 1.510 | | 1.590 |
| N,N-dimethyl-4-[4-(2-naphthyl)pyrimidin-2-yl]-1,4-diazepane-1-sulfonamide | | | | 0.630 |
| 2-{methyl[4-(2-naphthyl)pyrimidin-2-yl]amino}ethanol | | | | |
| {(2S)-1-[4-(2-naphthyl)pyrimidin-2-yl]pyrrolidin-2-yl}methanol | | | 2.050 | 2.015 |
| 1-[4-(2-naphthyl)pyrimidin-2-yl]pyrrolidin-3-ol | | | | |
| 4-[4-(2-naphthyl)pyrimidin-2-yl]morpholine | | | | |
| {1-[4-(2-naphthyl)pyrimidin-2-yl]piperidin-2-yl}methanol | | | | |
| {1-[4-(2-naphthyl)pyrimidin-2-yl]piperidin-3-yl}methanol | | | | |
| 1-[4-(2-naphthyl)pyrimidin-2-yl]piperidin-4-ol | 1.285 | 2.224 | 1.350 | 0.280 |
| N-(2-methoxyethyl)-4-(2-naphthyl)pyrimidin-2-amine | | | | |
| 2-{[4-(2-naphthyl)pyrimidin-2-yl]amino}ethanol | | | | |
| 1-[4-(2-naphthyl)pyrimidin-2-yl]piperidin-3-ol | | | | |
| {(2R)-1-[4-(2-naphthyl)pyrimidin-2-yl]pyrrolidin-2-yl}methanol | | | 1.850 | 1.780 |
| N-(2-methoxyethyl)-N-methyl-4-(2-naphthyl)pyrimidin-2-amine | | | | |
| 1-[4-(2-naphthyl)pyrimidin-2-yl]piperidine-4-carbothioamide | | 2.070 | 1.740 | 1.990 |
| 1-[4-(2-naphthyl)pyrimidin-2-yl]piperidine-4-carbonitrile | 1.075 | 5.015 | 2.790 | 1.293 |
| N,N-dimethyl-1-{1-[4-(2-naphthyl)pyrimidin-2-yl]piperidin-4-yl}methanamine | | | 1.850 | |
| 2-[4-(1H-imidazol-1-ylmethyl)piperidin-1-yl]-4-(2-naphthyl)pyrimidine | 11.680 | 4.630 | 1.130 | 3.340 |
| 4-(2-naphthyl)-2-[4-(pyrrolidin-1-ylmethyl)piperidin-1-yl]pyrimidine | 3.178 | 3.393 | 2.070 | 0.707 |
| N-ethyl-N-({1-[4-(2-naphthyl)pyrimidin-2-yl]piperidin-4-yl}methyl)ethanamine | | | 1.140 | 0.020 |
| 4-(2-naphthyl)-2-[4-(piperidin-1-ylmethyl)piperidin-1-yl]pyrimidine | 8.587 | 3.290 | 1.093 | 1.687 |
| N-methyl-1-{1-[4-(2-naphthyl)pyrimidin-2-yl]piperidin-4-yl}methanamine | | 2.840 | 1.490 | 0.010 |
| N-({1-[4-(2-naphthyl)pyrimidin-2-yl]piperidin-4-yl}methyl)ethanamine | 3.300 | 4.200 | 1.330 | 0.010 |
| 2-methyl-N-({1-[4-(2-naphthyl)pyrimidin-2-yl]piperidin-4-yl}methyl)propan-1-amine | | 1.480 | 1.400 | 0.140 |
| N,N-dimethyl-4-[4-(2-naphthyl)pyrimidin-2-yl]-1,4-diazepane-1-carboxamide | | 1.520 | 1.200 | 2.260 |
| 4-[4-(2-naphthyl)pyrimidin-2-yl]-1,4-diazepane-1-carboxamide | 0.320 | 2.050 | 1.500 | 1.790 |
| 1-benzoyl-4-[4-(2-naphthyl)pyrimidin-2-yl]-1,4-diazepane | | | 1.060 | 0.350 |
| 1-[(4-methylphenyl)sulfonyl]-4-[4-(2-naphthyl)pyrimidin-2-yl]-1,4-diazepane | | | 1.760 | |
| N-methyl-4-(2-naphthyl)pyrimidin-2-amine | | | 1.340 | |
| N,N-diethyl-4-(2-naphthyl)pyrimidin-2-amine | | 1.260 | 1.750 | 1.680 |
| 4-(2-naphthyl)-N-propylpyrimidin-2-amine | | | 1.280 | |
| N-butyl-4-(2-naphthyl)pyrimidin-2-amine | | | 1.510 | |
| N-isopropyl-4-(2-naphthyl)pyrimidin-2-amine | | | 1.640 | |
| N-(sec-butyl)-4-(2-naphthyl)pyrimidin-2-amine | | | 1.010 | 1.340 |
| N-isobutyl-4-(2-naphthyl)pyrimidin-2-amine | | | 1.770 | |
| N-(tert-butyl)-4-(2-naphthyl)pyrimidin-2-amine | | | 1.750 | |
| N-benzyl-4-(2-naphthyl)pyrimidin-2-amine | | | 1.710 | |
| 4-(2-naphthyl)-N-(2-phenylethyl)pyrimidin-2-amine | | | 1.910 | |
| N-cyclopentyl-4-(2-naphthyl)pyrimidin-2-amine | | | 1.950 | 0.830 |
| N-cyclohexyl-4-(2-naphthyl)pyrimidin-2-amine | | 1.230 | 1.210 | 1.510 |
| 4-(2-naphthyl)-2-pyrrolidin-1-ylpyrimidine | | | 1.970 | |
| 4-(2-naphthyl)-2-piperidin-1-ylpyrimidine | | | 0.920 | 1.790 |
| 2-(4-methylpiperidin-1-yl)-4-(2-naphthyl)pyrimidine | | | 1.510 | |
| 1-[4-(2-naphthyl)pyrimidin-2-yl]azepane | | | 1.540 | |
| N-{1-[4-(2-naphthyl)pyrimidin-2-yl]azepan-4-yl}acetamide | | 1.350 | 1.260 | 2.910 |
| 2,2,2-trifluoro-N-{1-[4-(2-naphthyl)pyrimidin-2-yl]azepan-4-yl}acetamide | | | 1.100 | 1.870 |
| N,N-dimethyl-N′-{1-[4-(2-naphthyl)pyrimidin-2-yl]azepan-4-yl}urea | | | 1.740 | 1.750 |
| N-{1-[4-(2-naphthyl)pyrimidin-2-yl]azepan-4-yl}benzamide | | | 1.230 | 1.070 |
| N-{1-[4-(2-naphthyl)pyrimidin-2-yl]azepan-4-yl}urea | 20.360 | 1.500 | 1.620 | 2.890 |
| N-ethyl-N′-{1-[4-(2-naphthyl)pyrimidin-2-yl]azepan-4-yl}urea | | 1.320 | 2.330 | 2.560 |
| N-{1-[4-(2-naphthyl)pyrimidin-2-yl]azepan-4-yl}methanesulfonamide | | 1.120 | 2.040 | 2.210 |
| 4-methyl-N-{1-[4-(2-naphthyl)pyrimidin-2-yl]azepan-4-yl}benzenesulfonamide | | 0.780 | 1.490 | 2.260 |
| N,N-dimethyl-N′-{1-[4-(2-naphthyl)pyrimidin-2-yl]azepan-4-yl}sulfamide | | | 1.120 | 1.010 |
| N,N-dimethyl-N′-(2-{4-[4-(2-naphthyl)pyrimidin-2-yl]piperazin-1-yl}ethyl)urea | | | 1.980 | |
| N-(2-{4-[4-(2-naphthyl)pyrimidin-2-yl]piperazin-1-yl}ethyl)methanesulfonamide | 5.400 | 1.700 | 1.310 | 1.930 |
| N-(2-{4-[4-(2-naphthyl)pyrimidin-2-yl]piperazin-1-yl}ethyl)dicarbonimidic diamide | | | 1.220 | 0.490 |
| N-ethyl-N′-(2-{4-[4-(2-naphthyl)pyrimidin-2-yl]piperazin-1-yl}ethyl)urea | | | 1.090 | 0.280 |
| N-isopropyl-N′-(2-{4-[4-(2-naphthyl)pyrimidin-2-yl]piperazin-1-yl}ethyl)urea | | | 1.060 | 0.100 |
| diethyl (2-{4-[4-(2-naphthyl)pyrimidin-2-yl]piperazin-1-yl}ethyl)imidodicarbonate | | | 1.720 | |
| N-{(3R)-1-[4-(2-naphthyl)pyrimidin-2-yl]pyrrolidin-3-yl}acetamide | | 1.610 | 1.620 | 0.200 |
| 2,2,2-trifluoro-N-{(3R)-1-[4-(2-naphthyl)pyrimidin-2-yl]pyrrolidin-3-yl}acetamide | | | 1.970 | |
| methyl {(3R)-1-[4-(2-naphthyl)pyrimidin-2-yl]pyrrolidin-3-yl}carbamate | | 0.890 | 2.200 | 0.430 |
| N,N-dimethyl-N′-{(3R)-1-[4-(2-naphthyl)pyrimidin-2-yl]pyrrolidin-3-yl}urea | | | 1.790 | 0.260 |
| N-{(3R)-1-[4-(2-naphthyl)pyrimidin-2-yl]pyrrolidin-3-yl}pyrrolidine-1-carboxamide | | | 1.410 | 0.270 |
| N-{(3R)-1-[4-(2-naphthyl)pyrimidin-2-yl]pyrrolidin-3-yl}urea | 4.300 | 1.550 | 2.090 | 1.630 |
| N,N-dimethyl-N′-{(3R)-1-[4-(2-naphthyl)pyrimidin-2-yl]pyrrolidin-3-yl}sulfamide | | | 1.420 | 0.820 |
| N-{(3R)-1-[4-(2-naphthyl)pyrimidin-2-yl]pyrrolidin-3-yl}methanesulfonamide | | | 1.260 | 0.820 |
| 4-methyl-N-{(3R)-1-[4-(2-naphthyl)pyrimidin-2-yl]pyrrolidin-3-yl}benzenesulfonamide | | | 1.440 | 0.090 |
| N-{(3R)-1-[4-(2-naphthyl)pyrimidin-2-yl]pyrrolidin-3-yl}morpholine-4-carboxamide | | | 1.910 | |
| ethyl {(3R)-1-[4-(2-naphthyl)pyrimidin-2-yl]pyrrolidin-3-yl}carbamate | | | 1.210 | 0.690 |
| N-isopropyl-N′-{(3R)-1-[4-(2-naphthyl)pyrimidin-2-yl]pyrrolidin-3-yl}urea | | | 1.190 | 1.090 |
| N,N-diethyl-N′-{(3R)-1-[4-(2-naphthyl)pyrimidin-2-yl]pyrrolidin-3-yl}urea | | 1.270 | 1.220 | 1.800 |
| N-{(3R)-1-[4-(2-naphthyl)pyrimidin-2-yl]pyrrolidin-3-yl}guanidine | | | 1.630 | 0.650 |
| 4-chloro-N-{(3R)-1-[4-(2-naphthyl)pyrimidin-2-yl]pyrrolidin-3-yl}benzamide | | | 1.680 | |
| 4-cyano-N-{(3R)-1-[4-(2-naphthyl)pyrimidin-2-yl]pyrrolidin-3-yl}benzamide | | | | 1.290 |
| N-({1-[4-(2-naphthyl)pyrimidin-2-yl]piperidin-4-yl}methyl)acetamide | 1.900 | 2.273 | 1.630 | 2.707 |
| N-({1-[4-(2-naphthyl)pyrimidin-2-yl]piperidin-4-yl}methyl)benzamide | | | 1.690 | |
| methyl ({1-[4-(2-naphthyl)pyrimidin-2-yl]piperidin-4-yl}methyl)carbamate | | | 1.210 | 1.590 |
| N,N-dimethyl-N′-({1-[4-(2-naphthyl)pyrimidin-2-yl]piperidin-4-yl}methyl)urea | 4.030 | 1.690 | 1.120 | 2.360 |
| N-({1-[4-(2-naphthyl)pyrimidin-2-yl]piperidin-4-yl}methyl)pyrrolidine-1-carboxamide | | | 0.910 | 1.130 |
| N-ethyl-N′-({1-[4-(2-naphthyl)pyrimidin-2-yl]piperidin-4-yl}methyl)urea | | | 1.700 | 1.340 |
| N-({1-[4-(2-naphthyl)pyrimidin-2-yl]piperidin-4-yl}methyl)urea | | | 1.290 | 1.180 |
| N,N-dimethyl-N′-({1-[4-(2-naphthyl)pyrimidin-2-yl]piperidin-4-yl}methyl)sulfamide | | | 1.050 | 1.110 |
| N-({1-[4-(2-naphthyl)pyrimidin-2-yl]piperidin-4-yl}methyl)methanesulfonamide | | | 1.120 | 0.480 |
| 4-methyl-N-({1-[4-(2-naphthyl)pyrimidin-2-yl]piperidin-4-yl}methyl)benzenesulfonamide | | | 1.070 | 0.410 |
| N-({1-[4-(2-naphthyl)pyrimidin-2-yl]piperidin-4-yl}methyl)pyrimidin-2-amine | | | 1.280 | 1.480 |
| N-{(3S)-1-[4-(2-naphthyl)pyrimidin-2-yl]pyrrolidin-3-yl}acetamide | 6.880 | 4.375 | 1.025 | 1.500 |
| 2,2,2-trifluoro-N-{(3S)-1-[4-(2-naphthyl)pyrimidin-2-yl]pyrrolidin-3-yl}acetamide | 25.900 | 3.620 | 1.590 | 1.660 |
| N-{(3S)-1-[4-(2-naphthyl)pyrimidin-2-yl]pyrrolidin-3-yl}benzamide | | | 1.130 | 0.640 |
| methyl {(3S)-1-[4-(2-naphthyl)pyrimidin-2-yl]pyrrolidin-3-yl}carbamate | | | 1.030 | 1.630 |
| N,N-dimethyl-N′-{(3S)-1-[4-(2-naphthyl)pyrimidin-2-yl]pyrrolidin-3-yl}urea | | | 0.940 | 1.650 |
| N-{(3S)-1-[4-(2-naphthyl)pyrimidin-2-yl]pyrrolidin-3-yl}pyrrolidine-1-carboxamide | | | 1.890 | |
| N-ethyl-N′-{(3S)-1-[4-(2-naphthyl)pyrimidin-2-yl]pyrrolidin-3-yl}urea | | | 1.030 | 0.600 |
| N-{(3S)-1-[4-(2-naphthyl)pyrimidin-2-yl]pyrrolidin-3-yl}urea | | | | 1.470 |
| N,N-dimethyl-N′-{(3S)-1-[4-(2-naphthyl)pyrimidin-2-yl]pyrrolidin-3-yl}sulfamide | | | 1.870 | |
| N-{(3S)-1-[4-(2-naphthyl)pyrimidin-2-yl]pyrrolidin-3-yl}methanesulfonamide | 13.800 | 1.420 | 0.680 | 1.560 |
| 4-methyl-N-{(3S)-1-[4-(2-naphthyl)pyrimidin-2-yl]pyrrolidin-3-yl}benzenesulfonamide | | | 1.240 | |
| N-{(3S)-1-[4-(2-naphthyl)pyrimidin-2-yl]pyrrolidin-3-yl}morpholine-4-carboxamide | | | 0.930 | 1.720 |
| ethyl {(3S)-1-[4-(2-naphthyl)pyrimidin-2-yl]pyrrolidin-3-yl}carbamate | | | 1.020 | 1.940 |
| N-isopropyl-N′-{(3S)-1-[4-(2-naphthyl)pyrimidin-2-yl]pyrrolidin-3-yl}urea | | | 1.120 | 0.790 |
| N,N-diethyl-N′-{(3S)-1-[4-(2-naphthyl)pyrimidin-2-yl]pyrrolidin-3-yl}urea | | | 1.830 | |
| 4-chloro-N-{(3S)-1-[4-(2-naphthyl)pyrimidin-2-yl]pyrrolidin-3-yl}benzamide | | | 1.780 | |
| 4-cyano-N-{(3S)-1-[4-(2-naphthyl)pyrimidin-2-yl]pyrrolidin-3-yl}benzamide | | | 0.840 | 1.240 |
| di-tert-butyl {(Z)-[({1-[4-(2-naphthyl)pyrimidin-2-yl]piperidin-4- | | | 0.940 | 1.350 |
| yl}methyl)amino]methylylidene}biscarbamate | | | | |
| N-({1-[4-(2-naphthyl)pyrimidin-2-yl]piperidin-4-yl}methyl)guanidine | | | 1.270 | 0.020 |
| N-(3-morpholin-4-ylpropyl)-4-(2-naphthyl)pyrimidin-2-amine | 23.800 | 2.120 | 1.680 | 3.270 |
| 4-({4-[4-(2-naphthyl)pyrimidin-2-yl]piperazin-1-yl}acetyl)morpholine | | 1.180 | 1.030 | 2.100 |
| 4-(2-{4-[4-(2-naphthyl)pyrimidin-2-yl]piperazin-1-yl}ethyl)morpholine | 4.600 | 2.400 | 1.535 | 3.490 |
| N,N-dimethyl-2-{4-[4-(2-naphthyl)pyrimidin-2-yl]piperazin-1-yl}ethanamine | | 2.440 | 2.550 | 0.010 |
| 2-(4-methylpiperazin-1-yl)-4-(2-naphthyl)pyrimidine | 15.500 | 2.960 | 1.160 | 1.810 |
| 4-(2-naphthyl)-2-(4-pyridin-2-ylpiperazin-1-yl)pyrimidine | | | 1.800 | |
| 4-(2-naphthyl)-2-(4-pyrimidin-2-ylpiperazin-1-yl)pyrimidine | | | 1.020 | 1.240 |
| 1-{1-[4-(2-naphthyl)pyrimidin-2-yl]piperidin-4-yl}-1,3-dihydro-2H-benzimidazol-2-one | | | 1.030 | 0.010 |
| N-(trans-4-{[4-(2-naphthyl)pyrimidin-2-yl]amino}cyclohexyl)acetamide | 18.900 | 3.695 | 1.900 | 3.200 |
| methyl (trans-4-{[4-(2-naphthyl)pyrimidin-2-yl]amino}cyclohexyl)carbamate | 12.300 | 2.490 | 2.530 | 1.660 |
| N,N-dimethyl-N′-(trans-4-{[4-(2-naphthyl)pyrimidin-2-yl]amino}cyclohexyl)urea | | 2.270 | 2.780 | 1.610 |
| N-ethyl-N′-(trans-4-{[4-(2-naphthyl)pyrimidin-2-yl]amino}cyclohexyl)urea | | 2.740 | 2.840 | 2.350 |
| N,N-dimethyl-N′-(trans-4-{[4-(2-naphthyl)pyrimidin-2-yl]amino}cyclohexyl)sulfamide | | 1.740 | 1.650 | 1.740 |
| N-(trans-4-{[4-(2-naphthyl)pyrimidin-2-yl]amino}cyclohexyl)methanesulfonamide | 3.300 | 2.940 | 1.790 | 2.380 |
| 4-methyl-N-(trans-4-{[4-(2-naphthyl)pyrimidin-2- | | 1.230 | 1.780 | 1.130 |
| yl]amino}cyclohexyl)benzenesulfonamide | | | | |
| 5-(dimethylamino)-N-(trans-4-{[4-(2-naphthyl)pyrimidin-2- | | | 1.430 | 1.340 |
| yl]amino}cyclohexyl)naphthalene-1-sulfonamide | | | | |
| 4-cyano-N-(3-{[4-(2-naphthyl)pyrimidin-2-yl]amino}cyclohexyl)benzamide | | | 1.150 | 0.240 |
| tert-butyl (3-{[4-(2-naphthyl)pyrimidin-2-yl]amino}cyclohexyl)carbamate | | | 1.070 | 0.300 |
| N-(1-acetylpiperidin-4-yl)-N-methyl-4-(2-naphthyl)pyrimidin-2-amine | | 1.650 | 1.370 | 0.370 |
| N-methyl-4-(2-naphthyl)-N-[1-(trifluoroacetyl)piperidin-4-yl]pyrimidin-2-amine | | | 1.030 | 1.060 |
| N-(1-benzoylpiperidin-4-yl)-N-methyl-4-(2-naphthyl)pyrimidin-2-amine | | | 1.850 | |
| methyl 4-{methyl[4-(2-naphthyl)pyrimidin-2-yl]amino}piperidine-1-carboxylate | | | 1.430 | 1.470 |
| N,N-dimethyl-4-{methyl[4-(2-naphthyl)pyrimidin-2-yl]amino}piperidine-1-carboxamide | | | 1.370 | 0.680 |
| N-methyl-4-(2-naphthyl)-N-[1-(pyrrolidin-1-ylcarbonyl)piperidin-4-yl]pyrimidin-2-amine | | | 1.810 | |
| N,N-diethyl-4-{methyl[4-(2-naphthyl)pyrimidin-2-yl]amino}piperidine-1-carboxamide | | | 0.860 | 1.320 |
| N-ethyl-4-{methyl[4-(2-naphthyl)pyrimidin-2-yl]amino}piperidine-1-carboxamide | | | 1.200 | 1.450 |
| 4-{methyl[4-(2-naphthyl)pyrimidin-2-yl]amino}piperidine-1-carboxamide | | | 1.650 | 0.630 |
| N-methyl-N-[1-(methylsulfonyl)piperidin-4-yl]-4-(2-naphthyl)pyrimidin-2-amine | | | 1.240 | 0.770 |
| N-methyl-N-{1-[(4-methylphenyl)sulfonyl]piperidin-4-yl}-4-(2-naphthyl)pyrimidin-2- | | | 1.650 | |
| amine | | | | |
| N-methyl-4-(2-naphthyl)-N-(1-pyrimidin-2-ylpiperidin-4-yl)pyrimidin-2-amine | | | 1.700 | |
| N-methyl-4-(2-naphthyl)-N-(1-propylpiperidin-4-yl)pyrimidin-2-amine | | | 1.740 | |
| N-(1-benzylpiperidin-4-yl)-N-methyl-4-(2-naphthyl)pyrimidin-2-amine | | | 1.550 | 1.240 |
| 2-(4-{methyl[4-(2-naphthyl)pyrimidin-2-yl]amino}piperidin-1-yl)acetamide | | 2.215 | 2.620 | 0.020 |
| N-methyl-4-(2-naphthyl)-N-{1-[2-(trityloxy)ethyl]piperidin-4-yl}pyrimidin-2-amine | | | 1.140 | 1.020 |
| 4-{methyl[4-(2-naphthyl)pyrimidin-2-yl]amino}piperidine-1-carbaldehyde | | | 1.730 | 1.290 |
| 4-{methyl[4-(2-naphthyl)pyrimidin-2-yl]amino}piperidine-1-carboximidamide | | | 1.740 | |
| N-[(1-acetylazetidin-3-yl)methyl]-4-(2-naphthyl)pyrimidin-2-amine | | | 1.800 | |
| 4-(2-naphthyl)-N-{[1-(trifluoroacetyl)azetidin-3-yl]methyl}pyrimidin-2-amine | | | 1.500 | |
| N-[(1-benzoylazetidin-3-yl)methyl]-4-(2-naphthyl)pyrimidin-2-amine | | | 1.500 | |
| 3-({[4-(2-naphthyl)pyrimidin-2-yl]amino}methyl)azetidine-1-carboxamide | | | 1.090 | 1.580 |
| N-ethyl-3-({[4-(2-naphthyl)pyrimidin-2-yl]amino}methyl)azetidine-1-carboxamide | | | 1.570 | |
| N-{[1-(methylsulfonyl)azetidin-3-yl]methyl}-4-(2-naphthyl)pyrimidin-2-amine | | | | 1.520 |
| N-({1-[(4-methylphenyl)sulfonyl]azetidin-3-yl}methyl)-4-(2-naphthyl)pyrimidin-2-amine | | | 1.450 | 1.200 |
| N,N-dimethyl-3-({[4-(2-naphthyl)pyrimidin-2-yl]amino}methyl)azetidine-1-sulfonamide | | | 1.470 | 1.140 |
| N-({1-[4-(2-naphthyl)pyrimidin-2-yl]azetidin-3-yl}methyl)acetamide | | | 0.690 | 1.760 |
| 2,2,2-trifluoro-N-({1-[4-(2-naphthyl)pyrimidin-2-yl]azetidin-3-yl}methyl)acetamide | | | 1.770 | |
| N,N-dimethyl-N′-({1-[4-(2-naphthyl)pyrimidin-2-yl]azetidin-3-yl}methyl)urea | | | 0.990 | 1.430 |
| methyl ({1-[4-(2-naphthyl)pyrimidin-2-yl]azetidin-3-yl}methyl)carbamate | | | 1.280 | 1.080 |
| N-({1-[4-(2-naphthyl)pyrimidin-2-yl]azetidin-3-yl}methyl)urea | | | 1.460 | 1.640 |
| N-ethyl-N′-({1-[4-(2-naphthyl)pyrimidin-2-yl]azetidin-3-yl}methyl)urea | | | 1.860 | |
| N-({1-[4-(2-naphthyl)pyrimidin-2-yl]azetidin-3-yl}methyl)methanesulfonamide | | | 1.100 | 1.380 |
| 4-methyl-N-({1-[4-(2-naphthyl)pyrimidin-2-yl]azetidin-3-yl}methyl)benzenesulfonamide | | | 1.160 | 0.280 |
| N-(2-{1-[4-(2-naphthyl)pyrimidin-2-yl]piperidin-4-yl}ethyl)acetamide | | 1.360 | 2.270 | 1.000 |
| 2,2,2-trifluoro-N-(2-{1-[4-(2-naphthyl)pyrimidin-2-yl]piperidin-4-yl}ethyl)acetamide | | | 1.770 | 1.040 |
| methyl (2-{1-[4-(2-naphthyl)pyrimidin-2-yl]piperidin-4-yl}ethyl)carbamate | | | 1.390 | 0.890 |
| N,N-dimethyl-N′-(2-{1-[4-(2-naphthyl)pyrimidin-2-yl]piperidin-4-yl}ethyl)urea | | | 1.480 | 0.180 |
| N,N-diethyl-N′-(2-{1-[4-(2-naphthyl)pyrimidin-2-yl]piperidin-4-yl}ethyl)urea | | | 1.130 | 0.330 |
| N-ethyl-N′-(2-{1-[4-(2-naphthyl)pyrimidin-2-yl]piperidin-4-yl}ethyl)urea | | | 1.780 | 1.870 |
| N-isopropyl-N′-(2-{1-[4-(2-naphthyl)pyrimidin-2-yl]piperidin-4-yl}ethyl)urea | | | 1.460 | 1.580 |
| N-(2-{1-[4-(2-naphthyl)pyrimidin-2-yl]piperidin-4-yl}ethyl)urea | | 1.112 | | |
| N-cyclohexyl-N′-(2-{1-[4-(2-naphthyl)pyrimidin-2-yl]piperidin-4-yl}ethyl)urea | | | | |
| N-(2-{1-[4-(2-naphthyl)pyrimidin-2-yl]piperidin-4-yl}ethyl)pyrrolidine-1-carboxamide | | | | |
| N-(2-{1-[4-(2-naphthyl)pyrimidin-2-yl]piperidin-4-yl}ethyl)morpholine-4-carboxamide | | | | |
| N,N-dimethyl-N′-(2-{1-[4-(2-naphthyl)pyrimidin-2-yl]piperidin-4-yl}ethyl)sulfamide | | | | |
| N-(2-{1-[4-(2-naphthyl)pyrimidin-2-yl]piperidin-4-yl}ethyl)methanesulfonamide | | 0.880 | | |
| benzyl (2-{1-[4-(2-naphthyl)pyrimidin-2-yl]piperidin-4-yl}ethyl)carbamate | | | 0.860 | 1.490 |
| N-(2-{1-[4-(2-naphthyl)pyrimidin-2-yl]piperidin-4-yl}ethyl)-N′-phenylurea | | | 1.000 | 1.500 |
| N-(2-{1-[4-(2-naphthyl)pyrimidin-2-yl]piperidin-4-yl}ethyl)benzamide | | | 0.910 | 1.950 |
| N-[(3S)-1-acetylpyrrolidin-3-yl]-4-(2-naphthyl)pyrimidin-2-amine | | | 1.150 | 1.660 |
| N-[(3S)-1-benzoylpyrrolidin-3-yl]-4-(2-naphthyl)pyrimidin-2-amine | | | 1.230 | 0.000 |
| methyl (3S)-3-{[4-(2-naphthyl)pyrimidin-2-yl]amino}pyrrolidine-1-carboxylate | 2.765 | 2.725 | 1.650 | 2.120 |
| (3S)—N,N-dimethyl-3-{[4-(2-naphthyl)pyrimidin-2-yl]amino}pyrrolidine-1-carboxamide | 2.700 | 2.945 | 2.050 | 3.050 |
| (3S)—N-ethyl-3-{[4-(2-naphthyl)pyrimidin-2-yl]amino}pyrrolidine-1-carboxamide | | 2.500 | 2.490 | 2.810 |
| (3S)-3-{[4-(2-naphthyl)pyrimidin-2-yl]amino}pyrrolidine-1-carboxamide | 1.515 | 2.750 | 2.050 | 2.940 |
| (3S)—N,N-dimethyl-3-{[4-(2-naphthyl)pyrimidin-2-yl]amino}pyrrolidine-1-sulfonamide | 3.960 | 2.050 | 1.090 | 2.010 |
| N-[(3S)-1-(methylsulfonyl)pyrrolidin-3-yl]-4-(2-naphthyl)pyrimidin-2-amine | 2.895 | 2.585 | 1.410 | 2.260 |
| N-{(3S)-1-[(4-methylphenyl)sulfonyl]pyrrolidin-3-yl}-4-(2-naphthyl)pyrimidin-2-amine | | | 1.220 | 1.010 |
| (3S)-3-{[4-(2-naphthyl)pyrimidin-2-yl]amino}pyrrolidine-1-carbaldehyde | 6.800 | 3.165 | 1.800 | 3.510 |
| N-[(3S)-1-(morpholin-4-ylcarbonyl)pyrrolidin-3-yl]-4-(2-naphthyl)pyrimidin-2-amine | | | 1.120 | 1.940 |
| ethyl (3S)-3-{[4-(2-naphthyl)pyrimidin-2-yl]amino}pyrrolidine-1-carboxylate | | | 1.280 | 1.540 |
| N-[(3R)-1-acetylpyrrolidin-3-yl]-4-(2-naphthyl)pyrimidin-2-amine | 14.200 | 2.980 | 1.210 | 2.065 |
| N-[(3R)-1-benzoylpyrrolidin-3-yl]-4-(2-naphthyl)pyrimidin-2-amine | | | 1.840 | |
| methyl (3R)-3-{[4-(2-naphthyl)pyrimidin-2-yl]amino}pyrrolidine-1-carboxylate | | 1.360 | | 2.350 |
| (3R)—N,N-dimethyl-3-{[4-(2-naphthyl)pyrimidin-2-yl]amino}pyrrolidine-1-carboxamide | | | | 1.330 |
| (3R)—N-ethyl-3-{[4-(2-naphthyl)pyrimidin-2-yl]amino}pyrrolidine-1-carboxamide | | 1.460 | | 2.010 |
| (3R)-3-{[4-(2-naphthyl)pyrimidin-2-yl]amino}pyrrolidine-1-carboxamide | 8.710 | 5.295 | | 2.420 |
| (3R)—N,N-dimethyl-3-{[4-(2-naphthyl)pyrimidin-2-yl]amino}pyrrolidine-1-sulfonamide | 3.160 | 2.510 | | 2.310 |
| N-[(3R)-1-(methylsulfonyl)pyrrolidin-3-yl]-4-(2-naphthyl)pyrimidin-2-amine | 3.550 | 3.380 | | 3.460 |
| N-{(3R)-1-[(4-methylphenyl)sulfonyl]pyrrolidin-3-yl}-4-(2-naphthyl)pyrimidin-2-amine | | | | 1.580 |
| (3R)-3-{[4-(2-naphthyl)pyrimidin-2-yl]amino}pyrrolidine-1-carbaldehyde | 7.170 | 5.305 | 1.030 | 2.690 |
| N-[(3R)-1-(morpholin-4-ylcarbonyl)pyrrolidin-3-yl]-4-(2-naphthyl)pyrimidin-2-amine | | 1.340 | | 2.350 |
| ethyl (3R)-3-{[4-(2-naphthyl)pyrimidin-2-yl]amino}pyrrolidine-1-carboxylate | | | | 1.300 |
| N-({1-[4-(2-naphthyl)pyrimidin-2-yl]pyrrolidin-3-yl}methyl)acetamide | | | 0.950 | 1.500 |
| N-({1-[4-(2-naphthyl)pyrimidin-2-yl]pyrrolidin-3-yl}methyl)benzamide | | | 1.060 | 1.030 |
| methyl ({1-[4-(2-naphthyl)pyrimidin-2-yl]pyrrolidin-3-yl}methyl)carbamate | | | 1.040 | 1.080 |
| N,N-dimethyl-N′-({1-[4-(2-naphthyl)pyrimidin-2-yl]pyrrolidin-3-yl}methyl)urea | | | 0.840 | 1.150 |
| N-ethyl-N′-({1-[4-(2-naphthyl)pyrimidin-2-yl]pyrrolidin-3-yl}methyl)urea | | | 0.930 | 1.200 |
| N-({1-[4-(2-naphthyl)pyrimidin-2-yl]pyrrolidin-3-yl}methyl)urea | 19.360 | 1.720 | 1.140 | 2.340 |
| N,N-dimethyl-N′-({1-[4-(2-naphthyl)pyrimidin-2-yl]pyrrolidin-3-yl}methyl)sulfamide | | | 0.990 | 1.200 |
| N-({1-[4-(2-naphthyl)pyrimidin-2-yl]pyrrolidin-3-yl}methyl)methanesulfonamide | | | 0.880 | 1.630 |
| N-({1-[4-(2-naphthyl)pyrimidin-2-yl]pyrrolidin-3-yl}methyl)formamide | | | 1.090 | 1.680 |
| N-{[(3R)-1-acetylpyrrolidin-3-yl]methyl}-4-(2-naphthyl)pyrimidin-2-amine | | | | |
| methyl (3R)-3-({[4-(2-naphthyl)pyrimidin-2-yl]amino}methyl)pyrrolidine-1-carboxylate | | | | |
| (3R)-3-({[4-(2-naphthyl)pyrimidin-2-yl]amino}methyl)pyrrolidine-1-carbaldehyde | | | | |
| N-{[(3S)-1-acetylpyrrolidin-3-yl]methyl}-4-(2-naphthyl)pyrimidin-2-amine | | | | |
| methyl (3S)-3-({[4-(2-naphthyl)pyrimidin-2-yl]amino}methyl)pyrrolidine-1-carboxylate | | | | |
| (3S)-3-({[4-(2-naphthyl)pyrimidin-2-yl]amino}methyl)pyrrolidine-1-carbaldehyde | | | | |
| 4-{[4-(2-naphthyl)pyrimidin-2-yl]methyl}-1,4-diazepane-1-carbaldehyde | | | 1.150 | 1.130 |
| 1-(1-{[4-(2-naphthyl)pyrimidin-2-yl]methyl}piperidin-4-yl)methanamine | | | 1.410 | 1.770 |
| N-(4-methoxybenzyl)-4-(2-naphthyl)pyrimidin-2-amine | | | | 1.120 |
| ({1-[4-(2-naphthyl)pyrimidin-2-yl]piperidin-4-yl}methyl)amine | 1.975 | 3.390 | 2.228 | 0.143 |
| tert-butyl 4-{[4-(2-naphthyl)pyrimidin-2-yl]amino}piperidine-1-carboxylate | | | 1.000 | 1.410 |
| 4-(2-naphthyl)-N-piperidin-4-ylpyrimidin-2-amine | 4.125 | 3.723 | 1.468 | 1.755 |
| 4-(2-naphthyl)-N-(piperidin-4-ylmethyl)pyrimidin-2-amine | | 6.120 | 1.330 | 1.357 |
| tert-butyl 4-({[4-(2-naphthyl)pyrimidin-2-yl]amino}methyl)piperidine-1-carboxylate | | | 0.930 | 1.180 |
| 4-[4-(2-naphthyl)pyrimidin-2-yl]-1,4-diazepane-1-carbaldehyde | | | 0.870 | 1.880 |
| 1-[4-(2-naphthyl)pyrimidin-2-yl]-1,4-diazepane | | | 1.360 | 0.850 |
| 4-(2-naphthyl)-N-(pyridin-3-ylmethyl)pyrimidin-2-amine | 5.900 | 1.960 | 1.550 | 1.410 |
| 4-(2-naphthyl)-N-(2-pyridin-3-ylethyl)pyrimidin-2-amine | | | 1.150 | 1.040 |
| 4-(2-naphthyl)-N-(2-pyridin-4-ylethyl)pyrimidin-2-amine | 0.980 | 2.765 | 1.960 | 0.375 |
| 4-(2-naphthyl)-N-(pyridin-4-ylmethyl)pyrimidin-2-amine | 2.415 | 2.070 | 1.410 | 1.700 |
| 4-(2-naphthyl)-N-(pyridin-2-ylmethyl)pyrimidin-2-amine | | 2.690 | 1.720 | 1.550 |
| 1-[4-(2-naphthyl)pyrimidin-2-yl]piperidine-4-carboxamide | | 1.660 | 1.620 | 1.150 |
| tert-butyl {1-[4-(2-naphthyl)pyrimidin-2-yl]piperidin-4-yl}carbamate | | | 0.930 | 1.910 |
| tert-butyl 4-[4-(2-naphthyl)pyrimidin-2-yl]piperazine-1-carboxylate | | | 0.980 | 1.940 |
| {1-[4-(2-naphthyl)pyrimidin-2-yl]piperidin-4-yl}methanol | | | 1.810 | 0.020 |
| {1-[4-(2-naphthyl)pyrimidin-2-yl]piperidin-4-yl}methyl 4-methylbenzenesulfonate | | | | |
| 2-[4-(2-naphthyl)pyrimidin-2-yl]-1,2,3,4-tetrahydroisoquinoline | | | 0.950 | 1.380 |
| 4-({[4-(2-naphthyl)pyrimidin-2-yl]amino}methyl)benzenesulfonamide | | | 1.300 | 1.060 |
| (2-{4-[4-(2-naphthyl)pyrimidin-2-yl]piperazin-1-yl}ethyl)amine | | | 1.000 | 1.790 |
| 1-[4-(2-naphthyl)pyrimidin-2-yl]piperidin-4-amine | 0.970 | 1.580 | 2.110 | 0.000 |
| 4-(2-naphthyl)-2-piperazin-1-ylpyrimidine | | 2.050 | 2.640 | 0.000 |
| N,N-dimethyl-3-{4-[4-(2-naphthyl)pyrimidin-2-yl]piperazin-1-yl}propan-1-amine | | | 1.870 | |
| tert-butyl {1-[4-(2-naphthyl)pyrimidin-2-yl]azepan-4-yl}carbamate | | | 1.340 | 0.330 |
| 1-[4-(2-naphthyl)pyrimidin-2-yl]azepan-4-amine | | | 1.950 | |
| tert-butyl {(3S)-1-[4-(2-naphthyl)pyrimidin-2-yl]pyrrolidin-3-yl}carbamate | | | 1.510 | |
| tert-butyl {(3R)-1-[4-(2-naphthyl)pyrimidin-2-yl]pyrrolidin-3-yl}carbamate | | | 1.950 | |
| tert-butyl ({1-[4-(2-naphthyl)pyrimidin-2-yl]piperidin-3-yl}methyl)carbamate | | | 0.910 | 1.940 |
| ({1-[4-(2-naphthyl)pyrimidin-2-yl]piperidin-3-yl}methyl)amine | 2.681 | 5.433 | 2.895 | 0.005 |
| trans-N-[4-(2-naphthyl)pyrimidin-2-yl]cyclohexane-1,4-diamine | 3.700 | 4.730 | 1.910 | 3.950 |
| {1-[4-(2-naphthyl)pyrimidin-2-yl]azepan-4-yl}formamide | 2.250 | 1.800 | 1.505 | 2.050 |
| (3R)-1-[4-(2-naphthyl)pyrimidin-2-yl]pyrrolidin-3-amine | | | 1.620 | 0.010 |
| (3S)-1-[4-(2-naphthyl)pyrimidin-2-yl]pyrrolidin-3-amine | | | 1.480 | 0.000 |
| tert-butyl 4-{methyl[4-(2-naphthyl)pyrimidin-2-yl]amino}piperidine-1-carboxylate | | | 0.980 | 1.920 |
| 2-{1-[4-(2-naphthyl)pyrimidin-2-yl]piperidin-4-yl}ethanol | | 1.840 | 1.540 | 1.640 |
| N-[4-(2-naphthyl)pyrimidin-2-yl]azepan-4-amine | 16.110 | 6.080 | 1.110 | 4.870 |
| N-methyl-4-(2-naphthyl)-N-piperidin-4-ylpyrimidin-2-amine | 1.800 | 2.320 | 2.645 | 1.350 |
| tert-butyl 3-({[4-(2-naphthyl)pyrimidin-2-yl]amino}methyl)azetidine-1-carboxylate | | | 1.800 | 1.020 |
| N,N-dimethyl-1-[4-(2-naphthyl)pyrimidin-2-yl]pyrrolidin-3-amine | 4.110 | 5.503 | 2.000 | 1.905 |
| N,N-diethyl-1-[4-(2-naphthyl)pyrimidin-2-yl]pyrrolidin-3-amine | 28.200 | 3.680 | 1.060 | 2.670 |
| tert-butyl ({1-[4-(2-naphthyl)pyrimidin-2-yl]azetidin-3-yl}methyl)carbamate | | | 1.410 | 0.460 |
| N-(azetidin-3-ylmethyl)-4-(2-naphthyl)pyrimidin-2-amine | | 3.140 | 1.455 | 1.150 |
| 1-{1-[4-(2-naphthyl)pyrimidin-2-yl]azetidin-3-yl}methanamine | 22.900 | 3.080 | 2.560 | 0.710 |
| 4-(2-naphthyl)-2-(4-pyridin-4-ylpiperazin-1-yl)pyrimidine | | | | |
| 2-{1-[4-(2-naphthyl)pyrimidin-2-yl]piperidin-4-yl}ethyl methanesulfonate | | | 1.020 | 1.180 |
| 1-[4-(2-naphthyl)pyrimidin-2-yl]piperidin-4-one | | | 1.430 | 1.190 |
| 2-[4-(2-azidoethyl)piperidin-1-yl]-4-(2-naphthyl)pyrimidine | | | 1.000 | 1.670 |
| 2-azetidin-1-yl-4-(2-naphthyl)pyrimidine | | | 1.370 | 1.580 |
| 2-{1-[4-(2-naphthyl)pyrimidin-2-yl]piperidin-4-yl}ethanamine | | 1.420 | 2.155 | 0.005 |
| 1-[4-(2-naphthyl)pyrimidin-2-yl]piperidine-4-carboxylic acid | | | 1.780 | 0.570 |
| 1-[4-(2-naphthyl)pyrimidin-2-yl]piperidine-4-carbaldehyde | | | 1.510 | 0.250 |
| 4-(3-{4-[4-(2-naphthyl)pyrimidin-2-yl]piperazin-1-yl}propyl)morpholine | | | 1.290 | 0.010 |
| tert-butyl (3S)-3-{[4-(2-naphthyl)pyrimidin-2-yl]amino}pyrrolidine-1-carboxylate | | 1.270 | 2.110 | 0.000 |
| 2-[4-(2-furoyl)piperazin-1-yl]-4-(2-naphthyl)pyrimidine | | 1.630 | 1.490 | 3.620 |
| 8-[4-(2-naphthyl)pyrimidin-2-yl]-1,4-dioxa-8-azaspiro[4.5]decane | | | 1.500 | 1.740 |
| tert-butyl (3R)-3-{[4-(2-naphthyl)pyrimidin-2-yl]amino}pyrrolidine-1-carboxylate | | 0.800 | 2.380 | 0.010 |
| 4-(2-naphthyl)-N-[(3S)-pyrrolidin-3-yl]pyrimidin-2-amine | | 1.360 | 2.840 | 0.890 |
| 4-(2-naphthyl)-N-[(3R)-pyrrolidin-3-yl]pyrimidin-2-amine | 9.233 | 6.560 | 1.760 | 3.980 |
| (3R)—N-methyl-1-[4-(2-naphthyl)pyrimidin-2-yl]pyrrolidin-3-amine | | | 1.200 | |
| (3S)—N-methyl-1-[4-(2-naphthyl)pyrimidin-2-yl]pyrrolidin-3-amine | | | 1.750 | |
| tert-butyl ({1-[4-(2-naphthyl)pyrimidin-2-yl]pyrrolidin-3-yl}methyl)carbamate | | | 1.090 | 0.170 |
| 1-[4-(2-naphthyl)pyrimidin-2-yl]-4-(nitromethyl)piperidin-4-ol | | | 1.190 | 1.490 |
| 4-(aminomethyl)-1-[4-(2-naphthyl)pyrimidin-2-yl]piperidin-4-ol | 1.180 | 2.950 | 1.600 | 0.000 |
| tert-butyl (3R)-3-({[4-(2-naphthyl)pyrimidin-2-yl]amino}methyl)pyrrolidine-1-carboxylate | | | 1.830 | |
| methyl [(tert-butoxycarbonyl)amino]{1-[4-(2-naphthyl)pyrimidin-2-yl]piperidin-4- | | | 1.010 | 1.240 |
| ylidene}acetate | | | | |
| tert-butyl 4-(2-methoxy-1-{[4-(2-naphthyl)pyrimidin-2-yl]amino}-2-oxoethyl)piperidine-1- | | | 1.620 | |
| carboxylate | | | | |
| 4-(2-naphthyl)-N-[(3S)-pyrrolidin-3-ylmethyl]pyrimidin-2-amine | 3.740 | 6.810 | | |
| 1-[4-(2-naphthyl)pyrimidin-2-yl]-4-(1-nitroethyl)piperidin-4-ol | | | | |
| tert-butyl (3S)-3-({[4-(2-naphthyl)pyrimidin-2-yl]amino}methyl)pyrrolidine-1-carboxylate | | | | 0.010 |
| 4-(2-naphthyl)-N-[(3R)-pyrrolidin-3-ylmethyl]pyrimidin-2-amine | 3.880 | 7.510 | | 0.060 |
| 4-(2-naphthyl)-2-[4-(1-nitroethyl)piperidin-1-yl]pyrimidine | | | | 1.440 |
| methyl [(tert-butoxycarbonyl)amino]{1-[4-(2-naphthyl)pyrimidin-2-yl]piperidin-4- | | | | 0.710 |
| yl}acetate | | | | |
| 1-[4-(2-naphthyl)pyrimidin-2-yl]piperidine-4-carbonitrile | | | | 4.200 |
| methyl amino{1-[4-(2-naphthyl)pyrimidin-2-yl]piperidin-4-yl}acetate | 3.300 | 2.360 | | 0.030 |
| methyl (2R)-[(tert-butoxycarbonyl)amino]{1-[4-(2-naphthyl)pyrimidin-2-yl]piperidin-4- | | | | 0.770 |
| yl}acetate | | | | |
| methyl (2S)-[(tert-butoxycarbonyl)amino]{1-[4-(2-naphthyl)pyrimidin-2-yl]piperidin-4- | | | | 0.700 |
| yl}acetate | | | | |
| methyl 1-[4-(2-naphthyl)pyrimidin-2-yl]piperidine-4-carboxylate | | | | 1.390 |
| methyl (2R)-amino{1-[4-(2-naphthyl)pyrimidin-2-yl]piperidin-4-yl}acetate | 8.860 | 3.200 | 1.680 | 1.055 |
| methyl (2S)-amino{1-[4-(2-naphthyl)pyrimidin-2-yl]piperidin-4-yl}acetate | | 1.590 | | 0.570 |
| tert-butyl (1-{1-[4-(2-naphthyl)pyrimidin-2-yl]piperidin-4-yl}ethyl)carbamate | | | | 0.810 |
| 1-{1-[4-(2-naphthyl)pyrimidin-2-yl]piperidin-4-yl}ethanamine | 1.093 | 2.891 | 2.333 | 0.006 |
| 4-methyl-1-[4-(2-naphthyl)pyrimidin-2-yl]piperidin-4-ol | | | | 1.090 |
| 1-[4-(2-naphthyl)pyrimidin-2-yl]piperidine-4-carboximidamide | | | | 0.000 |
| tert-butyl ((1S)-1-{1-[4-(2-naphthyl)pyrimidin-2-yl]piperidin-4-yl}ethyl)carbamate | | | | 1.080 |
| (1R)-1-{1-[4-(2-naphthyl)pyrimidin-2-yl]piperidin-4-yl}ethanamine | 0.870 | 2.280 | | 0.000 |
| (1S)-1-{1-[4-(2-naphthyl)pyrimidin-2-yl]piperidin-4-yl}ethanamine | 0.865 | 1.940 | | 0.000 |
| (3S)—N,N-dimethyl-1-[4-(2-naphthyl)pyrimidin-2-yl]pyrrolidin-3-amine | 3.005 | 3.390 | | 0.080 |
| (3R)—N,N-dimethyl-1-[4-(2-naphthyl)pyrimidin-2-yl]pyrrolidin-3-amine | 3.557 | 5.193 | 2.880 | 0.065 |
| di-tert-butyl ((E)-{4-[4-(2-naphthyl)pyrimidin-2-yl]piperazin-1- | | | | |
| yl}methylylidene)biscarbamate | | | | |
| 2-[4-(azidomethyl)piperidin-1-yl]-4-(2-naphthyl)pyrimidine | | | | |
| N-[2-(4-methylphenyl)ethyl]-4-(2-naphthyl)pyrimidin-2-amine | | | | |
| 1-[4-(2-naphthyl)pyrimidin-2-yl]piperidine-3-carboxamide | 1.265 | 2.230 | 2.175 | 2.700 |
| 4-[4-(2-naphthyl)pyrimidin-2-yl]piperazine-1-carboximidamide | | | 1.100 | 0.050 |
| 1-{1-[4-(2-naphthyl)pyrimidin-2-yl]pyrrolidin-3-yl}methanamine | 2.256 | 4.063 | 4.530 | 0.010 |
| 4-(2-naphthyl)-2-[4-(nitromethyl)piperidin-1-yl]pyrimidine | | | 1.500 | 0.680 |
| (1R,5S,6s)-3-[4-(2-naphthyl)pyrimidin-2-yl]-3-azabicyclo[3.1.0]hexan-6-amine | 4.000 | 5.480 | 1.920 | 0.000 |
| (2R)-2-amino-2-{1-[4-(2-naphthyl)pyrimidin-2-yl]piperidin-4-yl}ethanol | | 1.660 | 2.960 | 0.000 |
| ethyl 1-[4-(2-naphthyl)pyrimidin-2-yl]piperidine-4-carboxylate | | 1.950 | 1.640 | 1.910 |
| ethyl {1-[4-(2-naphthyl)pyrimidin-2-yl]piperidin-4-yl}acetate | | | 1.260 | 1.060 |
| N-methyl-N-{(3S)-1-[4-(2-naphthyl)pyrimidin-2-yl]pyrrolidin-3-yl}acetamide | 2.150 | 2.533 | 2.375 | 2.890 |
| {(3S)-1-[4-(2-naphthyl)pyrimidin-2-yl]piperidin-3-yl}methanol | | 1.840 | 2.800 | 1.810 |
| {(3R)-1-[4-(2-naphthyl)pyrimidin-2-yl]piperidin-3-yl}methanol | | 1.800 | 1.980 | 1.080 |
| 4-(2-{4-[4-(2-naphthyl)pyrimidin-2-yl]piperazin-1-yl}ethyl)morpholine | | | | 3.000 |
| N-({1-[4-(2-naphthyl)pyrimidin-2-yl]piperidin-4-yl}methyl)acetamide | | | | 2.500 |
| tert-butyl ((1R)-2-hydroxy-1-{1-[4-(2-naphthyl)pyrimidin-2-yl]piperidin-4- | | | 1.130 | 0.350 |
| yl}ethyl)carbamate | | | | |
| tert-butyl ((1S)-2-hydroxy-1-{1-[4-(2-naphthyl)pyrimidin-2-yl]piperidin-4- | | | 1.220 | 0.440 |
| yl}ethyl)carbamate | | | | |
| 4-(2-naphthyl)-2-[4-(pyrrolidin-1-ylmethyl)piperidin-1-yl]pyrimidine | 3.5 | 3.6 | | 4.800 |
| tert-butyl ((1R)-2-hydroxy-2-methyl-1-{1-[4-(2-naphthyl)pyrimidin-2-yl]piperidin-4- | | | 1.790 | |
| yl}propyl)carbamate | | | | |
| (1R)-1-amino-2-methyl-1-{1-[4-(2-naphthyl)pyrimidin-2-yl]piperidin-4-yl}propan-2-ol | | | 1.780 | 0.000 |
| 4-(2-naphthyl)-2-[4-(piperidin-1-ylmethyl)piperidin-1-yl]pyrimidine | 8.0 | 3.4 | | 2.800 |
| (4R)-5,5-dimethyl-4-{1-[4-(2-naphthyl)pyrimidin-2-yl]piperidin-4-yl}-1,3-oxazolidin-2- | | 1.585 | 1.980 | 1.390 |
| one | | | | |
| (4R)-4-{1-[4-(2-naphthyl)pyrimidin-2-yl]piperidin-4-yl}-1,3-oxazolidin-2-one | | | 1.400 | 0.010 |
| (3R)—N-methyl-1-[4-(2-naphthyl)pyrimidin-2-yl]pyrrolidin-3-amine | | | 1.200 | |
| 1-{1-[4-(6-vinyl-2-naphthyl)pyrimidin-2-yl]piperidin-4-yl}methanamine | | | 1.210 | |
| 1-(1-{[4-(2-naphthyl)pyrimidin-2-yl]carbonyl}piperidin-4-yl)methanamine | | 1.380 | 0.460 | 1.420 |
| tert-butyl {(3S)-1-[4-(2-naphthyl)pyrimidin-2-yl]pyrrolidin-3-yl}carbamate | | | 1.510 | |
| (3S)—N-methyl-1-[4-(2-naphthyl)pyrimidin-2-yl]pyrrolidin-3-amine | | | 1.750 | |
| 4-({1-[4-(2-naphthyl)pyrimidin-2-yl]piperidin-4-yl}methyl)morpholine | 4.975 | 2.380 | 0.760 | 1.960 |
| 2,2,2-trifluoro-N-({1-[4-(2-naphthyl)pyrimidin-2-yl]azetidin-3-yl}methyl)acetamide | | | 1.770 | |
| (3S)-1-[4-(2-naphthyl)pyrimidin-2-yl]-N-(2,2,2-trifluoroethyl)pyrrolidin-3-amine | | | 0.830 | 1.380 |
| 2,2,2-trifluoro-N-(2-{(2R)-1-[4-(2-naphthyl)pyrimidin-2-yl]pyrrolidin-2- | 1.190 | 1.535 | 0.840 | 2.300 |
| yl}ethyl)acetamide | | | | |
| N-ethyl-N′-({1-[4-(2-naphthyl)pyrimidin-2-yl]azetidin-3-yl}methyl)urea | | | 0.860 | 1.060 |
| 4-(2-naphthyl)-2-[(2S)-2-(pyrrolidin-1-ylcarbonyl)pyrrolidin-1-yl]pyrimidine | | | 0.870 | 1.070 |
| 1-(1-{4-[6-(2-methoxyphenyl)-2-naphthyl]pyrimidin-2-yl}piperidin-4-yl)methanamine | | | 1.880 | |
| 4-({1-[4-(2-naphthyl)pyrimidin-2-yl]pyrrolidin-3-yl}methyl)thiomorpholine | | | 1.900 | |
| 2-{(2S)-2-[(4-methylpiperazin-1-yl)carbonyl]pyrrolidin-1-yl}-4-(2-naphthyl)pyrimidine | | | 0.900 | 1.600 |
| 4-(2-naphthyl)-2-[(2S)-2-(piperidin-1-ylcarbonyl)pyrrolidin-1-yl]pyrimidine | | | 1.910 | |
| (3R)—N,N-dimethyl-1-{1-[4-(2-naphthyl)pyrimidin-2-yl]-D-prolyl}pyrrolidin-3-amine | | 1.630 | 0.930 | 1.855 |
| N-tert-butyl-1-[4-(2-naphthyl)pyrimidin-2-yl]-L-prolinamide | | | 1.940 | |
| tert-butyl 4-({(3R)-1-[4-(2-naphthyl)pyrimidin-2-yl]pyrrolidin-3-yl}methyl)piperazine-1- | | | 1.950 | |
| carboxylate | | | | |
| 1-[4-(2-naphthyl)pyrimidin-2-yl]azepan-4-amine | | | 1.950 | |
| N,N-dimethyl-N′-({1-[4-(2-naphthyl)pyrimidin-2-yl]azetidin-3-yl}methyl)urea | | | 0.990 | 1.430 |
| N,N-dimethyl-1-[4-(2-naphthyl)pyrimidin-2-yl]-L-prolinamide | | | 1.000 | 1.240 |
| 1-(1-{4-[6-(4-methoxyphenyl)-2-naphthyl]pyrimidin-2-yl}piperidin-4-yl)methanamine | | | | 1.900 |
| 4-(2-naphthyl)-2-[(2R)-2-(piperidin-1-ylcarbonyl)pyrrolidin-1-yl]pyrimidine | | | 1.010 | |
| 2-{(2R)-2-[(4-methylpiperazin-1-yl)carbonyl]pyrrolidin-1-yl}-4-(2-naphthyl)pyrimidine | | 1.600 | 1.010 | 2.090 |
| N,N-dimethyl-1-[4-(2-naphthyl)pyrimidin-2-yl]-D-prolinamide | | | 1.020 | 1.200 |
| N-ethyl-N-(2-hydroxyethyl)-1-[4-(2-naphthyl)pyrimidin-2-yl]-L-prolinamide | | | 1.030 | 1.630 |
| 4-(2-naphthyl)-2-[(2R)-2-(pyrrolidin-1-ylcarbonyl)pyrrolidin-1-yl]pyrimidine | | | 1.050 | 1.100 |
| (3S)—N,N-dimethyl-1-{1-[4-(2-naphthyl)pyrimidin-2-yl]-D-prolyl}pyrrolidin-3-amine | | 2.115 | 1.055 | 2.430 |
| 4-({1-[4-(2-naphthyl)pyrimidin-2-yl]pyrrolidin-3-yl}methyl)morpholine | 4.800 | 2.990 | 1.070 | 3.100 |
| 1-({(3S)-1-[4-(2-naphthyl)pyrimidin-2-yl]pyrrolidin-3-yl}methyl)piperidin-2-one | | 2.000 | 1.070 | 2.450 |
| (3R)—N-cyclohexyl-N-methyl-1-[4-(2-naphthyl)pyrimidin-2-yl]pyrrolidin-3-amine | | | 1.070 | 1.770 |
| 2,2,2-trifluoro-N-{1-[4-(2-naphthyl)pyrimidin-2-yl]azepan-4-yl}acetamide | | | 1.100 | 1.870 |
| N-({1-[4-(2-naphthyl)pyrimidin-2-yl]azetidin-3-yl}methyl)methanesulfonamide | | | 1.100 | 1.380 |
| (3R)—N,N-dimethyl-1-{1-[4-(2-naphthyl)pyrimidin-2-yl]-L-prolyl}pyrrolidin-3-amine | | 2.335 | 1.100 | 4.430 |
| 2,2,2-trifluoro-N-({1-[4-(6-formyl-2-naphthyl)pyrimidin-2-yl]piperidin-4- | | | 1.110 | 0.040 |
| yl}methyl)acetamide | | | | |
| N,N-dimethyl-N′-{1-[4-(2-naphthyl)pyrimidin-2-yl]azepan-4-yl}sulfamide | | | 1.120 | 1.010 |
| N-methyl-1-[4-(2-naphthyl)pyrimidin-2-yl]-D-prolinamide | | | 1.130 | 1.320 |
| 1-({(3R)-1-[4-(2-naphthyl)pyrimidin-2-yl]pyrrolidin-3-yl}methyl)piperidin-2-one | 6.850 | 2.330 | 1.140 | 2.330 |
| 4-methyl-N-({1-[4-(2-naphthyl)pyrimidin-2-yl]azetidin-3-yl}methyl)benzenesulfonamide | | | 1.160 | 0.280 |
| 4-({1-[4-(2-naphthyl)pyrimidin-2-yl]piperidin-4-yl}methyl)piperazin-2-one | | | 1.160 | 1.490 |
| tert-butyl 4-({(3S)-1-[4-(2-naphthyl)pyrimidin-2-yl]pyrrolidin-3-yl}methyl)piperazine-1- | | | 1.160 | 0.850 |
| carboxylate | | | | |
| (3S)—N,N-dimethyl-1-{1-[4-(2-naphthyl)pyrimidin-2-yl]-L-prolyl}pyrrolidin-3-amine | | 2.175 | 1.160 | 3.370 |
| N-tert-butyl-1-[4-(2-naphthyl)pyrimidin-2-yl]-D-prolinamide | | | 1.160 | 0.600 |
| 4-{(3S)-1-[4-(2-naphthyl)pyrimidin-2-yl]pyrrolidin-3-yl}morpholine | 7.500 | 2.405 | 1.170 | 3.060 |
| 4-{(3R)-1-[4-(2-naphthyl)pyrimidin-2-yl]pyrrolidin-3-yl}morpholine | | 1.290 | 1.200 | 2.450 |
| 4-(2-naphthyl)-2-[(3R)-3-(piperidin-1-ylmethyl)pyrrolidin-1-yl]pyrimidine | | | 1.210 | 1.340 |
| 1-(1-{4-[6-(2-thienyl)-2-naphthyl]pyrimidin-2-yl}piperidin-4-yl)methanamine | | | 1.210 | 0.030 |
| (3S)—N-cyclohexyl-N-methyl-1-[4-(2-naphthyl)pyrimidin-2-yl]pyrrolidin-3-amine | | | 1.220 | 0.010 |
| N-{1-[4-(2-naphthyl)pyrimidin-2-yl]azepan-4-yl}benzamide | | | 1.230 | 1.070 |
| 4-({1-[4-(2-naphthyl)pyrimidin-2-yl]pyrrolidin-3-yl}methyl)piperazin-2-one | | | 1.230 | 0.460 |
| N-{(3S)-1-[4-(2-naphthyl)pyrimidin-2-yl]pyrrolidin-3-yl}-N-(2,2,2- | | | 1.230 | 0.000 |
| trifluoroethyl)acetamide | | | | |
| 4-{(3R)-1-[4-(2-naphthyl)pyrimidin-2-yl]pyrrolidin-3-yl}piperazin-2-one | 5.736 | 2.874 | 1.248 | 2.330 |
| 1-{(3R)-1-[4-(2-naphthyl)pyrimidin-2-yl]pyrrolidin-3-yl}azepane | | | 1.250 | 1.300 |
| N-{1-[4-(2-naphthyl)pyrimidin-2-yl]azepan-4-yl}acetamide | | 1.350 | 1.260 | 2.910 |
| N-methyl-N-({(3R)-1-[4-(2-naphthyl)pyrimidin-2-yl]pyrrolidin-3- | | | 1.270 | 1.600 |
| yl}methyl)cyclohexanamine | | | | |
| 2-[(3S)-3-(1H-imidazol-1-ylmethyl)pyrrolidin-1-yl]-4-(2-naphthyl)pyrimidine | | 2.430 | 1.270 | 4.290 |
| 4-{(3S)-1-[4-(2-naphthyl)pyrimidin-2-yl]pyrrolidin-3-yl}piperazin-2-one | 6.885 | 3.585 | 1.280 | 3.160 |
| methyl ({1-[4-(2-naphthyl)pyrimidin-2-yl]azetidin-3-yl}methyl)carbamate | | | 1.280 | 1.080 |
| 2-[3-(1H-imidazol-1-ylmethyl)pyrrolidin-1-yl]-4-(2-naphthyl)pyrimidine | | | 1.280 | 0.380 |
| 2-[(3S)-3-(1H-imidazol-1-yl)pyrrolidin-1-yl]-4-(2-naphthyl)pyrimidine | 3.930 | 3.800 | 1.290 | 3.920 |
| 4-(2-naphthyl)-2-[(3R)-3-piperidin-1-ylpyrrolidin-1-yl]pyrimidine | | | 1.290 | 0.060 |
| 2-[(3R)-3-(1H-imidazol-1-yl)pyrrolidin-1-yl]-4-(2-naphthyl)pyrimidine | | 1.960 | 1.310 | 3.900 |
| 1-{(3S)-1-[4-(2-naphthyl)pyrimidin-2-yl]pyrrolidin-3-yl}piperidin-4-ol | | | 1.310 | 0.260 |
| 2-[3-(chloromethyl)pyrrolidin-1-yl]-4-(2-naphthyl)pyrimidine | | | 1.320 | 0.490 |
| 1-({1-[4-(2-naphthyl)pyrimidin-2-yl]piperidin-4-yl}methyl)pyrrolidin-2-one | | | 1.330 | 0.810 |
| 1-{1-[4-(6-propoxy-2-naphthyl)pyrimidin-2-yl]piperidin-4-yl}methanamine | | | 1.330 | 0.000 |
| 4-(2-naphthyl)-2-[(3S)-3-(piperidin-1-ylmethyl)pyrrolidin-1-yl]pyrimidine | | | 1.340 | 1.060 |
| 2-[(3R)-3-methoxypyrrolidin-1-yl]-4-(2-naphthyl)pyrimidine | 0.659 | 1.990 | 1.360 | 3.100 |
| N-methyl-N-({(3S)-1-[4-(2-naphthyl)pyrimidin-2-yl]pyrrolidin-3- | | 3.720 | 1.370 | 2.270 |
| yl}methyl)cyclohexanamine | | | | |
| (3′R)-1′-[4-(2-naphthyl)pyrimidin-2-yl]-1,3′-bipyrrolidine | | 2.760 | 1.380 | 2.620 |
| (3S)—N-(2-morpholin-4-ylethyl)-1-[4-(2-naphthyl)pyrimidin-2-yl]pyrrolidin-3-amine | | | 1.390 | 0.910 |
| tert-butyl ({1-[4-(2-naphthyl)pyrimidin-2-yl]azetidin-3-yl}methyl)carbamate | | | 1.410 | 0.460 |
| 2-(ethyl{(3S)-1-[4-(2-naphthyl)pyrimidin-2-yl]pyrrolidin-3-yl}amino)ethanol | 3.885 | 6.695 | 1.420 | 5.580 |
| (3′R)-1′-[4-(2-naphthyl)pyrimidin-2-yl]-1,3′-bipyrrolidin-2-one | 4.510 | 1.935 | 1.430 | 2.120 |
| 6-{2-[4-(aminomethyl)piperidin-1-yl]pyrimidin-4-yl}-2-naphthyl acetate | | | 1.435 | 1.995 |
| 1-{(3R)-1-[4-(2-naphthyl)pyrimidin-2-yl]pyrrolidin-3-yl}piperidin-4-ol | | | 1.440 | 0.140 |
| 2,2,2-trifluoro-N-(2-{(2S)-1-[4-(2-naphthyl)pyrimidin-2-yl]pyrrolidin-2-yl}ethyl)acetamide | | | 1.440 | 0.890 |
| 1-({1-[4-(2-naphthyl)pyrimidin-2-yl]piperidin-4-yl}methyl)azepane | | | 1.450 | 0.000 |
| (3′S)-1′-[4-(2-naphthyl)pyrimidin-2-yl]-1,3′-bipyrrolidine | | | 1.450 | 0.830 |
| N-({1-[4-(2-naphthyl)pyrimidin-2-yl]azetidin-3-yl}methyl)urea | | | 1.460 | 1.640 |
| 2-[(3S)-3-methoxypyrrolidin-1-yl]-4-(2-naphthyl)pyrimidine | 2.580 | 1.755 | 1.480 | 2.270 |
| 2-methyl-N-({1-[4-(2-naphthyl)pyrimidin-2-yl]piperidin-4-yl}methyl)propan-2-amine | 10.600 | 3.890 | 1.490 | 5.760 |
| 4-methyl-N-{1-[4-(2-naphthyl)pyrimidin-2-yl]azepan-4-yl}benzenesulfonamide | | 0.780 | 1.490 | 2.260 |
| (1-{(3R)-1-[4-(2-naphthyl)pyrimidin-2-yl]pyrrolidin-3-yl}piperidin-3-yl)methanol | | | 1.490 | 0.000 |
| 4-(2-naphthyl)-2-[(3R)-3-(pyrrolidin-1-ylmethyl)pyrrolidin-1-yl]pyrimidine | 7.667 | 6.053 | 1.498 | 6.073 |
| (3R)—N,N-dimethyl-1-({(3S)-1-[4-(2-naphthyl)pyrimidin-2-yl]pyrrolidin-3- | | | 1.530 | 0.030 |
| yl}methyl)pyrrolidin-3-amine | | | | |
| 1-{1-[4-(6-isobutoxy-2-naphthyl)pyrimidin-2-yl]piperidin-4-yl}methanamine | | | 1.540 | 0.000 |
| N-methyl-N-{(3R)-1-[4-(2-naphthyl)pyrimidin-2-yl]pyrrolidin-3-yl}acetamide | 10.000 | 2.285 | 1.550 | 2.710 |
| 1-({1-[4-(2-naphthyl)pyrimidin-2-yl]piperidin-4-yl}methyl)piperidin-2-one | | | 1.550 | 0.730 |
| (3R)—N-(tert-butyl)-1-[4-(2-naphthyl)pyrimidin-2-yl]pyrrolidin-3-amine | | | 1.560 | 0.290 |
| N-ethyl-N-({1-[4-(2-naphthyl)pyrimidin-2-yl]pyrrolidin-3-yl}methyl)ethanamine | | | 1.580 | 0.000 |
| 1-{1-[5-(2-naphthyl)pyridazin-3-yl]piperidin-4-yl}methanamine | | | 1.580 | 0.400 |
| 1-{1-[6-(2-naphthyl)pyrazin-2-yl]piperidin-4-yl}methanamine | 1.160 | 2.920 | 1.600 | 0.000 |
| N-{1-[4-(2-naphthyl)pyrimidin-2-yl]azepan-4-yl}urea | 20.360 | 1.500 | 1.620 | 2.890 |
| 1-{1-[4-(6-phenyl-2-naphthyl)pyrimidin-2-yl]piperidin-4-yl}methanamine | | | 1.630 | 0.000 |
| 4-(2-naphthyl)-2-[(3S)-3-piperidin-1-ylpyrrolidin-1-yl]pyrimidine | | | 1.650 | 0.000 |
| 4-(2-naphthyl)-2-[3-(piperidin-1-ylmethyl)pyrrolidin-1-yl]pyrimidine | | | 1.660 | 0.010 |
| (1-{(3R)-1-[4-(2-naphthyl)pyrimidin-2-yl]pyrrolidin-3-yl}piperidin-4-yl)methanol | | | 1.690 | 0.010 |
| (3R)—N-(2-morpholin-4-ylethyl)-1-[4-(2-naphthyl)pyrimidin-2-yl]pyrrolidin-3-amine | | | 1.710 | 0.210 |
| (1-{(3S)-1-[4-(2-naphthyl)pyrimidin-2-yl]pyrrolidin-3-yl}piperidin-4-yl)methanol | | | 1.720 | 0.940 |
| 2-[3-(azetidin-1-ylmethyl)pyrrolidin-1-yl]-4-(2-naphthyl)pyrimidine | | 2.500 | 1.730 | 0.000 |
| (3aR*,6aS*)-2-[4-(2-naphthyl)pyrimidin-2-yl]octahydropyrrolo[3,4-c]pyrrole | | | 1.730 | 0.010 |
| N,N-dimethyl-N′-{1-[4-(2-naphthyl)pyrimidin-2-yl]azepan-4-yl}urea | | | 1.740 | 1.750 |
| 2-{(3S)-3-[(4-methylpiperazin-1-yl)methyl]pyrrolidin-1-yl}-4-(2-naphthyl)pyrimidine | | | 1.800 | 0.000 |
| 2-(ethyl{(3R)-1-[4-(2-naphthyl)pyrimidin-2-yl]pyrrolidin-3-yl}amino)ethanol | | 3.430 | 1.800 | 5.830 |
| 4-(2-naphthyl)-2-[3-(pyrrolidin-1-ylmethyl)pyrrolidin-1-yl]pyrimidine | 7.587 | 7.000 | 1.870 | 0.000 |
| 6-{2-[4-(aminomethyl)piperidin-1-yl]pyrimidin-4-yl}-2-naphthol | | | 1.880 | 0.015 |
| (1-{(3S)-1-[4-(2-naphthyl)pyrimidin-2-yl]pyrrolidin-3-yl}piperidin-3-yl)methanol | | | 1.880 | 0.010 |
| 4-(2-naphthyl)-2-[(3S)-3-(pyrrolidin-1-ylmethyl)pyrrolidin-1-yl]pyrimidine | | 4.050 | 1.900 | 5.180 |
| 2-[(3R)-3-(1H-imidazol-1-ylmethyl)pyrrolidin-1-yl]-4-(2-naphthyl)pyrimidine | | 3.290 | 1.950 | 3.040 |
| 2-methyl-N-({1-[4-(2-naphthyl)pyrimidin-2-yl]pyrrolidin-3-yl}methyl)propan-2-amine | 1.368 | 3.106 | 1.993 | 0.018 |
| N-{1-[4-(2-naphthyl)pyrimidin-2-yl]azepan-4-yl}methanesulfonamide | | 1.120 | 2.040 | 2.210 |
| 1-{(3R)-1-[4-(2-naphthyl)pyrimidin-2-yl]pyrrolidin-3-yl}piperidin-3-ol | 3.640 | 5.120 | 2.060 | 1.050 |
| (3S)—N,N-dimethyl-1-({(3S)-1-[4-(2-naphthyl)pyrimidin-2-yl]pyrrolidin-3- | | 1.660 | 2.080 | 0.010 |
| yl}methyl)pyrrolidin-3-amine | | | | |
| {1-[4-(2-naphthyl)pyrimidin-2-yl]pyrrolidin-3-yl}methanol | 0.925 | 1.975 | 2.130 | 1.420 |
| 2-methyl-N-({(3R)-1-[4-(2-naphthyl)pyrimidin-2-yl]pyrrolidin-3-yl}methyl)propan-2- | | 2.300 | 2.240 | 0.010 |
| amine | | | | |
| (3R)—N,N-dimethyl-1-({(3R)-1-[4-(2-naphthyl)pyrimidin-2-yl]pyrrolidin-3- | | 1.440 | 2.240 | 0.020 |
| yl}methyl)pyrrolidin-3-amine | | | | |
| N,N-dimethyl-1-{(3S)-1-[4-(2-naphthyl)pyrimidin-2-yl]pyrrolidin-3-yl}methanamine | | 3.620 | 2.260 | 2.260 |
| 1-{1-[4-(6-methoxy-2-naphthyl)pyrimidin-2-yl]piperidin-4-yl}methanamine | 1.060 | 3.233 | 2.275 | 0.000 |
| N-ethyl-N′-{1-[4-(2-naphthyl)pyrimidin-2-yl]azepan-4-yl}urea | | 1.320 | 2.330 | 2.560 |
| 1-({1-[4-(2-naphthyl)pyrimidin-2-yl]pyrrolidin-3-yl}methyl)azepane | | 2.260 | 2.330 | 0.000 |
| (3R)-1-({1-[4-(2-naphthyl)pyrimidin-2-yl]pyrrolidin-3-yl}methyl)piperidin-3-ol | 1.030 | 3.745 | 2.340 | 0.010 |
| (3S)—N-(tert-butyl)-1-[4-(2-naphthyl)pyrimidin-2-yl]pyrrolidin-3-amine | | 2.690 | 2.390 | 0.000 |
| 2-methyl-N-({(3S)-1-[4-(2-naphthyl)pyrimidin-2-yl]pyrrolidin-3-yl}methyl)propan-2- | | 4.610 | 2.440 | 0.060 |
| amine | | | | |
| (3S,3′S)-N,N-dimethyl-1′-[4-(2-naphthyl)pyrimidin-2-yl]-1,3′-bipyrrolidin-3-amine | | 2.030 | 2.470 | 0.020 |
| 1-{(3S)-1-[4-(2-naphthyl)pyrimidin-2-yl]pyrrolidin-3-yl}piperidin-3-ol | 3.550 | 6.735 | 2.480 | 0.130 |
| 2-{(3R)-3-[(4-methylpiperazin-1-yl)methyl]pyrrolidin-1-yl}-4-(2-naphthyl)pyrimidine | | 2.180 | 2.500 | 0.010 |
| (3S)—N,N-dimethyl-1-({(3R)-1-[4-(2-naphthyl)pyrimidin-2-yl]pyrrolidin-3- | | 2.260 | 2.550 | 0.020 |
| yl}methyl)pyrrolidin-3-amine | | | | |
| 1-{1-[4-(2-naphthyl)pyrimidin-2-yl]azetidin-3-yl}methanamine | 22.900 | 3.080 | 2.560 | 0.710 |
| (3S,3′R)-N,N-dimethyl-1′-[4-(2-naphthyl)pyrimidin-2-yl]-1,3′-bipyrrolidin-3-amine | 1.110 | 3.175 | 2.640 | 0.000 |
| 1-{(4R)-1-[4-(2-naphthyl)pyrimidin-2-yl]azepan-4-yl}methanamine | 1.190 | 3.950 | 2.660 | 0.000 |
| 2-{3-[(4-methylpiperazin-1-yl)methyl]pyrrolidin-1-yl}-4-(2-naphthyl)pyrimidine | | 1.340 | 2.700 | 0.000 |
| (3R,3′R)-N,N-dimethyl-1′-[4-(2-naphthyl)pyrimidin-2-yl]-1,3′-bipyrrolidin-3-amine | | 1.710 | 2.710 | 0.010 |
| 2-{(2R)-1-[4-(2-naphthyl)pyrimidin-2-yl]pyrrolidin-2-yl}ethanamine | | 2.270 | 2.720 | 0.010 |
| N-methyl-1-{(3S)-1-[4-(2-naphthyl)pyrimidin-2-yl]pyrrolidin-3-yl}methanamine | 2.351 | 4.773 | 2.740 | 0.503 |
| N-methyl-1-{(3R)-1-[4-(2-naphthyl)pyrimidin-2-yl]pyrrolidin-3-yl}methanamine | | 1.030 | 2.760 | 0.010 |
| 1-{1-[6-(2-naphthyl)pyrimidin-4-yl]piperidin-4-yl}methanamine | 1.586 | 3.226 | 2.793 | 1.000 |
| 2-[ethyl({1-[4-(2-naphthyl)pyrimidin-2-yl]pyrrolidin-3-yl}methyl)amino]ethanol | 2.257 | 3.635 | 2.800 | 0.010 |
| N,N-dimethyl-1-{(3R)-1-[4-(2-naphthyl)pyrimidin-2-yl]pyrrolidin-3-yl}methanamine | 1.020 | 3.620 | 2.830 | 0.010 |
| N-methyl-N-({1-[4-(2-naphthyl)pyrimidin-2-yl]piperidin-4-yl}methyl)cyclohexanamine | | 2.160 | 2.870 | 0.030 |
| 1-{1-[4-(6-methyl-2-naphthyl)pyrimidin-2-yl]piperidin-4-yl}methanamine | 1.220 | 2.820 | 2.880 | 0.000 |
| (3R)—N,N-dimethyl-1-[4-(2-naphthyl)pyrimidin-2-yl]pyrrolidin-3-amine | 3.557 | 5.193 | 2.880 | 0.065 |
| 2-[ethyl({(3R)-1-[4-(2-naphthyl)pyrimidin-2-yl]pyrrolidin-3-yl}methyl)amino]ethanol | 2.920 | 3.895 | 2.930 | 0.150 |
| (3′S)-1′-[4-(2-naphthyl)pyrimidin-2-yl]-1,3′-bipyrrolidin-2-one | 0.189 | 2.290 | 3.040 | 3.620 |
| 1-{1-[5-(2-naphthyl)pyrimidin-2-yl]piperidin-4-yl}methanamine | 1.200 | 2.900 | 3.100 | |
| 1-{1-[2-(2-naphthyl)pyrimidin-4-yl]piperidin-4-yl}methanamine | 0.787 | 5.673 | 3.220 | 0.003 |
| 4-(2-naphthyl)-2-[(3R)-3-piperazin-1-ylpyrrolidin-1-yl]pyrimidine | | 2.510 | 3.440 | 0.000 |
| 2-[ethyl({(3S)-1-[4-(2-naphthyl)pyrimidin-2-yl]pyrrolidin-3-yl}methyl)amino]ethanol | | 3.540 | 3.480 | 0.040 |
| 1-({1-[4-(2-naphthyl)pyrimidin-2-yl]pyrrolidin-3-yl}methyl)piperidin-4-ol | 5.530 | 2.680 | 3.520 | 0.100 |
| 2-[(3R)-3-(4-methylpiperazin-1-yl)pyrrolidin-1-yl]-4-(2-naphthyl)pyrimidine | | 1.020 | 3.670 | 0.010 |
| 1-{(4S)-1-[4-(2-naphthyl)pyrimidin-2-yl]azepan-4-yl}methanamine | 1.065 | 3.640 | 3.860 | 0.000 |
| 1-{(3S)-1-[4-(2-naphthyl)pyrimidin-2-yl]pyrrolidin-3-yl}azepane | | 3.220 | 3.970 | 0.000 |
| 2-{(2S)-1-[4-(2-naphthyl)pyrimidin-2-yl]pyrrolidin-2-yl}ethanamine | | 2.950 | 4.080 | 0.020 |
| (3R,3′S)-N,N-dimethyl-1′-[4-(2-naphthyl)pyrimidin-2-yl]-1,3′-bipyrrolidin-3-amine | 1.073 | 4.637 | 4.170 | 0.000 |
| 2-{1-[4-(2-naphthyl)pyrimidin-2-yl]pyrrolidin-3-yl}ethanamine | 0.700 | 5.940 | 4.230 | 0.000 |
| (3S)-1-({1-[4-(2-naphthyl)pyrimidin-2-yl]pyrrolidin-3-yl}methyl)piperidin-3-ol | 1.010 | 6.950 | 4.340 | 0.000 |
| 2-[ethyl({1-[4-(2-naphthyl)pyrimidin-2-yl]piperidin-4-yl}methyl)amino]ethanol | | 2.760 | 4.930 | 0.010 |
| 4-(2-naphthyl)-2-[4-(piperazin-1-ylmethyl)piperidin-1-yl]pyrimidine | | 2.510 | 5.170 | 0.010 |
| 2-{4-[(4-methylpiperazin-1-yl)methyl]piperidin-1-yl}-4-(2-naphthyl)pyrimidine | 1.190 | 5.430 | 5.460 | 0.000 |
| 2-[(3S)-3-(4-methylpiperazin-1-yl)pyrrolidin-1-yl]-4-(2-naphthyl)pyrimidine | 0.696 | 7.175 | 5.690 | 0.010 |
| 4-(2-naphthyl)-2-[(3S)-3-piperazin-1-ylpyrrolidin-1-yl]pyrimidine | 1.100 | 7.520 | 6.160 | 0.000 |
| 1-(1-{4-[6-(piperidin-1-ylmethyl)-2-naphthyl]pyrimidin-2-yl}piperidin-4-yl)methanamine | | | 1.4 | |
| 1-(1-{4-[6-(morpholin-4-ylmethyl)-2-naphthyl]pyrimidin-2-yl}piperidin-4-yl)methanamine | | | 1.1 | |
| {(1S,4R)-2-[4-(2-naphthyl)pyrimidin-2-yl]-2-azabicyclo[2.2.1]heptane-5,5- | | | 1.2 | |
| diyl}dimethanamine | | | | |
| 1-{(1R,4R,5R)-2-[4-(2-naphthyl)pyrimidin-2-yl]-2-azabicyclo[2.2.1]hept-5- | | | 1.4 | |
| yl}methanamine | | | | |
| 1-{(1R,4R,5S)-2-[4-(2-naphthyl)pyrimidin-2-yl]-2-azabicyclo[2.2.1]hept-5- | | | 1.8 | |
| yl}methanamine | | | | |
| N-methyl-N-[2-(methyl{(3R)-1-[4-(2-naphthyl)pyrimidin-2-yl]pyrrolidin-3- | | | | 4.4 |
| yl}amino)ethyl]acetamide | | | | |
| 2,2,2-trifluoro-N-methyl-N-[2-(methyl{(3R)-1-[4-(2-naphthyl)pyrimidin-2-yl]pyrrolidin-3- | | | 1.5 | |
| yl}amino)ethyl]acetamide | | | | |
| N-methyl-N-[2-(methyl{(3R)-1-[4-(2-naphthyl)pyrimidin-2-yl]pyrrolidin-3- | | | | 2.5 |
| yl}amino)ethyl]propanamide | | | | |
| N,2-dimethyl-N-[2-(methyl{(3R)-1-[4-(2-naphthyl)pyrimidin-2-yl]pyrrolidin-3- | | | | 1.5 |
| yl}amino)ethyl]propanamide | | | | |
| N-methyl-N-[2-(methyl{(3R)-1-[4-(2-naphthyl)pyrimidin-2-yl]pyrrolidin-3- | | | | 1.9 |
| yl}amino)ethyl]methanesulfonamide | | | | |
| 1,1-diethyl-3-methyl-3-[2-(methyl{(3R)-1-[4-(2-naphthyl)pyrimidin-2-yl]pyrrolidin-3- | | | 1.93 | |
| yl}amino)ethyl]urea | | | | |
| methyl methyl[2-(methyl{(3R)-1-[4-(2-naphthyl)pyrimidin-2-yl]pyrrolidin-3- | | | | 1.2 |
| yl}amino)ethyl]carbamate | | | | |
| N-methyl-N-[2-(methyl{(3R)-1-[4-(2-naphthyl)pyrimidin-2-yl]pyrrolidin-3- | | | | 1.3 |
| yl}amino)ethyl]ethanesulfonamide | | | | |
| N,N,N′-trimethyl-N′-[2-(methyl{(3R)-1-[4-(2-naphthyl)pyrimidin-2-yl]pyrrolidin-3- | | | 1.3 | |
| yl}amino)ethyl]sulfamide | | | | |
| N-methyl-N-[2-(methyl{(3R)-1-[4-(2-naphthyl)pyrimidin-2-yl]pyrrolidin-3- | | | 1.3 | |
| yl}amino)ethyl]benzamide | | | | |
| N-methyl-N-[2-(methyl{(3R)-1-[4-(2-naphthyl)pyrimidin-2-yl]pyrrolidin-3- | | | | 1.4 |
| yl}amino)ethyl]benzenesulfonamide | | | | |
| 1-{1-[4-(2-naphthyl)pyrimidin-2-yl]-1,2,3,4-tetrahydroquinolin-4-yl}methanamine | | 1.820 | | |
| 2,2,2-trifluoro-N-({1-[4-(6-methoxy-2-naphthyl)pyrimidin-2-yl]piperidin-4- | | | | |
| yl}methyl)acetamide | | | | |
| 2,2,2-trifluoro-N-({1-[4-(6-hydroxy-2-naphthyl)pyrimidin-2-yl]piperidin-4- | | | | |
| yl}methyl)acetamide | | | | |
| (3aR,7aS)-5-[4-(2-naphthyl)pyrimidin-2-yl]octahydro-1H-pyrrolo[3,4-c]pyridine | | | 1.8 | |
| 1-{4-[4-(2-naphthyl)pyrimidin-2-yl]phenyl}methanamine | | | 2.5 | |
| 2-[4-(2-naphthyl)pyrimidin-2-yl]-2,8-diazaspiro[4.5]decane | | | 1.7 | |
| (3R)-1-{(3S)-1-[4-(2-naphthyl)pyrimidin-2-yl]pyrrolidin-3-yl}piperidin-3-ol | | | | 2.9 |
| (3S)-1-{(3S)-1-[4-(2-naphthyl)pyrimidin-2-yl]pyrrolidin-3-yl}piperidin-3-ol | 0.93 | | | |
| (3R)-1-{(3R)-1-[4-(2-naphthyl)pyrimidin-2-yl]pyrrolidin-3-yl}piperidin-3-ol | 2.2 | | | |
| (3S)-1-{(3R)-1-[4-(2-naphthyl)pyrimidin-2-yl]pyrrolidin-3-yl}piperidin-3-ol | 2.4 | | | |
| 1-{8-[4-(2-naphthyl)pyrimidin-2-yl]-8-azabicyclo[3.2.1]oct-3-yl}methanamine | | | 1.5 | |
| 1-{8-[4-(2-naphthyl)pyrimidin-2-yl]-8-azabicyclo[3.2.1]oct-3-yl}methanamine | 0.55 | | | |
| (3R,3′R)-1′-[4-(2-naphthyl)pyrimidin-2-yl]-1,3′-bipyrrolidin-3-ol | | | | 2.5 |
| (3S,3′R)-1′-[4-(2-naphthyl)pyrimidin-2-yl]-1,3′-bipyrrolidin-3-ol | | | | 3.0 |
| (3R,3′R)-1′-[4-(2-naphthyl)pyrimidin-2-yl]-1,3′-bipyrrolidin-3-amine | | | 1.39 | |
| (3S,3′R)-1′-[4-(2-naphthyl)pyrimidin-2-yl]-1,3′-bipyrrolidin-3-amine | | | 1.2 | |
| (1S,4S)-2-methyl-5-{(3R)-1-[4-(2-naphthyl)pyrimidin-2-yl]pyrrolidin-3-yl}-2,5- | | | 1.5 | |
| diazabicyclo[2.2.1]heptane | | | | |
| N,N-dimethyl-N′-{(3R)-1-[4-(2-naphthyl)pyrimidin-2-yl]pyrrolidin-3-yl}ethane-1,2- | | | 2.5 | |
| diamine | | | | |
| N,N′-dimethyl-N-{(3R)-1-[4-(2-naphthyl)pyrimidin-2-yl]pyrrolidin-3-yl}ethane-1,2- | | | 1.2 | |
| diamine | | | | |
| 3-(methyl{(3R)-1-[4-(2-naphthyl)pyrimidin-2-yl]pyrrolidin-3-yl}amino)propan-1-ol | | | | 3.3 |
| N,N,N′-trimethyl-N′-{(3R)-1-[4-(2-naphthyl)pyrimidin-2-yl]pyrrolidin-3-yl}ethane-1,2- | | | 2.4 | |
| diamine | | | | |
| 2-{(3R)-1-[4-(2-naphthyl)pyrimidin-2-yl]pyrrolidin-3-yl}octahydro-2H-pyrido[1,2- | | | 1.3 | |
| a]pyrazine | | | | |
| 3-(methyl{(3S)-1-[4-(2-naphthyl)pyrimidin-2-yl]pyrrolidin-3-yl}amino)propan-1-ol | | | 1.1 | |
| 1-{(1S,4S,5S)-2-[4-(2-naphthyl)pyrimidin-2-yl]-2-azabicyclo[2.2.1]hept-5-yl}methanamine | | | 1.2 | |
| 1-{(1S,4S,5R)-2-[4-(2-naphthyl)pyrimidin-2-yl]-2-azabicyclo[2.2.1]hept-5- | | | 1.1 | |
| yl}methanamine | | | | |
| (3R,3′S)-1′-[4-(2-naphthyl)pyrimidin-2-yl]-1,3′-bipyrrolidin-3-ol | | | 2.1 | |
| (3S,3′S)-1′-[4-(2-naphthyl)pyrimidin-2-yl]-1,3′-bipyrrolidin-3-ol | | | 2.1 | |
| N,N-dimethyl-N′-{(3S)-1-[4-(2-naphthyl)pyrimidin-2-yl]pyrrolidin-3-yl}ethane-1,2- | | | 1.5 | |
| diamine | | | | |
| N,N,N′-trimethyl-N′-{(3S)-1-[4-(2-naphthyl)pyrimidin-2-yl]pyrrolidin-3-yl}ethane-1,2- | | | 1.2 | |
| diamine | | | | |
| (3R)-1-({1-[4-(2-naphthyl)pyrimidin-2-yl]pyrrolidin-3-yl}methyl)pyrrolidin-3-ol | | | 1.2 | |
| (3S)-1-({1-[4-(2-naphthyl)pyrimidin-2-yl]pyrrolidin-3-yl}methyl)pyrrolidin-3-ol | | | 3.2 | |
| N,N,N′-trimethyl-N′-{(3R)-1-[4-(2-naphthyl)pyrimidin-2-yl]pyrrolidin-3-yl}propane-1,3- | | | 1.3 | |
| diamine | | | | |
| N,N,N′-trimethyl-N′-{(3S)-1-[4-(2-naphthyl)pyrimidin-2-yl]pyrrolidin-3-yl}propane-1,3- | | | 2.0 | |
| diamine | | | | |
| tert-butyl [(trans-4-{[4-(2-naphthyl)pyrimidin-2-yl]oxy}cyclohexyl)methyl]carbamate | | | | |
| 1-(trans-4-{[4-(2-naphthyl)pyrimidin-2-yl]oxy}cyclohexyl)methanamine | 0.46 | | | |
| 5-{(3R)-1-[4-(2-naphthyl)pyrimidin-2-yl]pyrrolidin-3-yl}octahydropyrrolo[3,4-b]pyrrole | | | 2.2 | |
| 5-{(3S)-1-[4-(2-naphthyl)pyrimidin-2-yl]pyrrolidin-3-yl}octahydropyrrolo[3,4-b]pyrrole | | | 2.2 | |
| benzyl [(cis-4-{methyl[4-(2-naphthyl)pyrimidin-2-yl]amino}cyclohexyl)methyl]carbamate | | | | |
| N-[trans-4-(aminomethyl)cyclohexyl]-N-methyl-4-(2-naphthyl)pyrimidin-2-amine | | | 1.4 | |
| N-[cis-4-(aminomethyl)cyclohexyl]-N-methyl-4-(2-naphthyl)pyrimidin-2-amine | | | 2.7 | |
| tert-butyl ({cis-4-[(4-naphthalen-2-ylpyrimidin-2-yl)oxy]cyclohexyl}methyl)carbamate | | | | |
| 1-{cis-4-[(4-naphthalen-2-ylpyrimidin-2-yl)oxy]cyclohexyl}methanamine | | | 4.2 | |
| 1-[trans-4-(4-naphthalen-2-ylpyrimidin-2-yl)cyclohexyl]methanamine | | | 3.5 | |
| 5-(4-naphthalen-2-ylpyrimidin-2-yl)octahydropyrrolo[3,4-b]pyrrole | | | 1.5 | |
| tert-butyl {2-[1-(4-naphthalen-2-ylpyrimidin-2-yl)azetidin-3-yl]ethyl}carbamate | | | | 1.6 |
| 2-[1-(4-naphthalen-2-ylpyrimidin-2-yl)azetidin-3-yl]ethanamine | | | 2.4 | |
| 1-{cis-4-[4-(2-naphthyl)pyrimidin-2-yl]cyclohexyl}methanamine | | | 3.4 | |
| N-ethyl-N,N′-dimethyl-N′-{(3R)-1-[4-(2-naphthyl)pyrimidin-2-yl]pyrrolidin-3-yl}ethane- | | | 1.8 | |
| 1,2-diamine | | | | |
| N,N′-dimethyl-N-{(3R)-1-[4-(2-naphthyl)pyrimidin-2-yl]pyrrolidin-3-yl}-N′-propylethane- | | | 3.4 | |
| 1,2-diamine | | | | |
| N-isopropyl-N,N′-dimethyl-N′-{(3R)-1-[4-(2-naphthyl)pyrimidin-2-yl]pyrrolidin-3- | | | 2.2 | |
| yl}ethane-1,2-diamine | | | | |
| N-benzyl-N,N′-dimethyl-N′-{(3R)-1-[4-(2-naphthyl)pyrimidin-2-yl]pyrrolidin-3-yl}ethane- | | | 1.6 | |
| 1,2-diamine | | | | |

A = MAX FOLD Induction/DMSO CONTROL
B = TCF ACTIVITY/DMSO CONTROL Fold induction @ 2.0 uM
C = TCF ACTIVITY/DMSO CONTROL Fold induction @ 20.0 uM

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound of Formula (A): 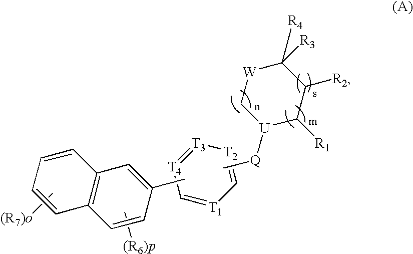
or a pharmaceutically acceptable salt thereof, wherein
T₁, T₂, T₃ and T₄ are independently CH or N, wherein two of T₁, T₂, T₃ and T₄ are N and the remaining two of T₁, T₂, T₃ and T₄ are CH;
Q is a bond, O, N(CH₂)ᵣR₈ or CR₈R₉;
U is N or CR₁₀;
W is CHR₅, O, or NR₅;
each R₁ is independently H or C₁-C₆ alkyl;
R₂ is C₁-C₁₀ alkyl optionally substituted with one or two substitutents independently selected form a group consisting of NR₁₁R₁₂, COR₁₁, CO₂R₁₁, CONR₁₁R₁₂, OR₁₁, SOₓR₁₁ and SO₂NR₁₁R₁₂; or R₁ and R₂ when taken together with the ring to which they are attached form a C₈-C₁₂ bicyclic cycloakyl or an 8- to 12-membered bicyclic heterocycle;
R₃ is H, halogen, OR₁₁ or C₁-C₁₀ alkyl optionally substituted with one or two substitutents independently selected form a group consisting of NO₂, NR₁₁R₁₂, COR₁₁, CO₂R₁₁, CONR₁₁R₁₂, OR₁₁, SOₓR₁₁ and SO₂NR₁₁R₁₂; or R₂ and R₃ when taken together with the ring to which they are attached form a C₈-C₁₂ bicyclic cycloakyl or an 8- to 12-membered bicyclic heterocycle;
R₄ is H, halogen, OR₁₁, NR₁₁R₁₂, C₁₋₆ alkyl optionally substituted with at least one and up to two substitutents independently selected form a group consisting of NR₁₀R₁₁, COR₁₀, CO₂R₁₀, CONR₁₀R₁₁, OR₁₀, SOₓR₁₀ and SO₂NR₁₀R₁₁; or R₃ and R₄ when taken together with the carbon to which they are attached to form a C₃-C₈ monocyclic cycloalkyl, or a 3- to 7-membered monocyclic heterocycle;
R₅ is independently H, 5-12-membered heteroaryl, OH, CN, OR₁₀, NR₁₁R₁₂, COR₁₁, CO₂R₁₁, CONR₁₁R₁₂, CSNR₁₁R₁₂, SOₓR₁₁, SO₂NR₁₁R_{12,} NHSO₂R_{11,} NHSO_{2,} NR₁₁R₁₂, NHCONR₁₁R₁₂, NHC(═NR₁₁)NR₁₁R₁₂, N₃ or C₁₋C₆ alkyl optionally substituted with halogen, R_{11,} OR₁₀, or NR₁₁R₁₂; or R₅ and R₄ when taken together with the carbon to which they are attached to form a C₃-C₈ monocyclic cycloalkyl or a 3- to 7-membered monocyclic heterocycle;
or R₅ and R₂ when taken together with the ring to which they are attached to form an C₈-C₁₂ bicyclic cycloalkyl or an 8- to 12-membered bicyclic heterocycle; or R₅ and R₁ when taken together with the ring to which they are attached form an C₈-C₁₂ bicyclic cycloalkyl or an 8- to 12-membered bicyclic heterocycle;
R₆ and R₇ are independently H, halogen, CN, NO₂, R₁₁, OR₁₁, SOₓR₁₁, NR₁₁R₁₂;
R₈, R₉ and R₁₀ are independently H, C₁-C₆ alkyl optionally substituted with aryl or with CO₂R₁₃, or R₈ and R₉ taken together are ═O;
R₁₁ is H; C₂-C₆ alkenyl; C₁-C₆ alkyl optionally substituted with OR₁₃, N R₁₃R₁₄, halogen or with 3-7-membered monocyclic heterocycle; cycloalkyl or monocyclic or bicyclic heterocycle; aryl optionally substituted with halogen, NR₁₃R₁₄, CN or C₁-C₆ alkyl; arylalkyl, COR₁₃, CO₂R₁₃, CONR₁₃R₁₄, SO₂R₁₃, SO₂NR₁₃R₁₄ or C(═NR₁₃)NR₁₃R₁₄;
R₁₂ is H; C₁-C₆ alkyl; aryl optionally substituted with C₁-C₆ alkyl; arylalkyl, COR₁₃, CO₂R₁₃, CONR₁₃R₁₄, SO₂R₁₃, SO₂NR₁₃R₁₄ or C(═NR₁₃)NR₁₃R₁₄; or R₁₁ and R₁₂ when taken together with N to which they are attached form a 3- to 7-membered monocyclic heterocycle; or 8-12-membered bicyclic heterocycle, wherein the monocyclic heterocycle, or the bicyclic heterocycle is optionally substituted with one or two alkyl, ═O, NR₁₃R₁₄, OR₁₃ or CH₂OR₁₃;
R₁₃ is H, C₁-C₆ alkyl optionally substituted with halogen, CO—C₁-C₆ alkyl optionally substituted with halogen, CO-aryl, SO₂C₁-C₆ alkyl, SO₂-aryl, SO₂-di(C₁-C₆)alkylamino, di(C₁-C₆)alkylamino, COO—C₁-C₆ alkyl, COO-aryl optionally substituted with alkyl, NHCOO-arylalkyl, aryl optionally substituted with alkyl;
R₁₄ is H or C₁-C₆ alkyl; or R₁₃ and R₁₄ when taken together with the N to which they are attached to form a 3- to 7-membered monocyclic heterocycle;
m, n, o, p, and x are independently 0,1 or 2;
s is 0 or 1; and
r is 0, 1, 2 or 3.

2. The compound of claim 1, wherein R₇ is H or OR₁₁.

3. The compound of claim 1, wherein R₅ is 5-10-membered heteroaryl.

4. The compound of claim 3, wherein R₅ is 5-7-membered heteroaryl

5. The compound of claim 1, wherein Q is a bond or N(CH₂)ᵣR₈.

6. The compound of claim 5, wherein Q is a bond.

7. The compound of claim 1, wherein r is 0, 1 or 2.

8. The compound of claim 1, wherein R₈ is H.

9. The compound of claim 1, wherein Formula (A) is Formula (AI): 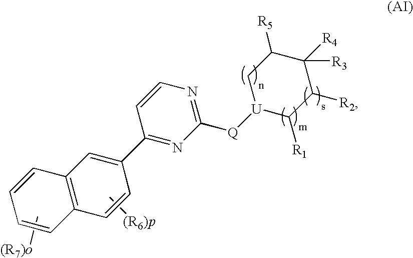
wherein Q, U, R₁, R₂, R₃, R₄, R₅, R₆, R₇, m, n, o, p and s are as defined in claim 1.

10. The compound of claim 1, wherein the 3- to 7-membered heterocycle formed by R₃ and R₄ is a 5, 6, or 7-membered heteroaryl.

11. The compound of claim 1, wherein the 3- to 7-membered heterocycle formed by R₅ and R₄ is a 5, 6, or 7-membered heteroaryl.

12. The compound of claim 1, wherein the 3- to 7-membered heterocycle formed by R₁₁ and R₁₂ is a 5, 6, or 7-membered heteroaryl.

13. The compound of claim 1, wherein 3- to 7-membered heterocycle formed by R₁₃ and R₁₄ is a 5, 6, or 7-membered heteroaryl.

14. The compound of claim 1, wherein the R₁₁ is Cl-C₆ alkyl optionally substituted with a 5-7 membered heteroaryl.

15. The compound of claim 1, wherein 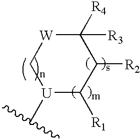
is selected from the group consisting of 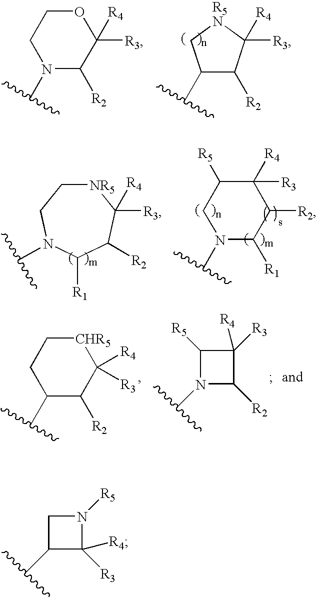
wherein R₁, R₂, R₃, R₄, R₅, m, n and s are as defined in claim 1.

16. The compound of claim 1, wherein 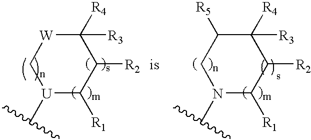
wherein R₁, R₂, R₃, R₄, R₅, s, m, and n are as defined in claim 1.

17. The compound of claim 16, wherein n=1 and m=1.

18. The compound of claim 16, wherein n=1 and m=0.

19. The compound of claim 16, where n=2 and m=0.

20. The compound of claim 16, wherein R₂ is CH₂OR₁₁.

21. The compound of claim 16, wherein R₃ is OR₁₁ or CH₂OR₁₁.

22. The compound of claim 16, wherein R₄ is OR₁₁.

23. The compound of claim 16, wherein R₅ is CN; N R₁₁R₁₂; C(S)N R₁₁R₁₂; or alkyl optionally substituted with N R₁₁R₁₂, monocyclic heterocycle, or bicyclic heterocycle.

24. The compound of claim 16, wherein 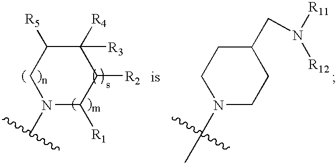
wherein R₁, R₂, R₃, R₄, R₅, R₁₁, R₁₂, s, m and n are as defined in claim 16.

25. The compound of claim 16, wherein 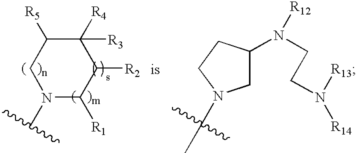
wherein R₁, R₂, R₃, R₄, R₅, R₁₂, R₁₃, R₁₄, s, m, and n are as defined in claim 16.

26. The compound of claim 25, wherein 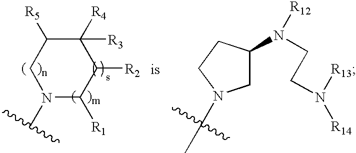
wherein R₁, R₂, R₃, R₄, R₅, R₁₂, R₁₃, R₁₄, s, m, and n are as defined in claim 25.

27. The compound of claim 15, wherein 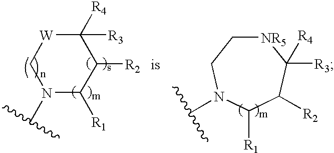
wherein m is 0 or 1.

28. The compound of claim 1, wherein Formula (A) is 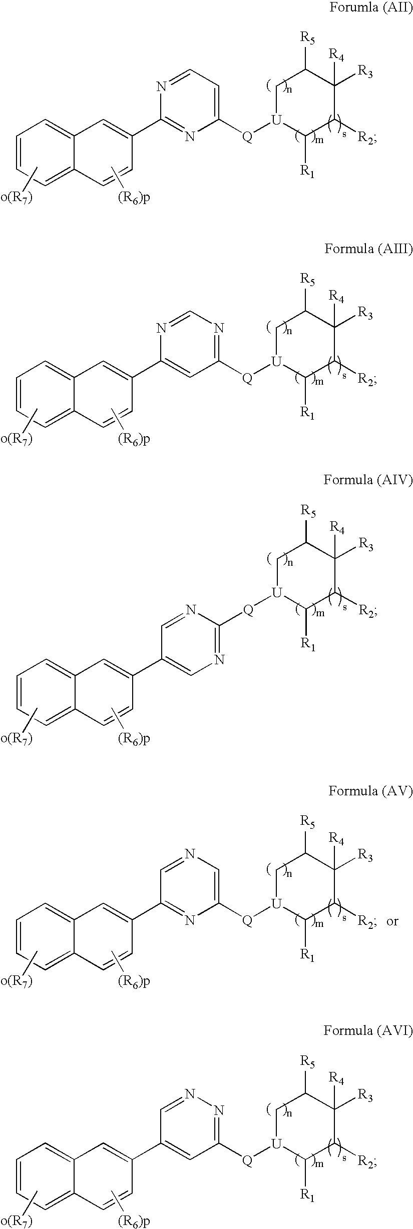
wherein Q, U, R₁, R₂, R₃, R₄, R₅, R₆, R₇, m, n, o, p and s are as defined in claim 1.

29. The compound of claim 1, wherein Formula (A) is Formula (I): 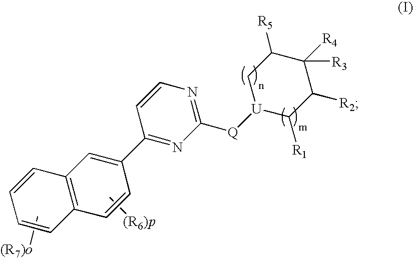
wherein
Q is a bond or CR₈R₉;
U is N or CR₁₀;
R₁ is H or C₁₋C₆ alkyl;
R₂ is C₁-C₁₀ alkyl substituted with 0, 1 or 2 of NR₁₁R₁₂, COR₁₁, CO₂R₁₁, CONR₁₁R₁₂, OR₁₁, S(O)ₓR₁₁, or SO₂NR₁₁R₁₂; or R₁ and R₂ when taken together with the ring to which they are attached form an 8- to 12-membered bicyclic heterocycle;
R₃ is H, halogen, C₁₋C₁₀ alkyl substituted with 0, 1 or 2 of NR₁₁R₁₂, COR₁₁, CO₂R₁₁, CONR₁₁R₁₂, OR₁₁, S(O)ₓR₁₁, or SO₂NR₁₁R₁₂; or R₂ and R₃ when taken together with the ring to which they are attached form an 8- to 12-membered bicyclic heterocycle or homocycle;
R₄ is H, halogen, OR₁₁, NR₁₁R₁₂, C₁₋C₆ alkyl substituted with at least one and up to two of NR₁₀R₁₁, COR₁₀, CO₂R₁₀, CONR₁₀R₁₁, OR₁₀, S(O)ₓR₁₀, or SO₂NR₁₀R₁₁; or R₃ and R₄ when taken together with the carbon to which they are attached form a C₃-C₈ monocyclic cycloalkyl or a 3- to 7-membered monocyclic heterocycle;
R₅ is H, OR₁₀, NR₁₀R₁₁ or C₁₋C₆ alkyl optionally substituted with OR₁₀, or NR₁₀R₁₁; or R₅ and R₄ when taken together form a C₃-C₈ monocyclic cycloalkyl or a 3- to 7-membered monocyclic heterocycle; or R₅ and R₂, as well as R₅ and R₁, when taken together along with the ring to which they are attached form an C₈-C₁₂ bicyclic cycloalkyl or an 8- to 12-membered bicyclic heterocycle;
R₆ and R₇ are independently H, halogen, CN, NO₂, R₁₁, OR₁₁, S(O)ₓR₁₁, or NR₁₁R₁₂;
R₈ and R₉ are ═O or independently H or C₁₋C₆ alkyl
R₁₀ is H or C₁₋C₆ alkyl;
R₁₁ is H, C₁₋C₆ alkyl, aryl, or alkylaryl;
R₁₂ is H, C₁₋C₆ alkyl, aryl, alkylaryl, COR₁₃, CO₂R₁₃, CONR₁₃R₁₄, SO₂R₁₃; or R₁₁ and R₁₂ when taken together with the N to which they are attached form a C₃-C₈ monocyclic cycloalkyl, a 3- to 7-membered monocyclic heterocycle, an C₈-C₁₂ bicyclic cycloalkyl, or an 8- to 12-membered bicyclic heterocycle, all optionally substituted with R₁₁ and OR₁₁;
R₁₃ is H or C₁₋C₆ alkyl;
R₁₄ is H or C₁₋C₆ alkyl; or R₁₃ and R₁₄ when taken together with the N to which they are attached form a C₃-C₈ monocyclic cycloalkyl or a 3- to 7-membered monocyclic heterocycle;
m, n, o and p are independently 0, 1 or 2; and
x is 0, 1, or 2.

30. A compound of the Formula (II): 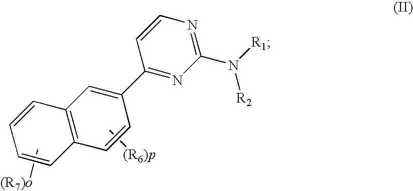
or pharmaceutically acceptable salts thereof,
wherein
R₁ and R₂ are independently —H; C₁-C₆ alkyl optionally substituted with C1-C6 alkoxy, hydroxyl, or C6-C10 aryl; C(O)C₁-C₆ alkyl, C(O)NC₁-C₆ alkyl, a C₃-C₈ monocyclic cycloalkyl, or a 3- to 7-membered monocyclic heterocycle, with the proviso that R₁ and R₂ are not both H;
R₆ and R₇ are independently H, halogen, CN, NO₂, R₁₁, OR₁₁, S(O)ₓR₁₁, or NR₁₁R₁₂;
R₁₁ is H, C₁-C₆ alkyl, aryl or alkylaryl;
R₁₂ is H, C₁-C₆ alkyl, aryl, alkylaryl, COR₁₃, CO₂R₁₃, CONR₁₃ R₁₄, or SO₂R₁₃; or R₁₁ and R₁₂ when taken together with the N to which they are attached form a 3- to 7-membered monocyclic heterocycle or an 8- to 12-membered bicyclic heterocycle;
R₁₃ is H or C₁₋C₆ alkyl;
R₁₄ is H or C₁₋C₆ alkyl; or R₁₃ and R₁₄ when taken together with the N to which they are attached form a 3- to 7-membered monocyclic heterocycle;
o and p are independently 0, 1 or 2; and
x is 0, 1, or 2.

31. The compound of claim 1, or a pharmaceutically acceptable salt thereof, wherein the compound is
tert-butyl {(3S)-1-[4-(2-naphthyl)pyrimidin-2-yl]pyrrolidin-3-yl}carbamate;
(3S)-1-[4-(2-naphthyl)pyrimidin-2-yl]pyrrolidin-3-amine;
4-[4-(2-naphthyl)pyrimidin-2-yl]-1,4-diazepane-1-carbaldehyde;
1-[4-(2-naphthyl)pyrimidin-2-yl]-1,4-diazepane;
1-[4-(2-naphthyl)pyrimidin-2-yl]azepan-4-amine;
tert-butyl {1-[4-(2-naphthyl)pyrimidin-2-yl]azepan-4-yl}carbamate;
tert-butyl ({1-[4-(2-naphthyl)pyrimidin-2-yl]azetidin-3-yl}methyl)carbamate;
1-{1-[4-(2-naphthyl)pyrimidin-2-yl]azetidin-3-yl }methanamine;
tert-butyl ({1-[4-(2-naphthyl)pyrimidin-2-yl]pyrrolidin-3-yl}methyl)carbamate;
1-{1-[4-(2-naphthyl)pyrimidin-2-yl]pyrrolidin-3-yl}methanamine;
4-(2-{4-[4-(2-naphthyl)pyrimidin-2-yl]piperazin-1-yl}ethyl)morpholine;
4-({4-[4-(2-naphthyl)pyrimidin-2-yl]piperazin-1-yl}acetyl)morpholine;
N,N-dimethyl-2-{4-[4-(2-naphthyl)pyrimidin-2-yl]piperazin-1-yl}ethanamine;
2-(4-methylpiperazin-1-yl)-4-(2-naphthyl)pyrimidine;
4-(2-naphthyl)-2-(4-pyridin-2-ylpiperazin-1-yl)pyrimidine;
4-(2-naphthyl)-2-(4-pyrimidin-2-ylpiperazin-1-yl)pyrimidine;
1-{1-[4-(2-naphthyl)pyrimidin-2-yl]piperidin-4-yl}-1,3-dihydro-2H-benzimidazol-2-one;
2-[4-(2-naphthyl)pyrimidin-2-yl]-1,2,3,4-tetrahydroisoquinoline;
(2-{4-[4-(2-naphthyl)pyrimidin-2-yl]piperazin-1-yl}ethyl)amine;
N,N-dimethyl-3-{4-[4-(2-naphthyl)pyrimidin-2-yl]piperazin-1-yl}propan-1-amine;
4-(2-naphthyl)-2-(4-pyridin-4-ylpiperazin-1-yl)pyrimidine;
4-(3-{4-[4-(2-naphthyl)pyrimidin-2-yl]piperazin-1-yl}propyl)morpholine;
2-[4-(2-furoyl)piperazin-1-yl]-4-(2-naphthyl)pyrimidine;
tert-butyl {(3R)-1-[4-(2-naphthyl)pyrimidin-2-yl]pyrrolidin-3-yl}carbamate;
(3R)-1-[4-(2-naphthyl)pyrimidin-2-yl]pyrrolidin-3-amine;
N,N-dimethyl-1-[4-(2-naphthyl)pyrimidin-2-yl]pyrrolidin-3-amine;
N,N-diethyl-1-[4-(2-naphthyl)pyrimidin-2-yl]pyrrolidin-3-amine;
(3R)-N-methyl-1-[4-(2-naphthyl)pyrimidin-2-yl]pyrrolidin-3-amine;
(3S)-N-methyl-1-[4-(2-naphthyl)pyrimidin-2-yl]pyrrolidin-3-amine;
(3S)-N,N-dimethyl-1-[4-(2-naphthyl)pyrimidin-2-yl]pyrrolidin-3-amine;
(3R)-N,N-dimethyl-1-[4-(2-naphthyl)pyrimidin-2-yl]pyrrolidin-3-amine;
N-methyl-N-{(3S)-1-[4-(2-naphthyl)pyrimidin-2-yl]pyrrolidin-3-yl}acetamide;
{1-[4-(2-naphthyl)pyrimidin-2-yl]piperidin-4-yl}methanol;
2-{1-[4-(2-naphthyl)pyrimidin-2-yl]piperidin-4-yl}ethanol;
ethyl 1-[4-(2-naphthyl)pyrimidin-2-yl]piperidine-4-carboxylate;
ethyl {1-[4-(2-naphthyl)pyrimidin-2-yl]piperidin-4-yl}acetate;
{(2S)-1-[4-(2-naphthyl)pyrimidin-2-yl]pyrrolidin-2-yl}methanol;
1-[4-(2-naphthyl)pyrimidin-2-yl]pyrrolidin-3-ol;
4-[4-(2-naphthyl)pyrimidin-2-yl]morpholine;
{1-[4-(2-naphthyl)pyrimidin-2-yl]piperidin-2-yl}methanol;
{1-[4-(2-naphthyl)pyrimidin-2-yl]piperidin-3-yl}methanol;
1-[4-(2-naphthyl)pyrimidin-2-yl]piperidin-4-ol;
1-[4-(2-naphthyl)pyrimidin-2-yl]piperidin-3-ol;
{(2R)-1-[4-(2-naphthyl)pyrimidin-2-yl]pyrrolidin-2-yl}methanol;
4-(2-naphthyl)-2-pyrrolidin-1-ylpyrimidine;
4-(2-naphthyl)-2-piperidin-1-ylpyrimidine;
2-(4-methylpiperidin-1-yl)-4-(2-naphthyl)pyrimidine;
1-[4-(2-naphthyl)pyrimidin-2-yl]azepane;
tert-butyl {1-[4-(2-naphthyl)pyrimidin-2-yl]piperidin-4-yl}carbamate;
tert-butyl 4-[4-(2-naphthyl)pyrimidin-2-yl]piperazine-1-carboxylate;
1-[4-(2-naphthyl)pyrimidin-2-yl]piperidin-4-amine;
4-(2-naphthyl)-2-piperazin-1-ylpyrimidine;
tert-butyl ({1-[4-(2-naphthyl)pyrimidin-2-yl]piperidin-3-yl}methyl)carbamate;
({1-[4-(2-naphthyl)pyrimidin-2-yl]piperidin-3-yl}methyl)amine;
(1R,5S,6s)-3-[4-(2-naphthyl)pyrimidin-2-yl]-3-azabicyclo[3.1.0]hexan-6-amine;
({1-[4-(2-naphthyl)pyrimidin-2-yl]piperidin-4-yl}methyl)amine;
1-[4-(2-naphthyl)pyrimidin-2-yl]piperidine-4-carboxamide;
8-[4-(2-naphthyl)pyrimidin-2-yl]-1,4-dioxa-8-azaspiro[4.5]decane;
methyl 1-[4-(2-naphthyl)pyrimidin-2-yl]piperidine-4-carboxylate;
1-[4-(2-naphthyl)pyrimidin-2-yl]piperidine-3-carboxamide;
{(3S)-1-[4-(2-naphthyl)pyrimidin-2-yl]piperidin-3-yl}methanol;
{(3R)-1-[4-(2-naphthyl)pyrimidin-2-yl]piperidin-3-yl}methanol;
N-{(3S)-1-[4-(2-naphthyl)pyrimidin-2-yl]pyrrolidin-3-yl}acetamide;
N-({1-[4-(2-naphthyl)pyrimidin-2-yl]piperidin-4-yl}methyl)guanidine;
N-ethyl-4-[4-(2-naphthyl)pyrimidin-2-yl]-1,4-diazepane-1-carboxamide;
methyl 4-[4-(2-naphthyl)pyrimidin-2-yl]-1,4-diazepane-1-carboxylate;
ethyl 4-[4-(2-naphthyl)pyrimidin-2-yl]-1,4-diazepane-1-carboxylate;
1-acetyl-4-[4-(2-naphthyl)pyrimidin-2-yl]-1,4-diazepane;
1-[4-(2-naphthyl)pyrimidin-2-yl]-4-propionyl-1,4-diazepane;
1-[4-(2-naphthyl)pyrimidin-2-yl]-4-(trifluoroacetyl)-1,4-diazepane;
N,N-diethyl-4-[4-(2-naphthyl)pyrimidin-2-yl]-1,4-diazepane-1-carboxamide;
1-(methylsulfonyl)-4-[4-(2-naphthyl)pyrimidin-2-yl]-1,4-diazepane;
N,N-dimethyl-4-[4-(2-naphthyl)pyrimidin-2-yl]-1,4-diazepane-1-sulfonamide;
N,N-dimethyl-4-[4-(2-naphthyl)pyrimidin-2-yl]-1,4-diazepane-1-carboxamide;
4-[4-(2-naphthyl)pyrimidin-2-yl]-1,4-diazepane-1-carboxamide;
1-benzoyl-4-[4-(2-naphthyl)pyrimidin-2-yl]-1,4-diazepane;
1-[(4-methylphenyl)sulfonyl]-4-[4-(2-naphthyl)pyrimidin-2-yl]-1,4-diazepane;
N-{1-[4-(2-naphthyl)pyrimidin-2-yl]azepan-4-yl}acetamide;
2,2,2-trifluoro-N-{1-[4-(2-naphthyl)pyrimidin-2-yl]azepan-4-yl}acetamide;
N,N-dimethyl-N′-{1-[4-(2-naphthyl)pyrimidin-2-yl]azepan-4-yl}urea;
N-{1-[4-(2-naphthyl)pyrimidin-2-yl]azepan-4-yl}benzamide;
N-{1-[4-(2-naphthyl)pyrimidin-2-yl]azepan-4-yl}urea;
N-ethyl-N′-{1-[4-(2-naphthyl)pyrimidin-2-yl]azepan-4-yl}urea;
N-{1-[4-(2-naphthyl)pyrimidin-2-yl]azepan-4-yl}methanesulfonamide;
4-methyl-N-{1-[4-(2-naphthyl)pyrimidin-2-yl]azepan-4-yl}benzenesulfonamide;
N,N-dimethyl-N′-{1-[4-(2-naphthyl)pyrimidin-2-yl]azepan-4-yl}sulfamide;
N-({1-[4-(2-naphthyl)pyrimidin-2-yl]azetidin-3-yl}methyl)acetamide;
2,2,2-trifluoro-N-({1-[4-(2-naphthyl)pyrimidin-2-yl]azetidin-3-yl}methyl)acetamide;
N,N-dimethyl-N′-({1-[4-(2-naphthyl)pyrimidin-2-yl]azetidin-3-yl}methyl)urea;
methyl ({1-[4-(2-naphthyl)pyrimidin-2-yl]azetidin-3-yl}methyl)carbamate;
N-({1-[4-(2-naphthyl)pyrimidin-2-yl]azetidin-3-yl}methyl)urea;
N-ethyl-N′-({1-[4-(2-naphthyl)pyrimidin-2-yl]azetidin-3-yl}methyl)urea;
N-({1-[4-(2-naphthyl)pyrimidin-2-yl]azetidin-3-yl}methyl)methanesulfonamide;
4-methyl-N-({1-[4-(2-naphthyl)pyrimidin-2-yl]azetidin-3-yl}methyl)benzenesulfonamide;
{1-[4-(2-naphthyl)pyrimidin-2-yl]azepan-4-yl}formamide;
N,N-dimethyl-N′-(2-{4-[4-(2-naphthyl)pyrimidin-2-yl]piperazin-1-yl}ethyl)urea;
N-(2-{4-[4-(2-naphthyl)pyrimidin-2-yl]piperazin-1-yl}ethyl)methanesulfonamide;
N-(2-{4-[4-(2-naphthyl)pyrimidin-2-yl]piperazin-1-yl}ethyl)dicarbonimidic diamide;
N-ethyl-N′-(2-{4-[4-(2-naphthyl)pyrimidin-2-yl]piperazin-1-yl}ethyl)urea;
N-isopropyl-N′-(2-{4-[4-(2-naphthyl)pyrimidin-2-yl]piperazin-1-yl}ethyl)urea;
diethyl (2-{4-[4-(2-naphthyl)pyrimidin-2-yl]piperazin-1-yl}ethyl)imidodicarbonate;
N-{(3R)-1-[4-(2-naphthyl)pyrimidin-2-yl]pyrrolidin-3-yl}acetamide;
2,2,2-trifluoro-N-{(3R)-1-[4-(2-naphthyl)pyrimidin-2-yl]pyrrolidin-3-yl}acetamide;
methyl {(3R)-1-[4-(2-naphthyl)pyrimidin-2-yl]pyrrolidin-3-yl}carbamate;
N,N-dimethyl-N′-{(3R)-1-[4-(2-naphthyl)pyrimidin-2-yl]pyrrolidin-3-yl}urea;
N-{(3R)-1-[4-(2-naphthyl)pyrimidin-2-yl]pyrrolidin-3-yl}pyrrolidine-1-carboxamide
N-{(3R)-1-[4-(2-naphthyl)pyrimidin-2-yl]pyrrolidin-3-yl}urea;
N,N-dimethyl-N′-{(3R)-1-[4-(2-naphthyl)pyrimidin-2-yl]pyrrolidin-3-yl}sulfamide;
N-{(3R)-1-[4-(2-naphthyl)pyrimidin-2-yl]pyrrolidin-3-yl}methanesulfonamide;
4-methyl-N-{(3R)-1-[4-(2-naphthyl)pyrimidin-2-yl]pyrrolidin-3-yl}benzenesulfonamide;
N-{(3R)-1-[4-(2-naphthyl)pyrimidin-2-yl]pyrrolidin-3-yl}morpholine-4-carboxamide;
ethyl {(3R)-1-[4-(2-naphthyl)pyrimidin-2-yl]pyrrolidin-3-yl}carbamate;
N-isopropyl-N′-{(3R)-1-[4-(2-naphthyl)pyrimidin-2-yl]pyrrolidin-3-yl}urea;
N,N-diethyl-N′-{(3R)-1-[4-(2-naphthyl)pyrimidin-2-yl]pyrrolidin-3-yl}urea;
N-{(3R)-1-[4-(2-naphthyl)pyrimidin-2-yl]pyrrolidin-3-yl}guanidine;
4-chloro-N-{(3R)-1-[4-(2-naphthyl)pyrimidin-2-yl]pyrrolidin-3-yl}benzamide;
4-cyano-N-{(3R)-1-[4-(2-naphthyl)pyrimidin-2-yl]pyrrolidin-3-yl}benzamide;
N-{(3S)-1-[4-(2-naphthyl)pyrimidin-2-yl]pyrrolidin-3-yl}acetamide;
2,2,2-trifluoro-N-{(3S)-1-[4-(2-naphthyl)pyrimidin-2-yl]pyrrolidin-3-yl}acetamide;
N-{(3S)-1-[4-(2-naphthyl)pyrimidin-2-yl]pyrrolidin-3-yl}benzamide;
methyl {(3S)-1-[4-(2-naphthyl)pyrimidin-2-yl]pyrrolidin-3-yl}carbamate;
N,N-dimethyl-N′-{(3S)-1-[4-(2-naphthyl)pyrimidin-2-yl]pyrrolidin-3-yl}urea;
N-{(3S)-1-[4-(2-naphthyl)pyrimidin-2-yl]pyrrolidin-3-yl}pyrrolidine-1-carboxamide
N-ethyl-N′-{(3S)-1-[4-(2-naphthyl)pyrimidin-2-yl]pyrrolidin-3-yl}urea;
N-{(3S)-1-[4-(2-naphthyl)pyrimidin-2-yl]pyrrolidin-3-yl}urea;
N,N-dimethyl-N′-{(3S)-1-[4-(2-naphthyl)pyrimidin-2-yl]pyrrolidin-3-yl}sulfamide;
N-{(3S)-1-[4-(2-naphthyl)pyrimidin-2-yl]pyrrolidin-3-yl}methanesulfonamide;
4-methyl-N-{(3S)-1-[4-(2-naphthyl)pyrimidin-2-yl]pyrrolidin-3-yl}benzenesulfonamide;
N-{(3S)-1-[4-(2-naphthyl)pyrimidin-2-yl]pyrrolidin-3-yl}morpholine-4-carboxamide;
ethyl {(3S)-1-[4-(2-naphthyl)pyrimidin-2-yl]pyrrolidin-3-yl}carbamate;
N-isopropyl-N′-{(3S)-1-[4-(2-naphthyl)pyrimidin-2-yl]pyrrolidin-3-yl}urea;
N,N-diethyl-N′-{(3S)-1-[4-(2-naphthyl)pyrimidin-2-yl]pyrrolidin-3-yl}urea;
4-chloro-N-{(3S)-1-[4-(2-naphthyl)pyrimidin-2-yl]pyrrolidin-3-yl}benzamide;
4-cyano-N-{(3S)-1-[4-(2-naphthyl)pyrimidin-2-yl]pyrrolidin-3-yl}benzamide;
N-({1-[4-(2-naphthyl)pyrimidin-2-yl]pyrrolidin-3-yl}methyl)acetamide;
N-({1-[4-(2-naphthyl)pyrimidin-2-yl]pyrrolidin-3-yl}methyl)benzamide;
methyl ({1-[4-(2-naphthyl)pyrimidin-2-yl]pyrrolidin-3-yl}methyl)carbamate;
N,N-dimethyl-N′-({1-[4-(2-naphthyl)pyrimidin-2-yl]pyrrolidin-3-yl}methyl)urea;
N-ethyl-N′-({1-[4-(2-naphthyl)pyrimidin-2-yl]pyrrolidin-3-yl}methyl)urea;
N-({1-[4-(2-naphthyl)pyrimidin-2-yl]pyrrolidin-3-yl}methyl)urea;
N,N-dimethyl-N′-({1-[4-(2-naphthyl)pyrimidin-2-yl]pyrrolidin-3-yl}methyl)sulfamide;
N-({1-[4-(2-naphthyl)pyrimidin-2-yl]pyrrolidin-3-yl}methyl)methanesulfonamide;
N-({1-[4-(2-naphthyl)pyrimidin-2-yl]pyrrolidin-3-yl}methyl)formamide;
N-(2-{1-[4-(2-naphthyl)pyrimidin-2-yl]piperidin-4-yl}ethyl)acetamide;
2,2,2-trifluoro-N-(2-{1-[4-(2-naphthyl)pyrimidin-2-yl]piperidin-4-yl}ethyl)acetamide;
methyl (2-{1-[4-(2-naphthyl)pyrimidin-2-yl]piperidin-4-yl}ethyl)carbamate;
N,N-dimethyl-N′-(2-{1-[4-(2-naphthyl)pyrimidin-2-yl]piperidin-4-yl}ethyl)urea;
N,N-diethyl-N′-(2-{1-[4-(2-naphthyl)pyrimidin-2-yl]piperidin-4-yl}ethyl)urea;
N-ethyl-N′-(2-{1-[4-(2-naphthyl)pyrimidin-2-yl]piperidin-4-yl}ethyl)urea;
N-isopropyl-N′-(2-{1-[4-(2-naphthyl)pyrimidin-2-yl]piperidin-4-yl}ethyl)urea;
N-(2-{1-[4-(2-naphthyl)pyrimidin-2-yl]piperidin-4-yl}ethyl)urea;
N-cyclohexyl-N′-(2-{1-[4-(2-naphthyl)pyrimidin-2-yl]piperidin-4-yl}ethyl)urea;
N-(2-{1-[4-(2-naphthyl)pyrimidin-2-yl]piperidin-4-yl}ethyl)pyrrolidine-1-carboxamide;
N-(2-{1-[4-(2-naphthyl)pyrimidin-2-yl]piperidin-4-yl}ethyl)morpholine-4-carboxamide;
N,N-dimethyl-N′-(2-{1-[4-(2-naphthyl)pyrimidin-2-yl]piperidin-4-yl}ethyl)sulfamide;
N-(2-{1-[4-(2-naphthyl)pyrimidin-2-yl]piperidin-4-yl}ethyl)methanesulfonamide;
benzyl (2-{1-[4-(2-naphthyl)pyrimidin-2-yl]piperidin-4-yl}ethyl)carbamate;
N-(2-{1-[4-(2-naphthyl)pyrimidin-2-yl]piperidin-4-yl}ethyl)-N′-phenylurea;
N-(2-{1-[4-(2-naphthyl)pyrimidin-2-yl]piperidin-4-yl}ethyl)benzamide;
N-({1-[4-(2-naphthyl)pyrimidin-2-yl]piperidin-4-yl}methyl)acetamide;
N-({1-[4-(2-naphthyl)pyrimidin-2-yl]piperidin-4-yl}methyl)acetamide;
N-({1-[4-(2-naphthyl)pyrimidin-2-yl]piperidin-4-yl}methyl)benzamide;
methyl ({1-[4-(2-naphthyl)pyrimidin-2-yl]piperidin-4-yl}methyl)carbamate;
N,N-dimethyl-N′-({1-[4-(2-naphthyl)pyrimidin-2-yl]piperidin-4-yl}methyl)urea;
N-({1-[4-(2-naphthyl)pyrimidin-2-yl]piperidin-4-yl}methyl)pyrrolidine-1-carboxamide
N-ethyl-N′-({1-[4-(2-naphthyl)pyrimidin-2-yl]piperidin-4-yl}methyl)urea;
N-({1-[4-(2-naphthyl)pyrimidin-2-yl]piperidin-4-yl}methyl)urea;
N,N-dimethyl-N′-({1-[4-(2-naphthyl)pyrimidin-2-yl]piperidin-4-yl}methyl)sulfamide;
N-({1-[4-(2-naphthyl)pyrimidin-2-yl]piperidin-4-yl}methyl)methanesulfonamide;
4-methyl-N-({1-[4-(2-naphthyl)pyrimidin-2-yl]piperidin-4-yl}methyl)benzenesulfonamide;
N-({1-[4-(2-naphthyl)pyrimidin-2-yl]piperidin-4-yl}methyl)pyrimidin-2-amine;
di-tert-butyl {(Z)-[({1-[4-(2-naphthyl)pyrimidin-2-yl]piperidin-4-yl}methyl) amino]methylylidene}biscarbamate;
di-tert-butyl ((E)-{4-[4-(2-naphthyl)pyrimidin-2-yl]piperazin-1-yl}methylylidene)biscarbamate;
4-[4-(2-naphthyl)pyrimidin-2-yl]piperazine-1-carboximidamide;
{1-[4-(2-naphthyl)pyrimidin-2-yl]piperidin-4-yl}methyl 4-methylbenzenesulfonate;
2-{1-[4-(2-naphthyl)pyrimidin-2-yl]piperidin-4-yl}ethyl methanesulfonate;
2-[4-(2-azidoethyl)piperidin-1-yl]-4-(2-naphthyl)pyrimidine;
N,N-dimethyl-1-{1-[4-(2-naphthyl)pyrimidin-2-yl]piperidin-4-yl}methanamine;
N-methyl-1-{1-[4-(2-naphthyl)pyrimidin-2-yl]piperidin-4-yl}methanamine;
2-[4-(1H-imidazol-1-ylmethyl)piperidin-1-yl]-4-(2-naphthyl)pyrimidine;
4-(2-naphthyl)-2-[4-(pyrrolidin-1-ylmethyl)piperidin-1-yl]pyrimidine;
N-ethyl-N-({1-[4-(2-naphthyl)pyrimidin-2-yl]piperidin-4-yl}methyl)ethanamine;
4-(2-naphthyl)-2-[4-(piperidin-1-ylmethyl)piperidin-1-yl]pyrimidine;
4-(2-naphthyl)-2-[4-(piperidin-1-ylmethyl)piperidin-1-yl]pyrimidine;
N-({1-[4-(2-naphthyl)pyrimidin-2-yl]piperidin-4-yl}methyl)ethanamine;
2-methyl-N-({1-[4-(2-naphthyl)pyrimidin-2-yl]piperidin-4-yl}methyl)propan-1-amine;
2-{1-[4-(2-naphthyl)pyrimidin-2-yl]piperidin-4-yl}ethanamine;
1-[4-(2-naphthyl)pyrimidin-2-yl]piperidin-4-one;
1-[4-(2-naphthyl)pyrimidin-2-yl]piperidine-4-carbaldehyde;
1-[4-(2-naphthyl)pyrimidin-2-yl]piperidine-4-carboxylic acid;
4-methyl-1-[4-(2-naphthyl)pyrimidin-2-yl]piperidin-4-ol;
1-[4-(2-naphthyl)pyrimidin-2-yl]-4-(nitromethyl)piperidin-4-ol;
4-(aminomethyl)-1-[4-(2-naphthyl)pyrimidin-2-yl]piperidin-4-ol;
1-[4-(2-naphthyl)pyrimidin-2-yl]-4-(1-nitroethyl)piperidin-4-ol;
4-(2-naphthyl)-2-[4-(1-nitroethyl)piperidin-1-yl]pyrimidine;
4-(2-naphthyl)-2-[4-(nitromethyl)piperidin-1-yl]pyrimidine;
tert-butyl ((1S)-1-{1-[4-(2-naphthyl)pyrimidin-2-yl]piperidin-4-yl}ethyl)carbamate;
tert-butyl (1-{1-[4-(2-naphthyl)pyrimidin-2-yl]piperidin-4-yl}ethyl)carbamate;
1-{1-[4-(2-naphthyl)pyrimidin-2-yl]piperidin-4-yl}ethanamine;
(1R)-1-{1-[4-(2-naphthyl)pyrimidin-2-yl]piperidin-4-yl}ethanamine;
(1S)-1-{1-[4-(2-naphthyl)pyrimidin-2-yl]piperidin-4-yl}ethanamine;
4-{[4-(2-naphthyl)pyrimidin-2-yl]methyl}-1,4-diazepane-1-carbaldehyde;
1-(1-{[4-(2-naphthyl)pyrimidin-2-yl]methyl}piperidin-4-yl)methanamine;
1-[4-(2-naphthyl)pyrimidin-2-yl]piperidine-4-carbonitrile;
1-[4-(2-naphthyl)pyrimidin-2-yl]piperidine-4-carbothioamide;
2-azetidin-1-yl-4-(2-naphthyl)pyrimidine;
2-azetidin-1-yl-4-(2-naphthyl)pyrimidine;
2-[4-(azidomethyl)piperidin-1-yl]-4-(2-naphthyl)pyrimidine;
1-[4-(2-naphthyl)pyrimidin-2-yl]piperidine-4-carboximidamide;
methyl [(tert-butoxycarbonyl)amino]{1-[4-(2-naphthyl)pyrimidin-2-yl]piperidin-4-ylidene}acetate;
methyl [(tert-butoxycarbonyl)amino]{1-[4-(2-naphthyl)pyrimidin-2-yl]piperidin-4-yl}acetate;
methyl (2R)-[(tert-butoxycarbonyl)amino]{1-[4-(2-naphthyl)pyrimidin-2-yl]piperidin-4-yl}acetate;
methyl (2S)-[(tert-butoxycarbonyl)amino]{1-[4-(2-naphthyl)pyrimidin-2-yl]piperidin-4-yl}acetate;
methyl amino {1-[4-(2-naphthyl)pyrimidin-2-yl]piperidin-4-yl}acetate;
methyl (2R)-amino {1-[4-(2-naphthyl)pyrimidin-2-yl]piperidin-4-yl}acetate;
methyl (2S)-amino {1-[4-(2-naphthyl)pyrimidin-2-yl]piperidin-4-yl}acetate;
(2R)-2-amino-2-{1-[4-(2-naphthyl)pyrimidin-2-yl]piperidin-4-yl}ethanol;
tert-butyl ((1R)-2-hydroxy-1-{1-[4-(2-naphthyl)pyrimidin-2-yl]piperidin-4-yl}ethyl)carbamate;
tert-butyl ((1S)-2-hydroxy-1-{1-[4-(2-naphthyl)pyrimidin-2-yl]piperidin-4-yl}ethyl)carbamate;
tert-butyl ((1R)-2-hydroxy-2-methyl-1-{1-[4-(2-naphthyl)pyrimidin-2-yl]piperidin-4-yl}propyl)carbamate;
(1R)-1-amino-2-methyl-1-{1-[4-(2-naphthyl)pyrimidin-2-yl]piperidin-4yl}propan-2-ol;
(4R)-5,5-dimethyl-4-{1-[4-(2-naphthyl)pyrimidin-2-yl]piperidin-4-yl}-1,3-oxazolidin-2-one;
(4R)-4-{1-[4-(2-naphthyl)pyrimidin-2-yl]piperidin-4-yl}-1,3-oxazolidin-2-one; or
(1-(4-(naphthalen-2-yl)pyrimidin-2-yl)piperidin-4-yl)methanol.

32. The compound of claim 30, or a pharmaceutically acceptable salt thereof, wherein the compound is
N-[4-(2-naphthyl)pyrimidin-2-yl]azepan-4-amine;
tert-butyl 3-({[4-(2-naphthyl)pyrimidin-2-yl]amino }methyl)azetidine-1-carboxylate;
N-(azetidin-3-ylmethyl)-4-(2-naphthyl)pyrimidin-2-amine;
tert-butyl (3S)-3-({[4-(2-naphthyl)pyrimidin-2-yl]amino }methyl)pyrrolidine-1-carboxylate;
4-(2-naphthyl)-N-[(3R)-pyrrolidin-3-ylmethyl]pyrimidin-2-amine;
N-(2-morpholin-4-ylethyl)-4-(2-naphthyl)pyrimidin-2-amine;
N-(3-morpholin-4-ylpropyl)-4-(2-naphthyl)pyrimidin-2-amine;
4-(2-naphthyl)-N-(pyridin-3-ylmethyl)pyrimidin-2-amine;
4-(2-naphthyl)-N-(pyridin-4-ylmethyl)pyrimidin-2-amine;
4-(2-naphthyl)-N-(pyridin-2-ylmethyl)pyrimidin-2-amine;
4-({[4-(2-naphthyl)pyrimidin-2-yl]amino }methyl)benzenesulfonamide;
4-(2-naphthyl)-N-(2-pyridin-3-ylethyl)pyrimidin-2-amine;
4-(2-naphthyl)-N-(2-pyridin-4-ylethyl)pyrimidin-2-amine;
tert-butyl (3S)-3-{[4-(2-naphthyl)pyrimidin-2-yl]amino}pyrrolidine-1-carboxylate;
tert-butyl (3R)-3-{[4-(2-naphthyl)pyrimidin-2-yl]amino}pyrrolidine-1-carboxylate;
4-(2-naphthyl)-N-[(3S)-pyrrolidin-3-yl]pyrimidin-2-amine;
4-(2-naphthyl)-N-[(3R)-pyrrolidin-3-yl]pyrimidin-2-amine;
tert-butyl (3R)-3-({[4-(2-naphthyl)pyrimidin-2-yl]amino }methyl)pyrrolidine-1-carboxylate;
4-(2-naphthyl)-N-[(3S)-pyrrolidin-3-ylmethyl]pyrimidin-2-amine;
4-(2-naphthyl)-N-(piperidin-4-ylmethyl)pyrimidin-2-amine;
tert-butyl 4-({[4-(2-naphthyl)pyrimidin-2-yl]amino }methyl)piperidine-1-carboxylate;
trans-N-[4-(2-naphthyl)pyrimidin-2-yl]cyclohexane-1,4-diamine;
N-(4-methoxybenzyl)-4-(2-naphthyl)pyrimidin-2-amine;
N-[2-(4-methylphenyl)ethyl]-4-(2-naphthyl)pyrimidin-2-amine;
2-{methyl[4-(2-naphthyl)pyrimidin-2-yl]amino}ethanol;
N-(2-methoxyethyl)-4-(2-naphthyl)pyrimidin-2-amine;
2-{[4-(2-naphthyl)pyrimidin-2-yl]amino }ethanol;
N-(2-methoxyethyl)-N-methyl-4-(2-naphthyl)pyrimidin-2-amine;
N-methyl-4-(2-naphthyl)pyrimidin-2-amine;
N,N-diethyl-4-(2-naphthyl)pyrimidin-2-amine;
4-(2-naphthyl)-N-propylpyrimidin-2-amine;
N-butyl-4-(2-naphthyl)pyrimidin-2-amine;
N-isopropyl-4-(2-naphthyl)pyrimidin-2-amine;
N-(sec-butyl)-4-(2-naphthyl)pyrimidin-2-amine;
N-isobutyl-4-(2-naphthyl)pyrimidin-2-amine;
N-(tert-butyl)-4-(2-naphthyl)pyrimidin-2-amine;
N-benzyl-4-(2-naphthyl)pyrimidin-2-amine;
4-(2-naphthyl)-N-(2-phenylethyl)pyrimidin-2-amine;
N-cyclopentyl-4-(2-naphthyl)pyrimidin-2-amine;
N-cyclohexyl-4-(2-naphthyl)pyrimidin-2-amine;
tert-butyl 4-{[4-(2-naphthyl)pyrimidin-2-yl]amino}piperidine-1-carboxylate;
4-(2-naphthyl)-N-piperidin-4-ylpyrimidin-2-amine;
tert-butyl 4- {methyl[4-(2-naphthyl)pyrimidin-2-yl]amino}piperidine-1-carboxylate;
N-methyl-4-(2-naphthyl)-N-piperidin-4-ylpyrimidin-2-amine;
tert-butyl 4-(2-methoxy-1-{[4-(2-naphthyl)pyrimidin-2-yl]amino}-2-oxoethyl)piperidine-1-carboxylate;
4-{methyl[4-(2-naphthyl)pyrimidin-2-yl]amino}piperidine-1-carbaldehyde;
N-(1-benzylpiperidin-4-yl)-N-methyl-4-(2-naphthyl)pyrimidin-2-amine;
N-[(1-acetylazetidin-3-yl)methyl]-4-(2-naphthyl)pyrimidin-2-amine;
4-(2-naphthyl)-N-{[1-(trifluoroacetyl)azetidin-3-yl]methyl}pyrimidin-2-amine;
N-[(1-benzoylazetidin-3-yl)methyl]-4-(2-naphthyl)pyrimidin-2-amine;
3-({[4-(2-naphthyl)pyrimidin-2-yl]amino}methyl)azetidine-1-carboxamide;
N-ethyl-3-({[4-(2-naphthyl)pyrimidin-2-yl]amino}methyl)azetidine-1-carboxamide;
N-{[1-(methylsulfonyl)azetidin-3-yl]methyl}-4-(2-naphthyl)pyrimidin-2-amine;
N-({1-[(4-methylphenyl)sulfonyl]azetidin-3-yl}methyl)-4-(2-naphthyl)pyrimidin-2-amine;
N,N-dimethyl-3-({[4-(2-naphthyl)pyrimidin-2-yl]amino}methyl)azetidine-1-sulfonamide;
N-[(3S)-1-acetylpyrrolidin-3-yl]-4-(2-naphthyl)pyrimidin-2-amine;
N-[(3S)-1-benzoylpyrrolidin-3-yl]-4-(2-naphthyl)pyrimidin-2-amine;
methyl (3S)-3-{[4-(2-naphthyl)pyrimidin-2-yl]amino}pyrrolidine-1-carboxylate;
(3S)-N,N-dimethyl-3-{[4-(2-naphthyl)pyrimidin-2-yl]amino}pyrrolidine-1-carboxamide;
(3S)-N-ethyl-3-{[4-(2-naphthyl)pyrimidin-2-yl]amino}pyrrolidine-1-carboxamide;
(3S)-3-{[4-(2-naphthyl)pyrimidin-2-yl]amino}pyrrolidine-1-carboxamide;
(3S)-N,N-dimethyl-3-{[4-(2-naphthyl)pyrimidin-2-yl]amino}pyrrolidine-1-sulfonamide;
N-[(3S)-1-(methylsulfonyl)pyrrolidin-3-yl]-4-(2-naphthyl)pyrimidin-2-amine;
N-{(3S)-1-[(4-methylphenyl)sulfonyl]pyrrolidin-3-yl}-4-(2-naphthyl)pyrimidin-2-amine;
(3S)-3-{[4-(2-naphthyl)pyrimidin-2-yl]amino}pyrrolidine-1-carbaldehyde;
N-[(3S)-1-(morpholin-4-ylcarbonyl)pyrrolidin-3-yl]-4-(2-naphthyl)pyrimidin-2-amine;
ethyl (3S)-3-{[4-(2-naphthyl)pyrimidin-2-yl]amino}pyrrolidine-1-carboxylate;
N-[(3R)-1-acetylpyrrolidin-3-yl]-4-(2-naphthyl)pyrimidin-2-amine;
N-[(3R)-1-benzoylpyrrolidin-3-yl]-4-(2-naphthyl)pyrimidin-2-amine;
methyl (3R)-3-{[4-(2-naphthyl)pyrimidin-2-yl]amino}pyrrolidine-1-carboxylate;
(3R)-N,N-dimethyl-3-{[4-(2-naphthyl)pyrimidin-2-yl]amino}pyrrolidine-1-carboxamide;
(3R)-N-ethyl-3-{[4-(2-naphthyl)pyrimidin-2-yl]amino}pyrrolidine-1-carboxamide;
(3R)-3-{[4-(2-naphthyl)pyrimidin-2-yl]amino}pyrrolidine-1-carboxamide;
(3R)-N,N-dimethyl-3-{[4-(2-naphthyl)pyrimidin-2-yl]amino}pyrrolidine-1-sulfonamide;
N-[(3R)-1-(methylsulfonyl)pyrrolidin-3-yl]-4-(2-naphthyl)pyrimidin-2-amine;
N-{(3R)-1-[(4-methylphenyl)sulfonyl]pyrrolidin-3-yl}-4-(2-naphthyl)pyrimidin-2-amine;
(3R)-3-{[4-(2-naphthyl)pyrimidin-2-yl]amino}pyrrolidine-1-carbaldehyde;
N-[(3R)-1-(morpholin-4-ylcarbonyl)pyrrolidin-3-yl]-4-(2-naphthyl)pyrimidin-2-amine;
ethyl (3R)-3-{[4-(2-naphthyl)pyrimidin-2-yl]amino}pyrrolidine-1-carboxylate;
N-{[(3R)-1-acetylpyrrolidin-3-yl]methyl}-4-(2-naphthyl)pyrimidin-2-amine;
methyl (3R)-3-({[4-(2-naphthyl)pyrimidin-2-yl]amino}methyl)pyrrolidine-1-carboxylate;
(3R)-3-({[4-(2-naphthyl)pyrimidin-2-yl]amino}methyl)pyrrolidine-1-carbaldehyde;
N-{[(3S)-1-acetylpyrrolidin-3-yl]methyl}-4-(2-naphthyl)pyrimidin-2-amine;
methyl (3S)-3-({[4-(2-naphthyl)pyrimidin-2-yl]amino}methyl)pyrrolidine-1-carboxylate;
(3S)-3-({[4-(2-naphthyl)pyrimidin-2-yl]amino}methyl)pyrrolidine-1-carbaldehyde;
N-[(1-acetylpiperidin-4-yl)methyl]-4-(2-naphthyl)pyrimidin-2-amine;
N,N-dimethyl-4-({[4-(2-naphthyl)pyrimidin-2-yl]amino}methyl)piperidine-1-carboxamide;
N,N-diethyl-4-({[4-(2-naphthyl)pyrimidin-2-yl]amino}methyl)piperidine-1-carboxamide;
4-(2-naphthyl)-N-{[1-(pyrrolidin-1-ylcarbonyl)piperidin-4-yl]methyl}pyrimidin-2-amine;
N-{[1-(methylsulfonyl)piperidin-4-yl]methyl}-4-(2-naphthyl)pyrimidin-2-amine;
N,N-dimethyl-4-({[4-(2-naphthyl)pyrimidin-2-yl]amino}methyl)piperidine-1-sulfonamide;
4-({[4-(2-naphthyl)pyrimidin-2-yl]amino}methyl)piperidine-1-carboxamide;
N-ethyl-4-({[4-(2-naphthyl)pyrimidin-2-yl]amino}methyl)piperidine-1-carboxamide;
N-isopropyl-4-({[4-(2-naphthyl)pyrimidin-2-yl]amino}methyl)piperidine-1-carboxamide;
N-cyclohexyl-4-({[4-(2-naphthyl)pyrimidin-2-yl]amino}methyl)piperidine-1-carboxamide;
N-[(1-benzoylpiperidin-4-yl)methyl]-4-(2-naphthyl)pyrimidin-2-amine;
N-[(1-ethylpiperidin-4-yl)methyl]-4-(2-naphthyl)pyrimidin-2-amine;
N-[(1-benzylpiperidin-4-yl)methyl]-4-(2-naphthyl)pyrimidin-2-amine;
4-(2-naphthyl)-N-{[1-(phenylacetyl)piperidin-4-yl]methyl}pyrimidin-2-amine;
N-({1-[(4-methylphenyl)sulfonyl]piperidin-4-yl}methyl)-4-(2-naphthyl)pyrimidin-2-amine;
N-(trans-4-{[4-(2-naphthyl)pyrimidin-2-yl]amino}cyclohexyl)acetamide;
methyl (trans-4-{[4-(2-naphthyl)pyrimidin-2-yl]amino}cyclohexyl)carbamate;
N,N-dimethyl-N′-(trans-4-{[4-(2-naphthyl)pyrimidin-2-yl]amino}cyclohexyl)urea;
N-ethyl-N′-(trans-4-{[4-(2-naphthyl)pyrimidin-2-yl]amino}cyclohexyl)urea;
N,N-dimethyl-N′-(trans-4-{[4-(2-naphthyl)pyrimidin-2-yl]amino}cyclohexyl)sulfamide;
N-(trans-4-{[4-(2-naphthyl)pyrimidin-2-yl]amino}cyclohexyl)methanesulfonamide;
4-methyl-N-(trans-4-{[4-(2-naphthyl)pyrimidin-2-yl]amino}cyclohexyl)benzenesulfonamide;
5-(dimethylamino)-N-(trans-4-{[4-(2-naphthyl)pyrimidin-2-yl]amino}cyclohexyl)naphthalene-1-sulfonamide;
4-cyano-N-(3-{[4-(2-naphthyl)pyrimidin-2-yl]amino}cyclohexyl)benzamide;
N-(1-acetylpiperidin-4-yl)-4-(2-naphthyl)pyrimidin-2-amine;
N,N-dimethyl-4-{[4-(2-naphthyl)pyrimidin-2-yl]amino}piperidine-1-carboxamide;
N,N-diethyl-4-{[4-(2-naphthyl)pyrimidin-2-yl]amino}piperidine-1-carboxamide;
4-(2-naphthyl)-N-[1-(pyrrolidin-1-ylcarbonyl)piperidin-4-yl]pyrimidin-2-amine;
N-[1-(morpholin-4-ylcarbonyl)piperidin-4-yl]-4-(2-naphthyl)pyrimidin-2-amine;
N-[1-(methylsulfonyl)piperidin-4-yl]-4-(2-naphthyl)pyrimidin-2-amine;
N,N-dimethyl-4-{[4-(2-naphthyl)pyrimidin-2-yl]amino}piperidine-1-sulfonamide;
4-{[4-(2-naphthyl)pyrimidin-2-yl]amino}piperidine-1-carboxamide;
N-ethyl-4-{[4-(2-naphthyl)pyrimidin-2-yl]amino}piperidine-1-carboxamide;
N-isopropyl-4-{[4-(2-naphthyl)pyrimidin-2-yl]amino}piperidine-1-carboxamide;
N-cyclohexyl-4-{[4-(2-naphthyl)pyrimidin-2-yl]amino}piperidine-1-carboxamide;
N-(1-benzoylpiperidin-4-yl)-4-(2-naphthyl)pyrimidin-2-amine;
N-(1-acetylpiperidin-4-yl)-N-methyl-4-(2-naphthyl)pyrimidin-2-amine;
N-methyl-4-(2-naphthyl)-N-[1-(trifluoroacetyl)piperidin-4-yl]pyrimidin-2-amine;
N-(1-benzoylpiperidin-4-yl)-N-methyl-4-(2-naphthyl)pyrimidin-2-amine;
methyl 4-{methyl[4-(2-naphthyl)pyrimidin-2-yl]amino}piperidine-1-carboxylate;
N,N-dimethyl-4-{methyl[4-(2-naphthyl)pyrimidin-2-yl]amino}piperidine-1-carboxamide;
N-methyl-4-(2-naphthyl)-N-[1-(pyrrolidin-1-ylcarbonyl)piperidin-4-yl]pyrimidin-2-amine;
N,N-diethyl-4-{methyl[4-(2-naphthyl)pyrimidin-2-yl]amino}piperidine-1-carboxamide;
N-ethyl-4-{methyl[4-(2-naphthyl)pyrimidin-2-yl]amino}piperidine-1-carboxamide;
4-{methyl[4-(2-naphthyl)pyrimidin-2-yl]amino}piperidine-1-carboxamide;
N-methyl-N-[1-(methylsulfonyl)piperidin-4-yl]-4-(2-naphthyl)pyrimidin-2-amine;
N-methyl-N-{1-[(4-methylphenyl)sulfonyl]piperidin-4-yl}-4-(2-naphthyl)pyrimidin-2-amine;
N-methyl-4-(2-naphthyl)-N-(1-pyrimidin-2-ylpiperidin-4-yl)pyrimidin-2-amine;
N-methyl-4-(2-naphthyl)-N-(1-propylpiperidin-4-yl)pyrimidin-2-amine;
2-(4-{methyl[4-(2-naphthyl)pyrimidin-2-yl]amino}piperidin-1-yl)acetamide;
N-methyl-4-(2-naphthyl)-N-{1-[2-(trityloxy)ethyl]piperidin-4-yl}pyrimidin-2-amine;
4-{methyl[4-(2-naphthyl)pyrimidin-2-yl]amino}piperidine-1-carboximidamide;
tert-butyl (3-{[4-(2-naphthyl)pyrimidin-2-yl]amino}cyclohexyl)carbamate; or
4-(2-naphthyl)-2-[4-(pyrrolidin-1-ylmethyl)piperidin-1-yl]pyrimidine.

33. A compound of claim 1, or a pharmaceutically acceptable salt thereof, wherein the compound is:
1-{1-[2-(2-naphthyl)pyrimidin-4-yl]piperidin-4-yl}methanamine;
1-{1-[6-(2-naphthyl)pyrimidin-4-yl]piperidin-4-yl}methanamine;
1-{1-[5-(2-naphthyl)pyrimidin-2-yl]piperidin-4-yl}methanamine;
1-{1-[6-(2-naphthyl)pyrazin-2-yl]piperidin-4-yl}methanamine;
1-{1-[5-(2-naphthyl)pyridazin-3-yl]piperidin-4-yl}methanamine;
1-{1-[4-(2-naphthyl)pyrimidin-2-yl]-1,2,3,4-tetrahydroquinolin-4-yl}methanamine;
1-{(4S)-1-[4-(2-naphthyl)pyrimidin-2-yl]azepan-4-yl}methanamine;
1-{(4R) 1-[4-(2-naphthyl)pyrimidin-2-yl]azepan-4-yl}methanamine;
1-[4-(2-naphthyl)pyrimidin-2-yl]azepan-4-amine;
N-{1-[4-(2-naphthyl)pyrimidin-2-yl]azepan-4-yl}acetamide;
2,2,2-trifluoro-N-{1-[4-(2-naphthyl)pyrimidin-2-yl]azepan-4-yl}acetamide;
N,N-dimethyl-N′-{1-[4-(2-naphthyl)pyrimidin-2-yl]azepan-4-yl}urea;
N-{1-[4-(2-naphthyl)pyrimidin-2-yl]azepan-4-yl}benzamide;
N-{1-[4-(2-naphthyl)pyrimidin-2-yl]azepan-4-yl}urea;
N-ethyl-N′-{1-[4-(2-naphthyl)pyrimidin-2-yl]azepan-4-yl}urea;
N-{1-[4-(2-naphthyl)pyrimidin-2-yl]azepan-4-yl}methanesulfonamide;
4-methyl-N-{1-[4-(2-naphthyl)pyrimidin-2-yl]azepan-4-yl}benzenesulfonamide;
N,N-dimethyl-N′-{1-[4-(2-naphthyl)pyrimidin-2-yl]azepan-4-yl}sulfamide;
{(1S,4R)-2-[4-(2-naphthyl)pyrimidin-2-yl]-2-azabicyclo[2.2.1]heptane-5,5-diyl}dimethanamine;
1-{(1R,4R,5R)-2-[4-(2-naphthyl)pyrimidin-2-yl]-2-azabicyclo[2.2.1]hept-5-yl}methanamine;
1-{(1R,4R,5S)-2-[4-(2-naphthyl)pyrimidin-2-yl]-2-azabicyclo[2.2.1]hept-5-yl}methanamine;
1-{(1S,4S,5S)-2-[4-(2-naphthyl)pyrimidin-2-yl]-2-azabicyclo[2.2.1]hept-5-yl}methanamine;
1-{(1S,4S,5R)-2-[4-(2-naphthyl)pyrimidin-2-yl]-2-azabicyclo[2.2.1]hept-5-yl}methanamine;
1-{8-[4-(2-naphthyl)pyrimidin-2-yl]-8-azabicyclo[3.2.1]oct-3-yl}methanamine;
1-{8-[4-(2-naphthyl)pyrimidin-2-yl]-8-azabicyclo[3.2.1]oct-3-yl}methanamine;
2-[4-(2-naphthyl)pyrimidin-2-yl]-2, 8-diazaspiro[4.5]decane;
N,N,N′-trimethyl-N′-{(3R)-1-[4-(2-naphthyl)pyrimidin-2-yl]pyrrolidin-3-yl}ethane-1,2-diamine;
(3aR* ,6aS)-2-[4-(2-naphthyl)pyrimidin-2-yl]octahydropyrrolo[3,4-c]pyrrole;
5-(4-naphthalen-2-ylpyrimidin-2-yl)octahydropyrrolo [3,4-b]pyrrole;
1-({1-[4-(2-naphthyl)pyrimidin-2-yl]pyrrolidin-3-yl}methyl)piperidin-4-ol;
(3S)-1-({1-[4-(2-naphthyl)pyrimidin-2-yl]pyrrolidin-3-yl}methyl)piperidin-3-ol;
(3R)-1-({1-[4-(2-naphthyl)pyrimidin-2-yl]pyrrolidin-3-yl}methyl)piperidin-3-ol;
(3S)-1-({1-[4-(2-naphthyl)pyrimidin-2-yl]pyrrolidin-3-yl}methyl)pyrrolidin-3-ol;
(3R)-1-({1-[4-(2-naphthyl)pyrimidin-2-yl]pyrrolidin-3-yl}methyl)pyrrolidin-3-ol;
(3S,3′R)-1′-[4-(2-naphthyl)pyrimidin-2-yl]-1,3′-bipyrrolidin-3-ol;
2-{(3R)-1-[4-(2-naphthyl)pyrimidin-2-yl]pyrrolidin-3-yl}octahydro-2H-pyrido[1,2-a]pyrazine;
(3S,3′R)-1′-[4-(2-naphthyl)pyrimidin-2-yl]-1,3′-bipyrrolidin-3-amine;
3-(methyl {(3R)-1-[4-(2-naphthyl)pyrimidin-2-yl]pyrrolidin-3-yl}amino)propan-1-ol;
N,N-dimethyl-N′-{(3R)-1-[4-(2-naphthyl)pyrimidin-2-yl]pyrrolidin-3-yl}ethane-1,2-diamine;
N,N′-dimethyl-N-{(3R)-1-[4-(2-naphthyl)pyrimidin-2-yl]pyrrolidin-3-yl}ethane-1,2-diamine;
(3R,3′R)-1′-[4-(2-naphthyl)pyrimidin-2-yl]-1,3′-bipyrrolidin-3-ol;
(3R,3′R)-1′-[4-(2-naphthyl)pyrimidin-2-yl]-1,3′-bipyrrolidin-3-amine;
(1S,4S)-2-methyl-5-{(3R)-1-[4-(2-naphthyl)pyrimidin-2-yl]pyrrolidin-3-yl}-2,5-diazabicyclo[2.2.1]heptane;
5-{(3R)-1-[4-(2-naphthyl)pyrimidin-2-yl]pyrrolidin-3-yl}octahydropyrrolo[3,4b]-pyrrole;
N,N,N′-trimethyl-N′-{(3R)-1-[4-(2-naphthyl)pyrimidin-2-yl]pyrrolidin-3-yl}propane-1,3-diamine;
(3R,3′S)-1′-[4-(2-naphthyl)pyrimidin-2-yl]-1,3′-bipyrrolidin-3-ol;
(3S,3′S)-1′-[4-(2-naphthyl)pyrimidin-2-yl]-1,3′-bipyrrolidin-3-ol;
N,N-dimethyl-N′-{(3S)-1-[4-(2-naphthyl)pyrimidin-2-yl]pyrrolidin-3-yl}ethane-1,2-diamine;
N,N,N′-trimethyl-N′-{(3S)-1-[4-(2-naphthyl)pyrimidin-2-yl]pyrrolidin-3-yl}ethane-1,2-diamine
3-(methyl{(3S)-1-[4-(2-naphthyl)pyrimidin-2-yl]pyrrolidin-3-yl}amino)propan-1-ol;
5-{(3S)-1-[4-(2-naphthyl)pyrimidin-2-yl]pyrrolidin-3-yl}octahydropyrrolo[3,4-b]pyrrole;
N,N,N′-trimethyl-N′-{(3S)-1-[4-(2-naphthyl)pyrimidin-2-yl]pyrrolidin-3-yl}propane-1,3-diamine;
2-(ethyl{(3S)-1-[4-(2-naphthyl)pyrimidin-2-yl]pyrrolidin-3-yl}amino)ethanol;
2-[(3S)-3-(1H-imidazol-1-yl)pyrrolidin-1-yl]-4-(2-naphthyl)pyrimidine;
(3S)-N-cyclohexyl-N-methyl-1-[4-(2-naphthyl)pyrimidin-2-yl]pyrrolidin-3-amine;
(3S)-N-(tert-butyl)-1-[4-(2-naphthyl)pyrimidin-2-yl]pyrrolidin-3-amine;
4-(2-naphthyl)-2-[(3S)-3-piperazin-1-ylpyrrolidin-1-yl]pyrimidine;
4-{(3S)-1-[4-(2-naphthyl)pyrimidin-2-yl]pyrrolidin-3-yl}morpholine;
4-{(3S)-1-[4-(2-naphthyl)pyrimidin-2-yl]pyrrolidin-3-yl}piperazin-2-one;
1-{(3S)-1-[4-(2-naphthyl)pyrimidin-2-yl]pyrrolidin-3-yl}azepane;
(3′S)-1′-[4-(2-naphthyl)pyrimidin-2-yl]-1,3′-bipyrrolidine;
4-(2-naphthyl)-2-[(3S)-3-piperidin-1-ylpyrrolidin-1-yl]pyrimidine;
2-[(3S)-3-(4-methylpiperazin-1-yl)pyrrolidin-1-yl]-4-(2-naphthyl)pyrimidine;
(1-{(3S)-1-[4-(2-naphthyl)pyrimidin-2-yl]pyrrolidin-3-yl}piperidin-3-yl)methanol;
(1-{(3S)-1-[4-(2-naphthyl)pyrimidin-2-yl]pyrrolidin-3-yl}piperidin-4-yl)methanol;
1-{(3S)-1-[4-(2-naphthyl)pyrimidin-2-yl]pyrrolidin-3-yl}piperidin-3-ol;
(3S)-N-(2-morpholin-4-ylethyl)-1-[4-(2-naphthyl)pyrimidin-2-yl]pyrrolidin-3-amine;
1-{(3S)-1-[4-(2-naphthyl)pyrimidin-2-yl]pyrrolidin-3-yl}piperidin-4-ol;
(3R,3′S)-N,N-dimethyl-1′-[4-(2-naphthyl)pyrimidin-2-yl]-1,3′-bipyrrolidin-3-amine;
(3S,3′S)-N,N-dimethyl-1′-[4-(2-naphthyl)pyrimidin-2-yl]-1,3′-bipyrrolidin-3-amine;
(3R)-1-{(3S)-1-[4-(2-naphthyl)pyrimidin-2-yl]pyrrolidin-3-yl}piperidin-3-ol;
(3S)-1-{(3S)-1-[4-(2-naphthyl)pyrimidin-2-yl]pyrrolidin-3-yl}piperidin-3ol;
2-(ethyl{(3R)-1-[4-(2-naphthyl)pyrimidin-2-yl]pyrrolidin-3-yl}amino)ethanol;
2-[(3R)-3-(1H-imidazol-1-yl)pyrrolidin-1-yl]-4-(2-naphthyl)pyrimidine;
(3R)-N-cyclohexyl-N-methyl-1-[4-(2-naphthyl)pyrimidin-2-yl]pyrrolidin-3-amine;
(3R)-N-(tert-butyl)-1-[4-(2-naphthyl)pyrimidin-2-yl]pyrrolidin-3-amine;
4-(2-naphthyl)-2-[(3R)-3-piperazin-1-ylpyrrolidin-1-yl]pyrimidine;
4-{(3R)-1-[4-(2-naphthyl)pyrimidin-2-yl]pyrrolidin-3-yl}morpholine;
4-{(3R)-1-[4-(2-naphthyl)pyrimidin-2-yl]pyrrolidin-3-yl}piperazin-2-one;
1-{(3R)-1-[4-(2-naphthyl)pyrimidin-2-yl]pyrrolidin-3-yl}azepane;
(3′R)-1′-[4-(2-naphthyl)pyrimidin-2-yl]-1,3′-bipyrrolidine;
4-(2-naphthyl)-2-[(3R)-3-piperidin-1-ylpyrrolidin-1-yl]pyrimidine;
2-[(3R)-3-(4-methylpiperazin-1-yl)pyrrolidin-1-yl]-4-(2-naphthyl)pyrimidine;
(1-{(3R)-1-[4-(2-naphthyl)pyrimidin-2-yl]pyrrolidin-3-yl}piperidin-3-yl)methanol;
(1-{(3R)-1-[4-(2-naphthyl)pyrimidin-2-yl]pyrrolidin-3-yl}piperidin-4-yl)methanol;
1-{(3R)-1-[4-(2-naphthyl)pyrimidin-2-yl]pyrrolidin-3-yl}piperidin-3-ol;
(3R)-N-(2-morpholin-4-ylethyl)-1-[4-(2-naphthyl)pyrimidin-2-yl]pyrrolidin-3-amine;
1-{(3R)-1-[4-(2-naphthyl)pyrimidin-2-yl]pyrrolidin-3-yl}piperidin-4-ol;
(3R,3′R)-N,N-dimethyl-1′-[4-(2-naphthyl)pyrimidin-2-yl]-1,3′-bipyrrolidin-3-amine;
(3S,3′R)-N,N-dimethyl-1′-[4-(2-naphthyl)pyrimidin-2-yl]-1,3′-bipyrrolidin-3-amine;
(3R)-1-{(3R)-1-[4-(2-naphthyl)pyrimidin-2-yl]pyrrolidin-3-yl}piperidin-3-ol;
(3S)-1-{(3R)-1-[4-(2-naphthyl)pyrimidin-2-yl]pyrrolidin-3-yl}piperidin-3-ol;
{1-[4-(2-naphthyl)pyrimidin-2-yl]pyrrolidin-3-yl}methanol;
1-({1-[4-(2-naphthyl)pyrimidin-2-yl]pyrrolidin-3-yl}methyl)azepane;
4-(2-naphthyl)-2-[3-(piperidin-1-ylmethyl)pyrrolidin-1-yl]pyrimidine;
4-(2-naphthyl)-2-[3-(pyrrolidin-1-ylmethyl)pyrrolidin-1-yl]pyrimidine;
2-[3-(azetidin-1-ylmethyl)pyrrolidin-1-yl]-4-(2-naphthyl)pyrimidine;
4-({1-[4-(2-naphthyl)pyrimidin-2-yl]pyrrolidin-3-yl}methyl)morpholine;
4-({1-[4-(2-naphthyl)pyrimidin-2-yl]pyrrolidin-3-yl}methyl)thiomorpholine;
N-ethyl-N-({1-[4-(2-naphthyl)pyrimidin-2-yl]pyrrolidin-3-yl}methyl)ethanamine;
2-methyl-N-({1-[4-(2-naphthyl)pyrimidin-2-yl]pyrrolidin-3-yl}methyl)propan-2-amine;
2-[ethyl({1-[4-(2-naphthyl)pyrimidin-2-yl]pyrrolidin-3-yl}methyl) amino]ethanol;
2-{3-[(4-methylpiperazin-1-yl)methyl]pyrrolidin-1-yl}-4-(2-naphthyl)pyrimidine;
4-({1-[4-(2-naphthyl)pyrimidin-2-yl]pyrrolidin-3-yl}methyl)piperazin-2-one;
2-[3-(1H-imidazol-1-ylmethyl)pyrrolidin-1-yl]-4-(2-naphthyl)pyrimidine;
2-[3-(chloromethyl)pyrrolidin-1-yl]-4-(2-naphthyl)pyrimidine;
N,N-dimethyl-1-{(3S)-1-[4-(2-naphthyl)pyrimidin-2-yl]pyrrolidin-3-yl}methanamine;
4-(2-naphthyl)-2-[(3S)-3-(piperidin-1-ylmethyl)pyrrolidin-1-yl]pyrimidine;
4-(2-naphthyl)-2-[(3S)-3-(pyrrolidin-1-ylmethyl)pyrrolidin-1-yl]pyrimidine;
N-methyl-1-{(3S)-1-[4-(2-naphthyl)pyrimidin-2-yl]pyrrolidin-3-yl}methanamine;
N-methyl-N-({(3S)-1-[4-(2-naphthyl)pyrimidin-2-yl]pyrrolidin-3-yl}methyl)cyclohexanamine;
1-({(3R)-1-[4-(2-naphthyl)pyrimidin-2-yl]pyrrolidin-3-yl}methyl)piperidin-2-one;
tert-butyl 4-({(3S)-1-[4-(2-naphthyl)pyrimidin-2-yl]pyrrolidin-3-yl}methyl)piperazine-1-carboxylate;
2-methyl-N-({(3S)-1-[4-(2-naphthyl)pyrimidin-2-yl]pyrrolidin-3-yl}methyl)propan-2-amine;
2-[ethyl({(3S)-1-[4-(2-naphthyl)pyrimidin-2-yl]pyrrolidin-3-yl}methyl)amino]ethanol;
2-{(3S)-3-[(4-methylpiperazin-1-yl)methyl]pyrrolidin-1-yl}-4-(2-naphthyl)pyrimidine;
(3S)-N,N-dimethyl-1-({(3S)-1-[4-(2-naphthyl)pyrimidin-2-yl]pyrrolidin-3-yl}methyl)pyrrolidin-3-amine;
2-[(3R)-3-(1H-imidazol-1-ylmethyl)pyrrolidin-1-yl]-4-(2-naphthyl)pyrimidine;
(3R)-N,N-dimethyl-1-({(3S)-1-[4-(2-naphthyl)pyrimidin-2-yl]pyrrolidin-3-yl}methyl)pyrrolidin-3-amine;
N,N-dimethyl-1-{(3R)-1-[4-(2-naphthyl)pyrimidin-2-yl]pyrrolidin-3-yl}methanamine;
4-(2-naphthyl)-2-[(3R)-3-(piperidin-1-ylmethyl)pyrrolidin-1-yl]pyrimidine;
4-(2-naphthyl)-2-[(3R)-3-(pyrrolidin-1-ylmethyl)pyrrolidin-1-yl]pyrimidine;
N-methyl-1-{(3R)-1-[4-(2-naphthyl)pyrimidin-2-yl]pyrrolidin-3-yl}methanamine;
N-methyl-N-({(3R)-1-[4-(2-naphthyl)pyrimidin-2-yl]pyrrolidin-3-yl}methyl)cyclohexanamine;
1-({(3S)-1-[4-(2-naphthyl)pyrimidin-2-yl]pyrrolidin-3-yl}methyl)piperidin-2-one;
tert-butyl 4-({(3R)-1-[4-(2-naphthyl)pyrimidin-2-yl]pyrrolidin-3-yl}methyl)piperazine-1-carboxylate;
2-methyl-N-({(3R)-1-[4-(2-naphthyl)pyrimidin-2-yl]pyrrolidin-3-yl}methyl)propan-2-amine;
2-[ethyl({(3R)-1-[4-(2-naphthyl)pyrimidin-2-yl]pyrrolidin-3-yl}methyl)amino]ethanol;
2-{(3R)-3-[(4-methylpiperazin-1-yl)methyl]pyrrolidin-1-yl}-4-(2-naphthyl)pyrimidine;
(3S)-N,N-dimethyl-1-({(3R)-1-[4-(2-naphthyl)pyrimidin-2-yl]pyrrolidin-3-yl}methyl)pyrrolidin-3-amine;
2-[(3S)-3-(1H-imidazol-1-ylmethyl)pyrrolidin-1-yl]-4-(2-naphthyl)pyrimidine;
(3R)-N,N-dimethyl-1-({(3R)-1-[4-(2-naphthyl)pyrimidin-2-yl]pyrrolidin-3-yl}methyl)pyrrolidin-3-amine;
N,N-dimethyl-1-[4-(2-naphthyl)pyrimidin-2-yl]-L-prolinamide;
4-(2-naphthyl)-2-[(2S)-2-(piperidin-1-ylcarbonyl)pyrrolidin-1-yl]pyrimidine;
4-(2-naphthyl)-2-[(2S)-2-(pyrrolidin-1-ylcarbonyl)pyrrolidin-1-yl]pyrimidine;
N-tert-butyl-1-[4-(2-naphthyl)pyrimidin-2-yl]-L-prolinamide;
N-ethyl-N-(2-hydroxyethyl)-1-[4-(2-naphthyl)pyrimidin-2-yl]-L-prolinamide;
2-{(2S)-2-[(4-methylpiperazin-1-yl)carbonyl]pyrrolidin-1-yl}-4-(2-naphthyl)pyrimidine;
(3S)-N,N-dimethyl-1-{1-[4-(2-naphthyl)pyrimidin-2-yl]-L-prolyl}pyrrolidin-3-amine;
(3R)-N,N-dimethyl-1-{1-[4-(2-naphthyl)pyrimidin-2-yl]-L-prolyl}pyrrolidin-3-amine;
N,N-dimethyl-1-[4-(2-naphthyl)pyrimidin-2-yl]-D-prolinamide;
4-(2-naphthyl)-2-[(2R)-2-(piperidin-1-ylcarbonyl)pyrrolidin-1-yl]pyrimidine;
4-(2-naphthyl)-2-[(2R)-2-(pyrrolidin-1-ylcarbonyl)pyrrolidin-1-yl]pyrimidine;
N-methyl-1-[4-(2-naphthyl)pyrimidin-2-yl]-D-prolinamide;
N-tert-butyl-1-[4-(2-naphthyl)pyrimidin-2-yl]-D-prolinamide;
2-{(2R)-2-[(4-methylpiperazin-1-yl)carbonyl]pyrrolidin-1-yl}-4-(2-naphthyl)pyrimidine;
(3S)-N,N-dimethyl-1-{1-[4-(2-naphthyl)pyrimidin-2-yl]-D-prolyl}pyrrolidin-3-amine;
(3R)-N,N-dimethyl-1-{1-[4-(2-naphthyl)pyrimidin-2-yl]-D-prolyl}pyrrolidin-3-amine;
(3′S)-1′-[4-(2-naphthyl)pyrimidin-2-yl]-1,3′-bipyrrolidin-2-one;
(3′R)-1′-[4-(2-naphthyl)pyrimidin-2-yl]-1,3′-bipyrrolidin-2-one;
tert-butyl {(3S)-1-[4-(2-naphthyl)pyrimidin-2-yl]pyrrolidin-3-yl}carbamate;
2-[(3R)-3-methoxypyrrolidin-1-yl]-4-(2-naphthyl)pyrimidine;
2-[(3S)-3-methoxypyrrolidin-1-yl]-4-(2-naphthyl)pyrimidine;
2,2,2-trifluoro-N-(2-{(2S)-1-[4-(2-naphthyl)pyrimidin-2-yl]pyrrolidin-2-yl}ethyl)acetamide;
2,2,2-trifluoro-N-(2-{(2R)-1-[4-(2-naphthyl)pyrimidin-2-yl]pyrrolidin-2-yl}ethyl)acetamide;
(3R)-N-methyl-1-[4-(2-naphthyl)pyrimidin-2-yl]pyrrolidin-3-amine;
(3S)-N-methyl-1-[4-(2-naphthyl)pyrimidin-2-yl]pyrrolidin-3-amine;
(3R)-N,N-dimethyl-1-[4-(2-naphthyl)pyrimidin-2-yl]pyrrolidin-3-amine;
(3S)-1-[4-(2-naphthyl)pyrimidin-2-yl]-N-(2,2,2-trifluoroethyl)pyrrolidin-3-amine;
N-{(3S)-1-[4-(2-naphthyl)pyrimidin-2-yl]pyrrolidin-3-yl}-N-(2,2,2-trifluoroethyl)acetamide;
2-{(2R)-1-[4-(2-naphthyl)pyrimidin-2-yl]pyrrolidin-2-yl}ethanamine;
2-{(2S)-1-[4-(2-naphthyl)pyrimidin-2-yl]pyrrolidin-2-yl}ethanamine;
2-{1-[4-(2-naphthyl)pyrimidin-2-yl]pyrrolidin-3-yl}ethanamine;
N-methyl-N-{(3R)-1-[4-(2-naphthyl)pyrimidin-2-yl]pyrrolidin-3-yl}acetamide;
N-ethyl-N,N′-dimethyl-N′-{(3R)-1-[4-(2-naphthyl)pyrimidin-2-yl]pyrrolidin-3-yl}ethane-1,2-diamine;
N,N′-dimethyl-N-{(3R)-1-[4-(2-naphthyl)pyrimidin-2-yl]pyrrolidin-3-yl}-N′-propylethane-1,2-diamine;
N-isopropyl-N,N′-dimethyl-N′-{(3R)-1-[4-(2-naphthyl)pyrimidin-2-yl]pyrrolidin-3-yl}ethane-1,2-diamine;
N-benzyl-N,N′-dimethyl-N′-{(3R)-1-[4-(2-naphthyl)pyrimidin-2-yl]pyrrolidin-3-yl}ethane-1,2-diamine;
N-methyl-N-[2-(methyl{(3R)-1-[4-(2-naphthyl)pyrimidin-2-yl]pyrrolidin-3-yl}amino)ethyl]acetamide;
2,2,2-trifluoro-N-methyl-N-[2-(methyl{(3R)-1-[4-(2-naphthyl)pyrimidin-2-yl]pyrrolidin-3-yl}amino)ethyl]acetamide;
N-methyl-N-[2-(methyl{(3R)-1-[4-(2-naphthyl)pyrimidin-2-yl]pyrrolidin-3-yl}amino)ethyl]propanamide;
N,2-dimethyl-N-[2-(methyl{(3R)-1-[4-(2-naphthyl)pyrimidin-2-yl]pyrrolidin-3-yl}amino)ethyl]propanamide;
N-methyl-N-[2-(methyl{(3R)-1-[4-(2-naphthyl)pyrimidin-2-yl]pyrrolidin-3-yl}amino)ethyl]methanesulfonamide;
b 1,1-diethyl-3-methyl-3-[2-(methyl{(3R)-1-[4-(2-naphthyl)pyrimidin-2-yl]pyrrolidine-3-yl}amino)ethyl]urea;
methyl methyl[2-(methyl{(3R)-1-[4-(2-naphthyl)pyrimidin-2-yl]pyrrolidin-3-yl}amino)ethyl]carbamate;
N-methyl-N-[2-(methyl{(3R)-1-[4-(2-naphthyl)pyrimidin-2-yl]pyrrolidin-3-yl}amino)ethyl]ethanesulfonamide;
N,N,N′-trimethyl-N′-[2-(methyl{(3R)-1-[4-(2-naphthyl)pyrimidin-2-yl]pyrrolidin-3-yl}amino)ethyl]sulfamide;
N-methyl-N-[2-(methyl{(3R)-1-[4-(2-naphthyl)pyrimidin-2-yl]pyrrolidin-3-yl}amino)ethyl]benzamide;
N-methyl-N-[2-(methyl{(3R)-1-[4-(2-naphthyl)pyrimidin-2-yl]pyrrolidin-3-yl}amino)ethyl]benzenesulfonamide;
tert-butyl ({1-[4-(2-naphthyl)pyrimidin-2-yl]azetidin-3-yl}methyl)carbamate;
1-{1-[4-(2-naphthyl)pyrimidin-2-yl]azetidin-3-yl}methanamine;
methyl ({1-[4-(2-naphthyl)pyrimidin-2-yl]azetidin-3-yl}methyl)carbamate;
2,2,2-trifluoro-N-({1-[4-(2-naphthyl)pyrimidin-2-yl]azetidin-3-yl}methyl)acetamide;
N,N-dimethyl-N′-({1-[4-(2-naphthyl)pyrimidin-2-yl]azetidin-3-yl}methyl)urea;
N-({1-[4-(2-naphthyl)pyrimidin-2-yl]azetidin-3-yl}methyl)urea;
N-ethyl-N′-({1-[4-(2-naphthyl)pyrimidin-2-yl]azetidin-3-yl}methyl)urea;
N-({1-[4-(2-naphthyl)pyrimidin-2-yl]azetidin-3-yl}methyl)methanesulfonamide;
4-methyl-N-({1-[4-(2-naphthyl)pyrimidin-2-yl]azetidin-3-yl}methyl)benzenesulfonamide;
tert-butyl {2-[1-(4-naphthalen-2-ylpyrimidin-2-yl)azetidin-3-yl]ethyl}carbamate;
2-[1-(4-naphthalen-2-ylpyrimidin-2-yl)azetidin-3-yl]ethanamine;
tert-butyl [(trans-4-{[4-(2-naphthyl)pyrimidin-2-yl]oxy}cyclohexyl)methyl]carbamate;
1-(trans-4-{[4-(2-naphthyl)pyrimidin-2-yl]oxy}cyclohexyl)methanamine;
tert-butyl ({cis-4-[(4-naphthalen-2-ylpyrimidin-2-yl)oxy]cyclohexyl}methyl)carbamate;
1-{cis-4-[(4-naphthalen-2-ylpyrimidin-2-yl)oxy]cyclohexyl}methanamine;
N-[trans-4-(aminomethyl)cyclohexyl]-N-methyl-4-(2-naphthyl)pyrimidin-2-amine;
benzyl [(cis-4-{methyl[4-(2-naphthyl)pyrimidin-2-yl]amino}cyclohexyl)methyl]carbamate;
N-[cis-4-(aminomethyl)cyclohexyl]-N-methyl-4-(2-naphthyl)pyrimidin-2-amine;
1-{4-[4-(2-naphthyl)pyrimidin-2-yl]phenyl}methanamine;
1-[trans-4-(4-naphthalen-2-ylpyrimidin-2-yl)cyclohexyl]methanamine;
1-{cis-4-[4-(2-naphthyl)pyrimidin-2-yl]cyclohexyl}methanamine;
1-{1-[4-(6-methoxy-2-naphthyl)pyrimidin-2-yl]piperidin-4-yl}methanamine;
1-{1-[4-(6-propoxy-2-naphthyl)pyrimidin-2-yl]piperidin-4-yl}methanamine;
1-{1-[4-(6-isobutoxy-2-naphthyl)pyrimidin-2-yl]piperidin-4-yl}methanamine;
6-{2-[4-(aminomethyl)piperidin-1-yl]pyrimidin-4-yl}-2-naphthyl acetate;
6-{2-[4-(aminomethyl)piperidin-1-yl]pyrimidin-4-yl}-2-naphthol;
2,2,2-trifluoro-N-({1-[4-(6-hydroxy-2-naphthyl)pyrimidin-2-yl]piperidin-4-yl}methyl)acetamide;
2,2,2-trifluoro-N-({1-[4-(6-methoxy-2-naphthyl)pyrimidin-2-yl]piperidin-4-yl}methyl)acetamide;
1-(1-{4-[6-(2-thienyl)-2-naphthyl]pyrimidin-2-yl}piperidin-4-yl)methanamine;
1-(1-{4-[6-(2-methoxyphenyl)-2-naphthyl]pyrimidin-2-yl}piperidin-4-yl)methanamine;
1-(1-{4-[6-(4-methoxyphenyl)-2-naphthyl]pyrimidin-2-yl}piperidin-4-yl)methanamine;
1-{1-[4-(6-phenyl-2-naphthyl)pyrimidin-2-yl]piperidin-4-yl}methanamine;
2,2,2-trifluoro-N-({1-[4-(6-formyl-2-naphthyl)pyrimidin-2-yl]piperidin-4-yl}methyl)acetamide;
1-{1-[4-(6-vinyl-2-naphthyl)pyrimidin-2-yl]piperidin-4-yl}methanamine;
1-{1-[4-(6-methyl-2-naphthyl)pyrimidin-2-yl]piperidin-4-yl}methanamine;
1-(1-{4-[6-(piperidin-1-ylmethyl)-2-naphthyl]pyrimidin-2-yl}piperidin-4-yl)methanamine;
1-(1-{4-[6-(morpholin-4-ylmethyl)-2-naphthyl]pyrimidin-2-yl}piperidin-4-yl)methanamine;
2-{4-[(4-methylpiperazin-1-yl)methyl]piperidin-1-yl}-4-(2-naphthyl)pyrimidine;
N-methyl-N-({1-[4-(2-naphthyl)pyrimidin-2-yl]piperidin-4-yl}methyl)cyclohexanamine;
1-({1-[4-(2-naphthyl)pyrimidin-2-yl]piperidin-4-yl}methyl)azepane;
2-[ethyl({1-[4-(2-naphthyl)pyrimidin-2-yl]piperidin-4-yl}methyl)amino]ethanol;
2-methyl-N-({1-[4-(2-naphthyl)pyrimidin-2-yl]piperidin-4-yl}methyl)propan-2-amine;
1-({1-[4-(2-naphthyl)pyrimidin-2-yl]piperidin-4-yl}methyl)pyrrolidin-2-one;
1-({1-[4-(2-naphthyl)pyrimidin-2-yl]piperidin-4-yl}methyl)piperidin-2-one;
4-(2-naphthyl)-2-[4-(piperazin-1-ylmethyl)piperidin-1-yl]pyrimidine;
4-({1-[4-(2-naphthyl)pyrimidin-2-yl]piperidin-4-yl}methyl)morpholine;
4-({1-[4-(2-naphthyl)pyrimidin-2-yl]piperidin-4-yl}methyl)piperazin-2-one;
1-(1-{[4-(2-naphthyl)pyrimidin-2-yl]carbonyl}piperidin-4-yl)methanamine; or
(3aR,7aS)-5-[4-(2-naphthyl)pyrimidin-2-yl]octahydro-1H-pyrrolo[3,4-c]pyridine.

34. A compound of claim 1, or a pharmaceutically acceptable salt thereof, wherein the compound is N,N,N′-trimethyl-N′-{(3R)-1-[4-(2-naphthyl)pyrimidin-2-yl]pyrrolidin-3-yl}ethane-1,2-diamine.

35. A compound of claim 1, or a pharmaceutically acceptable salt thereof, wherein the compound is ({1-[4-(2-naphthyl)pyrimidin-2-yl]piperidin-4-yl}methyl)amine.

36. A composition comprising the compound or pharmaceutically acceptable salt of the compound of claim 1 or 30 and a pharmaceutically acceptable carrier.

37. The composition of claim 36, wherein the pharmaceutically acceptable carrier is suitable for oral administration and the composition comprises an oral dosage form.

38. A method of treating a canonical Wnt-β-catenin cellular messaging system related disorder, comprising administering to a mammal in need thereof a compound or a pharmaceutically acceptable salt thereof the compound of claim 1 or 30 in an amount effect to treat a canonical Wnt-β-catenin cellular messaging system related disorder.

39. The method of claim 38, wherein the canonical Wnt-β-catenin cellular messaging system related disorder is selected from the group consisting of bone disorders, cancer, and Alzheimer's disease.

40. The method of claim 39, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

41. The method of claim 39, wherein the bone disorder is selected from the group consisting of osteoarthritis, osteolysis from multiple myeloma, osteoporosis, and rheumatoid arthritis.

42. The method of claim 38, wherein the mammal is human.

43. A method of synthesizing a compound of claim 9 or 30, comprising: reacting a compound of the Formula 4: 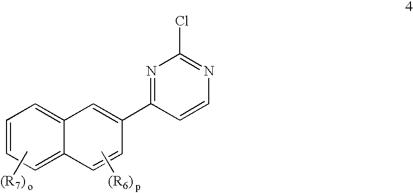
wherein
R₆ and R₇ are independently H, halogen, CN, NO₂, R₁₁, OR₁₁, S(O)ₓR₁₁, or NR₁₁R₁₂;
R₁₁ is H, C₁₋C₆ alkyl, aryl, or alkylaryl;
R₁₂ is H, C₁₋C₆ alkyl, aryl, alkylaryl, COR₁₃, CO₂R₁₃, CONR₁₃R₁₄, SO₂R₁₃; or R₁₁ and R₁₂ when taken together with the N to which they are attached form a C₃-C₈ monocyclic cycloalkyl, a 3- to 7-membered monocyclic heterocycle, an C₈-C₁₂ bicyclic cycloalkyl, or an 8- to 12-membered bicyclic heterocycle, all optionally substituted with R₁₁ and OR₁₁;
R₁₃ is H or C₁₋C₆ alkyl;
R₁₄ is H or C₁₋C₆ alkyl; or R₁₃ and R₁₄ when taken together with the N to which they are attached form a C₃-C₈ monocyclic cycloalkyl or a 3- to 7-membered monocyclic heterocycle;
o and p are independently 0, 1 or 2;
x is 0, 1, or 2;
with a compound of formula:
HX
wherein X is a primary alkyl or aryl amine, a secondary amine, a cyclicamine, an O(alkyl), an O(aryl), an S(alkyl), or an S(aryl);
under conditions effective to substitute Cl with the X of formula HX thereby providing a compound having the Formula 5: 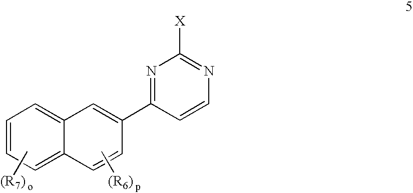
or pharmaceutically acceptable salts thereof.

44. A process for preparing a compound of Formula (a) 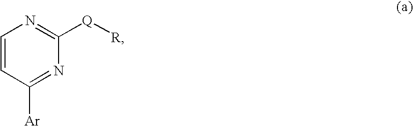
comprising:
reacting a compound of Formula (b) 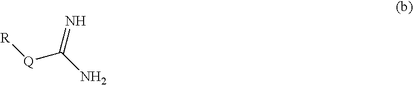
with a compound of Formula (c) 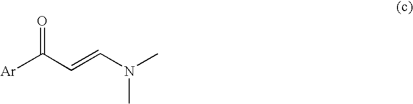
in the presence of a base
to provide a compound of Formula (a),
wherein Ar is 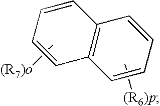
Q is N(CH₂)ᵣR₈ or CR₈R₉;
R is 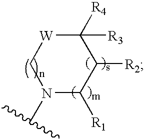
wherein U, R₁, R₂, R₃, R₄, W, R₆, R₇, R₈, R₉, m, n, o, p and s are as defined in claim 1.

45. The process of claim 44, the process further comprising preparing a compound of Formula (c) 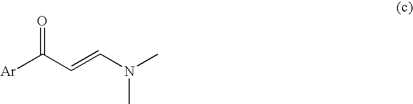
by reacting a compound of Formula (d) 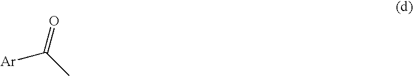
with DMF in the presence of DMA, to form a compound of Formula (c).

46. The process of claim 44, wherein the base is EtONa.
